(19) United States
(12) Patent Application Publication
(10) Pub. No.: US 2026/0166087 A1
(43) Pub. Date: Jun. 18, 2026

(54) COMPOSITIONS AND METHODS FOR TREATING ACUTE RESPIRATORY DISTRESS SYNDROME

(71) Applicant: Direct Biologics, LLC, Austin, TX (US)

(72) Inventor: Timothy Alexander Moseley, Fallbrook, CA (US)

(21) Appl. No.: 19/370,240

(22) Filed: Oct. 27, 2025

Related U.S. Application Data
(60) Provisional application No. 63/716,426, filed on Nov. 5, 2024.

(51) Int. Cl. A61K 35/28; A61K 9/50; A61K 38/17; A61K 38/18; A61K 38/30; A61K 38/45; A61K 38/48; A61P 11/00

(52) U.S. Cl. CPC A61K 35/28; A61K 9/5068; A61K 38/17; A61K 38/1709; A61K 38/177; A61K 38/179; A61K 38/1793; A61K 38/1841; A61K 38/185; A61K 38/30; A61K 38/45; A61K 38/4873; A61P 11/00; C12Y 208/02023; C12Y 304/22001

(57) ABSTRACT

Compositions and methods for treating ARDS are provided, including compositions comprising one or more proteins and one or more extracellular vesicles.

### CROSS REFERENCE

This application claims the benefit of U.S. Provisional Application No. 63/712,117 filed on Oct. 25, 2024, and U.S. Provisional Application No. 63/716,426 filed on Nov. 5, 2024, the entirety of which is hereby incorporated by reference herein.

### BACKGROUND

Acute respiratory distress syndrome (ARDS) is a common cause of life-threatening respiratory failure in critically ill patients defined by the acute onset of noncardiogenic pulmonary oedema, hypoxemia and bilateral opacities on chest imaging. ARDS is present in approximately 10% of all patients in intensive care units worldwide. Despite recent improvements in treatment, mortality remains high at 30-40% in most studies.

### SUMMARY

Provided herein are compositions comprising extracellular vesicles and/or one or more proteins. The compositions may be used for treatment of ARDS.

In one aspect, provided herein are methods of treating ARDS in a subject in need thereof, the method comprising administering to the subject a composition, wherein the ARDS is caused by a pulmonary insult or non-pulmonary insult, wherein the nonpulmonary insult is a blood transfusion, trauma, pancreatitis, drug reaction, burn, cardiopulmonary bypass, or noncardiogenic shock. In some embodiments, the ARDS is caused by the pulmonary insult. In some embodiments, wherein the pulmonary insult is aspiration, smoking, ventilation, lung contusion from trauma, thoracic surgery, drowning, pulmonary vasculitis, or fat embolism. In some embodiments, the administering occurs within 7 days, or within 7-14 days of the pulmonary or nonpulmonary insult. In some embodiments, the subject is not infected with COVID-19. In some embodiments, the subject does not have Long COVID-19 (Post Acute Sequalae of COVID-19, PASC), optionally wherein the subject does not have signs, symptoms, and conditions that continue or develop after acute COVID-19 infection. In some embodiments, the subject is not diagnosed with an infection within 1 week of the administering. In some embodiments, the subject is not suffering from an infection. In some embodiments, the subject has influenza. In some embodiments, the ARDS is not caused by bacterial pneumonia, viral pneumonia, fungal pneumonia, or parasitic pneumonia. In some embodiments, the subject has met the Berlin criteria for moderate to severe ARDS.

In some embodiments, the composition is administered intravenously. In some embodiments, the administering comprises infusion over 60 minutes on a first day. In some embodiments, the administering further comprises infusion of the composition on a second day. In some embodiments, the second day is one day after the first day, two days after the first day, or three days after the first day. In some embodiments, the infusion on the second day occurs if the subject does not have a SpO2 of at least about 93% between the administering and the infusion on the second day. In some embodiments, the administering comprises infusion of the composition on a third day. In some embodiments, the third day is two days after the second day.

In some embodiments, within 28 days after the administering, the subject does not undergo ventilator treatment for at least about 1, 2, 3, 4, 5, 6, or 7 days. In some embodiments, within 28 days after the administering, the subject is not in the intensive care unit for at least about 1, 2, 3, 4, 5, 6, or 7 days. In some embodiments, within 28 days after the administering, the subject is not treated with oxygen for at least about 1, 2, 3, 4, 5, 6, or 7 days.

In some embodiments, after the administering, the subject experiences improved tissue oxygenation. In some embodiments, after the administering, the subject experiences improved end-organ functioning. In some embodiments, after the administering, the subject has an improvement in partial pressure of arterial oxygen to fraction of inspired oxygen (PaO2/FiO2) ratio as compared to prior to the administering, optionally as measured from arterial blood gas or imputed from SpO2 daily. In some embodiments, after the administering, the subject has an oxygenation saturation of at least about 93% on room air. In some embodiments, after the administering, a biomarker in the subject is lower than prior to the administering. In some embodiments, the biomarker is measured from the blood of the subject. In some embodiments, after the administering the level of C-reactive protein (CRP) in the subject is less than prior to the administering. In some embodiments, after the administering the level of plasminogen activator inhibitor-1 (PAI-1) in the subject is less than prior to the administering. In some embodiments, after the administering the level of interleukin-8 (IL-8) in the subject is less than prior to the administering. In some embodiments, after the administering the level of interleukin-6 (IL-6) in the subject is less than prior to the administering. In some embodiments, after the administering the level of interleukin-1beta (IL-1b) in the subject is less than prior to the administering. In some embodiments, after the administering the level of sTNFrc in the subject is less than prior to the administering. In some embodiments, after the administering the level of D-dimer in the subject is less than prior to the administering. In some embodiments, after the administering the level of ferritin in the subject is less than prior to the administering. In some embodiments, after the administering the level of neutrophils in the subject is less than prior to the administering. In some embodiments, after the administering the Sequential Organ Failure Assessment (SOFA) Score change from prior to the administering decreases. In some embodiments, after the administering is about 15 to about 29 days after the administering. In some embodiments, the administering occurs within 72 hours of the subject being diagnosed with ARDS. In some embodiments, the administering occurs within 48 hours of the subject being diagnosed with ARDS.

In some embodiments, the ARDS is acute ARDS. In some embodiments, the acute ARDS comprises dyspnea or worsening of hypoxemic respiratory failure following a predisposing risk factor. In some embodiments, the predisposing risk factor is pneumonia, nonpulmonary infection, trauma, transfusion, aspiration or shock.

In some embodiments, the administering occurs after chest imaging, wherein the chest imaging is indicative of a bilateral opacity. In some embodiments, the bilateral opacity is not due to effusion, atelectasis, or nodule.

In some embodiments, prior to the administering the subject has a PaO2/FiO2 (P/F ratio) of less than or equal to 200 mm Hg. In some embodiments, prior to the administering, the subject is treated with invasive or noninvasive mechanical ventilation. In some embodiments, the mechanical ventilation has a minimum Positive End Expiratory Pressure (PEEP) of about 5 cm H2O. In some embodiments, prior to the administering, the subject is treated with continuous positive airway pressure (CPAP). In some embodiments, the continuous positive airway pressure is performed at 5 cm H2O. In some embodiments, prior to the administering, the subject is treated with high flow nasal oxygen (HFNO) at a level at least about 30 L/min. In some embodiments, prior to the administering, the subject is in respiratory failure. In some embodiments, the respiratory failure is not due to cardiac failure or fluid overload.

In some embodiments, the ARDS subtype is hyperinflammatory ARDS. In some embodiments, the ARDS subtype is hypoinflammatory ARDS. In some embodiments, the ARDS is characterized by serum bicarbonate, sTNFR1, and IL-6.

In some embodiments, the composition comprises one or more proteins and/or one or more extracellular vesicles (EVs). In some embodiments, the total protein concentration of the one or more proteins is about 10 to about 40 μg per ml of the composition. In some embodiments, the total protein concentration of the one or more proteins is about 1.5 to about 6 μg per ml of the composition. In some embodiments, the concentration of the one or more extracellular vesicles is about 10 billion to about 250 billion EVs per ml of the composition. In some embodiments, the concentration of the one or more extracellular vesicles is at about 1 billion to about 40 billion EVs per ml of the composition. In some embodiments, the one or more proteins comprise Ferritin, IGFBP-4 (Insulin-like growth factor binding protein-4), IL-1 R6 (Interleukin 1 Receptor 6), LAMP2 (Lysosome-associated membrane glycoprotein 2), bIG-H3 (Transforming growth factor-beta-induced protein ig-h3), GPR115 (Adhesion G protein-coupled receptor F4), CD63 antigen, CD109 antigen, Serpin F1 (Pigment epithelium-derived factor), IGFBP-6 (Insulin-like growth factor binding protein-6), HS3ST4 (Heparan sulfate glucosamine 3-O-sulfotransferase 4), OPN (Osteopontin), PAI-1 (Plasminogen activator inhibitor-1 or SERPINE 1), Cathepsin B, IGFBP-2 (Insulin-like growth factor binding protein-2), Semaphorin 6C, IGF-2 (Insulin-like growth factor-2), Sortilin, Serpin B6, Dkk-3 (Dickkopf-related protein 3), CNTF (Ciliary neurotrophic factor), TSP-1 (Thrombospondin 1), GM-CSF Ra (Granulocyte-macrophage colony-stimulating factor receptor subunit alpha), Thrombomodulin, Endoglycan, (podocalyxin-like protein 2), IGFBP-3 (Insulin-like binding protein-3), RGM-C (Hemojuvelin), PF4 (Platelet Factor 4), MIF (Macrophage migration inhibitory factor), TGM4 (Protein-glutamine gamma-glutamyltransferase 4), Periostin, Furin, TIMP-1 (Tissue inhibitor of MMPs 1), Decorin, PCK1 (Phosphoenolpyruvate carboxykinase, cytosolic), CD9 antigen, CD99 antigen, CA2 (Carbonic anhydrase 2), PRDX4 (Peroxidredoxin-4), Transferrin, DcR3 (Tumor necrosis factor receptor superfamily member 6B), GP73 (Golgi membrane protein 1), CD81 antigen, Lumican, or TIMP-2 (Tissue Inhibitor of MMPs 2), or a combination of two or more thereof. In some embodiments, the one or more proteins comprise uPAR (CD87), VEGF (vascular endothelial growth factor), thrombomodulin (CD141, thrombin cofactor), CD97 (G protein-coupled receptor), IGFBP2 (insulin growth factor binding protein 2), TSLP (thymic stromal lymphoprotein), NCAM (neuronal cell adhesion molecule), NUP85 (nucleoporin 85), MIF (macrophage inhibitory factor), TNF-alpha RI (tumor necrosis factor-alpha receptor inhibitor), IL1-R6 (interleukin 1 receptor 6), PF4 (platelet factor 4), IGFBP-4 (insulin growth factor binding), bIG-H3 (TGFB induced protein), serpin F1 (secreted multifunctional protein), DKK3 (dickkopf-related protein 3), cathepsin B (catabolic protease), TIMP-1 (collagenase inhibitor), TIMP-2 (collagenase inhibitor), FAP-A (fibroblast activation protein), semaphoring 6c (signal regulator of tissue formation), IGF2 (insulin-like growth factor 2), or FGF-16 (fibroblast growth factor 16), or a combination of two or more thereof. In some embodiments, the one or more proteins comprise at least one of the proteins of Table 1. In some embodiments, the one or more proteins comprises TIMP1. In some embodiments, the TIMP1 is present at about 200 pg/mL to about 80 ng/mL of the composition or about 30 pg/mL to about 12 ng/mL. In some embodiments, the one or more proteins comprises OPN. In some embodiments, the OPN is present at about 200 pg/mL to about 80 ng/mL of the composition or about 30 pg/mL to about 12 ng/mL. In some embodiments, the one or more proteins comprises IGFBP4. In some embodiments, the IGFBP4 is present at about 200 pg/mL to about 80 ng/mL of the composition or about 30 pg/mL to about 12 ng/mL. In some embodiments, the one or more proteins comprises osteonectin. In some embodiments, the osteonectin is present at about 200 pg/mL to about 80 ng/mL of the composition or about 30 pg/mL to about 12 ng/mL. In some embodiments, the concentration of the one or more proteins is measured by ELISA. In some embodiments, the concentration of the one or more extracellular vesicles is measured by nanoparticle tracking analysis (NTA). In some embodiments, the one or more extracellular vesicles comprise extracellular vesicles that are CD63+, CD9−, and CD81−. In some embodiments, at least 50% of the extracellular vesicles are CD63+, and fewer than 50% of the extracellular vesicles are CD9+ or CD81+. In some embodiments, the one or more extracellular vesicles have an average diameter of about 30 nm to about 170 nm. In some embodiments, the diameter is measured by nanoparticle tracking analysis (NTA). In some embodiments, the analysis comprises light scatter and fluorescence evaluation, optionally via NanoSight. In some embodiments, the EVs are characterized by single particle interferometric reflectance imaging sensor technology to visualize and quantify fluorescent antibody-labeled particles. In some embodiments, the molecular weight of the one or more proteins and the one or more extracellular vesicles is greater than about 10 kDa (kilodaltons). In some embodiments, the one or more proteins and/or the one or more extracellular vesicles have a size of less than about 0.2 microns.

In some embodiments, the one or more extracellular vesicles are obtained from a bone-marrow MSC (BM-MSC) cell. In some embodiments, the BM-MSC is obtained from an iliac crest aspiration of a single donor. In some embodiments, the BM-MSC is capable of undergoing trilineage differentiation in vitro toward adipocyte, osteoblast, and chondrocyte phenotypes. In some embodiments, the BM-MSCs are positive for CD73, CD105, CD166, and CD90. In some embodiments, the BM-MSCs are negative for CD14, CD31, CD34, and CD45.

In some embodiments, the composition comprises one or more RNA molecules. In some embodiments, the one or more RNA molecules comprises hsa-miR-125b-5p, hsa-miR-145-5p, hsa-miR-191-5p, hsa-miR-199a-3p, hsa-miR-21-5p, hsa-miR-221-3p, hsa-miR-222-3p, hsa-miR-22-3p, hsa-miR-23a-3p, hsa-miR-23b-3p, hsa-miR-27a-3p, hsa-miR-27b-3p, hsa-miR-29a-3p, hsa-miR-29c-3p, hsa-miR-31-5p, hsa-miR-320a, hsa-miR-34a-5p, hsa-miR-423-3p, hsa-miR-424-5p, or hsa-miR-940, or a combination of two or more thereof. In some embodiments, the one or more RNA molecules are present within the one or more exosomes and/or attached to the one or more extracellular vesicles.

In some embodiments, the composition comprises saline, optionally 0.9% sodium chloride. In some embodiments, the saline is present in the composition at about 80% to about 95%, optionally about 85% saline. In some embodiments, the composition comprises sodium chloride, sodium lactate, potassium chloride, and calcium chloride.

In some embodiments, the composition comprises a saccharide, optionally a polysaccharide. In some embodiments, the saccharide is present in the composition at about 0.4 M. In some embodiments, the saccharide is present in the composition at about 60 mM.

In some embodiments, the composition is sterile by USP <71>. In some embodiments, the composition is endotoxin USP <85> free. In some embodiments, the composition is negative for mycoplasma DNA. In some embodiments, the composition is cell-free.

In some embodiments, the composition is stored between −80° C. and −60° C. prior to administration. In some embodiments, the composition is administered within 6 hours of thaw when maintained at ambient temperature. In some embodiments, the composition has a pH of about 6 to about 7.5.

### BRIEF DESCRIPTION OF THE DRAWINGS

The features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein), of which:

FIGS. 1A-D show acute phase reactants (CRP, ferritin, and D-dimer) and immune cell populations on day of treatment before IV administration of IMP and on day 5 post-treatment. Mean reductions of CRP, ferritin, and D-dimer reductions were 77% (P<0.001), 43% (P<0.001), and 42% (P<0.05), respectively. Mean reduction of ANC was 32% (P<0.001); total lymphocyte count increased by 36% (P<0.05) with CD3+, CD4+, and CD8+T lymphocytes increased by 46% (P<0.05), 45% (P<0.05), and 46% (P<0.001), respectively. ANC, absolute neutrophil count; CRP, C-reactive protein.

FIG. 2 shows a Consolidated Standards of Reporting Trials (CONSORT) diagram.

FIGS. 3A-B shows Kaplan-Meier Plots. Top: time to Death. NR=Not Reached. Time to Death is the interval in days from first dose of IMP to subject's death. The interval is censored to study discontinuation or completion of the subject is alive. Bottom: Time to Discharge (Full Analysis Set). Time to Discharge is the interval in days from first dose of IMP to discharge from hospital. The interval is censored to 60 days if the subject did not discharge.

FIG. 4 shows decline of Acute Phase Biomarkers Throughout the Study for patients remaining hospitalized. Serum levels of ferritin, neutrophils, C-reactive protein (CRP), and D-Dimer were measured by ELISA at Baseline and on days 4, 7, 10, 15, 29 and 61. Mean values for each biomarker were normalized to baseline (100%). On days 0, 4, 7, 10, 15, 29, 61, respectively, the number of remaining hospitalized patients with information available for each marker are: CRP 102, 96, 76, 56, 44, 34, 15; Ferritin 102, 95, 76, 54, 44, 34, 15; D-dimer 102, 95, 75, 56, 43, 33, 15; Neutrophils 99, 93, 69, 51, 43, 34, 15.

### DETAILED DESCRIPTION

Provided herein are compositions and methods for treating Acute Respiratory Distress Syndrome (ARDS).

### I. Compositions

Example compositions herein comprise an extracellular vesicle (EV) and/or a protein. The EV may originate from a mesenchymal stem cell (MSC). The protein may originate from a MSC. In an exemplary embodiment, the MSC is a bone marrow MSC (BM-MSC). The EV and/or protein may be purified or otherwise separated from the MSC growth and/or culturing condition from which the EV and/or protein was secreted into. Purified may include partially purified, such that some of the MSC growth and/or culturing condition is present in the composition. The composition may be formulated into an aqueous solution for intravenous administration.

The composition may comprise the one or more proteins. In some embodiments, the total protein concentration of the one or more proteins is about 10 to about 40 μg per ml of the composition. In some embodiments, the total protein concentration of the one or more proteins is about 1.5 to about 6 μg per ml of the composition. In some embodiments, the one or more proteins comprise Ferritin, IGFBP-4 (Insulin-like growth factor binding protein-4), IL-1 R6 (Interleukin 1 Receptor 6), LAMP2 (Lysosome-associated membrane glycoprotein 2), bIG-H3 (Transforming growth factor-beta-induced protein ig-h3), GPR115 (Adhesion G protein-coupled receptor F4), CD63 antigen, CD109 antigen, Serpin F1 (Pigment epithelium-derived factor), IGFBP-6 (Insulin-like growth factor binding protein-6), HS3ST4 (Heparan sulfate glucosamine 3-O-sulfotransferase 4), OPN (Osteopontin), PAI-1 (Plasminogen activator inhibitor-1 or SERPINE 1), Cathepsin B, IGFBP-2 (Insulin-like growth factor binding protein-2), Semaphorin 6C, IGF-2 (Insulin-like growth factor-2), Sortilin, Serpin B6, Dkk-3 (Dickkopf-related protein 3), CNTF (Ciliary neurotrophic factor), TSP-1 (Thrombospondin 1), GM-CSF Ra (Granulocyte-macrophage colony-stimulating factor receptor subunit alpha), Thrombomodulin, Endoglycan, (podocalyxin-like protein 2) IGFBP-3 (Insulin-like binding protein-3), RGM-C(Hemojuvelin), PF4 (Platelet Factor 4), MIF (Macrophage migration inhibitory factor), TGM4 (Protein-glutamine gamma-glutamyltransferase 4), Periostin, Furin, TIMP-1 (Tissue inhibitor of MMPs 1), Decorin, PCK1 (Phosphoenolpyruvate carboxykinase, cytosolic) CD9 antigen, CD99 antigen, CA2 (Carbonic anhydrase 2), PRDX4 (Peroxidredoxin-4), Transferrin, DcR3 (Tumor necrosis factor receptor superfamily member 6B), GP73 (Golgi membrane protein 1), CD81 antigen, Lumican, or TIMP-2 (Tissue Inhibitor of MMPs 2), or a combination of two or more thereof. In some embodiments, the one or more proteins comprise uPAR (CD87), VEGF (vascular endothelial growth factor), thrombomodulin (CD141, thrombin cofactor), CD97 (G protein-coupled receptor), IGFBP2 (insulin growth factor binding protein 2), TSLP (thymic stromal lymphoprotein), NCAM (neuronal cell adhesion molecule), NUP85 (nucleoporin 85), MIF (macrophage inhibitory factor), TNF-alpha RI (tumor necrosis factor-alpha receptor inhibitor), IL1-R6 (interleukin 1 receptor 6), PF4 (platelet factor 4), IGFBP-4 (insulin growth factor binding), bIG-H3 (TGFB induced protein), serpin F1 (secreted multifunctional protein), DKK3 (dickkopf-related protein 3), cathepsin B (catabolic protease), TIMP-1 (collagenase inhibitor), TIMP-2 (collagenase inhibitor), FAP-A (fibroblast activation protein), semaphoring 6c (signal regulator of tissue formation), IGF2 (insulin-like growth factor 2), or FGF-16 (fibroblast growth factor 16), or a combination of two or more thereof. In some embodiments, the one or more proteins comprise at least one of the proteins of Table 1. In some embodiments, the one or more proteins comprises TIMP1. In some embodiments, the TIMP1 is present at about 200 pg/mL to about 80 ng/mL of the composition or about 30 pg/mL to about 12 ng/mL. In some embodiments, the one or more proteins comprises OPN. In some embodiments, the OPN is present at about 200 pg/mL to about 80 ng/mL of the composition or about 30 pg/mL to about 12 ng/mL. In some embodiments, the one or more proteins comprises IGFBP4. In some embodiments, the IGFBP4 is present at about 200 pg/mL to about 80 ng/mL of the composition or about 30 pg/mL to about 12 ng/mL. In some embodiments, the one or more proteins comprises osteonectin. In some embodiments, the osteonectin is present at about 200 pg/mL to about 80 ng/mL of the composition or about 30 pg/mL to about 12 ng/mL. In some embodiments, the concentration of the one or more proteins is measured by ELISA.

The composition may comprise the extracellular vesicles. In some embodiments, the concentration of the one or more extracellular vesicles is about 10 billion to about 250 billion EVs per ml of the composition. In some embodiments, the concentration of the one or more extracellular vesicles is at about 1 billion to about 40 billion EVs per ml of the composition. In some embodiments, the concentration of the one or more extracellular vesicles is measured by nanoparticle tracking analysis (NTA). In some embodiments, the one or more extracellular vesicles are CD63+, CD9−, and CD81−. In some embodiments, at least about 85%, 86%, 87%, 88%, 89%, 90%, 91%, 92%, 93%, 94%, or 95% of the extracellular vesicles are CD63+, CD9−, and CD81−. In some embodiments, the one or more extracellular vesicles have an average diameter of about 30 nm to about 170 nm. In some embodiments, the diameter is measured by nanoparticle tracking analysis (NTA). In some embodiments, the analysis comprises light scatter and fluorescence evaluation (e.g., NanoSight, Malvern Panalytical Ltd., United Kingdom). In some embodiments, the EVs are characterized by single particle interferometric reflectance imaging sensor technology to visualize and quantify fluorescent antibody-labeled particles (e.g., Unchained Labs, Boston, MA). In some embodiments, the one or more extracellular vesicles are obtained from a bone-marrow MSC (BM-MSC) cell. In some embodiments, the BM-MSC is obtained from an iliac crest aspiration of a single donor. In some embodiments, the BM-MSC is capable of undergoing trilineage differentiation in vitro toward adipocyte, osteoblast, and chondrocyte phenotypes. In some embodiments, the BM-MSCs are positive for CD73, CD105, CD166, and CD90. In some embodiments, the BM-MSCs are negative for CD14, CD31, CD34, and CD45.

In some embodiments, the composition comprises one or more RNA molecules. In some embodiments, the one or more RNA molecules comprises hsa-miR-125b-5p, hsa-miR-145-5p, hsa-miR-191-5p, hsa-miR-199a-3p, hsa-miR-21-5p, hsa-miR-221-3p, hsa-miR-222-3p, hsa-miR-22-3p, hsa-miR-23a-3p, hsa-miR-23b-3p, hsa-miR-27a-3p, hsa-miR-27b-3p, hsa-miR-29a-3p, hsa-miR-29c-3p, hsa-miR-31-5p, hsa-miR-320a, hsa-miR-34a-5p, hsa-miR-423-3p, hsa-miR-424-5p, or hsa-miR-940, or a combination of two or more thereof. In some embodiments, the one or more RNA molecules are present within the one or more exosomes and/or attached to the one or more extracellular vesicles.

In some embodiments, the composition comprises saline (0.9% sodium chloride). In some embodiments, the saline is present in the composition at about 80% to about 95%, e.g., about 85% saline. In some embodiments, the composition comprises sodium chloride, sodium lactate, potassium chloride, and calcium chloride. In some embodiments, the molecular weight of the one or more proteins and the one or more extracellular vesicles is greater than about 10 kDa (kilodaltons). In some embodiments, the composition comprises a saccharide, optionally a polysaccharide. In some embodiments, the saccharide is present in the composition at about 0.4 M or about 60 mM. In some embodiments, the one or more proteins and/or the one or more extracellular vesicles have a size of less than about 0.2 microns. In some embodiments, the composition is sterile by USP <71>. In some embodiments, the composition is endotoxin USP <85> free. In some embodiments, the composition is negative for mycoplasma DNA. In some embodiments, the composition is cell-free. In some embodiments, the composition is stored between −80° C. and −60° C. In some embodiments, the composition is administered within 6 hours of thaw when maintained at ambient temperature. In some embodiments, the composition is present in a glass vial. In some embodiments, the composition is formulated for intravenous administration. In some embodiments, the composition has a pH of about 6 to about 7.5.

The composition may be used for the treatment of ARDS in a method comprising administering to the subject the composition. In some embodiments, the subject has met the Berlin criteria for moderate to severe ARDS. In some embodiments, the composition is administered intravenously. In some embodiments, the administering comprises infusion over 60 minutes on a first day. In some embodiments, the administering further comprises infusion of the composition on a second day. In some embodiments, the second day is one day after the first day (e.g., Day 1 and Day 2), two days after the first day (e.g., Day 1 and Day 3), or three days after the first day (e.g., Day 1 and Day 4). In some embodiments, the infusion on the second day occurs if the subject does not have a SpO2 of at least about 93% between the administering and the infusion on the second day. In some embodiments, the administering comprises infusion of the composition on a third day. In some embodiments, the third day is two days after the second day (e.g., Day 3 and Day 5). In some embodiments, within 28 days after the administering, the subject does not undergo ventilator treatment for at least about 1, 2, 3, 4, 5, 6, or 7 days. In some embodiments, within 28 days after the administering, the subject is not in the intensive care unit for at least about 1, 2, 3, 4, 5, 6, or 7 days. In some embodiments, within 28 days after the administering, the subject is not treated with oxygen for at least about 1, 2, 3, 4, 5, 6, or 7 days.

In some embodiments, after the administering, the subject experiences improved tissue oxygenation. In some embodiments, after the administering, the subject experiences improved end-organ functioning. In some embodiments, after the administering, the subject has an improvement in partial pressure of arterial oxygen to fraction of inspired oxygen (PaO2/FiO2) ratio as compared to prior to the administering (e.g., as measured from arterial blood gas or imputed from SpO2 daily). In some embodiments, after the administering, the subject has an oxygenation saturation of at least about 93% on room air. In some embodiments, after the administering, a biomarker in the subject is lower than prior to the administering. In some embodiments, the biomarker is measured from the blood of the subject. In some embodiments, after the administering the level of C-reactive protein (CRP) in the subject is less than prior to the administering. In some embodiments, after the administering the level of plasminogen activator inhibitor-1 (PAI-1) in the subject is less than prior to the administering. In some embodiments, after the administering the level of interleukin-8 (IL-8) in the subject is less than prior to the administering. In some embodiments, after the administering the level of interleukin-6 (IL-6) in the subject is less than prior to the administering. In some embodiments, after the administering the level of interleukin-1beta (IL-1b) in the subject is less than prior to the administering. In some embodiments, after the administering the level of sTNFrc in the subject is less than prior to the administering. In some embodiments, after the administering the level of D-dimer in the subject is less than prior to the administering. In some embodiments, after the administering the level of ferritin in the subject is less than prior to the administering. In some embodiments, after the administering the level of neutrophils in the subject is less than prior to the administering. In some embodiments, after the administering the Sequential Organ Failure Assessment (SOFA) Score change from prior to the administering decreases. In some embodiments, after the administering is about 15 to about 29 days after the administering. In some embodiments, the administering occurs within 72 hours of the subject being diagnosed with ARDS. In some embodiments, the administering occurs within 48 hours of the subject being diagnosed with ARDS.

In some embodiments, the ARDS is acute ARDS. In some embodiments, the acute ARDS comprises dyspnea or worsening of hypoxemic respiratory failure following a predisposing risk factor. In some embodiments, the predisposing risk factor is pneumonia, nonpulmonary infection, trauma, transfusion, aspiration or shock.

In some embodiments, the administering occurs within 7 days of a clinical insult. In some embodiments, the administering occurs after chest imaging, wherein the chest imaging is indicative of a bilateral opacity. In some embodiments, the bilateral opacity is not due to effusion, atelectasis, or nodule.

In some embodiments, prior to the administering the subject has a PaO2/FiO2 (P/F ratio) of less than or equal to 200 mm Hg. In some embodiments, prior to the administering, the subject is treated with invasive or noninvasive mechanical ventilation. In some embodiments, the mechanical ventilation has a minimum Positive End Expiratory Pressure (PEEP) of about 5 cm H2O. In some embodiments, prior to the administering, the subject is treated with continuous positive airway pressure (CPAP). In some embodiments, the continuous positive airway pressure is performed at 5 cm H2O. In some embodiments, prior to the administering, the subject is treated with high flow nasal oxygen (HFNO) at a level at least about 30 L/min. In some embodiments, prior to the administering, the subject is in respiratory failure. In some embodiments, the respiratory failure is not due to cardiac failure or fluid overload.

In some embodiments, the subject is not infected with COVID-19. In some embodiments, the subject does not have Long COVID-19 (Post Acute Sequalae of COVID-19, PASC), e.g., does not have signs, symptoms, and conditions that continue or develop after acute COVID-19 infection. In some embodiments, the subject is not diagnosed with an infection within 1 week of the administering. In some embodiments, the subject is not suffering from an infection.

In some embodiments, the subject has influenza.

In some embodiments, the ARDS is not caused by bacterial pneumonia, viral pneumonia, fungal pneumonia, or parasitic pneumonia.

In some embodiments, the ARDS subtype is hyperinflammatory ARDS.

In some embodiments, the ARDS subtype is hypoinflammatory ARDS.

In some embodiments, the ARDS is characterized by serum bicarbonate, sTNFR1, and IL-6.

In some embodiments, the ARDS is caused by a pulmonary insult. In some embodiments, the pulmonary insult is aspiration. In some embodiments, the pulmonary insult is smoking. In some embodiments, the pulmonary insult is non-protective ventilation. In some embodiments, the pulmonary insult is lung contusion from trauma. In some embodiments, the pulmonary insult is thoracic surgery. In some embodiments, the pulmonary insult is drowning. In some embodiments, the pulmonary insult is pulmonary vasculitis. In some embodiments, the pulmonary insult is fat embolism. In some embodiments, the administering occurs within 7-14 days of the pulmonary insult.

In some embodiments, the ARDS is caused by a nonpulmonary insult. In some embodiments, the nonpulmonary insult is a blood transfusion. In some embodiments, the nonpulmonary insult is trauma. In some embodiments, the nonpulmonary insult is pancreatitis. In some embodiments, the nonpulmonary insult is drug reaction. In some embodiments, the nonpulmonary insult is a burn. In some embodiments, the nonpulmonary insult is a cardiopulmonary bypass. In some embodiments, the nonpulmonary insult is noncardiogenic shock. In some embodiments, the administering occurs within 7-14 days of the nonpulmonary insult.

### Extracellular Vesicles

Extracellular vesicles (EV) are small membrane bound spheres containing proteins and RNA (of which exosomes are a subset). Exosomes are small lipid bilayer vesicles secreted by cells that lack a nucleus and cannot replicate. Other EV populations are derived directly from the plasma membrane or are formed during apoptosis (apoptotic bodies). Disclosed herein are compositions comprising an EV. In example embodiments, the EV is an exosome. Embodiments of an EV herein have a diameter of about 20 nm to about 200 nm. In some embodiments, the diameter is measured by nanoparticle tracking analysis (NTA).

The number of EVs within a composition may be about 10 billion to about 250 billion EVs per mL when suspended. The suspension may be diluted for intravenous administration, wherein the EVs within the composition may be about 1 billion to about 40 billion EVs per mL.

In some embodiments, the EV has a phenotype of CD63⁺ CD9⁻ and CD81⁻. In some embodiments, at least 70, 75, 80, 85, 90, 91, 92, 93, 94, or 95% of the EVs are CD63⁺ CD9⁻ and CD81⁻. In some embodiments, at least 50, 60, 70, 80, 85, 90, 91, 92, 93, 94, or 95% of the EVs are CD9⁻. In some embodiments, at least 50, 60, 70, 80, 85, 90, 91, 92, 93, 94, or 95% of the EVs are CD81⁻.

In some embodiments, the EV is produced from a MSC. The MSC may be a bone marrow MSC. The MSC may be a human MSC. In some embodiments, the EV is produced from a MSC that has the capacity to undergo trilineage differentiation in vitro toward adipocyte, osteoblast, and chondrocyte phenotypes. In some embodiments, the MSC is positive for CD73, CD105, CD166, and CD90 and is negative for CD14, CD31, CD34, and CD45.

In some embodiments, EVs are analyzed via light scatter and fluorescence evaluation (e.g., NanoSight, Malvern Panalytical Ltd., United Kingdom). In some embodiments, the EVs are characterized by single particle interferometric reflectance imaging sensor technology to visualize and quantify fluorescent antibody-labeled particles (e.g., NanoView Biosciences, Boston, MA).

In some embodiments, the EV comprises a peptide or protein. In some embodiments, the EV comprises a nucleic acid. Nucleic acids include ribonucleic acids (RNA), such as siRNA, shRNA, and microRNA (miRNA).

In some embodiments, the EV comprises one or more proteins (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 65, 70, 75, 80, 85, 90, 95, or 100). Non-limiting examples of the one or more proteins include: Ferritin, IGFBP-4 (Insulin-like growth factor binding protein-4), IL-1 R6 (Interleukin 1 Receptor 6), LAMP2 (Lysosome-associated membrane glycoprotein 2), bIG-H3 (Transforming growth factor-beta-induced protein ig-h3), GPR115 (Adhesion G protein-coupled receptor F4), CD63 antigen, CD109 antigen, Serpin F1 (Pigment epithelium-derived factor), IGFBP-6 (Insulin-like growth factor binding protein-6), HS3ST4 (Heparan sulfate glucosamine 3-O-sulfotransferase 4), OPN (Osteopontin), PAI-1 (Plasminogen activator inhibitor-1 or SERPINE 1), Cathepsin B, IGFBP-2 (Insulin-like growth factor binding protein-2), Semaphorin 6C, IGF-2 (Insulin-like growth factor-2), Sortilin, Serpin B6, Dkk-3 (Dickkopf-related protein 3), CNTF (Ciliary neurotrophic factor), TSP-1 (Thrombospondin 1), GM-CSF Ra (Granulocyte-macrophage colony-stimulating factor receptor subunit alpha), Thrombomodulin, Endoglycan, (podocalyxin-like protein 2) IGFBP-3 (Insulin-like binding protein-3), RGM-C(Hemojuvelin), PF4 (Platelet Factor 4), MIF (Macrophage migration inhibitory factor), TGM4 (Protein-glutamine gamma-glutamyltransferase 4), Periostin, Furin, TIMP-1 (Tissue inhibitor of MMPs 1), Decorin, PCK1 (Phosphoenolpyruvate carboxykinase, cytosolic) CD9 antigen, CD99 antigen, CA2 (Carbonic anhydrase 2), PRDX4 (Peroxidredoxin-4), Transferrin, DcR3 (Tumor necrosis factor receptor superfamily member 6B), GP73 (Golgi membrane protein 1), CD81 antigen, Lumican, or TIMP-2 (Tissue Inhibitor of MMPs 2), and the proteins of Table 1. An EV that comprises the one or more of the proteins may include the one or more proteins within the EV. An EV that comprises one or more of the proteins may include the one or more proteins anchored within the FV. An FV that comprises the one or more of the proteins may be associated with the outside of the FV.

Table 1 below provides additional information regarding non-limiting examples of proteins that may be comprised within an FV or FV composition of the present disclosure.

TABLE 1

| | | | | | Protein Names |
|---|---|---|---|---|---|
| | | | | | (Recommended, |
| | | | | | Short, Alternative, |
| | | Primary | | | CD Antigen Names |
| | Official | Protein | | | & Enzyme |
| | Gene | Accession | Official Full | Synonyms | Commission (EC) |
| Protein | Symbol | Number | Gene Name | (UniProtKB, | Numbers) |
| Name | (HGCN) | (UniProtKB) | (HGCN) | NCBI, IPA) | (UniProtKB) |
| Ferritin | FTL | P02792 | ferritin | FTL1, LFTD, | ferritin light chain, |
| | | | light chain | NBIA3, FL, FRIL, | ferritin L subunit |
| | | | | L-ferritin, | |
| | | | | YB24D08 | |
| | FTH1 | P02794 | ferritin | FHC, FTH, | ferritin heavy chain, |
| | | | heavy chain | FTHL6, HFE5, | ferritin H subunit, |
| | | | | NBIA9, PIG15, | cell proliferation- |
| | | | | PLIF, | inducing gene 15 |
| | | | | APOFERRITIN H | protein, ferritin |
| | | | | CHAIN, ferritin | heavy chain N- |
| | | | | heavy chain 1, H- | terminally |
| | | | | ferritin | processed, |
| | | | | | EC: 1.16.3.1 |
| | FTMT | Q8N4E7 | ferritin | MTF | ferritin |
| | | | mitochondrial | | mitochondrial, |
| | | | | | EC: 1.16.3.1 |
| IGFBP-4 | IGFBP4 | P22692 | insulin like | BP-4, HT29- | insulin-like growth |
| | | | growth factor | IGFBP, IBP4, | factor-binding |
| | | | binding protein 4 | IGFBP-4 | protein 4, IBP-4, |
| | | | | | IGF-binding protein |
| | | | | | 4, IGFBP-4 |
| IL-1 R6 | IL1RL2 | Q9HB29 | interleukin 1 | IL-1Rrp2, IL-36R, | interleukin-1 |
| | | | receptor like 2 | IL1R-rp2, | receptor-like 2, IL- |
| | | | | IL1RRP2 | 36 receptor (IL- |
| | | | | | 36R), interleukin-1 |
| | | | | | receptor-related |
| | | | | | protein 2 (IL-1Rrp2; |
| | | | | | ILIR-rp2), |
| | | | | | EC: 3.2.2.6 |
| GSTM1 | GSTM1 | P09488 | glutathione S- | GST1, GST1-1, | glutathione S- |
| | | | transferase mu 1 | GSTM1a-1a, | transferase Mu 1, |
| | | | | GSTM1b-1b, | GST HB subunit 4, |
| | | | | GTH4, GTM1, H- | GST class-mu 1, |
| | | | | B, MU, MU-1, | GSTM1-1, |
| | | | | glutathione S- | GSTM1a-1a, |
| | | | | transferase subunit | GSTM1b-1b, |
| | | | | 4, GSTM1-1, GST | GTH4, EC: 2.5.1.18 |
| | | | | N1, HB subunit 4 | |
| NUP85 | NUP85 | Q9BW27 | nucleoporin 85 | FROUNT, | nuclear pore |
| | | | | NPHS17, Nup75, | complex protein |
| | | | | NUP75, PCNT1 | Nup85, 85 kDa |
| | | | | | nucleoporin, |
| | | | | | FROUNT, |
| | | | | | nucleoporin Nup75, |
| | | | | | nucleoporin Nup85, |
| | | | | | pericentrin-1 |
| LAMP2 | LAMP2 | P13473 | lysosomal | CD107b, DND, | lysosome-associated |
| | | | associated | LAMP-2, | membrane |
| | | | membrane protein | LAMPB, LGP-96, | glycoprotein 2, |
| | | | 2 | LGP110 | LAMP-2, lysosome- |
| | | | | | associated |
| | | | | | membrane protein 2, |
| | | | | | CD107 antigen-like |
| | | | | | family member B, |
| | | | | | LGP-96, CD1076 |
| Meprin A | MEP1A | Q16819 | meprin A subunit | PPHA, meprin A | meprin A subunit |
| | | | alpha | subunit α, meprin | alpha, |
| | | | | A α | endopeptidase-2, N- |
| | | | | | benzoyl-L-tyrosyl- |
| | | | | | P-amino-benzoic |
| | | | | | acid hydrolase |
| | | | | | subunit alpha, |
| | | | | | PABA peptide |
| | | | | | hydrolase, PPH |
| | | | | | alpha, EC: 3.4.24.18 |
| | MEP1B | Q16820 | meprin A subunit | | meprin A subunit |
| | | | beta | | beta, endopeptidase- |
| | | | | | 2, meprin B, N- |
| | | | | | benzoyl-L-tyrosyl- |
| | | | | | P-amino-benzoic |
| | | | | | acid hydrolase |
| | | | | | subunit beta, PABA |
| | | | | | peptide hydrolase, |
| | | | | | PPH beta, |
| | | | | | EC: 3.4.24.63, |
| | | | | | endopeptidase-2, |
| | | | | | meprin A subunit β, |
| | | | | | meprin beta, meprin |
| | | | | | β |
| IL-1 F10 | IL1F10 | Q8WWZ1 | interleukin 1 | FIL1T, IL1HY2, | interleukin-1 family |
| | | | family member 10 | IL38, FIL1-theta, | member 10, IL- |
| | | | | FKSG75, IL- | 1F10, family of |
| | | | | 1HY2, IL-38, IL1- | interleukin 1-theta |
| | | | | theta, FIL1-θ, | (FIL1 theta), |
| | | | | IL1-θ | interleukin-1 HY2 |
| | | | | | (IL-1HY2), |
| | | | | | interleukin-1 theta |
| | | | | | (IL-1 theta), |
| | | | | | interluekin-38 (IL- |
| | | | | | 38) |
| bIG-H3 | TGFBI | Q15582 | transforming | BIGH3, CDB1, | transforming growth |
| | | | growth factor beta | CDG2, CDGG1, | factor-beta-induced |
| | | | induced | CSD, CSD1, | protein ig-h3, beta |
| | | | | CSD2, CSD3, | ig-h3, kerato- |
| | | | | EBMD, LCD1, | epithelin, RGD- |
| | | | | TGFBIP, | containing collagen- |
| | | | | transforming | associated protein |
| | | | | growth factor β | (RGD-CAP) |
| | | | | induced, β Ig-H3 | |
| GPR115 | ADGRF4 | Q8IZF3 | adhesion G | GPR115, PGR18 | adhesion G protein- |
| | | | protein-coupled | | coupled receptor F4, |
| | | | receptor F4 | | G-protein coupled |
| | | | | | receptor 115, G- |
| | | | | | protein coupled |
| | | | | | receptor PGR18 |
| TGFb1 | TGFB1 | P01137 | transforming | CED, DPD1, | transforming growth |
| | | | growth factor beta | IBDIMDE, LAP, | factor beta-1 |
| | | | 1 | TGF-beta1, TGFB, | proprotein, latency- |
| | | | | TGFbeta, TGF-β | associated peptide |
| | | | | 1, transforming | (LAP), transforming |
| | | | | beta-1 growth | growth factor beta-1 |
| | | | | factor, | (TGF-beta-1) |
| | | | | transforming | |
| | | | | growth factor β 1, | |
| | | | | transforming β-1 | |
| | | | | growth factor | |
| Ephrin-A4 | EFNA4 | P52798 | ephrin A4 | EFL4, EPLG4, | ephrin-A4, EPH- |
| | | | | LERK-4, LERK4 | related receptor |
| | | | | | tyrosine kinase |
| | | | | | ligand 4 (LERK-4) |
| CD109 | CD109 | Q6YHK3 | CD109 molecule | CPAMD7, p180, | CD109 antigen, 150 |
| | | | | r150 | kDa TGF-beta-1- |
| | | | | | binding protein, C3 |
| | | | | | and PZP-like alpha- |
| | | | | | 2-macroglobulin |
| | | | | | domain-containing |
| | | | | | protein 7, platelet- |
| | | | | | specific Gov |
| | | | | | antigen, p180, r150, |
| | | | | | CD109 |
| Serpin F1 | SERPINF1 | P36955 | serpin family F | EPC-1, OI12, OI6, | pigment epithelium- |
| | | | member 1 | PEDF, PIG35 | derived factor, |
| | | | | | PEDF, cell |
| | | | | | proliferation- |
| | | | | | inducing gene 35 |
| | | | | | protein, EPC-1, |
| | | | | | serpin F1 |
| IGFBP-6 | IGFBP6 | P24592 | insulin like | IBP6 | insulin-like growth |
| | | | growth factor | | factor-binding |
| | | | binding protein 6 | | protein 6, IBP-6, |
| | | | | | IGF-binding protein |
| | | | | | 6, IGFBP-6 |
| HS3ST4 | HS3ST4 | Q9Y661 | heparan sulfate- | 3OST4, 3-OST-4, | heparan sulfate |
| | | | glucosamine 3- | 30ST4, h3-OST-4, | glucosamine 3-O- |
| | | | sulfotransferase 4 | heparan sulphate- | sulfotransferase 4, |
| | | | | glucosamine 3- | heparan sulfate D- |
| | | | | sulphotransferase | glucosaminy1 3-O- |
| | | | | 4 | sulfotransferase (3- |
| | | | | | OST-4; heparan |
| | | | | | sulfate 3-O- |
| | | | | | sulfotransferase 4; |
| | | | | | h3-OST-4), |
| | | | | | EC: 2.8.2.23 |
| Aminopeptidase | ERAP2 | Q6P179 | endoplasmic | L-RAP, LRAP | endoplasmic |
| LRAP | | | reticulum | | reticulum |
| | | | aminopeptidase 2 | | aminopeptidase 2, |
| | | | | | leukocyte-derived |
| | | | | | arginine |
| | | | | | aminopeptidase (L- |
| | | | | | RAP), EC: 3.4.11.- |
| OPN | SPP1 | P10451 | secreted | BNSP, BSPI, | osteopontin, bone |
| | | | phosphoprotein 1 | ETA-1, OPN, | sialoprotein 1, |
| | | | | osteopontin, | nephropontin, |
| | | | | nephropontin, | secreted |
| | | | | SPP1 isoform 1, | phosphoprotein 1 |
| | | | | uropontin | (SPP-1), urinary |
| | | | | | stone protein, |
| | | | | | uropontin |
| PAI-1 | SERPINE1 | P05121 | serpin family E | PAI, PAI-1, PAI1, | plasminogen |
| | | | member 1 | PLANH1, beta | activator inhibitor 1, |
| | | | | migrating PLAS | PAI, PAI-1, |
| | | | | activator, beta- | endothelial |
| | | | | migrating | plasminogen |
| | | | | plasminogen | activator inhibitor, |
| | | | | activator inhibitor | serpin E1 |
| | | | | I, β migrating | |
| | | | | PLAS activator, | |
| | | | | β-migrating | |
| | | | | plasminogen | |
| | | | | activator inhibitor | |
| | | | | I | |
| DAPP1 | DAPP1 | Q9UN19 | dual adaptor of | BAM32 | dual adapter for |
| | | | phosphotyrosine | | phosphotyrosine and |
| | | | and 3- | | 3-phosphotyrosine |
| | | | phosphoinositides | | and 3- |
| | | | 1 | | phosphoinositide, |
| | | | | | hDAPP1, B |
| | | | | | lymphocyte adapter |
| | | | | | protein Bam32, B- |
| | | | | | cell adapter |
| | | | | | molecule of 32 kDa |
| GDF-9 | GDF9 | O60383 | growth | POF14, GDF-9 | growth/differentiation |
| | | | differentiation | | factor 9, GDF-9 |
| | | | factor 9 | | |
| Cathepsin B | CTSB | P07858 | cathepsin B | APPS, CPSB, | cathepsin B, APP |
| | | | | KWE, RECEUP, | secretase (APPS), |
| | | | | AC270285.4 | cathepsin B1, |
| | | | | | cathepsin B light |
| | | | | | chain, cathepsin B |
| | | | | | heavy chain, |
| | | | | | EC: 3.4.22.1 |
| IGFBP-2 | IGFBP2 | P18065 | insulin like | IBP2, IGF-BP53, | insulin-like growth |
| | | | growth factor | BP2 | factor-binding |
| | | | binding protein 2 | | protein 2, IBP-2, |
| | | | | | IGF-binding protein |
| | | | | | 2, IGFBP-2 |
| Semaphorin6C | SEMA6C | Q9H3T2 | semaphorin 6C | SEMAY, Sema-Y, | semaphorin-6C, |
| | | | | m-SemaY, m- | semaphorin-Y |
| | | | | SemaY2, | (Sema Y) |
| | | | | KIAA1869, | |
| | | | | semaphorin Y | |
| IGF-2 | IGF2 | P01344 | insulin like | C11orf43, GRDF, | insulin-like growth |
| | | | growth factor 2 | IGF-II, PP9974, | factor II, IGF-II, |
| | | | | SRS3, IGF2 | somatomedin-A, |
| | | | | isoform 1, | T3M-11-derived |
| | | | | somatomedin A | growth factor, |
| | | | | | insulin-like growth |
| | | | | | factor II, insulin-like |
| | | | | | growth factor II Ala- |
| | | | | | 25 Del, preptin |
| PDGF R | PDGFRA | P16234 | platelet derived | CD140A, PDGFR- | platelet-derived |
| alpha | | | growth factor | 2, PDGFR2, | growth factor |
| | | | receptor alpha | RHEPDGFRA, | receptor alpha, |
| | | | | PDGFR alpha, | PDGF-R-alpha, |
| | | | | Pdgf receptor-α, | PDGFR-alpha, |
| | | | | PDGFR α, platelet | alpha platelet- |
| | | | | derived growth | derived growth |
| | | | | factor receptor α | factor receptor, |
| | | | | | alpha-type platelet- |
| | | | | | derived growth |
| | | | | | factor receptor, |
| | | | | | CD140 antigen-like |
| | | | | | family member A, |
| | | | | | CD140a antigen, |
| | | | | | platelet-derived |
| | | | | | growth factor alpha |
| | | | | | receptor, platelet- |
| | | | | | derived growth |
| | | | | | factor receptor 2 |
| | | | | | (PDGFR-2), |
| | | | | | CD140a, |
| | | | | | EC: 2.7.10.1 |
| Sortilin | SORT1 | Q99523 | sortilin 1 | Gp95, LDLCQ6, | sortilin, 100 kDa NT |
| | | | | NT3, NTR3, | receptor, |
| | | | | NTSR3, sortilin, | glycoprotein 95 |
| | | | | glycoprotein 95 | (Gp95), neurotensin |
| | | | | | receptor 3 (NT3; |
| | | | | | NTR3) |
| Serpin B6 | SERPINB6 | P35237 | serpin family B | CAP, DFNB91, | serpin B6, |
| | | | member 6 | MSTP057, PI-6, | cytoplasmic |
| | | | | PI6, PTI, SPI3, | antiproteinase |
| | | | | Serpin6b | (CAP), peptidase |
| | | | | | inhibitor 6 (PI-6), |
| | | | | | placental thrombin |
| | | | | | inhibitor |
| Dkk-3 | DKK3 | Q9UBP4 | dickkopf WNT | CRRL, REIC, | dickkopf-related |
| | | | signaling pathway | RIG, Dickkopf | protein 3, dickkopf- |
| | | | inhibitor 3 | homolog 3 | 3, Dkk-3, hDkk-3 |
| CNTF | CNTF | P26441 | ciliary | HCNTF | ciliary neurotrophic |
| | | | neurotrophic | | factor, CNTF |
| | | | factor | | |
| TSP-1 | THBS1 | P07996 | thrombospondin 1 | THBS, THBS-1, | thrombospondin-1, |
| | | | | TSP, TSP-1, TSP1 | glycoprotein G |
| GM-CSF Ra | CSF2RA | P15509 | colony stimulating | CD116, CDw116, | granulocyte- |
| | | | factor 2 receptor | CSF2RX, | macrophage colony- |
| | | | subunit alpha | CSF2RAY, | stimulating factor |
| | | | | CSF2RX | receptor subunit |
| | | | | CSF2RY, GM- | alpha, GM-CSF-R- |
| | | | | CSF-R-alpha, | alpha, GMCSFR- |
| | | | | GMR, GMR- | alpha, GMR-alpha, |
| | | | | alpha, SMDP4, | CDw116, CD116 |
| | | | | alphaGMR, | |
| | | | | CSF2RA, CSF2R, | |
| | | | | colony stimulating | |
| | | | | factor 2 receptor | |
| | | | | subunit α, | |
| | | | | CSF2RAX, | |
| | | | | CSF2RAY, | |
| | | | | GMCSFR, | |
| | | | | GMCSFR-alpha, | |
| | | | | GM-CSF receptor, | |
| | | | | GM-CSF receptor | |
| | | | | alpha chain, GM- | |
| | | | | CSF receptor α | |
| | | | | chain, GMCSFR- | |
| | | | | α, Gm-Csfr a, | |
| | | | | GM-CSF-R-α, | |
| | | | | GMR-α, Gm-R α, | |
| | | | | MGMR alpha, | |
| | | | | MGMR α, SGMR | |
| | | | | alpha, SGMR α, | |
| | | | | SMDP4 | |
| Thrombomodulin | THBD | P07204 | thrombomodulin | AHUS6, BDCA-3, | thrombomodulin, |
| | | | | BDCA3, CD141, | TM, fetomodulin, |
| | | | | THPH12, THRM, | CD141 |
| | | | | TM | |
| Endoglycan | PODXL2 | Q9NZ53 | podocalyxin like 2 | EG, PODLX2 | podocalyxin-like |
| | | | | | protein 2, |
| | | | | | endoglycan |
| IGFBP-3 | IGFBP3 | P17936 | insulin like | BP-53, IBP3, | insulin-like growth |
| | | | growth factor | IGFBP-3, IBP-3, | factor-binding |
| | | | binding protein 3 | IGBP3, IGFI BP3 | protein 3, IBP-3, |
| | | | | | IGF-binding protein |
| | | | | | 3, IGFBP-3 |
| RGM-C | HJV | Q6ZVN8 | hemojuvelin BMP | HFE2, HFE2A, | hemojuvelin, |
| | | | co-receptor | JH, RGMC, | hemochromatosis |
| | | | | haemojuvelin | type 2 protein, |
| | | | | BMP co-receptor, | hemojuvelin BMP |
| | | | | LOC148738 | coreceptor, RGM |
| | | | | | domain family |
| | | | | | member C |
| PF4 | PF4 | P02776 | platelet factor 4 | CXCL4, PF-4, | platelet factor 4, PF- |
| | | | | SCYB4 | 4, C-X-C motif |
| | | | | | chemokine 4, |
| | | | | | iroplact, oncostatin- |
| | | | | | A, platelet factor 4 |
| | | | | | short form, |
| | | | | | endothelial cell |
| | | | | | growth inhibitor |
| MIF | MIF | P14174 | macrophage | GIF, MMIF, GLIF | macrophage |
| | | | migration | | migration inhibitory |
| | | | inhibitory factor | | factor, MIF, |
| | | | | | glycosylation- |
| | | | | | inhibiting factor |
| | | | | | (GIF), L- |
| | | | | | dopachrome |
| | | | | | isomerase, L- |
| | | | | | dopachrome |
| | | | | | tautomerase |
| | | | | | (EC: 5.3.3.12), |
| | | | | | phenylpyruvate |
| | | | | | tautomerase, |
| | | | | | EC: 5.3.2.1 |
| TGM4 | TGM4 | P49221 | transglutaminase 4 | TGP, hTGP | protein-glutamine |
| | | | | | gamma- |
| | | | | | glutamyltransferase |
| | | | | | 4, fibrinoligase, |
| | | | | | prostate |
| | | | | | transglutaminase, |
| | | | | | prostate-specific |
| | | | | | transglutaminase, |
| | | | | | transglutaminase P |
| | | | | | (TG(P); TGP; |
| | | | | | TGase P), |
| | | | | | transglutaminase-4 |
| | | | | | (TGase-4), |
| | | | | | EC: 2.3.2.13 |
| Periostin | POSTN | Q15063 | periostin | OSF-2, OSF2, | periostin, PN, |
| | | | | PDLPOSTN, PN, | osteoblast-specific |
| | | | | FLJ11382, OSF- | factor 2 (OSF-2) |
| | | | | 2P1 | |
| Furin | FURIN | P09958 | furin, paired basic | FUR, PACE, | furin, dibasic- |
| | | | amino acid | PCSK3, SPC1, | processing enzyme, |
| | | | cleaving enzyme | FURIN from | paired basic amino |
| | | | | PACE, | acid residue- |
| | | | | | cleaving enzyme |
| | | | | | (PACE), |
| | | | | | EC: 3.4.21.75 |
| TIMP-1 | TIMP1 | P01033 | TIMP | CLGI, EPA, EPO, | metalloproteinase |
| | | | metallopeptidase | HCI, TIMP, | inhibitor 1, |
| | | | inhibitor 1 | TIMP-1 | erythroid- |
| | | | | | potentiating activity |
| | | | | | (EPA), fibroblast |
| | | | | | collagenase inhibitor |
| | | | | | (collagenase |
| | | | | | inhibitor), tissue |
| | | | | | inhibitor of |
| | | | | | metalloproteinases 1 |
| | | | | | (TIMP-1) |
| PAPP-A | PAPPA | Q13219 | pappalysin 1 | ASBABP2, | pappalysin-1, |
| | | | | DIPLA1, IGFBP- | insulin-like growth |
| | | | | 4ase, PAPA, | factor-dependent |
| | | | | PAPP-A1, | IGF-binding protein |
| | | | | PAPPA1 | 4 protease (IGF- |
| | | | | | dependent IGFBP-4 |
| | | | | | protease; IGFBP- |
| | | | | | 4ase), pregnancy- |
| | | | | | associated plasma |
| | | | | | protein A (PAPP- |
| | | | | | A), EC: 3.4.24.79 |
| Decorin | DCN | P07585 | decorin | CSCD, DSPG2, | decorin, bone |
| | | | | PG40, PGII, | proteoglycan II, PG- |
| | | | | PGS2, SLRR1B, | S2, PG40 |
| | | | | bone proteoglycan | |
| | | | | II | |
| PCK1 | PCK1 | P35558 | phosphoenolpyruvate | PCKDC, PEPCK- | phosphoenolpyruvate |
| | | | carboxykinase | C, PEPCK1, | carboxykinase |
| | | | 1 | PEPCKC | cytosolic [GTP], |
| | | | | | PEPCK-C, serine- |
| | | | | | protein kinase PCK1 |
| | | | | | (EC: 2.7.11.-), |
| | | | | | EC: 4.1.1.32 |
| ArylsulfataseA | ARSA | P15289 | arylsulfatase A | ASA, MLD | arylsulfatase A, |
| | | | | | ASA, cerebroside- |
| | | | | | sulfatase, |
| | | | | | arylsulfatase A |
| | | | | | component B, |
| | | | | | arylsulfatase A |
| | | | | | component C, |
| | | | | | EC: 3.1.6.8 |
| CD99 | CD99 | P14209 | CD99 molecule | HBA71, MIC2, | CD99 antigen, |
| | | | (Xg blood group) | MIC2X, MIC2Y, | 12E7, E2 antigen, |
| | | | | MSK5X, | protein MIC2, T-cell |
| | | | | 2410026K10RIK, | surface glycoprotein |
| | | | | Pilr-I, T cell | E2, CD99 |
| | | | | surface | |
| | | | | glycoprotein E2 | |
| CA2 | CA2 | P00918 | carbonic | CA-II, CAC, | carbonic anhydrase |
| | | | anhydrase 2 | CAII, Car2, HEL- | 2, carbonate |
| | | | | 76, HEL-S-282, | dehydratase II, |
| | | | | carbonic | carbonic anhydrase |
| | | | | anhydrase isozyme | C (CAC), carbonic |
| | | | | II | anhydrase II (CA- |
| | | | | | II), cyanamide |
| | | | | | hydratase CA2 |
| | | | | | (EC: 4.2.1.69), |
| | | | | | EC: 4.2.1.1 |
| PRDX4 | PRDX4 | Q13162 | peroxiredoxin 4 | AOE37-2, | peroxiredoxin-4, |
| | | | | AOE372, HEL-S- | antioxidant enzyme |
| | | | | 97n, PRX-4, AOE | AOE372 (AOE37- |
| | | | | 37-2, Prx-IV, | 2), peroxiredoxin IV |
| | | | | TRANK | (Prx-IV), |
| | | | | | thioredoxin |
| | | | | | peroxidase AO372, |
| | | | | | thioredoxin- |
| | | | | | dependent peroxide |
| | | | | | reductase A0372, |
| | | | | | thioredoxin- |
| | | | | | dependent |
| | | | | | peroxiredoxin 4, |
| | | | | | EC: 1.11.1.24 |
| Transferrin | TF | P02787 | transferrin | HEL-S-71p, | serotransferrin, |
| | | | | PRO1557, | transferrin, beta-1 |
| | | | | PRO2086QTL1, | metal-binding |
| | | | | TF, ApoTf, | globulin, |
| | | | | apotransferrin, | siderophilin |
| | | | | TFQTL1, | |
| | | | | PRO2086 | |
| DcR3 | TNFRSF6B | O95407 | TNF receptor | DCR3, | tumor necrosis |
| | | | superfamily | DJ583P15.1.1, | factor receptor |
| | | | member 6B | M68, M68E, TR6, | superfamily member |
| | | | | tumor necrosis | 6B, decoy receptor 3 |
| | | | | factor receptor | (DcR3), decoy |
| | | | | superfamily | receptor for Fas |
| | | | | member 6B | ligand, M68 |
| GP73 | GOLM1 | Q8NBJ4 | golgi membrane | C9orf155, | golgi membrane |
| | | | protein 1 | GOLPH2, GP73, | protein 1, golgi |
| | | | | HEL46, | membrane protein |
| | | | | PSEC0257, | GP73, golgi |
| | | | | bA379P1.3, golgi | phosphoprotein 2 |
| | | | | phosphoprotein 2, | |
| | | | | LOC100293491 | |
| LAIR2 | LAIR2 | Q6ISS4 | leukocyte | CD306 | leukocyte-associated |
| | | | associated | | immunoglobulin- |
| | | | immunoglobulin | | like receptor 2, |
| | | | like receptor 2 | | LAIR-2, CD306 |
| ULBP-4 | RAET1E | Q8TD07 | retinoic acid early | LETAL, N2DL4, | retinoic acid early |
| | | | transcript 1E | N2DL-4, ULBP4, | transcript 1E, |
| | | | | NKG2DL4, | lymphocyte effector |
| | | | | NKG2DL42, RL- | toxicity activation |
| | | | | 4, bA350J20.7, | ligand, NKG2D |
| | | | | ULBP-4, NKG2D | ligand 4 (N2DL-4; |
| | | | | ligand 4 | NKG2DL4), RAE- |
| | | | | | 1-like transcript 4, |
| | | | | | UL16-binding |
| | | | | | protein 4 |
| Lumican | LUM | P51884 | lumican | LDC, SLRR2D | lumican, keratan |
| | | | | | sulfate proteoglycan |
| | | | | | lumican (KSPG |
| | | | | | lumican) |
| TIMP-2 | TIMP2 | P16035 | TIMP | CSC-21K, DDC8 | metalloproteinase |
| | | | metallopeptidase | | inhibitor 2, CSC- |
| | | | inhibitor 2 | | 21K, tissue inhibitor |
| | | | | | of |
| | | | | | metalloproteinases 2 |
| | | | | | (TIMP-2) |
| TFPI | TFPI | P10646 | tissue factor | EPI, LACI, TFI1, | tissue factor |
| | | | pathway inhibitor | TFPI1, TFI, TFP1 | pathway inhibitor, |
| | | | | | TFPI, extrinsic |
| | | | | | pathway inhibitor |
| | | | | | (EPI), lipoprotein- |
| | | | | | associated |
| | | | | | coagulation inhibitor |
| | | | | | (LACI) |
| SOX2 | SOX2 | P48431 | SRY-box | ANOP3, MCOPS3 | transcription factor |
| | | | transcription | | SOX-2 |
| | | | factor 2 | | |
| SLITRK5 | SLITRK5 | O94991 | SLIT and NTRK | LRRC11, | SLIT and NTRK- |
| | | | like family | bA364G4.2, | like protein 5, |
| | | | member 5 | LRCC11 | leucine-rich repeat- |
| | | | | | containing protein |
| | | | | | 11 |
| FAP | FAP | Q12884 | fibroblast | DPPIVA, | prolyl endopeptidase |
| | | | activation protein | FAPalpha, SIMP, | FAP, 170 kDa |
| | | | alpha | FAP, DPPIV, F19, | melanoma |
| | | | | FAPA, fibroblast | membrane-bound |
| | | | | activation protein | glatinase, dipeptidyl |
| | | | | α | peptidase FAP |
| | | | | | (EC: 3.4.14.5), |
| | | | | | fibroblast activation |
| | | | | | protein alpha |
| | | | | | (FAPalpha), gelatine |
| | | | | | degradation protease |
| | | | | | FAP (EC: 3.4.21.-), |
| | | | | | integral membrane |
| | | | | | serine protease, |
| | | | | | post-proline |
| | | | | | cleaving enzyme, |
| | | | | | serine integral |
| | | | | | membrane protease |
| | | | | | (SIMP), surface- |
| | | | | | expressed protease |
| | | | | | (seprase), |
| | | | | | antiplasmin- |
| | | | | | cleaving enzyme |
| | | | | | FAP soluble form |
| | | | | | (APCE), |
| | | | | | EC: 3.4.21.26 |
| Spinesin | TMPRSS5 | Q9H3S3 | transmembrane | SPINESIN | transmembrane |
| | | | serine protease 5 | | protease serine 5, |
| | | | | | spinesin, |
| | | | | | EC: 3.4.21.- |
| ENPP-2 | ENPP2 | Q13822 | ectonucleotide | ATX, ATX-X, | autotaxin, |
| | | | pyrophosphatase/ | AUTOTAXIN, | ectonucleotide |
| | | | phosphodiesterase | LysoPLD, NPP2, | pyrophosphatase/ |
| | | | 2 | PD-IALPHA, | phosphodiesterase |
| | | | | PDNP2, XSG17 | family member 2 |
| | | | | | (E-NPP 2), |
| | | | | | extracellular |
| | | | | | lysophospholipase D |
| | | | | | (LysoPLD), |
| | | | | | EC: 3.1.4.39, |
| | | | | | EC: 3.1.4.4 |
| CD97 | ADGRE5 | P48960 | adhesion G | CD97, TM7LN1, | adhesion G protein- |
| | | | protein-coupled | DAF receptor | coupled receptor E5, |
| | | | receptor E5 | | leukocyte antigen |
| | | | | | CD97, adhesion G |
| | | | | | protein-coupled |
| | | | | | receptor E5 subunit |
| | | | | | alpha, adhesion G |
| | | | | | protein-coupled |
| | | | | | receptor E5 subunit |
| | | | | | beta, CD97 |
| CTACK | CCL27 | Q9Y4X3 | C-C motif | ALP, CTACK, | C-C motif |
| | | | chemokine ligand | CTAK, ESKINE, | chemokine 27, CC |
| | | | 27 | ILC, PESKY, | chemokine ILC, |
| | | | | SCYA27 | cutaneous T-cell- |
| | | | | | attracting |
| | | | | | chemokine |
| | | | | | (CTACK), ESkine, |
| | | | | | IL-11 R-alpha-locus |
| | | | | | chemokine, |
| | | | | | skinkine, small- |
| | | | | | inducible cytokine |
| | | | | | A27 |
| Integrin | ITGA1 | P56199 | integrin subunit | CD49a, VLA1, | integrin alpha-1, |
| alpha 1 | | | alpha 1 | integrin subunit α | CD49 antigen-like |
| | | | | 1, integrin α 1 | family member A, |
| | | | | | laminin and |
| | | | | | collagen receptor, |
| | | | | | VLA-1, CD49a |
| EXTL3 | EXTL3 | O43909 | exostosin like | BOTV, EXTL1L, | exostosin-like 3, |
| | | | glycosyltransferase | EXTR1, ISDNA, | EXT-related protein |
| | | | 3 | REGR, RPR, | 1, glucuronyl- |
| | | | | KIAA0519, | galactosyl- |
| | | | | LOC105379344, | proteoglycan 4- |
| | | | | LOC105379345 | alpha-N- |
| | | | | | acetylglucosaminylt |
| | | | | | ransferase, |
| | | | | | hereditary multiple |
| | | | | | exostoses gene |
| | | | | | isolog, multiple |
| | | | | | exostosis-like |
| | | | | | protein 3, putative |
| | | | | | tumor suppressor |
| | | | | | protein EXTL3, |
| | | | | | EX: 2.4.1.223 |
| IL-18 BPa | IL18BP | O95998 | interleukin 18 | FVHa, IL18BP | interleukin-18- |
| | | | binding protein | isoform 2 | binding protein, IL- |
| | | | | | 18BP, tadekinig-alfa |
| PD-L2 | PDCD1LG2 | Q9BQ51 | programmed cell | B7DC, Btdc, | programmed cell |
| | | | death 1 ligand 2 | CD273, PD-L2, | death 1 ligand 2, |
| | | | | PDCD1L2, PDL2, | PD-1 ligand 2, PD- |
| | | | | bA574F11.2 | L2, PDCD1 ligand |
| | | | | | 2, programmed |
| | | | | | death ligand 2, |
| | | | | | butyrophilin B7-DC |
| | | | | | (B7-DC), CD273 |
| PSMA | FOLH1 | Q04609 | folate hydrolase 1 | FGCP, FOLH, | glutamate |
| | | | | GCP2, GCPII, | carboxypeptidase 2, |
| | | | | NAALAD1, PSM, | cell growth- |
| | | | | PSMA, mGCP, | inhibiting gene 27 |
| | | | | NAALADase I | protein, folate |
| | | | | | hydrolase 1, |
| | | | | | folypoly-gamma- |
| | | | | | glutamate |
| | | | | | carboxypeptidase |
| | | | | | (FGCP), glutamate |
| | | | | | carboxypeptidase II |
| | | | | | (GCPII), membrane |
| | | | | | glutatmate |
| | | | | | carboxypeptidase |
| | | | | | (mGCP), N- |
| | | | | | acetylated-alpha- |
| | | | | | linked acidic |
| | | | | | dipeptidase I |
| | | | | | (NAALADase I), |
| | | | | | prostate-specific |
| | | | | | membrane antigen |
| | | | | | (PSM; PSMA), |
| | | | | | pteroylpoly-gamma- |
| | | | | | glutamate |
| | | | | | carboxypeptidase, |
| | | | | | EC: 3.4.17.21 |
| IL-20 Ra | IL20RA | Q9UHF4 | interleukin 20 | CRF2-8, IL20R- | interleukin-20 |
| | | | receptor subunit | alpha, IL-20R1, | receptor subunit |
| | | | alpha | IL-20RA, IL-20R- | alpha, IL-20 |
| | | | | alpha, IL-20R-α, | receptor subunit |
| | | | | interleukin 20 | alpha, IL-20R-alpha, |
| | | | | receptor subunit α | IL-20RA, cytokine |
| | | | | | receptor class-II |
| | | | | | member 8, cytokine |
| | | | | | receptor family 2 |
| | | | | | member 8 (CRF2- |
| | | | | | 8), IL-20R1, |
| | | | | | ZcytoR7 |
| Glyoxalase | HAGH | Q16775 | hydroxyacylglutat | GLO2, GLO2D, | hydroxyacylglutathi |
| II | | | hione hydrolase | GLX2, GLXII, | one hydrolase |
| | | | | GLXIII, HAGH, | mitochondrial, |
| | | | | HAGH1, | glyoxalase II (Glx |
| | | | | glyoxalase II | II), EC: 3.1.2.6 |
| Trypsin 1 | PRSS1 | P07477 | serine protease 1 | TRP1, TRY1, | serine protease 1, |
| | | | | TRY4, TRYP1, | anionic trypsin I, |
| | | | | TRYGN16, | anionic trypsin-I, |
| | | | | trypsin 1, ectopic | beta-trypsin, |
| | | | | anionic trypsin I, | cationic |
| | | | | cationic | trypsinogen, |
| | | | | trypsinogen | pretrypsinogen I, |
| | | | | | trypsin I, trypsin-1, |
| | | | | | EC: 3.4.21.4, alpha- |
| | | | | | trypsin chain 1, |
| | | | | | alpha-trypsin chain |
| | | | | | 2 |
| IGF-2R | IGF2R | P11717 | insulin like | CD222, CI-M6PR, | cation-independent |
| | | | growth factor 2 | CIMPR, M6P-R, | mannose-6- |
| | | | receptor | M6P/IGF2R, MPR | phosphate receptor, |
| | | | | 300, MPR1, | CI Man-6-P |
| | | | | MPR300, MPRI, | receptor, CI-MPR, |
| | | | | IGF-IIR, mannose- | M6PR, 300 kDa |
| | | | | 6-phosphate | mannose 6- |
| | | | | receptor, type 2 | phosphate receptor |
| | | | | IGF receptor, type | (MPR 300), insulin- |
| | | | | II IGF receptor | like growth factor 2 |
| | | | | | receptor, insulin-like |
| | | | | | growth factor II |
| | | | | | receptor (IGF-II |
| | | | | | receptor), |
| | | | | | M6P/IGF2 receptor |
| | | | | | (M6P/IGF2R), |
| | | | | | CD222 |
| ADAMTSL-1 | ADAMTSL1 | Q8N6G6 | ADAMTS like 1 | ADAMTSL-1, | ADAMTS-like |
| | | | | ADAMTSR1, | protein 1, |
| | | | | C9orf94, | ADAMTSL-1, |
| | | | | PUNCTIN | punctin-1 |
| Erythropoietin | EPO | P01588 | erythropoietin | DBAL, ECYT5, | erythropoietin, |
| | | | | EP, MVCD2, | epoetin |
| | | | | epoetin, epoetin | |
| | | | | alfa, epoetin alpha- | |
| | | | | epbx, epoetin alfa | |
| | | | | hexal, epoetin | |
| | | | | alpha, epoetin α, | |
| | | | | epogen, ERYPO, | |
| | | | | erythropoietin alfa, | |
| | | | | heberitro, HX575, | |
| | | | | recombinant 1-165 | |
| | | | | erythropoietin | |
| | | | | glycoform alpha, | |
| | | | | recombinant 1-165 | |
| | | | | erythropoietin α, | |
| | | | | recombinant | |
| | | | | erythropoietin- | |
| | | | | alpha, recombinant | |
| | | | | erythropoietin-α, r- | |
| | | | | huEPO, rHuEPO- | |
| | | | | alpha, rHuEPO-α | |
| Plexin D1 | PLXND1 | Q9Y4D7 | plexin D1 | CHTD9, PLEXD1, | plexin-D1 |
| | | | | KIAA0620 | |
| DNMT3A | DNMT3A | Q9Y6K1 | DNA | DNMT3A2, | |
| | | | methyltransferase | HESJAS, | |
| | | | 3 alpha | M.HsaIIIA, TBRS, | |
| | | | | DNA | |
| | | | | methyltransferase | |
| | | | | 3 α, DNA MTase | |
| | | | | HsaIIIA, MmuIIIA | |
| BCL-2 | BCL2 | P10415 | BCL2 apoptosis | Bcl-2, PPP1R50 | apoptosis regulator |
| | | | regulator | | Bcl-2 |
| CL-P1 | COLEC12 | Q5KU26 | collectin | CLP1, NSR2, | collectin-12, |
| | | | subfamily member | SCARA4, SRCL | collectin placenta |
| | | | 12 | | protein 1 (CL-P1; |
| | | | | | hCL-P1), nurse cell |
| | | | | | scavenger receptor |
| | | | | | 2, scavenger |
| | | | | | receptor class A |
| | | | | | member 4, |
| | | | | | scavenger receptor |
| | | | | | with C-type lectin |
| Ephrin-B3 | EFNB3 | Q15768 | ephrin B3 | EFL6, EPLG8, | ephrin-B3, EPH- |
| | | | | LERK8 | related receptor |
| | | | | | transmembrane |
| | | | | | ligand ELK-L3, |
| | | | | | EPH-related |
| | | | | | receptor tyrosine |
| | | | | | kinase ligand 8 |
| | | | | | (LERK-8) |
| FABP6 | FABP6 | P51161 | fatty acid binding | I-15P, I-BABP, I- | gastrotropin, GT, |
| | | | protein 6 | BALB, I-BAP, | fatty acid-binding |
| | | | | ILBP, ILBP3, | protein 6, ileal lipid- |
| | | | | ILLBP | binding protein |
| | | | | | (ILBP), intestinal 15 |
| | | | | | kDa protein (I-15P), |
| | | | | | intestinal bile acid- |
| | | | | | binding protein (I- |
| | | | | | BABP) |
| CHI3L1 | CHI3L1 | P36222 | chitinase 3 like 1 | ASRT7, CGP-39, | chitinase-3-like |
| | | | | GP-39, GP39, HC- | protein 1, 39 kDa |
| | | | | gp39, HCGP-3P- | synovial protein, |
| | | | | YK-40, YKL-40, | cartilage |
| | | | | YKL40, YYL-40, | glycoprotein 39 |
| | | | | hCGP-39, | (CGP-39; GP-39; |
| | | | | cartilage | hCGP-39), YKL-40 |
| | | | | glycoprotein-39, | |
| | | | | HCGP-3P, YK-40 | |
| FCRLS | FCRL2 | Q96LA5 | Fc receptor like 2 | CD307b, FCRH2, | Fc receptor-like |
| | | | | IFGP4, IRTA4, | protein 2, FcR-like |
| | | | | SPAP1, SPAP1A, | protein 2, FcRL2, Fc |
| | | | | SPAP1B, SPAP1C | receptor homolog 2 |
| | | | | | (FcRH2), IFGP |
| | | | | | family protein 4, |
| | | | | | immunoglobulin |
| | | | | | receptor |
| | | | | | translocation- |
| | | | | | associated protein 4, |
| | | | | | SH2 domain- |
| | | | | | containing |
| | | | | | phosphatase anchor |
| | | | | | protein 1, CD307b |
| | FCRLB | Q6BAA4 | Fc receptor like B | FCRL2, FCRLM2, | Fc receptor-like B, |
| | | | | FCRLY, FREB-2, | Fc receptor homolog |
| | | | | FREB2, FcRY | expressed in B-cells |
| | | | | | protein 2 (FREB-2), |
| | | | | | Fc receptor-like and |
| | | | | | mucin-like protein |
| | | | | | 2, Fc receptor-like |
| | | | | | protein 2, Fc |
| | | | | | receptor-related |
| | | | | | protein Y (FcRY) |
| TFF3 | TFF3 | Q07654 | trefoil factor 3 | ITF, P1B, TFI, | trefoil factor 3, |
| | | | | P1.b, TIFF3 | intestinal trefoil |
| | | | | | factor (hITF), |
| | | | | | polypeptide P1.B |
| | | | | | (hP1.B) |
| Artemin | ARTN | Q5T4W7 | artemin | ART, ENOVIN, | artemin, enovin, |
| | | | | EVN, NBN | neublastin |
| DPPII | DPP3 | Q9NY33 | dipeptidyl | DPPIII | dipeptidyl peptidase |
| | | | peptidase 3 | | 3, dipeptidyl |
| | | | | | aminopeptidase III, |
| | | | | | dipeptidy1 |
| | | | | | arylamidase III, |
| | | | | | dipeptidyl peptidase |
| | | | | | III (DPP III), |
| | | | | | enkephalinase B, |
| | | | | | EC: 3.4.14.4 |
| cIAP-1 | BIRC2 | Q13490 | baculoviral IAP | API1, HIAP2, | baculoviral IAP |
| | | | repeat containing | Hiap-2, MIHB, | repeat-containing |
| | | | 2 | RNF48, c-IAP1, | protein 2, cellular |
| | | | | cIAP1, Hiap2 | inhibitor of |
| | | | | | apoptosis 1 (C- |
| | | | | | IAP1), IAP homolog |
| | | | | | B, inhibitor of |
| | | | | | apoptosis protein 2 |
| | | | | | (hIAP-2; hIAP2), |
| | | | | | RING finger protein |
| | | | | | 48, RING-type E3 |
| | | | | | ubiquitin transferase |
| | | | | | BIRC2, TNFR2- |
| | | | | | TRAF-signaling |
| | | | | | complex protein 2 |
| PDGF Rb | PDGFRB | P09619 | platelet derived | CD140B, IBGC4, | platelet-derived |
| | | | growth factor | IMF1, JTK12, | growth factor |
| | | | receptor beta | KOGS, PDGFR, | receptor beta, |
| | | | | PDGFR-1, | PDGF-R-beta, |
| | | | | PDGFR1, PENTT, | PDGFR-beta, beta |
| | | | | PDGFR-beta, | platelet-derived |
| | | | | PDGFR-β, | growth factor |
| | | | | PDGFbetaR, | receptor, beta-type |
| | | | | PDGFRbeta, Pdgf | platelet-derived |
| | | | | receptor β, | growth factor |
| | | | | PDGFRβ, platelet | receptor, CD140 |
| | | | | derived growth | antigen-like family |
| | | | | factor β | member B, platelet- |
| | | | | | derived growth |
| | | | | | factor receptor 1 |
| | | | | | (PDGFR-1), |
| | | | | | CD140b, |
| | | | | | EC: 2.7.10.1 |
| Pentraxin 3 | PTX3 | P26022 | pentraxin 3 | TNFAIP5, TSG- | pentraxin-related |
| | | | | 14, TSG14 | protein PTX3, |
| | | | | | pentaxin-related |
| | | | | | protein PTX3, tumor |
| | | | | | necrosis factor |
| | | | | | alpha-induced |
| | | | | | protein 5 (TNF |
| | | | | | alpha-induced |
| | | | | | protein 5), tumor |
| | | | | | necrosis factor- |
| | | | | | inducible gene 14 |
| | | | | | protein (TSG-14) |
| Angiotensinogen | AGT | P01019 | angiotensinogen | ANHU, | angiotensinogen, |
| | | | | SERPINA8, | serpin A8, |
| | | | | hFLT1, AGTN, | angiotensin-1, |
| | | | | ANG, | angiotensin1-10, |
| | | | | LOC105373166 | angiotensin I (Ang |
| | | | | | I), angiotensin-2, |
| | | | | | angiotensin 1-8, |
| | | | | | angiotensin II (Ang |
| | | | | | II), angiotensin-3, |
| | | | | | angiotensin 2-8, |
| | | | | | angiotensin III (Ang |
| | | | | | III), Des-Asp[1]- |
| | | | | | angiotensin II, |
| | | | | | angiotensin-4, |
| | | | | | angiotensin 3-8, |
| | | | | | angiotensin IV (Ang |
| | | | | | IV), angiotensin 1-9, |
| | | | | | angiotensin 1-7, |
| | | | | | angiotensin 1-5, |
| | | | | | angiotensin 1-4 |
| Follistatin | FST | P19883 | follistatin | FS | follistatin, FS, |
| | | | | | activin-binding |
| | | | | | protein |
| CF VII | F7 | P08709 | coagulation factor | SPCA, coagulation | coagulation factor |
| | | | VII | factor VIIa | VII, proconvertin, |
| | | | | (recombinant) | serum prothrombin |
| | | | | | conversion |
| | | | | | accelerator (SPCA), |
| | | | | | factor VII light |
| | | | | | chain, factor VII |
| | | | | | heavy chain, |
| | | | | | eptacog alfa, |
| | | | | | EC: 3.4.21.21 |
| Persephin | PSPN | O60542 | persephin | PSP | persephin, PSP |
| TRAIL R1 | TNFRSF | O00220 | TNF receptor | APO2, CD261, | tumor necrosis |
| | 10A | | superfamily | DR4, TRAILR-1, | factor receptor |
| | | | member 10a | TRAILR1, death | superfamily member |
| | | | | receptor 4, TRAIL | 10A, death receptor |
| | | | | receptor-1 | 4, TNF-related |
| | | | | | apoptosis-inducing |
| | | | | | ligand receptor 1 |
| | | | | | (TRAIL receptor 1; |
| | | | | | TRAIL-R1), CD261 |
| THAP11 | THAP11 | Q96EK4 | THAP domain | CTG-B43a, CTG- | THAP domain- |
| | | | containing 11 | B45d. | containing protein |
| | | | | HRIHFB2206, | 11 |
| | | | | MAHCL, RONIN, | |
| | | | | SCA51 | |
| CD200 | CD200 | P41217 | CD200 molecule | MOX1, MOX2, | OX-2 membrane |
| | | | | MRC, OX-2 | glycoprotein, |
| | | | | | CD200 |
| CLEC-2 | CLEC1B | Q9P126 | C-type lectin | 1810061I13Rik, | C-type lectin |
| | | | domain family 1 | CLEC2, CLEC2B, | domain family 1 |
| | | | member B | PRO1384, | member B, C-type |
| | | | | QDED721, CLEC- | lectin-like receptor 2 |
| | | | | 2 | (CLEC-2) |
| AMIGO | AMIGO1 | Q86WK6 | adhesion molecule | ALI2, AMIGO, | amphoterin-induced |
| | | | with Ig like | AMIGO-1, | protein 1, AMIGO- |
| | | | domain 1 | KIAA1163 | 1, alivin-2 |
| | AMIGO2 | Q86SJ2 | adhesion molecule | ALI1, AMIGO-2, | amphoterin-induced |
| | | | with Ig like | DEGA, | protein 2, AMIGO- |
| | | | domain 2 | AC004010, | 2, alivin-1, |
| | | | | LOC102724147 | differentially |
| | | | | | expressed in gastric |
| | | | | | adenocarcinomas |
| | | | | | (DEGA) |
| | AMIGO3 | Q86WK7 | adhesion molecule | ALI3, AMIGO-3, | amphoterin-induced |
| | | | with Ig like | KIAA1851 | protein 3, AMIGO- |
| | | | domain 3 | | 3, alivin-3 |
| IGFBP-5 | IGFBP5 | P24593 | insulin like | IBP5 | insulin-like growth |
| | | | growth factor | | factor-binding |
| | | | binding protein 5 | | protein 5, IBP-5, |
| | | | | | IGF-binding protein |
| | | | | | 5, IGFBP-5 |
| PON1 | PON1 | P27169 | paraoxonase 1 | ESA, MVCD5, | serum |
| | | | | PON, A-esterase 1 | paraoxonase/arylest |
| | | | | | erase 1, PON 1, |
| | | | | | aromatic esterase 1 |
| | | | | | (A-esterase 1), K- |
| | | | | | 45, serum |
| | | | | | aryldialkylphosphatase |
| | | | | | 1, EC: 3.1.1.2, |
| | | | | | EC: 3.1.1.81, |
| | | | | | EC: 3.1.8.1 |
| SOX7 | SOX7 | Q9BT81 | SRY-box | | transcription factor |
| | | | transcription | | SOX-7 |
| | | | factor 7 | | |
| GALNT10 | GALNT10 | Q86SR1 | polypeptide N- | GALNACT10, | polypeptide N- |
| | | | acetylgalactosami | PPGALNACT10, | acetylgalactosaminy |
| | | | nyltransferase 10 | PPGANTASE10 | Itransferase 10, |
| | | | | | polypeptide GalNAc |
| | | | | | transferase 10 |
| | | | | | (GalNAc-T10; pp- |
| | | | | | GaNTase 10), |
| | | | | | protein-UDP |
| | | | | | acetylgalactosaminy |
| | | | | | Itransferase 10, |
| | | | | | UDP- |
| | | | | | GalNAc: polypeptide |
| | | | | | N- |
| | | | | | acetylgalactosaminy |
| | | | | | ltransferase 10, |
| | | | | | EC: 2.4.1.41 |
| Visfatin | NAMPT | P43490 | nicotinamide | 1110035O14Rik, | nicotinamide |
| | | | phosphoribosyltra | PBEF, PBEF1, | phosphoribosyltrans |
| | | | nsferase | VF, VISFATIN, | ferase, |
| | | | | pre-B-cell colony | NAmPRTase, |
| | | | | enhancing factor, | Nampt, pre-B-cell |
| | | | | pre-B-cell colony | colony-enhancing |
| | | | | enhancing factor 1 | factor 1 (pre-B cell- |
| | | | | | enhancing factor), |
| | | | | | visfatin, EC: 2.4.2.12 |
| Progranulin | GRN | P28799 | granulin precursor | CLN11, FTD2, | progranulin, PGRN, |
| | | | | GEP, GP88, | acrogranin, epithelin |
| | | | | PCDGF, PEPI, | precursor, |
| | | | | PGRN, | glycoprotein of 88 |
| | | | | proepithelin, | kDa (GP88; |
| | | | | progranulin | glycoprotein 88), |
| | | | | | granulin precursor, |
| | | | | | PC cell-derived |
| | | | | | growth factor |
| | | | | | (PCDGF), |
| | | | | | proepithelin (PEPI), |
| | | | | | paragranulin, |
| | | | | | granulin-1 (granulin |
| | | | | | G), granulin-2 |
| | | | | | (granulin F), |
| | | | | | granulin-3 |
| | | | | | (epithelin-2; |
| | | | | | granulin B), |
| | | | | | granulin-4 |
| | | | | | (epithelin-1; |
| | | | | | granulin A), |
| | | | | | granulin-5 (granulin |
| | | | | | C), granulin-6 |
| | | | | | (granulin D), |
| | | | | | granulin-7 (granulin |
| | | | | | E) |
| PCSK2 | PCSK2 | P165419 | proprotein | NEC 2, NEC-2, | neuroendocrine |
| | | | convertase | NEC2, PC2, SPC2 | convertase 2, NEC |
| | | | subtilisin/kexin | | 2, KEX2-like |
| | | | type 2 | | endoprotease 2, |
| | | | | | prohormone |
| | | | | | convertase 2, |
| | | | | | proprotein |
| | | | | | convertase 2 (PC2), |
| | | | | | EC: 3.4.21.94 |
| GKN1 | GKN1 | Q9NS71 | gastrokine 1 | AMP18, BIRCD1, | gastrokine-1, 18 |
| | | | | CA11, FOV, | kDa antrum mucosa |
| | | | | foveolin | protein (AMP-18), |
| | | | | | protein CA11 |
| IL-18 | IL18 | Q14116 | interleukin 18 | IGIF, IL-18, IL- | interluekin-18, IL- |
| | | | | 1g, IL1F4, | 18, iboctadekin, |
| | | | | interferon-gamma- | interferon gamma- |
| | | | | inducing factor, | inducing factor |
| | | | | interferon-γ- | (IFN-gamma- |
| | | | | inducing factor | inducing factor), |
| | | | | | interleukin-1 gamma |
| | | | | | (IL-1 gamma) |
| Neprilysin | MME | P08473 | membrane | CALLA, CD10, | neprilysin, |
| | | | metalloendopeptid | CMT2T, NEP, | atriopeptidase, |
| | | | ase | SCA43, SFE, | common acute |
| | | | | EPN, Neutral | lymphocytic |
| | | | | Brush-Border | leukemia antigen |
| | | | | Endopeptidase | (CALLA), |
| | | | | | enkephalinase, |
| | | | | | neutral |
| | | | | | endopeptidase 24.11 |
| | | | | | (NEP; neutral |
| | | | | | endopeptidase), skin |
| | | | | | fibroblast elastase |
| | | | | | (SFE), CD10, |
| | | | | | EC: 3.4.24.11 |
| Stabilin-2 | STAB2 | Q8WWQ8 | stabilin 2 | FEEL2, FELE-2, | stabilin-2, FAS1 |
| | | | | FELL2, FEX2, | EGF-like and X-link |
| | | | | HARE, SCARH1, | domain-containing |
| | | | | FELL | adhesion molecule |
| | | | | | 2, fasciclin EGF-like |
| | | | | | laminin-type EGF- |
| | | | | | like and link |
| | | | | | domain-containing |
| | | | | | scavenger receptor 2 |
| | | | | | (FEEL-2), |
| | | | | | hyaluranon receptor |
| | | | | | for endocytosis, 190 |
| | | | | | kDa form stabilin-2 |
| | | | | | (190 kDa |
| | | | | | hyaluranon receptor |
| | | | | | for endocytosis) |
| IL-17 RD | IL17RD | Q8NFM7 | interluekin 17 | HH18, IL-17RD, | interleukin-17 |
| | | | receptor D | IL17RLM, SEF | receptor D, IL-17 |
| | | | | | receptor D, IL- |
| | | | | | 17RD, IL17Rhom, |
| | | | | | interleukin-17 |
| | | | | | receptor-like |
| | | | | | protein, Sef |
| | | | | | homolog (hSef) |
| Albumin | ALB | P02768 | albumin | FDAHT, HSA, | albumin |
| | | | | PRO0883, | |
| | | | | PRO0903, | |
| | | | | PRO1341, | |
| | | | | pRo2044, albumin | |
| | | | | isomer 1, albumin | |
| | | | | isomer 2, albumin | |
| | | | | isomer 3, serum | |
| | | | | albumin, serum | |
| | | | | albumin chain A | |
| Follistatin- | FSTL1 | Q12841 | follistatin like 1 | FRP, FSL1, | follistatin-related |
| like 1 | | | | MIR198, OCC-1, | protein 1, follistatin- |
| | | | | OCC1, tsc36 | like protein 1 |
| MMP-10 | MMP10 | P09238 | matrix | SL-2, STMY2, | stromelysin-2, SL-2, |
| | | | metallopeptidase | stromelysin 2 | matrix |
| | | | 10 | | metalloproteinase- |
| | | | | | 10 (MMP-10), |
| | | | | | transin-2, |
| | | | | | EC: 3.4.24.22 |
| FKBP51 | FKPB5 | Q13451 | FKBP prolyl | AIG61, FKBP54, | peptidy1-proly1 cis- |
| | | | isomerase 5 | P54, PPIase, Ptg- | trans isomerase |
| | | | | 10, FKBP51 | FKBP5, PPIase |
| | | | | | FKBP5, 51 kDa |
| | | | | | FK506-binding |
| | | | | | protein (51 kDa |
| | | | | | FKBP; FKBP-51), |
| | | | | | 54 kDa progesterone |
| | | | | | receptor-associated |
| | | | | | immunophilin, |
| | | | | | androgen-regulated |
| | | | | | protein 6, FF1 |
| | | | | | antigen, FK506- |
| | | | | | binding protein |
| | | | | | (FKBP-5), FKBP54 |
| | | | | | (p54), HSP90- |
| | | | | | binding |
| | | | | | immunophilin, |
| | | | | | rotamase |
| LRRC4 | LRRC4 | Q9HBW1 | leucine rich repeat | NAG14, NGL-2, | leucine-rich repeat- |
| | | | containing 4 | BAG | containing protein 4, |
| | | | | | brain tumor- |
| | | | | | associated protein |
| | | | | | BAG. |
| | | | | | nasopharyngeal |
| | | | | | carcinoma- |
| | | | | | associated gene 14 |
| | | | | | protein, netrin-G2 |
| | | | | | ligand (NGL-2) |
| Pref-1 | DLK1 | P80370 | delta like non- | DLK, DLK-1, | protein delta |
| | | | canonical Notch | Delta1, FA1, | homolog 1, DLK-1, |
| | | | ligand 1 | PREF1, Pref-1, | pG2, fetal antigen 1 |
| | | | | ZOG, pG2, delta, | (FA1) |
| | | | | δ, δ-like dlk | |
| | | | | homeotic, δ like | |
| | | | | non-canonical | |
| | | | | Notch ligand 1 | |
| Galectin-1 | LGALS1 | P09382 | galectin 1 | GAL1, GBP, | galectin-1, Gal-1, 14 |
| | | | | 14kDa lectin, beta | kDa laminin-binding |
| | | | | galactoside | protein (HLBP14), |
| | | | | binding lectin | 14 kDa lectin, beta- |
| | | | | precursor, | galactoside-binding |
| | | | | betaGBP, galaptin, | lectin L-14-I, |
| | | | | β galactoside | galaptin, HBL, HPL, |
| | | | | binding lectin | lactose-binding |
| | | | | precursor | lectin 1, lectin |
| | | | | | galactoside-binding |
| | | | | | soluble 1, putative |
| | | | | | MAPK-activating |
| | | | | | protein PM12, S- |
| | | | | | Lac lectin 1 |
| Troponin C | TNNC1 | P63316 | troponin C1, slow | CMD1Z, CMH13, | troponin C slow |
| | | | skeletal and | TN-C, TNC, | skeletal and cardiac |
| | | | cardiac type | TNNC, cardiac | muscles, TN-C |
| | | | | troponin C | |
| | TNNC2 | P02585 | troponin C2, fast | CFAP85, | troponin C skeletal |
| | | | skeletal type | CMYO15, | muscle |
| | | | | CMYP15, FAP85, | |
| | | | | MYONRI, TnCf | |
| UNC5H3 | UNC5C | O95185 | unc-5 netrin | UNC5H3, | netrin receptor |
| | | | receptor C | LOC101929250, | UNC5C, protein |
| | | | | Unc-5 homolog C | unc-5 homolog 3, |
| | | | | | protein unc-5 |
| | | | | | homolog C |
| FLRT2 | FLRT2 | O43155 | fibronectin leucine | KIAA0405, | leucine-rich repeat |
| | | | rich | LOC100506718, | transmembrane |
| | | | transmembrane | LOC102724348, | protein FLRT2, |
| | | | protein 2 | LOC107984662, | fibronectin-like |
| | | | | LOC124907389 | domain-containing |
| | | | | | leucine-rich |
| | | | | | transmembrane |
| | | | | | protein 2 |
| CD314 | KLRK1 | P26718 | killer cell lectin | CD314, | NKG2-D type II |
| | | | like receptor K1 | D12S2489E, KLR, | integral membrane |
| | | | | NKG2-D, | protein, killer cell |
| | | | | | lectin-like receptor |
| | | | | NKG2D, NK cell | subfamily K |
| | | | | receptor D | member 1, NK cell |
| | | | | | receptor D, NKG2- |
| | | | | | D-activating NK |
| | | | | | receptor, CD314 |
| Semaphorin | SEMA6B | Q9H3T3 | semaphorin 6B | EPM11, SEM- | semaphorin-6B, |
| 6B | | | | SEMA-Y, SEM- | semaphorin-Z |
| | | | | SEMA-Z, SEMA- | (Sema Z) |
| | | | | VIB, SEMAN, | |
| | | | | semaZ, SEMAZ | |
| Netrin-4 | NTN4 | Q9HB63 | netrin 4 | PRO3091 | netrin-4, beta-netrin, |
| | | | | | hepar-derived |
| | | | | | netrin-like protein |
| CD27 | CD70 | P32970 | CD70 molecule | CD27-L, CD27L, | CD70 antigen, |
| Ligand | | | | CD27LG, LPFS3, | CD27 ligand |
| | | | | TNFSF7, | (CD27-L), tumor |
| | | | | TNLG8A | necrosis factor |
| | | | | | ligand superfamily |
| | | | | | member 7, CD70 |
| IL-20 R beta | IL20RB | Q6UXL0 | interleukin 20 | DIRS1, FNDC6, | interleukin-20 |
| | | | receptor subunit | IL-20R2, | receptor subunit |
| | | | beta | interleukin 20 | beta, IL-20 receptor |
| | | | | receptor subunit β | subunit beta, IL- |
| | | | | | 20R-beta, IL-20RB, |
| | | | | | fibronectin type III |
| | | | | | domain containing 6 |
| | | | | | (FNDC6), IL-20R2 |
| Semaphorin | SEMA6A | Q9H2E6 | semaphorin 6A | HT018, SEMA1, | semaphorin-6A, |
| 6A | | | | SEMAQ, VIA, | semaphorin VIA |
| | | | | SEMA6A, | (Sema VIA), |
| | | | | KIAA1368, | semaphorin-6A-1 |
| | | | | SEMA6A1 | (SEMA6A-1) |
| TSK | TSKU | Q8WUA8 | tsukushi, small | E2IG4, LRRC54, | tsukushi, E2- |
| | | | leucine rich | TSK | induced gene 4 |
| | | | proteoglycan | | protein, leucine-rich |
| | | | | | repeat-containing |
| | | | | | protein 54 |
| Cytokeratin- | KRT8 | P05787 | keratin 8 | CARD2, CK-8, | keratin type II |
| 8 | | | | CK8, CYK8, | cytoskeletal 8, |
| | | | | K2C8, K8, KO | cytokeratin-8 (CK- |
| | | | | | 8), keratin-8 (K8), |
| | | | | | type-II keratin Kb8 |
| CHST3 | CHST3 | Q7LGC8 | carbohydrate | C6ST, C6ST1, | carbohydrate |
| | | | sulfotransferase 3 | HSD, | sulfotransferase 3, |
| | | | | carbohydrate | chondroitin 6-O- |
| | | | | sulphotransferase | sulfotransferase 1 |
| | | | | 3 | (C6ST-1), |
| | | | | | chondroitin 6- |
| | | | | | sulfotransferase |
| | | | | | (C6ST), |
| | | | | | galactose/N- |
| | | | | | acetylglucosamine/ |
| | | | | | N- |
| | | | | | acetylglucosamine |
| | | | | | 6-O-sulfotransferase |
| | | | | | 0 (GST-0), |
| | | | | | EC: 2.8.2.17, |
| | | | | | EC: 2.8.2.21 |
| Mc1-1 | MCL1 | Q07820 | MCL1 apoptosis | BCL2L3, EAT- | induced myeloid |
| | | | regulator, BCL2 | ES, MCL1L, | leukemia cell |
| | | | family member | MCL1S, Mcl-1, | differentiation |
| | | | | TM, bcl2-L-3, | protein Mcl-1, Bcl- |
| | | | | mcl1/EAT, EAT | 2-like protein 3 |
| | | | | | (Bcl2-L-3), Bcl-2- |
| | | | | | related protein |
| | | | | | EAT/mcl1, |
| | | | | | mcl1/EAT |
| DPPIV | DPP4 | P27487 | dipeptidyl | ADABP, ADCP2, | dipeptidyl peptidase |
| | | | peptidase 4 | CD26, DPPIV, | 4, ADABP, |
| | | | | TP103, adenosine | adenosine |
| | | | | deaminase | deaminase |
| | | | | binding, | complexing protein |
| | | | | glycylprolyl beta- | 2 (ADCP-2), |
| | | | | Naphthylamidase, | dipeptidyl peptidase |
| | | | | glycylprolyl β- | IV (DPP IV), T-cell |
| | | | | Naphthylamidase | activation antigen |
| | | | | | CD26, TP103, |
| | | | | | dipeptidyl peptidase |
| | | | | | 4 membrane form |
| | | | | | (dipeptidy1 |
| | | | | | peptidase IV |
| | | | | | membrane form), |
| | | | | | dipeptidyl peptidase |
| | | | | | 4 soluble form |
| | | | | | (dipeptidyl |
| | | | | | peptidase IV soluble |
| | | | | | form), CD26, |
| | | | | | EC: 3.4.14.5 |
| SREC-II | SCARF2 | Q96GP6 | scavenger receptor | NSR1, SREC-II, | scavenger receptor |
| | | | class F member 2 | SREC2, SRECRP- | class F member 2, |
| | | | | 1, VDEGS, | SRECRP-1, |
| | | | | SREPCR | scavenger receptor |
| | | | | | expressed by |
| | | | | | endothelial cells 2 |
| | | | | | protein (SREC-II) |
| Norrin | NDP | Q00604 | norrin cystine | EVR2, FEVR, ND | norrin, norrie |
| | | | knot growth factor | | disease protein, X- |
| | | | NDP | | linked exudative |
| | | | | | vitreoretinopathy 2 |
| | | | | | protein |
| JAM-C | JAM3 | Q9BX67 | junctional | JAM-2, JAM-3, | junctional adhesion |
| | | | adhesion molecule | JAM-C, JAMC | molecule C, JAM-C, |
| | | | 3 | | JAM-2, junctional |
| | | | | | adhesion molecule 3 |
| | | | | | (JAM-3), soluble |
| | | | | | form of JAM-C |
| | | | | | (sJAM-C) |
| Bc1-10 | BCL10 | O95999 | BCL10 immune | CARMEN, | B-cell |
| | | | signaling adaptor | CIPER, CLAP, | lymphoma/leukemia |
| | | | | IMD37, c-E10, | 10, B-cell |
| | | | | mE10, B cell | CCL/lymphoma 10 |
| | | | | CLL/lymphoma 10 | (Bc1-10), CARD- |
| | | | | | containing molecule |
| | | | | | enhancing NF- |
| | | | | | kappa-B, CARD- |
| | | | | | like apoptotic |
| | | | | | protein (hCLAP), |
| | | | | | CED-3/ICH-1 |
| | | | | | prodomain |
| | | | | | homologous E10- |
| | | | | | like regulator |
| | | | | | (CIPER), cellular |
| | | | | | homolog of |
| | | | | | vCARMEN |
| | | | | | (cCARMEN), |
| | | | | | cellular-E10 (c- |
| | | | | | E10), mammalian |
| | | | | | CARD-containing |
| | | | | | adapter molecule |
| | | | | | E10 (mE10) |
| Wnt-4 | WNT4 | P56705 | Wnt family | SERKAL, WNT-4 | protein Wnt-4 |
| | | | member 4 | | |
| LSECtin | CLEC4G | Q6UXB4 | C-type lectin | DTTR431, | C-type lectin |
| | | | domain family 4 | LP2698, LSECtin, | domain family 4 |
| | | | member G | UNQ431 | member G, liver and |
| | | | | | lymph node |
| | | | | | sinusoidal |
| | | | | | endothelial cell C- |
| | | | | | type lectin |
| | | | | | (LSECtin) |
| Kell | KEL | P23276 | Kell metallo- | CD238, ECE3, | Kell blood group |
| | | | endopeptidase | Kell, KELL | glycoprotein, |
| | | | (Kell blood group) | | CD238, EC: 3.4.24.- |
| TNF RI | TNFRSF | P19438 | TNF receptor | CD120a, FPF, | tumor necrosis |
| | 1A | | superfamily | TBP1, TNF-R, | factor receptor |
| | | | member 1A | TNF-R-I, TNF- | superfamily member |
| | | | | R55, TNFAR, | 1A, tumor necrosis |
| | | | | TNFR1, TNFR55, | factor receptor 1 |
| | | | | TNFR60, p55, | (TNF-R1), tumor |
| | | | | p55-R, p60, p55 | necrosis factor |
| | | | | TNF alpha | receptor type I |
| | | | | receptor, p55 TNF | (TNF-RI; TNFR-I), |
| | | | | α receptor | p55, p60, CD120a, |
| | | | | | tumor necrosis |
| | | | | | factor receptor |
| | | | | | superfamily member |
| | | | | | 1A membrane form, |
| | | | | | tumor necrosis |
| | | | | | factor-binding |
| | | | | | protein 1 (TBPI) |
| PTP1B | PTPN1 | P18031 | protein tyrosine | PTP1B, PTP1B | tyrosine-protein |
| | | | phosphatase non- | alpha, PTP1BB, | phosphatase non- |
| | | | receptor type 1 | PTP1B α | receptor type 1, |
| | | | | | protein-tyrosine |
| | | | | | phosphatase 1B |
| | | | | | (PTP-1B), |
| | | | | | EC: 3.1.3.48 |
| htPAPP-A | This is the heterotetrameric form of the | | | htPAPPA, | heterotetrameric |
| | protein PAPPA (PAPPA is row 47 of this | | | htPAPP-A, PAPP- | PAPPA, |
| | document). It consists of 2 PAPP-A | | | A/proMBP | heterotetrameric |
| | subunits, and 2 proMBP subunits. | | | | PAPP-A, htPAPPA |
| | | | | | heterotetrameric |
| | | | | | complex, htPAPP-A |
| | | | | | heterotetrameric |
| | | | | | complex, |
| | | | | | pregnancy- |
| | | | | | associated plasma |
| | | | | | protein |
| | | | | | A/proeosinophil |
| | | | | | major basic protein |
| | | | | | complex |
| | PRG2 | P13727 | proteoglycan 2, | BMPG, MBP, | bone marrow |
| | | | pro eosinophil | MBP1, proMBP | proteoglycan, |
| | | | major basic | | BMPG, |
| | | | protein | | proteoglycan 2, |
| | | | | | eosinophil granule |
| | | | | | major basic protein |
| | | | | | (EMBP; MBP), |
| | | | | | pregnancy- |
| | | | | | associated major |
| | | | | | basic protein |
| IDO | IDO1 | P14902 | indoleamine 2,3- | IDO, IDO-1, | indoleamine 2,3- |
| | | | dioxygenase 1 | INDO, IFN | idoxygenase 1, IDO- |
| | | | | gamma inducible | 1, indoleamine- |
| | | | | indoleamine 2-3 | pyrrole 2,3- |
| | | | | dioxygenase, IFN | |
| | | | | γ inducible | dioxygenase, |
| | | | | indoleamine 2-3 | EC: 1.13.11.52 |
| | | | | dioxy genase, | |
| | | | | indole 2,3 | |
| | | | | dioxygenase | |
| PDGF-CC | PDGFC | Q9NRA1 | platelet derived | FALLOTEIN, | PDGFC dimer, |
| | | | growth factor C | SCDGF | Pdgfc dimer, PDGF- |
| | | | | | C, fallotein, spinal |
| | | | | | cord-derived growth |
| | | | | | factor (SCDGF), |
| | | | | | VEGF-E, platelet- |
| | | | | | derived growth |
| | | | | | factor C latent form |
| | | | | | (PDGFC latent |
| | | | | | form), platelet- |
| | | | | | derived growth |
| | | | | | factor C receptor- |
| | | | | | binding form |
| | | | | | (PDGFC receptor- |
| | | | | | binding form) |
| Galanin | GAL | P22466 | galanin and | ETL8-GMAP, | galanin peptides, |
| | | | GMAP | GALN, GLNN, | galanin, galanin |
| | | | prepropeptide | GMAP, GAL1, | message-associated |
| | | | | ETL8, GAL- | peptide (GMAP) |
| | | | | GMAP | |
| Activin A | INHBA | P08476 | inhibin subunit | EDF, FRP, activin | inhibin beta A |
| | | | beta A | β A, beta A | chain, activin beta-A |
| | | | | inhibin, inhibin A, | chain, erythroid |
| | | | | inhibin beta A, | differentiation |
| | | | | inhibin subunit | protein (EDF), |
| | | | | beta A, inhibin | follicle-stimulating |
| | | | | subunit β A, | hormone (FSH)- |
| | | | | inhibin β a | releasing protein |
| | | | | subunit, β A | (FRP), FSH- |
| | | | | inhibin | releasing protein |
| TLR2 | TLR2 | O60603 | toll like receptor 2 | CD282, TIL4 | toll-like receptor 2, |
| | | | | | toll/interluekin-1 |
| | | | | | receptor-like protein |
| | | | | | 4, CD282 |
| SCCA2 | SERPINB4 | P48594 | serpin family B | LEUPIN, PI11, | serpin B4, leupin, |
| | | | member 4 | SCCA-2, SCCA1, | peptidase inhibitor |
| | | | | SCCA2 | 11 (PI-11), |
| | | | | | squamous cell |
| | | | | | carcinoma antigen 2 |
| | | | | | (SCCA-2) |
| FABP1 | FABP1 | P07148 | fatty acid binding | FABPL, L-FABP | fatty acid-binding |
| | | | protein 1 | | protein liver, fatty |
| | | | | | acid-binding protein |
| | | | | | 1, liver-type fatty |
| | | | | | acid-binding protein |
| | | | | | (L-FABP) |
| eNOS | NOS3 | P29474 | nitric oxide | ECNOS, eNOS | constitutive NOS |
| | | | synthase 3 | | (cNOS), EC-NOS, |
| | | | | | NOS type III |
| | | | | | (NOSIII), nitric |
| | | | | | oxide synthase |
| | | | | | endothelial |
| | | | | | (endothelial NOS; |
| | | | | | eNOS), |
| | | | | | EC: 1.14.13.39 |
| SHP-1 | PTPN6 | P29350 | protein tyrosine | HCP, HCPH, | tyrosine-protein |
| | | | phosphatase non- | HPTP1C, PTP-1C, | phosphatase non- |
| | | | receptor type 6 | SH-PTP1, SHP-1, | receptor type 6, |
| | | | | SHP-1L, SHP1, | hematopoietic cell |
| | | | | PTP1C | protein-tyrosine |
| | | | | | phosphatase, |
| | | | | | protein-tyrosine |
| | | | | | phosphatase 1C |
| | | | | | (PTP-1C), protein- |
| | | | | | tyrosine phosphatase |
| | | | | | SHP-1, SH-PTP1 |
| ICOS | ICOS | Q9Y6W8 | inducible T cell | AILIM, CD278, | inducible T-cell |
| | | | costimulator | CVID1 | costimulator, |
| | | | | | activation-inducible |
| | | | | | lymphocyte |
| | | | | | immunomediatory |
| | | | | | molecule, CD278 |
| C1qTNF9 | C1QTNF9 | P0C862 | C1q and TNF | AQL1A, CTRP9, | complement C1q |
| | | | related 9 | C1QTNF9A, | and tumor necrosis |
| | | | | AQL1 | factor-related |
| | | | | | protein 9A, |
| | | | | | complement C1q |
| | | | | | and tumor necrosis |
| | | | | | factor-related |
| | | | | | protein 9 |
| | C1QTNF9B | B2RNN3 | C1q and TNF | CTRP9B | complement C1q |
| | | | related 9B | | and tumor necrosis |
| | | | | | factor-related |
| | | | | | protein 9B, |
| | | | | | C1q/TNF-related |
| | | | | | protein 9B |
| | | | | | (CTRP9B), |
| | | | | | complement C1q |
| | | | | | and tumor necrosis |
| | | | | | factor-related |
| | | | | | protein 9-like |
| MMP-1 | MMP1 | P03956 | matrix | CLG, CLGN, | interstitial |
| | | | metallopeptidase 1 | MMP1a, | collagenase, |
| | | | | collagenase, | fibroblast |
| | | | | collagenase-1 | collagenase, matrix |
| | | | | | metalloproteinase-1 |
| | | | | | (MMP-1), 22 kDa |
| | | | | | interstitial |
| | | | | | collagenase, 27 kDa |
| | | | | | interstitial |
| | | | | | collagenase, |
| | | | | | EC: 3.4.24.7 |
| TC-PTP | PTPN2 | P17706 | protein tyrosine | PTN2, PTPT, TC- | tyrosine-protein |
| | | | phosphatase non- | PTP, TC45, TC48, | phosphatase non- |
| | | | receptor type 2 | TCELLPTP, | receptor type 2, T- |
| | | | | TCPTP, PTPase, T | cell protein-tyrosine |
| | | | | CELL PTPASE | phosphatase |
| | | | | | (TCPTP), |
| | | | | | EC: 3.1.3.48 |
| IL-24 | IL24 | Q13007 | interleukin 24 | C49A, FISP, | IL-24, melanoma |
| | | | | IL10B, MDA7, | differentiation- |
| | | | | MOB5, ST16 | associated gene 7 |
| | | | | | protein (MDA-7), |
| | | | | | suppression of |
| | | | | | tumorigenicity 16 |
| | | | | | protein |
| gp130 | IL6ST | P40189 | interleukin 6 | CD130, CDW130, | interluekin-6 |
| | | | cytokine family | GP130, HIES4, | receptor subunit |
| | | | signal transducer | HIES4A, HIES4B, | beta, IL-6 receptor |
| | | | | IL-6RB, IMD94, | subunit beta, IL-6R |
| | | | | STWS2, sGP130, | subunit beta; IL-6R- |
| | | | | interleukin-6 | beta, IL-6RB, |
| | | | | receptor β chain | CDw130, |
| | | | | | interleukin-6 signal |
| | | | | | transducer, |
| | | | | | membrane |
| | | | | | glycoprotein 130 |
| | | | | | (gp130), oncostatin- |
| | | | | | M receptor subunit |
| | | | | | alpha, CD130 |
| C-myc | MYC | P01106 | MYC proto- | MRTLC, | Myc proto-oncogene |
| | | | oncogene, bHLH | bHLHe39, c-Myc, | protein, class E |
| | | | transcription | BHLHE39, C- | basic helix-loop- |
| | | | factor | MYC-P64, MRTL, | helix protein 39 |
| | | | | MYCC | (bHLHe39), proto- |
| | | | | | oncogene c-Myc, |
| | | | | | transcription factor |
| | | | | | p64 |
| LILRB4 | LILRB4 | Q8NHJ6 | leukocyte | B4, CD85K, ILT- | leukocyte |
| | | | immunoglobulin | 3, ILT3, LIR-5, | immunoglobulin- |
| | | | like receptor B4 | LIR5 | like receptor |
| | | | | | subfamily B |
| | | | | | member 4, B4, |
| | | | | | CD85 antigen-like |
| | | | | | family member K, |
| | | | | | immunoglobulin- |
| | | | | | like transcript 3 |
| | | | | | (ILT-3), leukocyte |
| | | | | | immunoglobulin- |
| | | | | | like receptor 5 (LIR- |
| | | | | | 5), monocyte |
| | | | | | inhibitory receptor |
| | | | | | HM18, CD85k |
| BMP-2 | BMP2 | P12643 | bone morphogenic | BDA2A, SSFSC, | BMP-2, bone |
| | | | protein 2 | SSFSC1, BMP2A, | morphogenic protein |
| | | | | BDA2 | 2A (BMP-2A) |
| MIA | MIA | Q16674 | MIA SH3 domain | CD-RAP1, MIA1 | melanoma-derived |
| | | | containing | | growth regulatory |
| | | | | | protein, melanome |
| | | | | | inhibitory activity |
| | | | | | protein |
| CD34 | CD34 | P28906 | CD34 molecule | CD34 antigen, | hematopoietic |
| | | | | mesenchymal stem | progenitor cell |
| | | | | cell antigen | antigen CD34, |
| | | | | | mesenchymal stem |
| | | | | | cell antigen, CD34 |
| | | | | | antigen |
| CD63 | CD63 | P08962 | CD63 molecule | AD1, HOP-26, | CD63 antigen |
| | | | | ME491, MLA1, | |
| | | | | OMA81H, | |
| | | | | Pltgp40, | |
| | | | | TSPAN30, LIMP | |
| CD9 | CD9 | P21926 | CD9 molecule | BTCC-1, DRAP- | CD9 antigen, 5H9 |
| | | | | 27, MIC3, MRP-1, | antigen, cell growth- |
| | | | | TSPAN-29, | inhibiting gene 2 |
| | | | | TSPAN29, GIG2 | protein, leukocyte |
| | | | | | antigen MIC3, |
| | | | | | motility-related |
| | | | | | protein (MRP-1), |
| | | | | | tetraspanin-29 |
| | | | | | (Tspan-29), p24, |
| | | | | | CD9 |
| CD81 | CD81 | P60033 | CD81 molecule | CVID6, S5.7, | CD81 antigen, 26 |
| | | | | TAPA1, | kDa cell surface |
| | | | | TSPAN28 | protein TAPA-1, |
| | | | | | target of the |
| | | | | | antiproliferative |
| | | | | | antibody 1, |
| | | | | | tetraspanin-28 |
| | | | | | (Tspan-28), CD81 |
| IFNab R2 | IFNAR2 | P48551 | interferon alpha | IFN-R, IFN-R-2, | interferon alpha/beta |
| | | | beta receptor | IFN-alpha-REC, | receptor 2, IFN-R-2, |
| | | | subunit 2 | IFNABR, | IFN-alpha binding |
| | | | | IFNARB, IMD45, | protein, IFN- |
| | | | | IFN-α-REC, | alpha/beta receptor |
| | | | | interferon α and β | 2, interferon alpha |
| | | | | receptor subunit 2 | binding protein, type |
| | | | | | I inteerferon |
| | | | | | receptor 2 |
| Glypican 2 | GPC2 | Q8N158 | glypican 2 | | glypican-2, secreted |
| | | | | | glypican-2, |
| | | | | | cerebroglycan |
| MSP R | MST1R | Q04912 | macrophage | CD136, CDw136, | macrophage- |
| | | | stimulating 1 | NPCA3, PTK8, | stimulating protein |
| | | | receptor | RON, SEA, p185- | receptor, MSP |
| | | | | Ron, MSPR | receptor, CDw136, |
| | | | | | protein-tyrosine |
| | | | | | kinase 8, p185-Ron, |
| | | | | | macrophage- |
| | | | | | stimulating protein |
| | | | | | receptor alpha chain, |
| | | | | | macrophage- |
| | | | | | stimulating protein |
| | | | | | receptor beta chain, |
| | | | | | CD136, |
| | | | | | EC: 2.7.10.1, |
| | | | | | MSPR/Ron |
| DSCAM | DSCAM | O60469 | DS cell adhesion | CHD2, CHD2-42, | cell adhesion |
| | | | molecule | CHD2-52, | molecule DSCAM, |
| | | | | DSCAM1 | CHD2, down |
| | | | | | syndrome cell |
| | | | | | adhesion molecule |
| Matriptase | ST14 | Q9Y5Y6 | ST14 | ARCI11, CAP3, | suppressor of |
| | | | transmembrane | HAI, MT-SP1, | tumorigenicity 14 |
| | | | serine protease | MTSP1, PRSS14, | protein, matriptase, |
| | | | matriptase | SNC19, TADG15, | membrane-type |
| | | | | TMPRSS14 | serine protease 1 |
| | | | | | (MT-SP1), |
| | | | | | prostamin, serine |
| | | | | | protease 14, serine |
| | | | | | protease TADG-15, |
| | | | | | tumor-associated |
| | | | | | differentially- |
| | | | | | expressed gene 15 |
| | | | | | protein |
| KIR2DL3 | KIR2DL3 | P43628 | killer cell | CD158B2, | killer cell |
| | | | immunoglobulin | CD158b, GL183, | immunoglobulin- |
| | | | like receptor, two | KIR-023GB, KIR- | like receptor 2DL3, |
| | | | Ig domains and | K7b, KIR-K7c, | CD158 antigen-like |
| | | | long cytoplasmic | KIR2DL, | family member B2, |
| | | | tail 3 | KIR2DS5, | KIR-023GB, killer |
| | | | | KIRCL23, NKAT, | inhibitory receptor |
| | | | | NKAT2, | cl 2-3, NKAT2a, |
| | | | | NKAT2A, | NKAT2b, natural |
| | | | | NKAT2B, p58 | killer-associated |
| | | | | | transcript-2 (NKAT- |
| | | | | | 2), p58 natural killer |
| | | | | | cell receptor clone |
| | | | | | CL-6 (p58 NK |
| | | | | | receptor CL-6), |
| | | | | | p58.2 MHC class-I- |
| | | | | | specific NK |
| | | | | | receptor, CD158b2 |
| CD30 | TNFRSF8 | P28908 | TNF receptor | CD30, D1S166E, | tumor necrosis |
| | | | superfamily | Ki-1 | factor receptor |
| | | | member 8 | | superfamily member |
| | | | | | 8, CD30L, Ki-1 |
| | | | | | antigen, lymphocyte |
| | | | | | activation antigen |
| | | | | | CD30, CD30 |
| Siglec-10 | SIGLEC10 | Q96LC7 | sialic acid binding | PRO940, | sialic acid-binding |
| | | | Ig like lectin 10 | SIGLEC-10, | Ig-like lectin 10, |
| | | | | SLG2, | siglec-10, siglec-like |
| | | | | A630096C01RIK | protein 2 |
| CLEC-1 | CLEC1A | Q8NC01 | C-type lectin | CLEC-1, CLEC1 | C-type lectin |
| | | | domain family 1 | | domain family 1 |
| | | | member A | | member A, C-type |
| | | | | | lectin-like receptor 1 |
| | | | | | (CLEC-1) |
| TPP1 | TPP1 | O14773 | tripeptidy1 | CLN2, GIG1, | tripeptidyl-peptidase |
| | | | peptidase 1 | LPIC, SCAR7, | 1, TPP-1, cell |
| | | | | TPP-1 | growth-inhibiting |
| | | | | | gene 1 protein, |
| | | | | | lysosomal pepstatin- |
| | | | | | insensitive protease |
| | | | | | (LPIC), tripeptidy1 |
| | | | | | aminopeptidase, |
| | | | | | tripeptidyl-peptidase |
| | | | | | I (TPP-I), |
| | | | | | EC: 3.4.14.9 |
| Ubiquitin + 1 | UBB | P0CG47 | ubiquitin B | HEL-S-50 | ubiquitin + 1, |
| | | | | | ubiquitin(+1), |
| | | | | | polyubiquitin-B, |
| | | | | | ubiquitin |
| ANGPTL4 | ANGPTL4 | Q9BY76 | angiopoietin like 4 | ARP4, FIAF, | angiopoietin-related |
| | | | | HARP, HFARP, | protein 4, |
| | | | | NL2, PGAR, | angiopoietin-like |
| | | | | | protein 4, hepatic |
| | | | | TGQTL, UNQ171, | fibrinogen/angiopoietin- |
| | | | | pp1158, PP1158 | related protein |
| | | | | | (HFARP), |
| | | | | | ANGPTL4 N- |
| | | | | | terminal chain, |
| | | | | | ANGPTL4 C- |
| | | | | | terminal chain |
| TWEAK R | TNFRSF | Q9NP84 | TNF receptor | CD266, FN14, | tumor necrosis |
| | 12A | | superfamily | TWEAKR, | factor receptor |
| | | | member 12A | TNFRS12A | superfamily member |
| | | | | | 12A, fibroblast |
| | | | | | growth factor- |
| | | | | | inducible |
| | | | | | immediate-early |
| | | | | | response protein 14 |
| | | | | | (FGF-inducible 14), |
| | | | | | Tweak-receptor |
| | | | | | (TweakR), CD266 |
| Nidogen-1 | NID1 | P14543 | nidogen 1 | NID, ENTACTIN | nidogen-1, NID-1, |
| | | | | | entactin |
| CD2 | CD2 | P06729 | CD2 molecule | LFA-2, SRBC, | T-cell surface |
| | | | | T11, LFA3R, | antigen CD2, |
| | | | | LOC100128308 | erythrocyte receptor, |
| | | | | | LFA-2, LFA-3 |
| | | | | | receptor, rosette |
| | | | | | receptor, T-cell |
| | | | | | surface antigen |
| | | | | | T11/Leu-5, CD2 |
| Kallikrein 1 | KLK1 | P06870 | kallikrein 1 | KLKR, Klk6, hK1, | kallikrein-1, |
| | | | | tissue | kidney/pancreas/ |
| | | | | prokallikrein, | salivary gland |
| | | | | urinary | kallikrein, tissue |
| | | | | prokallikrein | kallikrein, |
| | | | | | EC: 3.4.21.35 |
| TSLP R | CRLF2 | Q9HC73 | cytokine receptor | CRLF2Y, TSLPR, | cytokine receptor- |
| | | | like factor 2 | ILXR, CRL2, | like factor 2, |
| | | | | CRL2Y | cytokine receptor- |
| | | | | | like 2, IL-XR, |
| | | | | | thymic stromal |
| | | | | | lymphopoietin |
| | | | | | protein receptor |
| | | | | | (TSLP receptor) |
| LAMP1 | LAMP1 | P11279 | lysosomal | CD107a, LAMPA, | lysosome-associated |
| | | | associated | LGP120 | membrane |
| | | | membrane protein | | glycoprotein 1, |
| | | | 1 | | LAMP-1, lysosome- |
| | | | | | associated |
| | | | | | membrane protein 1, |
| | | | | | CD107 antigen-like |
| | | | | | family member A, |
| | | | | | CD107a |
| TROY | TNFRSF | Q9NS68 | TNF receptor | TAJ, TAJ-alpha, | tumor necrosis |
| | 19 | | superfamily | TRADE, TROY, | factor receptor |
| | | | member 19 | TAJ-α | superfamily member |
| | | | | | 19, TRADE, |
| | | | | | toxicity and JNK |
| | | | | | inducer |
| VCAM-1 | VCAM1 | P19320 | vascular cell | CD106, INCAM- | vascular cell |
| | | | adhesion molecule | 100, VECAM1 | adhesion protein 1, |
| | | | 1 | | V-CAM 1, VCAM- |
| | | | | | 1, INCAM-100, |
| | | | | | soluble vascular cell |
| | | | | | adhesion molecule |
| | | | | | 1, CD106 |
| Siglec-11 | SIGLEC11 | Q96RL6 | sialic acid binding | | sialic acid-binding |
| | | | Ig like lectin 11 | | Ig-like lectin 11, |
| | | | | | sialic acid-binding |
| | | | | | lectin 11, Siglec-11 |
| S100A1 | S100A1 | P23297 | S100 calcium | S100, S100-alpha, | protein S100-A1, S- |
| | | | binding protein | S100A, S100-α | 100 protein alpha |
| | | | A1 | | chain, S-100 protein |
| | | | | | subunit alpha, S100 |
| | | | | | calcium-binding |
| | | | | | protein Al |
| PAR1 | F2R | P25116 | coagulation factor | CF2R, HTR, PAR- | proteinase-activated |
| | | | II thrombin | 1, PAR1, TR | receptor 1, PAR-1, |
| | | | receptor | | coagulation factor II |
| | | | | | receptor, thrombin |
| | | | | | receptor |
| Thyroid | TPO | P07202 | thyroid peroxidase | MSA, TDH2A, | TPO, EC: 1.11.1.8 |
| Peroxidase | | | | TPX | |
| Aminopeptidase | XPNPEP2 | O43895 | X-proly1 | AEACEI, APP2, | Xaa-Pro |
| P2 | | | aminopeptidase 2 | mAmP | aminopeptidase 2, |
| | | | | | aminoacylproline |
| | | | | | aminopeptidase, |
| | | | | | membrane-bound |
| | | | | | aminopeptidase P, |
| | | | | | membrane-bound |
| | | | | | APP, membrane- |
| | | | | | bound AmP, |
| | | | | | mAmP, X-Pro |
| | | | | | aminopeptidase 2 |
| IL-1 RI | IL1R1 | P14778 | interleukin 1 | CD121A, | interleukin-1 |
| | | | receptor type 1 | CRMO3, | receptor type 1, IL- |
| | | | | D2S1473, IL-1R- | 1R-1, IL-1RT-1, IL- |
| | | | | alpha, IL-1RT1, | 1RT1, CD121 |
| | | | | IL1R, IL1RA, | antigen-like family |
| | | | | | member A, |
| | | | | P80, IL1RT1, | interleukin-1 |
| | | | | IL1bRa, IL-1R-α | receptor alpha, IL- |
| | | | | | 1R-alpha, |
| | | | | | interleukin-1 |
| | | | | | receptor type I, p80, |
| | | | | | interleukin-1 |
| | | | | | receptor type 1 |
| | | | | | membrane form |
| | | | | | (mIL-1R1, mIL- |
| | | | | | 1RI), interleukin-1 |
| | | | | | receptor type 1 |
| | | | | | soluble form (sIL- |
| | | | | | 1R1, sIL-1RI), |
| | | | | | CD121a |
| ADAMS | ADAM2 | Q99965 | ADAM | CRYN1, CRYN2, | disintegrin and |
| | | | metallopeptidase | CT15, FTNB, PH- | metalloproteinase |
| | | | domain 2 | 30b, PH30, PH30- | domain-containing |
| | | | | beta, PH30-β, | protein 2, ADAM 2, |
| | | | | fertilin beta, | cancer/testis antigen |
| | | | | fertilin β | 15 (CT15), fertilin |
| | | | | | subunit beta, PH-30, |
| | | | | | PH30, PH-30-beta |
| | ADAM7 | Q9H2U9 | ADAM | ADAM 7, ADAM- | disintegrin and |
| | | | metallopeptidase | 7, EAPI, GP-83, | metalloproteinase |
| | | | domain 7 | GP83 | domain-containing |
| | | | | | protein 7, ADAM 7, |
| | | | | | sperm maturation- |
| | | | | | related glycoprotein |
| | | | | | GP-83 |
| | ADAM8 | P78325 | ADAM | CD156, CD156a, | disintegrin and |
| | | | metallopeptidase | MS2 | metalloproteinase |
| | | | domain 8 | | domain-containing |
| | | | | | protein 8, ADAM 8, |
| | | | | | cell surface antigen |
| | | | | | MS2, CD156a, |
| | | | | | EC: 3.4.24.- |
| | ADAM9 | Q13443 | ADAM | CORD9, MCMP, | disintegrin and |
| | | | metallopeptidase | MDC9, Mltng, | metalloproteinase |
| | | | domain 9 | KIAA0021, | domain-containing |
| | | | | MLTNG | protein 9, ADAM 9, |
| | | | | | cellular disintegrin- |
| | | | | | related protein, |
| | | | | | meltrin-gamma, |
| | | | | | meltrin gamma, |
| | | | | | meltrin γ, |
| | | | | | metalloprotease/ |
| | | | | | disintegrin/cysteine-rich |
| | | | | | protein 9, myeloma |
| | | | | | cell |
| | | | | | metalloproteinase, |
| | | | | | ADAM9 precursor, |
| | | | | | EC: 3.4.24.- |
| | ADAM10 | O14672 | ADAM | AD10, AD18, | disintegrin and |
| | | | metallopeptidase | CD156c, CDw156, | metalloproteinase |
| | | | domain 10 | HsT18717, | domain-containing |
| | | | | MADM, RAK, | protein 10, ADAM |
| | | | | kuz, KUZ | 10, CDw156, |
| | | | | | Kuzbanian protein |
| | | | | | homolog, |
| | | | | | mammalian |
| | | | | | disintegrin- |
| | | | | | metalloprotease, |
| | | | | | CD156c, |
| | | | | | disintegrin- |
| | | | | | metalloprotease, α |
| | | | | | Secretase, |
| | | | | | EC: 3.4.24.81 |
| | ADAM11 | O75078 | ADAM | MDC | disintegrin and |
| | | | metallopeptidase | | metalloproteinase |
| | | | domain 11 | | domain-containing |
| | | | | | protein 11, ADAM |
| | | | | | 11 |
| | ADAM12 | O43184 | ADAM | ADAM12-OT1, | disintegrin and |
| | | | metallopeptidase | CAR10, MCMP, | metalloproteinase |
| | | | domain 12 | MCMPMltna, | domain-containing |
| | | | | MLTN, MLTNA, | protein 12, ADAM |
| | | | | meltrin α | 12, meltrin-alpha, |
| | | | | | EC: 3.4.24.- |
| | ADAM15 | Q13444 | ADAM | MDC15, | disintegrin and |
| | | | metallopeptidase | METARGIDIN | metalloproteinase |
| | | | domain 15 | | domain-containing |
| | | | | | protein 15, ADAM |
| | | | | | 15, metalloprotease |
| | | | | | RGD disintegrin |
| | | | | | protein, |
| | | | | | metalloproteinase- |
| | | | | | like disintegrin-like |
| | | | | | and cysteine-rich |
| | | | | | protein 15 |
| | | | | | (MDC15), |
| | | | | | metargidin, |
| | | | | | EC: 3.4.24.- |
| | ADAM17 | P78536 | ADAM | ADAM18, | disintegrin and |
| | | | metallopeptidase | CD156B, CSVP, | metalloproteinase |
| | | | domain 17 | NISBD, NISBD1, | domain-containing |
| | | | | TACE, Tnfa | protein 17, ADAM |
| | | | | Convertase, Tnf-α- | 17, snake venom- |
| | | | | converting enzyme | like protease, TNF- |
| | | | | | alpha convertase, |
| | | | | | TNF-alpha- |
| | | | | | converting enzyme, |
| | | | | | CD156b |
| | ADAM18 | Q9Y3Q7 | ADAM | ADAM27, | disintegrin and |
| | | | metallopeptidase | tMDCIII, TMDC3 | metalloproteinase |
| | | | domain 18 | | domain-containing |
| | | | | | protein 18, ADAM |
| | | | | | 18, transmembrane |
| | | | | | metalloproteinase- |
| | | | | | like disintegrin-like |
| | | | | | and cysteine-rich |
| | | | | | protein III (tMDC |
| | | | | | III) |
| | ADAM19 | Q9H013 | ADAM | FKSG34, | disintegrin and |
| | | | metallopeptidase | MADDAM, | metalloproteinase |
| | | | domain 19 | MLTNB, | domain-containing |
| | | | | FKSG34, meltrin β | protein 19, ADAM |
| | | | | | 19, meltrin-beta, |
| | | | | | metalloprotease and |
| | | | | | disintegrin dendritic |
| | | | | | antigen marker |
| | | | | | (MADDAM), |
| | | | | | EC: 3.4.24.- |
| | ADAM20 | O43506 | ADAM | | disintegrin and |
| | | | metallopeptidase | | metalloproteinase |
| | | | domain 20 | | domain-containing |
| | | | | | protein 20, ADAM |
| | | | | | 20, EC: 3.4.24 .- |
| | ADAM21 | Q9UKJ8 | ADAM | ADAM 21, | disintegrin and |
| | | | metallopeptidase | ADAM31 | metalloproteinase |
| | | | domain 21 | | domain-containing |
| | | | | | protein 21, ADAM |
| | | | | | 21, EC: 3.4.24.- |
| | ADAM22 | Q9P0K1 | ADAM | ADAM 22, | disintegrin and |
| | | | metallopeptidase | DEE61, EIEE61, | metalloproteinase |
| | | | domain 22 | MDC2 | domain-containing |
| | | | | | protein 22, ADAM |
| | | | | | 22, |
| | | | | | metalloproteinase- |
| | | | | | disintegrin |
| | | | | | ADAM22-3, |
| | | | | | metalloproteinase- |
| | | | | | like disintegrin-like |
| | | | | | and cysteine-rich |
| | | | | | protein 2 |
| | ADAM23 | O75077 | ADAM | MDC-3, MDC3 | disintegrin and |
| | | | metallopeptidase | | metalloproteinase |
| | | | domain 23 | | domain-containing |
| | | | | | protein 23, ADAM |
| | | | | | 23, |
| | | | | | metalloproteinase- |
| | | | | | like disintegrin-like |
| | | | | | and cysteine-rich |
| | | | | | protein 3 (MDC-3) |
| | ADAM28 | Q9UKQ2 | ADAM | ADAM 28, MDC- | disintegrin and |
| | | | metallopeptidase | L, MDCL, eMDC | metalloproteinase |
| | | | domain 28 | II, eMDCII, | domain-containing |
| | | | | ADAM23 | protein 28, ADAM |
| | | | | | 28, epididymal |
| | | | | | metalloproteinase- |
| | | | | | like disintegrin-like |
| | | | | | and cysteine-rich |
| | | | | | protein II (eMDC |
| | | | | | II), |
| | | | | | metalloproteinase- |
| | | | | | like disintegrin-like |
| | | | | | and cysteine-rich |
| | | | | | protein L (MDC-L) |
| | ADAM29 | Q9UKF5 | ADAM | CT73, svph1 | disintegrin and |
| | | | metallopeptidase | | metalloproteinase |
| | | | domain 29 | | domain-containing |
| | | | | | protein 29, ADAM |
| | | | | | 29, cancer/testis |
| | | | | | antigen 73 (CT73) |
| | ADAM30 | Q9UKF2 | ADAM | svph4 | disintegrin and |
| | | | metallopeptidase | | metalloproteinase |
| | | | domain 30 | | domain-containing |
| | | | | | protein 30, ADAM |
| | | | | | 30, EC: 3.4.24.- |
| | ADAM32 | Q8TC27 | ADAM | | disintegrin and |
| | | | metallopeptidase | | metalloproteinase |
| | | | domain 32 | | domain-containing |
| | | | | | protein 32, ADAM |
| | | | | | 32 |
| | ADAM33 | Q9BZ11 | ADAM | C20orf153, | disintegrin and |
| | | | metallopeptidase | DJ964F7.1 | metalloproteinase |
| | | | domain 33 | | domain-containing |
| | | | | | protein 33, ADAM |
| | | | | | 33, EC: 3.4.24.- |
| | ADAMD | O15204 | ADAM like | M12.219, ADAM- | ADAM DEC1, a |
| | EC1 | | decysin 1 | LIKE | disintegrin and |
| | | | | | metalloproteinase |
| | | | | | domain-like protein |
| | | | | | decysin-1, ADAM- |
| | | | | | like protein decysin- |
| | | | | | 1, EC: 3.4.24.- |
| OSM R beta | OSMR | Q99650 | oncostatin M | IL-31R-beta, IL- | oncostatin-M- |
| | | | receptor | 31R-β, IL-31RB, | specific receptor |
| | | | | OSMRbeta, | subunit beta, |
| | | | | PLCA1, OSMRB | interleukin-31 |
| | | | | | receptor subunit |
| | | | | | beta, IL-31 receptor |
| | | | | | subunit beta, IL-31R |
| | | | | | subunit beta, IL- |
| | | | | | 31R-beta, IL-31RB |
| Thrombospondin-2 | THBS2 | P35442 | thrombospondin 2 | EDSCLL3, TSP2 | thrombospondin-2 |
| SMPD1 | SMPD1 | P17405 | sphingomyelin | ASM, ASMASE, | sphingomyelin |
| | | | phosphodiesterase | NPD | phosphodiesterase, |
| | | | 1 | | acid |
| | | | | | sphingomyelinase |
| | | | | | (aSMase), |
| | | | | | sphingomyelin |
| | | | | | phosphodiesterase |
| | | | | | processed form, |
| | | | | | EC: 3.1.4.12, |
| | | | | | EC: 3.1.4.3 |
| B2M | B2M | P61769 | beta-2- | AMYLD6, | beta-2- |
| | | | microglobulin | IMD43, MHC1D4 | microglobulin, beta- |
| | | | | | 2-microglobulin |
| | | | | | form pI 5.3, β-2- |
| | | | | | microglobulin |
| MFRP | MFRP | Q9BY79 | membrane | CTRP5, MCOP5, | membrane-type |
| | | | frizzled-related | NNO2, RD6 | frizzled-related |
| | | | protein | | protein |
| LRP-6 | LRP6 | O75581 | LDL receptor | ADCAD2, | low-density |
| | | | related protein 6 | STHAG7 | lipoprotein receptor- |
| | | | | | related protein 6, |
| | | | | | LRP-6 |
| ST3GAL1 | ST3GAL1 | Q11201 | ST3 beta- | Gal-NAc6S, | CMP-N- |
| | | | galactoside alpha- | SIAT4A, SIATFL, | acetylneuraminate- |
| | | | 2.3- | ST3GalA, | beta-galactosamide- |
| | | | sialytransferase 1 | ST3GalA.1, | alpha-2,3- |
| | | | | ST3GalIA, | sialyltransferase 1, |
| | | | | ST3GalIA 1, | alpha 2,3-ST 1, |
| | | | | ST3O, SIAT4, | beta-galactoside |
| | | | | LOC286167, | alpha-2,3- |
| | | | | sialyltransferase | sialyltransferase 1, |
| | | | | 4A, ST3 β- | Gal-NAc6S, Gal- |
| | | | | galactoside α-2,3- | beta-1,3-GalNAc- |
| | | | | sialytransferase 1 | alpha-2,3- |
| | | | | | sialyltransferase, |
| | | | | | monosialoganglioside |
| | | | | | sialyltransferase |
| | | | | | (EC: 2.4.3.2), |
| | | | | | SIATFL, ST3Gal I, |
| | | | | | ST3GalI, |
| | | | | | ST3GalA.1, ST3O, |
| | | | | | sialy ltransferase 4A, |
| | | | | | SIAT4-A |
| NCAM-1 | NCAM1 | P13591 | neural cell | CD56, MSK39, | N-CAM-1, NCAM- |
| (CD56) | | | adhesion molecule | NCAM | 1, CD56 |
| | | | 1 | | |
| Granzyme B | GZMB | P10144 | granzyme B | C11, CCPI, CGL- | C11, CTLA-1, |
| | | | | 1, CGL1, CSP-B, | cathepsin G-like 1 |
| | | | | CSPB, CTLA1, | (CTSGL1), |
| | | | | CTSGL1, HLP, | cytotoxic T- |
| | | | | SECT, GRB, GrB, | lymphocyte |
| | | | | GZMC | proteinase 2, |
| | | | | | lymphocyte |
| | | | | | protease, |
| | | | | | fragmentin-2, |
| | | | | | granzyme-2, human |
| | | | | | lymphocyte protein |
| | | | | | (HLP), SECT, T- |
| | | | | | cell serine protease |
| | | | | | 1-3E |
| Adiponectin | ADIPOQ | Q15848 | adiponectin, C1Q | ACDC, ACRP30, | adiponectin, 30 kDa |
| | | | and collagen | ADIPQTL1, | adipocyte |
| | | | domain containing | ADPN, APM-1, | complement-related |
| | | | | APM1, GBP28, | protein, adipocyte |
| | | | | ApN | complement-related |
| | | | | | 30 kDa protein |
| | | | | | (ACRP30), |
| | | | | | adipocyte C1q and |
| | | | | | collagen domain- |
| | | | | | containing protein, |
| | | | | | adipose most |
| | | | | | abundant gene |
| | | | | | transcript 1 protein |
| | | | | | (apM-1), gelatin- |
| | | | | | binding protein |
| IL-22BP | IL22RA2 | Q969J5 | interleukin 22 | CRF2-10, CRF2- | interleukin-22 |
| | | | receptor subunit | S1, CRF2X, IL- | receptor subunit |
| | | | alpha 2 | 22BP, IL-22R- | alpha-2, IL-22 |
| | | | | alpha-2, il-22R-α- | receptor subunit |
| | | | | 2, IL-22RA2, | alpha-2, IL-22R- |
| | | | | ZCYTOR16, | alpha-2, IL-22RA2, |
| | | | | interleukin 22 | cytokine receptor |
| | | | | receptor subunit α | class-II member 10, |
| | | | | 2 | cytokine receptor |
| | | | | | family 2 member 10 |
| | | | | | (CRF2-10), cytokine |
| | | | | | receptor family type |
| | | | | | 2 soluble 1 (CRF2- |
| | | | | | S1), interleukin-22- |
| | | | | | binding protein (IL- |
| | | | | | 22BP, IL22BP), |
| | | | | | zcytoR16 |
| TPST2 | TPST2 | O60704 | tyrosylprotein | TANGO13B, | protein-tyrosine |
| | | | sulfotransferase 2 | TPST-2 | sulfotransferase 2, |
| | | | | | tyrosylprotein |
| | | | | | sulfotransferase 2 |
| | | | | | (TPST-2), |
| | | | | | EC: 2.8.2.20, |
| | | | | | tyrosylprotein |
| | | | | | sulphotransferase 2 |
| PD-ECGF | TYMP | P19971 | thymidine | ECGF, ECGF1, | TP, gliostatin, |
| | | | phosphory lase | MEDPS1, | platelet-derived |
| | | | | MNGIE, | endothelial cell |
| | | | | MTDPS1, | growth factor (PD- |
| | | | | PDECGF, TP, | ECGF), TdRPase, |
| | | | | hPD-ECGF, | EC: 2.4.2.4 |
| | | | | GLIOSTATIN | |
| LH | CGA | P01215 | glycoprotein | CG-ALPHA, | glycoprotein |
| | | | hormones, alpha | FSHA, GPA1, | hormones alpha |
| | | | polypeptide | GPHA1, GPHa, | chain, anterior |
| | | | | HCG, LHA, | pituitary |
| | | | | TSHA, alphaGSU, | glycoprotein |
| | | | | CG-α, Fsh α, Hcg | hormones common |
| | | | | α, Tsh α, α, α gsu, | subunit alpha, |
| | | | | alpha | glycoprotein |
| | | | | | hormone alpha, |
| | | | | | glycoprotein |
| | | | | | hormones α |
| | | | | | polypeptide, |
| | | | | | glycoprotein |
| | | | | | hormone α, |
| | | | | | glycoprotein |
| | | | | | hormone α chain, |
| | | | | | glycoprotein |
| | | | | | hormone α subunit, |
| | | | | | gonadotropin α, |
| | | | | | choriogonadotropin |
| | | | | | alpha subunit, |
| | | | | | chorionic |
| | | | | | gonadotrophin |
| | | | | | subunit alpha (CG- |
| | | | | | alpha), follicle- |
| | | | | | stimulating hormone |
| | | | | | alpha chain (FSH- |
| | | | | | alpha), follitropin |
| | | | | | alpha chain, |
| | | | | | luteinizing hormone |
| | | | | | alpha chain (LSH- |
| | | | | | alpha), lutropin |
| | | | | | alpha chain, thyroid- |
| | | | | | stimulating hormone |
| | | | | | alpha chain (TSH- |
| | | | | | alpha), thyrotropin |
| | | | | | alpha chain, LH and |
| | | | | | FSH common alpha |
| | | | | | subunit, LH and |
| | | | | | FSH common α |
| | | | | | subunit |
| | LHB | P01229 | luteinizing | CGB4, HH23, | lutropin subunit |
| | | | hormone subunit | LSH-B, LSH-beta, | beta, lutropin beta |
| | | | beta | LH beta, LH β, | chain, luteinizing |
| | | | | LSH-β | hormone subunit |
| | | | | | beta (LH-B, LSH-B, |
| | | | | | LSH-beta), |
| | | | | | luteinizing hormone |
| | | | | | subunit β |
| LEDGF | PSIP1 | O75475 | PC4 and SRSF1 | DFS70, LEDGF, | PC4 and SFRS1- |
| | | | interacting protein | PAIP, PSIP2, p52, | interacting protein, |
| | | | 1 | p75 | CLL-associated |
| | | | | | antigen KW-7, |
| | | | | | dense fine speckles |
| | | | | | 70 kDa protein |
| | | | | | (DFS 70), lens |
| | | | | | epithelium-derived |
| | | | | | growth factor, |
| | | | | | transcriptional |
| | | | | | coactivator p75/p52 |
| Cyr61 | CCN1 | O00622 | cellular | CYR61, GIG1, | CCN family |
| | | | communication | IGFBP10 | member 1, cellular |
| | | | network factor 1 | | communication |
| | | | | | network factor 1, |
| | | | | | cysteine-rich |
| | | | | | angiogenic inducer |
| | | | | | 61, insulin-like |
| | | | | | growth factor- |
| | | | | | binding protein 10, |
| | | | | | IBP-10, IGF- |
| | | | | | binding protein 10, |
| | | | | | IGFBP-10, protein |
| | | | | | CYR61, protein |
| | | | | | GIG1 |
| ULBP-3 | ULBP3 | Q9BZM4 | UL16 binding | N2DL-3, | UL16-binding |
| | | | protein 3 | NKG2DL3, | protein 3, ALCAN- |
| | | | | | gamma, NKG2D |
| | | | | RAET1N, N2DL3, | ligand 3, N2DL-3, |
| | | | | NKG2D ligand 3 | NKG2DL3, retinoic |
| | | | | | acid early transcript |
| | | | | | 1N |
| IFNb | IFNB1 | P01574 | interferon beta 1 | IFB, IFF, IFN- | interferon beta, IFN- |
| | | | | beta, IFNB, | beta, fibroblast |
| | | | | IFNB1A, IFN | interferon, |
| | | | | beta, IFN β | interferon β 1, |
| | | | | | neoferon, |
| | | | | | recombinant human |
| | | | | | interferon β 1a, |
| | | | | | recombinantinterfer |
| | | | | | on beta 1a, |
| | | | | | recombinantinterfer |
| | | | | | on β 1a |
| THSD1 | THSD1 | Q9NS62 | thrombospondin | ANIB12, | thrombospondin |
| | | | type 1 domain | LMPHM13, | type-1 domain- |
| | | | containing 1 | TMTSP, | containing protein 1, |
| | | | | UNQ3010 | transmembrane |
| | | | | | molecule with |
| | | | | | thrombospondin |
| | | | | | module |
| FGF-23 | FGF23 | Q9GZV9 | fibroblast growth | ADHR, FGFN, | FGF-23, |
| | | | factor 23 | HFTC2, HPDR2, | phosphatonin, |
| | | | | HYPF, PHPTC | tumor-derived |
| | | | | | hypophosphatemia- |
| | | | | | inducing factor, |
| | | | | | fibroblast growth |
| | | | | | factor 23 N-terminal |
| | | | | | peptide, fibroblast |
| | | | | | growth factor 23 C- |
| | | | | | terminal peptide |
| LAMA4 | LAMA4 | Q16363 | laminin subunit | CMD1JJ, | laminin subunit |
| | | | alpha 4 | LAMA4*-1, | alpha-4, laminin-14 |
| | | | | LAMA3 | subunit alpha, |
| | | | | | laminin-8 subunit |
| | | | | | alpha, laminin-9 |
| | | | | | subunit alpha, |
| | | | | | laminin subunit α 4, |
| | | | | | laminin α 4 |
| Adipsin | CFD | P00746 | complement factor | ADIPSIN, ADN, | adipsin, C3 |
| | | | D | DF, PFD | convertase activator, |
| | | | | | properdin factor D, |
| | | | | | EC: 3.4.21.46 |
| AIF | AIFM1 | O95831 | apoptosis inducing | AIF, AUNX1, | apoptosis-inducing |
| | | | factor | CMT2D, CMTX4, | factor 1 |
| | | | mitochondria | COWCK, | mitochondrial, |
| | | | associated 1 | COXPD6, | programmed cell |
| | | | | DFNX5, NADMR, | |
| | | | | NAMSD, PDCD8, | death protein 8, |
| | | | | SEMDHL | EC: 1.6.99.- |
| SorCS2 | SORCS2 | Q96PQ0 | sortilin related | KIAA1329 | VSP10 domain- |
| | | | VPS10 domain | | containing receptor |
| | | | containing | | SorCS2, SorCS2 |
| | | | receptor 2 | | 122 kDa chain, |
| | | | | | SorCS2 104 kDa |
| | | | | | chain, SorCS2 18 |
| | | | | | kDa chain |
| SULT2A1 | SULT2A1 | Q06520 | sulfotransferase | DHEA-ST, | sulfotransferase |
| | | | family 2A | DHEA-ST8, | 2A1, ST2A1, bile |
| | | | member 1 | DHEAS, HST, | salt sulfotransferase |
| | | | | ST2, ST2A1, | (EC: 2.8.2.14), |
| | | | | ST2A3, STD, | dehydroepiandroster |
| | | | | SULT2A3, hSTa | one sulfotransferase |
| | | | | | (DHEA-ST, DHEA- |
| | | | | | ST8), |
| | | | | | hydroxysteroid |
| | | | | | sulfotransferase |
| | | | | | (HST), ST2, |
| | | | | | SULT2A3, alcohol |
| | | | | | sulfotransferase, |
| | | | | | alcohol |
| | | | | | sulphotransferase, |
| | | | | | sulphotransferase |
| | | | | | family 2A member |
| | | | | | 1, EC: 2.8.2.2 |
| CD39L2 | ENTPD6 | O75354 | ectonucleoside | CD39L2, IL6ST2, | NTPDase 6, CD39 |
| | | | triphosphate | IL-6SAG, | antigen-like 2, |
| | | | diphosphohydrolase | NTPDase-6, | EC: 3.6.1.6 |
| | | | 6 | dJ738P15.3 | |
| Insulin R | INSR | P06213 | insulin receptor | CD220, HHF5, IR, | insulin receptor |
| | | | | IR2 | kinase, IR, insulin |
| | | | | | receptor subunit |
| | | | | | alpha, insulin |
| | | | | | receptor subunit |
| | | | | | beta, CD220, |
| | | | | | EC: 2.7.10.1 |
| HIF-1 alpha | HIF1A | Q16665 | hypoxia inducible | HIF-1-alpha, HIF- | hypoxia-inducible |
| | | | factor 1 subunit | 1A, HIF-1alpha, | factor 1-alpha, |
| | | | alpha | HIF1, HIF1- | hypoxia inducible |
| | | | | ALPHA, MOP1, | factor 1 subunit α, |
| | | | | PASD8, | HIF-1-alpha, HIF1- |
| | | | | bHLHe78, | alpha, ARNT- |
| | | | | BHLHE78, Hif1- | interacting protein, |
| | | | | α, HIF-1-α | basic-helix-loop- |
| | | | | | helix-PAS protein |
| | | | | | MOP1, class E basic |
| | | | | | helix-loop-helix |
| | | | | | protein 78 |
| | | | | | (bHLHe78), |
| | | | | | member of PAS |
| | | | | | protein 1, PAS |
| | | | | | domain-containing |
| | | | | | protein 8 |
| OX40 | TNFSF4 | P23510 | TNF superfamily | CD134L, CD252, | tumor necrosis |
| Ligand | | | member 4 | GP34, OX-40L, | factor ligand |
| | | | | OX4OL, OX40L, | superfamily member |
| | | | | TNLG2B, TXGP1, | 4, glycoprotein |
| | | | | OX40 LIGAND | Gp34, OX40 ligand |
| | | | | | (OX40L), TAX |
| | | | | | transcriptionally- |
| | | | | | activated |
| | | | | | glycoprotein 1, |
| | | | | | CD252 |
| Pax3 | PAX3 | P23760 | paired box 3 | CDHS, HUP2, | paired box protein |
| | | | | PAX-3, WS1, | Pax-3, HuP2 |
| | | | | WS3 | |
| UCH-L3 | UCHL3 | P15374 | ubiquitin C- | UCH-L3 | ubiquitin carboxyl- |
| | | | terminal hydrolase | | terminal hydrolase |
| | | | L3 | | isozyme L3, UCH- |
| | | | | | L3, ubiquitin |
| | | | | | thioesterase L3, |
| | | | | | EC: 3.4.19.12 |
| cMASP3 | MASP1 | P48740 | MBL associated | 3MC1, CRARF, | mannan-binding |
| | | | serine protease 1 | CRARF1, MAP-1, | lectin serine |
| | | | | MAP1, MASP, | protease 1, |
| | | | | MASP-3, MASP3, | complement factor |
| | | | | MAp44, PRSS5, | MASP-3, |
| | | | | RaRF, MASP1/3 | complement- |
| | | | | | activating |
| | | | | | component of Ra- |
| | | | | | reactive factor, |
| | | | | | mannose-binding |
| | | | | | lectin-associated |
| | | | | | serine protease 1 |
| | | | | | (MASP-1), |
| | | | | | mannose-binding |
| | | | | | protein-associated |
| | | | | | serine protease, Ra- |
| | | | | | reactive factor |
| | | | | | serine protease p100 |
| | | | | | (RaRF), serine |
| | | | | | protease 5, mannan- |
| | | | | | binding lectin serine |
| | | | | | protease 1 heavy |
| | | | | | chain, mannan- |
| | | | | | binding lectin serine |
| | | | | | protease 1 light |
| | | | | | chain, EC: 3.4.21.- |
| Langerin | CD207 | Q9UJ71 | CD207 molecule | CLEC4K, | C-type lectin |
| | | | | Langerin | domain family 4 |
| | | | | | member K, langerin, |
| | | | | | CD207 |
| Desmin | DES | P17661 | desmin | CDCD3, CSM1, | |
| | | | | CSM2, LGMD1D, | |
| | | | | LGMD1E, | |
| | | | | LGMD2R, | |
| | | | | ARVD7, CMD1F | |
| SOX9 | SOX9 | P48436 | SRY-box | CMD1, CMPD1, | transcription factor |
| | | | transcription | SRA1, SRXX2, | SOX-9 |
| | | | factor 9 | SRXY10 | |
| ST6GAL1 | ST6GAL1 | P15907 | ST6 beta- | CDw75, SIAT1, | ST6 β-galactoside α- |
| | | | galactoside alpha- | ST6GalI, ST6N, | 2,6-sialyltransferase |
| | | | 2,6- | CD75, HST6GAL, | 1, beta-galactoside |
| | | | sialyltransferase 1 | LOC102723861 | alpha-2,6- |
| | | | | | sialytransferase 1, |
| | | | | | alpha 2,6-ST 1, B- |
| | | | | | cell antigen CD75, |
| | | | | | CMP-N- |
| | | | | | acetylneuraminate- |
| | | | | | beta-galactosamide- |
| | | | | | alpha-2,6- |
| | | | | | sialyltransferase 1, |
| | | | | | ST6Gal I (ST6GalI), |
| | | | | | sialyltransferase 1 |
| MEP1B | MEP1B | | | | |
| | is one of | | | | |
| | the | | | | |
| | subunits | | | | |
| | that | | | | |
| | makes up | | | | |
| | Meprin | | | | |
| | A. | | | | |
| | MEP1B | | | | |
| | is on line | | | | |
| | 11 of this | | | | |
| | document. | | | | |
| CD99-L2 | CD99L2 | Q8TCZ2 | CD99 molecule | CD99B, MIC2L1 | CD99 antigen-like |
| | | | like 2 | | protein 2, MIC2-like |
| | | | | | protein 1, CD99 |
| Plexin A4 | PLXNA4 | Q9HCM2 | plexin A4 | FAYV2820, | plexin-A4 |
| | | | | PLEXA4, | |
| | | | | PLXNA4A, | |
| | | | | PLXNA4B, | |
| | | | | PRO34003, | |
| | | | | KIAA1550 | |
| Semaphorin | SEMA4D | Q92854 | semaphorin 4D | A8, BB18, | semaphorin-4D, A8, |
| 4D | | | | C9orf164, CD100, | BB18, GR3, CD100 |
| | | | | COLL4, GR3, M- | |
| | | | | sema-G, SEMAJ, | |
| | | | | coll-4 | |
| ROBO2 | ROBO2 | Q9HCK4 | roundabout | SAX3, KIAA1568 | roundabout homolog |
| | | | guidance receptor | | 2 |
| | | | 2 | | |
| PDX-1 | PDX1 | P52945 | pancreatic and | GSF, IDX-1, IPF1, | pancreas/duodenum |
| | | | duodenal | IPF2, IUF1, | homeobox protein 1, |
| | | | homeobox 1 | MODY4, | PDX-1, glucose- |
| | | | | PAGEN1, PDX-1, | sensitive factor |
| | | | | STF-1, STF1 | (GSF), insulin |
| | | | | | promoter factor 1 |
| | | | | | (IPF-1), insulin |
| | | | | | upsteam factor 1 |
| | | | | | (IUF-1), |
| | | | | | islet/duodenum |
| | | | | | homeobox-1 (IDX- |
| | | | | | 1), somatostatin- |
| | | | | | transactivating |
| | | | | | factor 1 (STF-1) |
| APRIL | TNFSF13 | O75888 | TNF superfamily | APRIL, CD256, | tumor necrosis |
| | | | member 13 | TALL-2, TALL2, | factor ligand |
| | | | | TNLG7B, TRDL-1, | superfamily member |
| | | | | UNQ383/PRO715, | 13, A proliferation- |
| | | | | ZTNF2, TWE- | inducing ligand |
| | | | | PRIL | (APRIL), TNF- and |
| | | | | | APOL-related |
| | | | | | leukocyte expressed |
| | | | | | ligand 2 (TALL-2), |
| | | | | | TNF-related death |
| | | | | | ligand 1 (TRDL-1), |
| | | | | | CD256 |
| Neurturin | NRTN | Q99748 | neurturin | NTN | |
| Kremen-2 | KREMEN2 | Q8NCW0 | kringle containing | KRM2 | kremen protein 2, |
| | | | transmembrane | | Dickkopf receptor 2, |
| | | | protein 2 | | kringle domain- |
| | | | | | containing |
| | | | | | transmembrane |
| | | | | | protein 2, kringle- |
| | | | | | containing protein |
| | | | | | marking the eye and |
| | | | | | the nose |
| EMMPRIN | BSG | P35613 | basigin (Ok blood | 5F7, CD147, | basigin, 5F7, |
| | | | group) | EMMPRIN, | collagenase |
| | | | | EMPRIN, | stimulatory factor, |
| | | | | HAb18G, OK, | extracellular matrix |
| | | | | TCSF, BASIGIN, | metalloproteinase |
| | | | | BASIN, | inducer |
| | | | | HAb18G/CD147 | (EMMPRIN), |
| | | | | | hepatoma-associated |
| | | | | | antigen (HAb18G), |
| | | | | | leukocyte activation |
| | | | | | antigen M6, OK |
| | | | | | blood group antigen, |
| | | | | | tumor cell-derived |
| | | | | | collagenase |
| | | | | | stimulatory factor |
| | | | | | (TCSF), CD147 |
| Activin RIB | ACVR1B | P36896 | activin A receptor | ACTRIB, | activin receptor |
| | | | type 1B | ACVRLK4, | type-1B, activin |
| | | | | ALK4, SKR2 | receptor type IB |
| | | | | | (ACTR-IB), activin |
| | | | | | receptor-like kinase |
| | | | | | 4 (ALK-4), |
| | | | | | serine/threonine- |
| | | | | | protein, kinase |
| | | | | | receptor R2 (SKR2), |
| | | | | | EC: 2.7.11.30 |
| Neuroligin 2 | NLGN2 | Q8NFZ4 | neuroligin 2 | KIAA1366 | neuroligin-2 |
| Epiregulin | EREG | O14944 | epiregulin | EPR, ER, Ep | proepiregulin, EPR |
| CASA | CSN1S1 | P47710 | casein alpha s1 | CASA, CSN1 | alpha-S1-casein, |
| | | | | | casoxin-D, casein α |
| | | | | | s1 |
| MMP-12 | MMP12 | P39900 | matrix | HME, ME, MME, | macrophage |
| | | | metallopeptidase | MMP-12 | metalloelastase, |
| | | | 12 | | MME, macrophage |
| | | | | | elastase (ME, hME), |
| | | | | | matrix |
| | | | | | metalloproteinase- |
| | | | | | 12 (MMP-12), |
| | | | | | EC: 3.4.24.65 |
| GALNT2 | GALNT2 | Q10471 | polypeptide N- | CDG2T, GalNAc- | polypeptide GalNAc |
| | | | acetylgalactosami | T2, pp-GaNTase 2 | transferase 2 |
| | | | nyltransferase 2 | | (GalNAc-T2, pp- |
| | | | | | GaNTase2), protein- |
| | | | | | UDP |
| | | | | | acetylgalactosaminy |
| | | | | | ltransferase 2, UDP- |
| | | | | | GalNAc: polypeptide |
| | | | | | N- |
| | | | | | acetylgalactosaminy |
| | | | | | ltransferase 2, |
| | | | | | polypeptide N- |
| | | | | | acetylgalactosaminy |
| | | | | | ltransferase 2 |
| | | | | | soluble form, |
| | | | | | EC: 2.4.1.41 |
| CEACAM-5 | CEACAM5 | P06731 | CEA cell adhesion | CD66e, CEA | carcinoembryonic |
| | | | molecule 5 | | antigen-related cell |
| | | | | | adhesion molecule |
| | | | | | 5, carcinoembryonic |
| | | | | | antigen (CEA), |
| | | | | | meconium antigen |
| | | | | | 100, CD66e |
| VEGF R1 | FLT1 | P17948 | fms related | FLT, FLT-1, | vascular endothelial |
| | | | receptor tyrosine | VEGFR-1, | growth factor |
| | | | kinase 1 | VEGFR1, FRT | receptor 1, VEGFR- |
| | | | | | 1, fms-like tyrosine |
| | | | | | kinase 1 (FLT-1), |
| | | | | | tyrosine-protein |
| | | | | | kinase FRT, |
| | | | | | tyrosine-protein |
| | | | | | kinase receptor FLT |
| | | | | | (FLT), vascular |
| | | | | | permeability factor |
| | | | | | receptor, |
| | | | | | EC: 2.7.10.1 |
| DSPG3 | EPYC | Q99645 | epiphycan | DSPG3, PGLB, | dermatan sulfate |
| | | | | Pg-Lb, SLRR3B | proteoglycan 3, |
| | | | | | proteoglycan-Lb |
| | | | | | (PG-Lb), small |
| | | | | | chondroitin/dermatan |
| | | | | | sulfate |
| | | | | | proteoglycan |
| SorCS1 | SORCS1 | Q8WY21 | sortilin related | hSorCS, SORCS | VPS10 domain- |
| | | | VPS10 domain | | containing receptor |
| | | | containing | | SorCS1, hSorCS |
| | | | receptor 1 | | |
| Matrilin-2 | MATN2 | O00339 | matrilin 2 | UNQ193/PRO219 | matrilin-2 |
| sFRP-3 | FRZB | Q92765 | frizzled related | FRE, OS1, FZRB, | sereted frizzled- |
| | | | protein | hFIZ, FRITZ, | related protein 3, |
| | | | | FRP-3, FRZB1, | sFRP-3, frezzled, |
| | | | | SFRP3, SRFP3, | fritz, frizzled-related |
| | | | | FRZB-1, FRZB- | protein 1, FrzB-1 |
| | | | | PEN, FIZ, FRP | |
| p53 | TP53 | P04637 | tumor protein p53 | BCC7, BMFS5, | P53 cellular tumour |
| | | | | LFS1, P53, | antigen, tumour |
| | | | | TRP53, p53 | protein p53, cellular |
| | | | | | tumor antigen p53, |
| | | | | | antigen NY-CO-13, |
| | | | | | phosphoprotein p53, |
| | | | | | tumor suppressor |
| | | | | | p53 |
| EphB3 | EPHB3 | P54753 | EPH receptor B3 | EK2, ETK2, | ephrin type-B |
| | | | | HEK2, TYRO6 | receptor 3, EPH-like |
| | | | | | tyrosine kinase 2, |
| | | | | | EPH-like kinase 2, |
| | | | | | embryonic kinase 2 |
| | | | | | (EK2, hEK2), |
| | | | | | tyrosine-protein |
| | | | | | kinase TYRO6 |
| NCK1 | NCK1 | P16333 | NCK adaptor | NCK, NCKalpha, | SH2/SH3 adapter |
| | | | protein 1 | nck-1, Nck α | protein NCK1, |
| | | | | | cytoplasmic protein |
| | | | | | NCK1, NCK |
| | | | | | adapter protein 1 |
| | | | | | (Nck-1), SH2/SH3 |
| | | | | | adapter protein |
| | | | | | NCK-alpha |
| Semaphorin | SEMA7A | O75326 | semaphorin 7A | CD108, CDw108, | semaphorin-7A, |
| 7A | | | (JohnMiltonHagen | H-SEMA-K1, H- | CDw108, JMH |
| | | | blood group) | Sema-L, JMH, | blood group antigen, |
| | | | | PFIC11, | John-Milton-Hargen |
| | | | | SEMAK1, | human blood group |
| | | | | SEMAL | Ag, semaphorin-K1 |
| | | | | | (Sema K1), |
| | | | | | semaphorin-L |
| | | | | | (Sema L), CD108 |
| NKp80 | KLRF1 | Q9NZS2 | killer cell lectin | CLEC5C, NKp80, | killer cell lectin-like |
| | | | like receptor F1 | ML, NKP80 | receptor subfamily F |
| | | | | | member 1, lectin- |
| | | | | | like receptor F1, |
| | | | | | activating |
| | | | | | coreceptor NKp80, |
| | | | | | C-type lectin |
| | | | | | domain family 5 |
| | | | | | member C |
| Prolactin | PRL | P01236 | prolactin | GHA1 | |
| Cystatin B | CSTB | P04080 | cystatin B | CPI-B, CST6, | stefin B, stefin-B, |
| | | | | EPM1, EPM1A, | cystatin-B, CPI-B, |
| | | | | PME, STFB, ULD | liver thiol proteinase |
| | | | | | inhibitor |
| Sirtuin 1 | SIRT1 | Q96EB6 | sirtuin 1 | SIR2, SIR2L1, | NAD-dependent |
| | | | | SIR2alpha | protein deacetylase |
| | | | | | sirtuin-1, hSIRT1, |
| | | | | | NAD-dependent |
| | | | | | protein deacylase |
| | | | | | sirtuin-1 (EC: 2.3.1.-), |
| | | | | | regulatory protein |
| | | | | | SIR2 homolog 1, |
| | | | | | SIR2-like protein 1 |
| | | | | | (hSIR2), sirtT1 75 |
| | | | | | kDa fragment |
| | | | | | (75SirT1), |
| | | | | | EC: 2.3.1.286 |
| FGF-16 | FGF16 | O43320 | fibroblast growth | FGF-16, MF4 | FGF-16 |
| | | | factor 16 | | |
| FGF R5 | FGFRL1 | Q8N441 | fibroblast growth | FGFR-5, FGFR5, | fibroblast growth |
| | | | factor receptor | FHFR, | factor receptor-like |
| | | | like 1 | UNQ480/PRO943 | 1, FGF receptor-like |
| | | | | | protein 1, FGF |
| | | | | | homologous factor |
| | | | | | receptor, FGFR-like |
| | | | | | protein, fibroblast |
| | | | | | growth factor |
| | | | | | receptor 5 (FGFR-5) |
| NQO-1 | NQO1 | P15559 | NAD(P)H | DHQU, DIA4, | NAD(P)H |
| | | | quinone | DTD, NMOR1, | dehydrogenase |
| | | | dehydrogenase 1 | NMORI, QR1, | [quinone] 1, |
| | | | | NMO1, NMOR | azoreductase, DT- |
| | | | | | diaphorase (DTD), |
| | | | | | menadione |
| | | | | | reductase, |
| | | | | | NAD(P)H: quinone |
| | | | | | oxidoreductase 1, |
| | | | | | phylloquinone |
| | | | | | reductase, quinone |
| | | | | | reductase 1 (QR1), |
| | | | | | EC: 1.6.5.2 |
| Semaphorin | SEMA6D | Q8NFY4 | semaphorin 6D | KIAA1479 | semaphorin-6D |
| 6D | | | | | |
| FGF-3 | FGF3 | P11487 | fibroblast growth | HBGF-3, INT2, | FGF-3, heparin- |
| | | | factor 3 | FGF-3 | binding growth |
| | | | | | factor 3 (HBGF-3), |
| | | | | | proto-oncogene Int- |
| | | | | | 2 |
| GATA-4 | GATA4 | P43694 | GATA binding | ASD2, TACHD, | transcription factor |
| | | | protein 4 | TOF, VSD1 | GATA-4, GATA- |
| | | | | | binding factor 4 |
| VAP-A | VAPA | Q9P0L0 | VAMP associated | VAMP-A, VAP- | vesicle-associated |
| | | | protein A | 33, VAP-A, | membrane protein- |
| | | | | VAP33, hVAP-33, | associated protein |
| | | | | VAMP- | A, VAMP-A, |
| | | | | ASSOCIATED | VAMP-associated |
| | | | | | protein A, VAP-A, |
| | | | | | 33 kDa VAMP- |
| | | | | | associated protein |
| | | | | | (VAP-33) |
| CHST2 | CHST2 | Q9Y4C5 | carbohydrate | C6ST, GST-2, | carbohydrate |
| | | | sulfotransferase 2 | GST2, Gn6ST-1, | sulphotransferase 2, |
| | | | | HEL-S-75, | galactose/N- |
| | | | | glcNAc6ST-1, | acetylglucosamine/ |
| | | | | GN6ST, GST2 | N- |
| | | | | | acetylglucosamine |
| | | | | | 6-O-sulfotransferase |
| | | | | | 2 (GST-2), N- |
| | | | | | acetylglucosamine |
| | | | | | 6-O-sulfotransferase |
| | | | | | 1 (GlcNAc6ST-1, |
| | | | | | Gn6ST-1) |
| Pappalysin- | PAPPA2 | Q9BXP8 | pappalysin 2 | PAPP-A2, PAPP- | pappalysin-2, |
| 2 | | | | E, PAPPE, | pregnancy- |
| | | | | PLAC3, SSDA | associated plasma |
| | | | | | protein A2 (PAPP- |
| | | | | | A2), pregnancy- |
| | | | | | associated plasma |
| | | | | | protein E1 (PAPP- |
| | | | | | E) |
| Syndecan-3 | SDC3 | O75056 | syndecan 3 | SDCN, SYND3, | syndecan-3, SYND3 |
| | | | | KIAA0468 | |
| Jagged 1 | JAG1 | P78504 | jagged canonical | AGS, AGS1, | protein jagged-1, |
| | | | Notch ligand 1 | AHD, AWS, | jagged1, hJ1, |
| | | | | CD339, | CD339 |
| | | | | CMT2HH, DCHE, | |
| | | | | HJ1, JAGL1, JAG, | |
| | | | | Jagged1 | |
| AKR1C4 | AKR1C4 | P17516 | aldo-keto | 3-alpha-HSD, 3-α- | 3-alpha- |
| | | | reductase family 1 | HSD, C11, CDR, | hydroxysteroid |
| | | | member C4 | CHDR, DD-4, | dehydrogenase type |
| | | | | DD4, HAKRA | I (3-alpha-HSD1, |
| | | | | | EC: 1.1.1.357), |
| | | | | | 3alpha- |
| | | | | | hydroxysteroid 3- |
| | | | | | dehydrogenase, |
| | | | | | chlordecone |
| | | | | | reductase (CDR, |
| | | | | | EC: 1.1.1.225), |
| | | | | | dihydrodiol |
| | | | | | dehydrogenase 4 |
| | | | | | (DD-4, DD4), |
| | | | | | HAKRA, EC: 1.1.1.-, |
| | | | | | EC: 1.1.1.209, |
| | | | | | EC: 1.1.1.210, |
| | | | | | EC: 1.1.1.51, |
| | | | | | EC: 1.1.1.53, |
| | | | | | EC: 1.1.1.62 |
| Olfactomedin-2 | OLFM2 | O95897 | olfactomedin 2 | NOE2, NOELIN2, | noelin-2, |
| | | | | NOELIN2_V1, | olfactomedin-2 |
| | | | | OlfC | |
| Osteoadherin | OMD | Q99983 | osteomodulin | OSAD, SLRR2C, | KSPG |
| | | | | UNQ190/PRO216 | osteomodulin, |
| | | | | | osteoadherin |
| | | | | | (OSAD), keratan |
| | | | | | sulfate proteoglycan |
| | | | | | osteomodulin |
| | | | | | (KSPG |
| | | | | | osteomoduin) |
| NKp44 | NCR2 | O95944 | natural | CD336, LY95, | lymphocyte antigen |
| | | | cytotoxicity | NK-p44, NKP44, | 95 homolog, NK |
| | | | triggering receptor | dJ149M18.1 | cell-activating |
| | | | 2 | | receptor, natural |
| | | | | | killer cell p44- |
| | | | | | related protein (NK- |
| | | | | | p44, NKp44), |
| | | | | | CD336 |
| Thyroglobulin | TG | P01266 | thyroglobuin | AITD3, TGN | Tg |
| IL-21R | IL21R | Q9HBE5 | interleukin 21 | CD360, IMD56, | interleukin-21 |
| | | | receptor | NILR, | receptor, IL-21 |
| | | | | UNQ3121/PRO10 | receptor, IL-21R, |
| | | | | 273, IL21R alpha, | novel interleukin |
| | | | | IL21R α | receptor, CD360 |
| Chemerin | RARRES2 | Q99969 | retinoic acid | HP10433, TIG2, | retinoic acid |
| | | | receptor responder | CHEMERIN | receptor responder |
| | | | 2 | | protein 2, chemerin, |
| | | | | | RAR-responsive |
| | | | | | protein TIG2, |
| | | | | | tazarotene-induced |
| | | | | | gene 2 protein |
| EphAl | EPHA1 | P21709 | EPH receptor A1 | EPH, EPHT, | ephrin type-A |
| | | | | EPHT1, EPH1 | receptor 1, hEpha1, |
| | | | | | EPH tyrosine |
| | | | | | kinase, EPH |
| | | | | | tyrosine kinase 1, |
| | | | | | erythropoietin- |
| | | | | | producing hepatoma |
| | | | | | receptor, tyrosine- |
| | | | | | protein kinase |
| | | | | | receptor EPH, |
| | | | | | EC: 2.7.10.1 |
| CD48 | CD48 | P09326 | CD48 molecule | BCM1, BLAST, | CD48 antigen, |
| | | | | BLAST1, MEM- | BCM1 surface |
| | | | | 102, SLAMF2, | antigen, B- |
| | | | | hCD48, mCD48 | lymphocyte |
| | | | | | activation marker |
| | | | | | BLAST-1, BCM1 |
| | | | | | surface antigen, |
| | | | | | leukocyte antigen |
| | | | | | MEM-102, SLAM |
| | | | | | family member 2 |
| | | | | | (SLAMF2), |
| | | | | | signaling |
| | | | | | lymphocytic |
| | | | | | activation molecule |
| | | | | | 2, TCT.1, CD48 |
| MICB | MICB | Q29980 | MHC class I | PERB11.2, MIC-B | MIC-B |
| | | | polypeptide- | | |
| | | | related sequence | | |
| | | | B | | |
| FGF-5 | FGF5 | P12034 | fibroblast growth | HBGF-5, Smag- | FGF-5, heparin- |
| | | | factor 5 | 82, TCMGLY, | binding growth |
| | | | | FGF-5 | factor 5 (HBGF-5), |
| | | | | | Smag-82 |
| TRANCE | TNFSF11 | O14788 | TNF superfamily | CD254, ODF, | tumor necrosis |
| | | | member 11 | OPGL, OPTB2, | factor ligand |
| | | | | RANKL, | superfamily member |
| | | | | TNLG6B, | 11, osteoclast |
| | | | | TRANCE, | differentiation factor |
| | | | | hRANKL2, sOdf | (ODF), |
| | | | | | osteoprotegerin |
| | | | | | ligand (OPGL), |
| | | | | | receptor activator of |
| | | | | | nuclear factor |
| | | | | | kappa-B ligand |
| | | | | | (RANKL), TNF- |
| | | | | | related activation- |
| | | | | | induced cytokine |
| | | | | | (TRANCE), tumor |
| | | | | | necrosis factor |
| | | | | | ligand superfamily |
| | | | | | member 11 |
| | | | | | membrane form, |
| | | | | | tumor necrosis |
| | | | | | factor ligand |
| | | | | | superfamily member |
| | | | | | 11 soluble form, |
| | | | | | CD254 |
| CES2 | CES2 | O00748 | carboxylesterase 2 | iCE, CE-2, PCE-2, | cocaine esterase, |
| | | | | CES2A1, ICE | CE-2 (hCE-2, |
| | | | | | EC: 3.1.1.1), |
| | | | | | methylumbelliferyl- |
| | | | | | acetate deacetylase |
| | | | | | 2 (EC: 3.1.1.56), |
| | | | | | EC: 3.1.1.84 |
| ULBP-1 | ULBP1 | Q9BZM6 | UL16 binding | N2DL-1, | UL16-binding |
| | | | protein 1 | NKG2DL1, | protein 1, ALCAN- |
| | | | | RAET1I, N2DL1, | beta, NKG2D ligand |
| | | | | ALCAN-beta, | 1 (N2DL-1, |
| | | | | ALCAN-β | NKG2DL1), |
| | | | | | retinoic acid early |
| | | | | | transcript 1I |
| | | | | | (RAET1I) |
| Integrin | ITGA5 | P08648 | integrin subunit | CD49e, FNRA, | integrin alpha-5, |
| alpha 5 | | | alpha 5 | VLA-5, VLA5A | CD49 antigen-like |
| | | | | | family member E, |
| | | | | | fibronectin receptor |
| | | | | | subunit alpha, |
| | | | | | integrin alpha-F, |
| | | | | | VLA-5, integrin |
| | | | | | alpha-5 heavy chain, |
| | | | | | integrin alpha-5 |
| | | | | | light chain, CD49e, |
| | | | | | integrin subunit α 5, |
| | | | | | integrin α 5 |
| VAMP-2 | VAMP2 | P63027 | vesicle associated | NEDHAHM, | vesicle-associated |
| | | | membrane protein | SYB2, VAMP-2 | membrane protein 2, |
| | | | 2 | | VAMP-2, |
| | | | | | synaptobrevin-2, |
| | | | | | synaptobrevin 2 |
| FLRG | FSTL3 | O95633 | follistatin like 3 | FLRG, FSRP, | follistatin-related |
| | | | | UNQ674/PRO1308 | protein 3, follistatin- |
| | | | | | like protein 3, |
| | | | | | follistatin-related |
| | | | | | gene protein |
| Ret Midkine | MDK | P21741 | midkine | ARAP, MK, | MK, amphiregulin- |
| | | | | NEGF2, MK1 | associated protein |
| | | | | | (ARAP), |
| | | | | | midgestation and |
| | | | | | kidney protein, |
| | | | | | neurite outgrowth- |
| | | | | | promoting factor 2, |
| | | | | | neurite outgrowth- |
| | | | | | promoting protein |
| CD73 | NT5E | P21589 | 5′-nucleotidase | CALJA, CD73, | 5′-nucleotidase, 5′ |
| | | | ecto | E5NT, NT, NT5, | nucleotidase, 5′-NT, |
| | | | | NTE, eN, eNT | 5′- |
| | | | | | deoxynucleotidase, |
| | | | | | ecto-5′-nucleotidase, |
| | | | | | IMP-specific 5′- |
| | | | | | nucleotidase, |
| | | | | | thymidylate 5′- |
| | | | | | phosphatase, CD73, |
| | | | | | EC: 3.1.3.35, |
| | | | | | EC: 3.1.3.5, |
| | | | | | EC: 3.1.3.89, |
| | | | | | EC: 3.1.3.91, |
| | | | | | EC: 3.1.3.99 |
| TRACP | ACP5 | P13686 | acid phosphatase | HPAP, TRACP5a, | tartrate-resistant |
| | | | 5, tartrate | TRACP5b, TRAP, | acid phosphatase |
| | | | resistant | TRAcP, TrATPase | type 5, TR-AP, |
| | | | | | tartrate-resistant |
| | | | | | acid ATPase |
| | | | | | (TrATPase), type 5 |
| | | | | | acid phosphatase, |
| | | | | | EC: 3.1.3.2 |
| proGRP | GRP | P07492 | gastrin releasing | BN, preproGRP, | gastrin-releasing |
| | | | peptide | proGRP, GRP-10 | peptide, GRP, |
| | | | | | neuromedin-C |
| | | | | | (GRP-10, GRP18- |
| | | | | | 27) |
| GranzymeH | GZMH | P20718 | granzyme H | CCP-X, CGL-2, | cathepsin G-like 2 |
| | | | | CGL2, CSP-C, | (CTSGL2), CCP-X, |
| | | | | CTLA1, CTSGL2 | cytotoxic T- |
| | | | | | lymphocyte |
| | | | | | proteinase, cytotoxic |
| | | | | | serine protease C |
| | | | | | (CSP-C), |
| | | | | | EC: 3.4.21.- |
| PRX2 | PRDX2 | P32119 | peroxiredoxin 2 | HEL-S-2a, NKEF- | peroxiredoxin-2, |
| | | | | B, NKEFB, PRP, | natural killer cell- |
| | | | | PRX2, PRXII, | enhancing factor B |
| | | | | PTX1, TDPX1, | (NKEF-B), PRP, |
| | | | | TPX1, TSA | thiol-specific |
| | | | | | antioxidant protein |
| | | | | | (TSA), thioredoxin |
| | | | | | peroxidase 1, |
| | | | | | thioredoxin- |
| | | | | | dependent peroxide |
| | | | | | reductase 1, |
| | | | | | thioredoxin- |
| | | | | | dependent |
| | | | | | peroxiredoxin 2, |
| | | | | | EC: 1.11.1.24 |
| p27 | CDKN1B | P46527 | cyclin dependent | CDKN4, KIP1, | cyclin-dependent |
| | | | kinase inhibitor | MEN1B, MEN4, | kinase inhibitor 1B, |
| | | | 1B | P27KIP1, p27, | cyclin-dependent |
| | | | | P28-ICK | kinase inhibitor p27, |
| | | | | | p27Kip1 |
| Siglec-6 | SIGLEC6 | O43699 | sialic acid binding | CD327, CD33L, | sialic acid-binding |
| | | | Ig like lectin 6 | CD33L1, | Ig-like lectin 6, |
| | | | | CD33L2, | siglec-6, CD33 |
| | | | | CDW327, OBBP1 | antigen-like 1, |
| | | | | | CDw327, obesity- |
| | | | | | binding protein 1 |
| | | | | | (OB-BP1), CD327 |
| Dectin-1 | CLEC7A | Q9BXN2 | C-type lectin | BGR, CANDF4, | C-type lectin |
| | | | domain containing | CD369, | domain family 7 |
| | | | 7A | CLECSF12, | member A, beta- |
| | | | | DECTIN1, | glucan receptor, C- |
| | | | | SCARE2, | type lectin |
| | | | | UNQ539/PRO1082 | superfamily member |
| | | | | | 12, dendritic cell- |
| | | | | | associated C-type |
| | | | | | lectin 1, DC- |
| | | | | | associated C-type |
| | | | | | lectin 1, CD369 |
| CD51 | ITGAV | P06756 | integrin subunit | CD51, MSK8, | integrin alpha-V, |
| | | | alpha V | VNRA, VTNR, | vitronectin receptor, |
| | | | | alpha V, α V | vitronectin receptor |
| | | | | | subunit alpha, |
| | | | | | integrin alpha-V |
| | | | | | heavy chain, |
| | | | | | integrin alpha-V |
| | | | | | light chain, CD51, |
| | | | | | integrin subunit α V, |
| | | | | | integrin α v |
| Notch-1 | NOTCH1 | P46531 | notch receptor 1 | AOS5, AOVD1, | neurogenic locus |
| | | | | TAN1, hN1 | notch homolog |
| | | | | | protein 1, Notch 1, |
| | | | | | hN1, translocation- |
| | | | | | associated notch |
| | | | | | protein TAN-1, |
| | | | | | Notch 1 |
| | | | | | extracellular |
| | | | | | truncation (NEXT), |
| | | | | | Notch 1 intracellular |
| | | | | | domain (NICD) |
| Calreticulin | CALR | P27797 | calreticulin | CALR1, CRT, | calreticulin isoform |
| | | | | HEL-S-99n, RO, | 1, CRP55, |
| | | | | SSA, cC1qR, | calregulin, |
| | | | | CRTC | endoplasmic |
| | | | | | reticulum resident |
| | | | | | protein 60 (ERp60), |
| | | | | | HACBP, grp60 |
| DR3 | TNFRSF | Q93038 | TNF receptor | APO-3, APO3, | tumor necrosis |
| | 25 | | superfamily | DDR3, DR3, | factor receptor |
| | | | member 25 | GEF720, LARD, | superfamily member |
| | | | | PLEKHG5, | 25, Apo-3, |
| | | | | TNFRSF12, TR3, | apoptosis-inducing |
| | | | | TRAMP, WSL-1, | receptor AIR, |
| | | | | WSL-LR, WSL, | apoptosis-mediating |
| | | | | | receptor DR3, |
| | | | | WSL1, | apoptosis-mediating |
| | | | | UNQ455/PRO779 | receptor TRAMP, |
| | | | | | death receptor 3, |
| | | | | | lymphocyte- |
| | | | | | associated receptor |
| | | | | | of death (LARD), |
| | | | | | protein WSL, |
| | | | | | protein WSL-1 |
| DCTN1 | DCTN1 | Q14203 | dynactin subunit 1 | DAP-150, DP-150, | dynactin 1, 150 kDa |
| | | | | HMND14, P135 | dynein-associated |
| | | | | | polypeptide, DAP- |
| | | | | | 150 (DP-150), p135, |
| | | | | | p150-glued |
| CDC25B | CDC25B | P30305 | cell division cycle | CDC25HU2 | M-phase inducer |
| | | | 25B | | phosphatase 2, dual |
| | | | | | specificity |
| | | | | | phosphatase |
| | | | | | Cdc25B, |
| | | | | | EC: 3.1.3.48 |
| Osteoactivin | GPNMB | Q14956 | glycoprotein nmb | HGFIN, NMB, | transmembrane |
| | | | | PLCA3, | glycoprotein NMB, |
| | | | | UNQ1725/PRO9925 | hematopoietic |
| | | | | | growth factor |
| | | | | | inducible |
| | | | | | neurokinin-1 type |
| ACE | ACE | P12821 | angiotensin I | ACE1, CD143, | angiotensin- |
| | | | converting | DCP, DCP1 | converting enzyme |
| | | | enzyme | | angiotensin |
| | | | | | converting enzyme, |
| | | | | | ACE isoform 3, |
| | | | | | ACE, dipeptidyl |
| | | | | | carboxypeptidase I, |
| | | | | | kininase II, |
| | | | | | angiotensin- |
| | | | | | converting enzyme |
| | | | | | soluble form, |
| | | | | | CD143, EC: 3.4.15.1 |
| CA125 | MUC16 | Q8WXI7 | mucin 16, cell | CA125, | mucin-16, MUC-16, |
| | | | surface associated | LOC100293231, | ovarian cancer- |
| | | | | LOC100508913, | related tumor |
| | | | | LOC101060361 | marker CA125 (CA- |
| | | | | | 125), ovarian |
| | | | | | carcinoma antigen |
| | | | | | CA125 |
| HAO-1 | HAO1 | Q9UJM8 | hydroxyacid | GO, GOX, GOX1, | 2-hydroxyacid |
| | | | oxidase 1 | HAOX1 | oxidase 1, HAOX1, |
| | | | | | glycolate oxidase |
| | | | | | (GO, GOX), |
| | | | | | glyoxylate oxidase |
| | | | | | (EC: 1.2.3.5), |
| | | | | | EC: 1.1.3.15 |
| PSMA1 | PSMA1 | P25786 | proteasome 20S | HC2, HEL-S-275, | proteaseome subunit |
| | | | subunit alpha 1 | NU, PROS30, | alpha type-1, |
| | | | | PSC2 | proteasome 20S |
| | | | | | subunit α 1, 30 kDa |
| | | | | | prosomal protein |
| | | | | | (PROS-30), |
| | | | | | macropain subunit |
| | | | | | C2, multicatalytic |
| | | | | | endopeptidase |
| | | | | | complex subunit C2, |
| | | | | | proteasome |
| | | | | | component C2, |
| | | | | | proteasome nu |
| | | | | | chain, proteasome |
| | | | | | subunit alpha-6 |
| | | | | | (alpha-6) |
| FCRLB | FCRLB | Q6BAA4 | Fc receptor like B | FCRL2, FCRLM2, | Fc receptor-like B, |
| | | | | FCRLY, FREB-2, | Fc receptor homolog |
| | | | | FREB2, FcRY, | expressed in B-cells |
| | | | | FCRY | protein 2 (FREB-2), |
| | | | | | Fc receptor-like and |
| | | | | | mucin-like protein |
| | | | | | 2, Fc receptor-like |
| | | | | | protein 2, Fc |
| | | | | | receptor-related |
| | | | | | protein Y (FcRY) |
| BMP-9 | GDF2 | Q9UK05 | growth | BMP-9, BMP9, | growth/differentiation |
| | | | differentiation | HHT5 | factor 2, GDF-2, |
| | | | factor 2 | | bone morphogenic |
| | | | | | protein 9 (BMP-9) |
| CRIM1 | CRIM1 | Q9NZV1 | cysteine rich | CRIM-1, S52, | cysteine-rich motor |
| | | | transmembrane | INQ1886/PRO4330 | neuron 1 protein, |
| | | | BMP regulator 1 | | CRIM-1, cysteine- |
| | | | | | rich repeat- |
| | | | | | containing protein |
| | | | | | S52, processed |
| | | | | | cysteine-rich motor |
| | | | | | neuron 1 protein |
| LIF | LIF | P15018 | LIF interleukin 6 | CDF, DIA, | leukemia inhibitory |
| | | | family cytokine | HILDA, MLPLI | factor, |
| | | | | | differentiation- |
| | | | | | stimulating factor |
| | | | | | (D factor), |
| | | | | | melanoma-derived |
| | | | | | LPL inhibitor |
| | | | | | (MLPLI), |
| | | | | | emfilermin, |
| | | | | | cholinergic |
| | | | | | differentiation factor |
| | | | | | (CDF) |
| SPINK1 | SPINK1 | P00995 | serine peptidase | PCTT, PSTI, | serine protease |
| | | | inhibitor Kazal | PSTI1, Spink3, | inhibitor Kazal-type |
| | | | type 1 | TATI, TCP | 1, serine protease |
| | | | | | inhibitor Kazal type |
| | | | | | 1, pancreatic |
| | | | | | secretory trypsin |
| | | | | | inhibitor, tumor- |
| | | | | | associated trypsin |
| | | | | | inhibitor (TATI) |
| EphB6 | EPHB6 | O15197 | EPH receptor B6 | HEP, EPHB5 | ephrin type-B |
| | | | | | receptor 6, HEP, |
| | | | | | tyrosine-protein |
| | | | | | kinase-defective |
| | | | | | receptor EPH-6 |
| RGM-B | RGMB | Q6NW40 | repulsive guidance | DRAGON, | repulsive guidance |
| | | | molecule BMP | LOC285705 | molecule B, |
| | | | co-receptor b | | DRG11-responsive |
| | | | | | axonal guidance and |
| | | | | | outhgrowth of |
| | | | | | neurite (DRAGON) |
| HS3ST1 | HS3ST1 | O14792 | heparan sulfate- | 3OST, 3OST1 | heparan sulfate |
| | | | glucosamine 3- | | glucosamine 3-O- |
| | | | sulfotransferase 1 | | sulfotransferase 1, |
| | | | | | heparan sulfate D- |
| | | | | | glucosaminyl 3-O- |
| | | | | | sulfotransferase 1 |
| | | | | | (3-OST-1), heparan |
| | | | | | sulfate 3-O- |
| | | | | | sulfotransferase 1 |
| | | | | | (h3-OST-1), |
| | | | | | heparan sulfate D- |
| | | | | | glucosaminyl 3-O- |
| | | | | | sulfotransferase 1 |
| | | | | | precursor, heparan |
| | | | | | sulphate D- |
| | | | | | glucosaminyl 3-O- |
| | | | | | sulphotransferase 1 |
| | | | | | precursor, heparan |
| | | | | | sulphate- |
| | | | | | glucosamine 3- |
| | | | | | sulphotransferase 1 |
| ROR1 | ROR1 | Q01973 | receptor tyrosine | NTRKR1, | inactive tyrosine- |
| | | | kinase like orphan | dJ537F10.1 | protein kinase |
| | | | receptor 1 | | transmembrane |
| | | | | | receptor ROR1, |
| | | | | | neurotrophic |
| | | | | | tyrosine kinase |
| | | | | | receptor-related 1 |
| CMG-2 | ANTXR2 | P58335 | ANTXR cell | CMG-2, CMG2, | anthrax toxin |
| | | | adhesion molecule | HFS, ISH, JHF, | receptor 2, capillary |
| | | | 2 | ANTRX2, ATR2 | morphogenesis gene |
| | | | | | 2 protein (CMG-2) |
| 4-1BB | TNFSF9 | P41273 | TNF superfamily | 4-1BB-L, | tumor necrosis |
| Ligand | | | member 9 | CD137L, | factor ligand |
| | | | | TNLG5A | superfamily member |
| | | | | | 9, 4-1BB ligand (4- |
| | | | | | 1BBL) |
| L1CAM-2 | CHL1 | O00533 | cell adhesion | CALL, L1CAM2 | neural cell adhesion |
| | | | molecule L1 like | | molecule L1-like |
| | | | | | protein, close |
| | | | | | homolog of L1, |
| | | | | | processed neural |
| | | | | | cell adhesion |
| | | | | | molecule L1-like |
| | | | | | protein |
| p63 | TP63 | Q9H3D4 | tumor protein p63 | AIS, B(p51A), | tumor protein 63, |
| | | | | B(p51B), EEC3, | p63, chronic |
| | | | | KET, LMS, NBP, | ulcerative stomatitis |
| | | | | OFC8, RHS, | protein (CUSP), |
| | | | | SHFM4, TP53CP, | keratinocyte |
| | | | | TP53L, TP73L, | transcription factor |
| | | | | p40, p51, p53CP, | KET, |
| | | | | p63, P63, p73H, | transformation- |
| | | | | P73H, p73L, | related protein 63 |
| | | | | p63/p40 | (TP63), tumor |
| | | | | | protein p73-like |
| | | | | | (p73L), tumor |
| | | | | | protein 63 kDa with |
| | | | | | strong homology to |
| | | | | | p53, tumour protein |
| | | | | | 63 kDa with stron |
| | | | | | homology to p53, |
| | | | | | tumour protein p63, |
| | | | | | p40, p51 |
| Cathepsin V | CTSV | O60911 | cathepsin V | CATL2, CTSL2, | cathepsin L2, |
| | | | | CTSU, | cathepsin U, |
| | | | | UNQ268/PRO305 | EC: 3.4.22.43 |
| Testican 2 | SPOCK2 | Q92563 | SPARC | testican-2, | testican-2, |
| | | | (osteonectin), | KIAA0275, | SPARC/osteonectin |
| | | | cwcv and kazal | TICN2, | CWCV and Kazal- |
| | | | like domains | UNQ269/PRO306 | like domains |
| | | | proteoglycan 2 | | proteoglycan 2 |
| Glypican 5 | GPC5 | P78333 | glypican 5 | | glypican-5, secreted |
| | | | | | glypican-5 |
| CD6 | CD6 | P30203 | CD6 molecule | TP120 | T-cell differentiation |
| | | | | | antigen CD6, T12, |
| | | | | | TP120, soluble |
| | | | | | CD6, CD6 |
| Siglec-2 | CD22 | P20273 | CD22 molecule | SIGLEC-2, | B-cell receptor |
| | | | | SIGLEC2, | CD22, B- |
| | | | | FLJ22814 | lymphocyte cell |
| | | | | | adhesion molecule |
| | | | | | (BL-CAM), sialic |
| | | | | | acid-binding Ig-like |
| | | | | | lectin 2 (Siglec-2), |
| | | | | | T-cell surface |
| | | | | | antigen Leu-14, |
| | | | | | CD22 |
| Legumain | LGMN | Q99538 | legumain | AEP, LGMN1, | asparaginy1 |
| | | | | PRSC1 | endopeptidase |
| | | | | | (AEP), protease |
| | | | | | cysteine 1, |
| | | | | | EC: 3.4.22.34 |
| PRELP | PRELP | P51888 | proline and | MST161, | prolargin, proline- |
| | | | arginine rich end | MSTP161, | arginine-rich end |
| | | | leucine rich repeat | SLRR2A | leucine-rich repeat |
| | | | protein | | protein |
| CES1 | CES1 | P23141 | carboxylesterase 1 | ACAT, CE-1, | liver |
| | | | | CE1, CEH, CES2, | carboxylesterase 1, |
| | | | | HMSE, HMSE1, | liver microsomal |
| | | | | PCE-1, REH, | carboxylesterase, |
| | | | | SES1, TGH, hCE- | acyl-coenzyme |
| | | | | 1, CES1A1, | A: cholesterol |
| | | | | NCEH | acyltransferase |
| | | | | | (ACAT), brain |
| | | | | | carboxylesterase |
| | | | | | hBr1, |
| | | | | | carboxylesterase |
| | | | | | (CE-1, hCE-1, |
| | | | | | EC: 3.1.1.1), |
| | | | | | cholesteryl ester |
| | | | | | hydrolase (CEH, |
| | | | | | EC: 3.1.1.13), |
| | | | | | cocaine |
| | | | | | carboxylesterase, |
| | | | | | egasyn, HMSE, |
| | | | | | methylumbelliferyl- |
| | | | | | acetate deacetylase |
| | | | | | 1 (EC: 3.1.1.56), |
| | | | | | monocyte/macropha |
| | | | | | ge serine esterase, |
| | | | | | retinyl ester |
| | | | | | hydrolase (REH), |
| | | | | | serine esterase 1, |
| | | | | | triacylglycerol |
| | | | | | hydrolase (TGH) |
| TAZ | WWTR1 | Q9GZV5 | WW domain | TAZ, | WW domain- |
| | | | containing | DKFZp586I1419 | containing |
| | | | transcription | | transcription |
| | | | regulator 1 | | regulator protein 1, |
| | | | | | transcriptional |
| | | | | | coactivator with |
| | | | | | PDZ-binding motif |
| NSE | ENO2 | P09104 | enolase 2 | HEL-S-279, NSE | gamma-enolase, 2- |
| | | | | | phospho-D- |
| | | | | | glycerate hydro- |
| | | | | | lyase, neural |
| | | | | | enolase, neuron- |
| | | | | | specific enolase |
| | | | | | (NSE), EC: 4.2.1.11 |
| TECK | CCL25 | O15444 | C-C motif | Ck beta-15, Ck β- | C-C motif |
| | | | chemokine ligand | 15, Ckb15, | chemokine 25, |
| | | | 25 | SCYA25, TECK, | chemokine TECK, |
| | | | | TECKvar | small-inducible |
| | | | | | cytokine A25, |
| | | | | | thymus-expressed |
| | | | | | chemokine |
| HTRA2 | HTRA2 | O43464 | HtrA serine | MGCA8, OMI, | serine protease |
| | | | peptidase 2 | PARK13, PRSS25 | HTRA2 |
| | | | | | mitochondrial, high |
| | | | | | temperature |
| | | | | | requirement protein |
| | | | | | A2 (HtrA2), omi |
| | | | | | stress-regulated |
| | | | | | endoprotease, serine |
| | | | | | protease 25, serine |
| | | | | | proteinase OMI, |
| | | | | | EC: 3.4.21.108 |
| HIF-1 beta | ARNT | P27540 | aryl hydrocarbon | ARNT1, HIF-1- | ARNT protein, class |
| | | | receptor nuclear | beta, HIF-1-β, | E basic helix-loop- |
| | | | translocator | HIF-1beta, HIF1- | helix protein 2 |
| | | | | beta, HIF1-β, | (bHLHe2), dioxin |
| | | | | HIF1B, | receptor nuclear |
| | | | | HIF1BETA, | translocator, |
| | | | | TANGO, | hypoxia-inducible |
| | | | | bHLHe2, | factor 1-beta (HIF- |
| | | | | BHLHE2 | 1-beta, HIF1-beta) |
| TAFA1 | TAFA1 | Q7Z5A9 | TAFA chemokine | FAM19A1, | chemokine-like |
| | | | like family | TAFA-1 | protein TAFA-1 |
| | | | member 1 | | |
| Podocalyxin | PODXL | O00592 | podocalyxin like | PC, PDX, PCLP, | podocalyxin, |
| | | | | Gp200, gp135, | GCTM-2 antigen, |
| | | | | PCLP-1, PCLP1, | Gp200, |
| | | | | PODXL1, RCC | podocalyxin-like |
| | | | | antigen, TRA-1-60 | protein 1 (PC, |
| | | | | | PCLP-1) |
| RalA | RALA | P11233 | RAS like proto- | HINCONS, RAL | Ras-related protein |
| | | | oncogene A | | Ral-A, EC: 3.6.5.2 |
| CRELD2 | CRELD2 | Q6UXH1 | cysteine rich with | UNQ185/PRO211 | protein disulfide |
| | | | EGF like domains | | isomerase CRELD2, |
| | | | 2 | | cysteine-rich with |
| | | | | | EGF-like domain |
| | | | | | protein 2, |
| | | | | | EC: 5.3.4.1 |
| GRAP2 | GRAP2 | O75791 | GRB2 related | GADS, GRAP-2, | GRB2-related |
| | | | adaptor protein 2 | GRB2L, GRBLG, | adapter protein 2, |
| | | | | GRID, GRPL, | Grb2-related 2, |
| | | | | GrbX, Grf40, | adapter protein |
| | | | | Mona, P38 | GRID, GRB-2-like |
| | | | | | protein (GRB2L), |
| | | | | | GRBLG, GRBX, |
| | | | | | Grf40 adapter |
| | | | | | protein (Grf-40), |
| | | | | | growth factor |
| | | | | | receptor-binding |
| | | | | | protein, |
| | | | | | hematopoietic cell- |
| | | | | | associated adapter |
| | | | | | protein GrpL, P38, |
| | | | | | protein GADS, |
| | | | | | SH3-SH2-SH3 |
| | | | | | adapter Mona |
| SP-D | SFTPD | P35247 | surfactant protein | COLEC7, PSP-D, | pulmonary |
| | | | D | SFTP4, SP-D, | surfactant- |
| | | | | PSPD | associated protein |
| | | | | | D, PSP-D, SP-D, |
| | | | | | collectin-7, lung |
| | | | | | surfactant protein D |
| BID | BID | P55957 | BH3 interacting | FP497 | BH3-interacting |
| | | | domain death | | domain death |
| | | | agonist | | agonist, p22 BID |
| | | | | | (BID), BH3- |
| | | | | | interacting domain |
| | | | | | death agonist p15 |
| | | | | | (p15 BID), BH3- |
| | | | | | interacting domain |
| | | | | | death agonist p13 |
| | | | | | (p13 BID), BH3- |
| | | | | | interacting domain |
| | | | | | death agonist p11 |
| | | | | | (p11 BID) |
| GFR alpha- | GFRA2 | O00451 | GDNF family | GDNFRB, | GDNF family |
| 2 | | | receptor alpha 2 | NRTNR-ALPHA, | receptor alpha-2, |
| | | | | NRTNR-α, | GDNF family |
| | | | | NTNRA, RETL2, | receptor α 2, |
| | | | | TRNR2 | GFRA2 isoform 1, |
| | | | | | Gfr α 2, GDNF |
| | | | | | receptor alpha-2, |
| | | | | | GDNFR-alpha-2, |
| | | | | | GFR-alpha-2, |
| | | | | | GDNF receptor beta |
| | | | | | (GDNFR-beta), |
| | | | | | neurturin receptor |
| | | | | | alpha (NRTNR- |
| | | | | | alpha, NTNR- |
| | | | | | alpha), RET ligand |
| | | | | | 2, TGF-beta-related |
| | | | | | neurotrophic factor |
| | | | | | receptor 2 |
| Notch-3 | NOTCH3 | Q9UM47 | notch receptor 3 | CADASIL, | neurogenic locus |
| | | | | CADASIL1, | notch homolog |
| | | | | CASIL, IMF2, | protein 3, notch 3, |
| | | | | LMNS | notch 3 extracellular |
| | | | | | truncation, notch 3 |
| | | | | | intracellular domain |
| VEGF R3 | FLT4 | P35916 | fms related | CHTD7, FLT-4, | vascular endothelial |
| | | | receptor tyrosine | LMPH1A, | growth factor |
| | | | kinase 4 | LMPHM1, PCL, | receptor 3, VEGFR- |
| | | | | VEGFR-3, | 3, fms-like tyrosine |
| | | | | VEGFR3, FLT41 | kinase 4 (FLT-4), |
| | | | | | tyrosine-protein |
| | | | | | kinase receptor |
| | | | | | FLT4 |
| DLL4 | DLL4 | Q9NR61 | delta like | AOS6, delta4, | delta-like protein 4, |
| | | | canonical Notch | hdelta2, | Drosophila Delta |
| | | | ligand 4 | UNQ1895/PRO4341 | homolog 4 (Delta4), |
| | | | | | δ like canonical |
| | | | | | Notch ligand 4 |
| TGFb2 | TGFB2 | P61812 | transforming | G-TSF, LDS4, | transforming growth |
| | | | growth factor beta | TGF-beta2 | factor β 2, |
| | | | 2 | | transforming growth |
| | | | | | factor beta-2 |
| | | | | | proprotein, |
| | | | | | cetermin, plyergin, |
| | | | | | glioblastoma- |
| | | | | | derived T-cell |
| | | | | | suppressor factor |
| | | | | | (G-TSF), latency- |
| | | | | | associated peptide |
| | | | | | (LAP), transforming |
| | | | | | growth factor beta-2 |
| | | | | | (TGF-beta-2) |
| LIGHT | TNFSF14 | O43557 | TNF superfamily | CD258, HVEML, | tumor necrosis |
| | | | member 14 | LIGHT, LTg, | factor ligand |
| | | | | UNQ391/PRO726 | superfamily member |
| | | | | | 14, herpes virus |
| | | | | | entry mediator |
| | | | | | ligand (HVEM-L), |
| | | | | | herpesvirus entry |
| | | | | | mediator ligand, |
| | | | | | tumor necrosis |
| | | | | | factor ligand |
| | | | | | superfamily member |
| | | | | | 14 membrane form, |
| | | | | | tumor necrosis |
| | | | | | factor ligand |
| | | | | | superfamily member |
| | | | | | 14 soluble form, |
| | | | | | CD258 |
| XIAP | XIAP | P98170 | X-linked inhibitor | API3, BIRC4, | E3 ubiquitin-protein |
| | | | of apoptosis | IAP-3, IAP3, | ligase XIAP, |
| | | | | ILP1, MHIA, | apoptosis inhibitor |
| | | | | XLP2, hIAP-3, | 3, baculoviral IAP |
| | | | | hIAP3 | repeat-containing |
| | | | | | protein 4, IAP-like |
| | | | | | protein (ILP, hILP), |
| | | | | | inhibitor of |
| | | | | | apoptosis protein 3 |
| | | | | | (IAP-3, hIAP-3, |
| | | | | | hIAP3), RING-type |
| | | | | | E3 ubiquitin |
| | | | | | transferase XIAP, |
| | | | | | X-linked inhibitor of |
| | | | | | apoptosis protein |
| | | | | | (X-linked IAP) |
| ST8SIA1 | ST8SIA1 | Q92185 | ST8 alpha-N- | GD3S, SIAT8, | alpha-N- |
| | | | acetyl- | SIAT8-A, | acetylneuraminide |
| | | | neuraminide | SIAT8A, ST8SiaI, | alpha-2,8- |
| | | | alpha-2,8- | SiaI-T2 | sialyltransferase, |
| | | | sialyltransferase 1 | | alpha-2,8- |
| | | | | | sialyltransferase 8A, |
| | | | | | ganglioside GD3 |
| | | | | | synthase, |
| | | | | | ganglioside GT3 |
| | | | | | synthase, |
| | | | | | sialyltransferase 8A |
| | | | | | (SIAT8-A), |
| | | | | | sialyltransferase |
| | | | | | St8Sia I (ST8Sial), |
| | | | | | ST8 α-N-acetyl- |
| | | | | | neuraminide α-2,8- |
| | | | | | sialyltransferase 1, |
| | | | | | α-2,8 |
| | | | | | sialyltransferase, α- |
| | | | | | 2,8 ST, Sial-T2, |
| | | | | | alpha-2,8 ST |
| Cathepsin L | CTSL | P07711 | cathepsin L | CATL, MEP, | procathepsin L, |
| | | | | CSTL1, CTSL1 | cathepsin L1, major |
| | | | | | excreted protein |
| | | | | | (MEP), cathepsin L |
| | | | | | heavy chain, |
| | | | | | cathepsin L light |
| | | | | | chain, EC: 3.4.22.15 |
| 6Ckine | CCL21 | O00585 | C-C motif | 6Ckine, CKb9, | C-C motif |
| | | | chemokine ligand | ECL, SCYA21, | chemokine 21, |
| | | | 21 | SLC, TCA4, | 6Ckine, beta- |
| | | | | UNQ784/PRO1600 | chemokiine exodus- |
| | | | | | 2, secondary |
| | | | | | lymphoid-tissue |
| | | | | | chemokine (SLC), |
| | | | | | small-inducible |
| | | | | | cytokine A21 |
| MIS RII | AMHR2 | Q16671 | anti-Mullerian | AMHR, MISR2, | anti-Muellerian |
| | | | hormone receptor | MISRII, MRII, | hormone type-2 |
| | | | type 2 | MIS receptor | receptor, anti- |
| | | | | | Muellerian hormone |
| | | | | | type II receptor |
| | | | | | (AMH type II |
| | | | | | receptor), MIS type |
| | | | | | II receptor (MISRII, |
| | | | | | MRII), MIS receptor |
| Kallikrein 5 | KLK5 | Q9Y337 | kallikrein related | KLK-L2, KLKL2, | kallikrein-5, |
| | | | peptidase 5 | SCTE, | kallikrein-like |
| | | | | UNQ570/PRO1132 | protein 2 (KLK-L2), |
| | | | | | stratum corneum |
| | | | | | tryptic enzyme |
| TGM3 | TGM3 | Q08188 | transglutaminase 3 | TGE, UHS2, EC | protein-glutatmine |
| | | | | 2.3.2.13 | gamma- |
| | | | | | glutamyltransferase |
| | | | | | E, transglutaminase |
| | | | | | E, TG(E), TGE, |
| | | | | | TGase E, |
| | | | | | transglutaminase-3 |
| | | | | | (TGase-3), protein- |
| | | | | | glutamine gamma- |
| | | | | | glutamyltransferase |
| | | | | | E 50 kDa catalytic |
| | | | | | chain, protein- |
| | | | | | glutamine gamma- |
| | | | | | glutamyltransfease |
| | | | | | E 27 kDa non- |
| | | | | | catalytic chain |
| FCAR | FCAR | P24071 | Fc alpha receptor | CD89, CTB- | immunoglobulin |
| | | | | 61M7.2, | alpha Fc receptor, |
| | | | | FcalphaR, | IgA Fc receptor, |
| | | | | FcalphaRI | CD89, FC alpha |
| | | | | | receptor 1, Fc alpha |
| | | | | | RI, Fc α receptor, |
| | | | | | FC α receptor 1, Fc |
| | | | | | α RI |
| Contactin-2 | CNTN2 | Q02246 | contactin 2 | AXT, EPEO5, | contactin-2, axonal |
| | | | | FAME5, TAG-1, | glycoprotein TAG- |
| | | | | TAG1, TAX, | 1, axonin-1, |
| | | | | TAX1 | transient axonal |
| | | | | | glycoprotein (TAX- |
| | | | | | 1), axonin-1 |
| CD83 | CD83 | Q01151 | CD83 molecule | BL11, HB15 | CD83 antigen, |
| | | | | | hCD83, B-cell |
| | | | | | activation protein, |
| | | | | | cell surface protein |
| | | | | | HB15, CD83 |
| IL-1 R3 | IL1RAP | Q9NPH3 | interleukin 1 | C3orf13, IL- | interleukin-1 |
| | | | receptor accessory | 1RAcP, IL1R3, | receptor accessory |
| | | | protein | ILRAP | protein, IL-1 |
| | | | | | receptor accessory |
| | | | | | protein, IL-1RAcP, |
| | | | | | interleukin-1 |
| | | | | | receptor 3 (IL-1R-3, |
| | | | | | IL-1R3), EC: 3.2.2.6 |
| SALM4 | LRFN3 | Q9BTN0 | leucine rich repeat | FIGLER1, | leucine-rich repeat |
| | | | and fibronectin | SALM4, | and fibronectin |
| | | | type III domain | UNQ5865/PRO34192 | type-III domain- |
| | | | containing 3 | | containing protein 3, |
| | | | | | synaptic adhesion- |
| | | | | | like molecule 4 |
| GBA3 | GBA3 | Q9H227 | glucosylceramidase | CBG, CBGL1, | glucosylceramidase |
| | | | beta 3 | GLUC, KLRP | β 3 |
| | | | (gene/pseudogene) | | (gene/pseudogene), |
| | | | | | cytosolic beta- |
| | | | | | glucosidase, |
| | | | | | cytosolic beta- |
| | | | | | glucosidase-like |
| | | | | | protein 1, cytosolic |
| | | | | | galactosylceramidase |
| | | | | | (EC: 3.2.1.46), |
| | | | | | cytosolic |
| | | | | | glucosylceramidase |
| | | | | | (EC: 3.2.1.45), |
| | | | | | cytosolic |
| | | | | | glycosylceramidase |
| | | | | | (cytosolic GCase), |
| | | | | | glucosidase beta |
| | | | | | acid 3, |
| | | | | | glucosylceramidase |
| | | | | | beta 3, klotho- |
| | | | | | related protein |
| | | | | | (KLrP) |
| ROBO4 | ROBO4 | Q8WZ75 | roundabout | AOVD3, ECSM4, | roundabout homolog |
| | | | guidance receptor | MRB, | 4, magic roundabout |
| | | | 4 | UNQ421/PRO3674 | |
| OSCAR | OSCAR | Q8IYS5 | osteoclast | PIGR3, PIgR-3 | osteoclast- |
| | | | associated Ig-like | | associated |
| | | | receptor | | immunoglobulin- |
| | | | | | like receptor, |
| | | | | | osteoclast- |
| | | | | | associated receptor, |
| | | | | | hOSCAR, |
| | | | | | polymeric |
| | | | | | immunoglobulin |
| | | | | | receptor 3 (PIgR-3, |
| | | | | | PIgR3, Poly-Ig |
| | | | | | receptor 3) |
| VEGF | VEGFA | P15692 | vascular | L-VEGF, | vascular endothelial |
| | | | endothelial growth | MVCD1, VEGF, | growth factor A |
| | | | factor A | VPF | long form, L-VEGF, |
| | | | | | vascular |
| | | | | | permeability factor |
| | | | | | (VPF), N-VEGF |
| IGSF3 | IGSF3 | O75054 | immunoglobulin | EWI-3, LCDD, | IgSF3, Glu-Trp-Ile |
| | | | superfamily | V8, EWI3, | EWI motif- |
| | | | member 3 | KIAA0466 | containing protein 3 |
| | | | | | (EWI-3) |
| Biglycan | BGN | P21810 | biglycan | DSPG1, MRLS, | bone/cartilage |
| | | | | PG-S1, PGI, | proteoglycan I, PG- |
| | | | | SEMDX, | S1 |
| | | | | SLRR1A | |
| Neudesin | NENF | Q9UMX5 | neudesin | CIR2, SCIRP10, | neudesin, cell |
| | | | neurotrophic | SPUF | immortalization- |
| | | | factor | | related protein 2, |
| | | | | | neuron-derived |
| | | | | | neurotrophic factor, |
| | | | | | protein GIG47, |
| | | | | | secreted protein of |
| | | | | | unknown function |
| | | | | | (SPUF protein) |
| ILT4 | LILRB2 | Q8N423 | leukocyte | CD85D, ILT-4, | leukocyte |
| | | | immunoglobulin | ILT4, LIR-2, | immunoglobulin- |
| | | | like receptor B2 | LIR2, MIR-10, | like receptor |
| | | | | MIR10, | subfamily B |
| | | | | LOC102724997 | member 2, LIR-2, |
| | | | | | leukocyte |
| | | | | | immunoglobulin- |
| | | | | | like receptor 2, |
| | | | | | CD85 antigen-like |
| | | | | | family member D, |
| | | | | | immunoglobulin- |
| | | | | | like transcript 4 |
| | | | | | (ILT-4), |
| | | | | | monocyte/macrophage |
| | | | | | immunoglobulin- |
| | | | | | like receptor 10 |
| | | | | | (MIR-10), CD85d |
| uPAR | PLAUR | Q03405 | plasminogen | CD87, U-PAR, | urokinase |
| | | | activator, | UPAR, URKRK, | plasminogen |
| | | | urokinase receptor | MO3 | activator surface |
| | | | | | receptor, U-PAR, |
| | | | | | uPAR, monocyte |
| | | | | | activation antigen |
| | | | | | Mo3, CD87, |
| | | | | | urokinase R, |
| | | | | | urokinase-type |
| | | | | | plasminogen |
| | | | | | activator receptor |
| Axl | AXL | P30530 | AXL receptor | ARK, JTK11, | tyrosine-protein |
| | | | tyrosine kinase | Tyro7, UFO, | kinase receptor |
| | | | | AXL3 | UFO, AXL |
| | | | | | oncogene, |
| | | | | | EC: 2.7.10.1 |
| WIF-1 | WIF1 | Q9Y5W5 | WNT inhibitory | WIF-1, | Wnt inhibitory |
| | | | factor 1 | UNQ191/PRO217 | factor 1, WIF-1 |
| IL-7 R alpha | IL 7R | P16871 | interleukin 7 | CD127, CDW127, | interleukin-7 |
| | | | receptor | IL-7R-alpha, IL- | receptor subunit |
| | | | | 7R-α, IL- | alpha, IL-7 receptor |
| | | | | 7RalphaA, | subunit alpha, IL-7R |
| | | | | IL7Ralpha, ILRA, | subunit alpha, IL- |
| | | | | IL7RA, IMD104, | 7R-alpha, IL-7RA, |
| | | | | sIL-7R, Inc-IL7R, | CDw127, CD127 |
| | | | | Il7r α | |
| GPR56 | ADGRG1 | Q9Y653 | adhesion G | BFPP, BPPR, | adhesion G-protein |
| | | | protein-coupled | CDCBM14B, | coupled receptor |
| | | | receptor G1 | CDCBM15A, | G1, G-protein |
| | | | | GPR56, TM7LN4, | coupled receptor 56, |
| | | | | TM7XN1, | protein TM7XN1, |
| | | | | | ADGRG1 N- |
| | | | | UNQ540/PRO1083 | terminal fragment |
| | | | | | (ADGRG1 NT, |
| | | | | | GPR56 N-terminal |
| | | | | | fragment, GPR56 |
| | | | | | NT, GPR56(N), |
| | | | | | GPR56 extracellular |
| | | | | | subunit, GPR56 |
| | | | | | subunit alpha), |
| | | | | | ADGRG1 C- |
| | | | | | terminal frgament |
| | | | | | (ADGRG1 CT, |
| | | | | | GPR56 C-trminal |
| | | | | | fragment, GPR56 |
| | | | | | CT, GPR56(C), |
| | | | | | GPR56 seven- |
| | | | | | transmembrane |
| | | | | | subunit, GPR56 |
| | | | | | 7TM, GPR56 |
| | | | | | subunit beta) |
| CEACAM-3 | CEACAM3 | P40198 | CEA cell adhesion | CD66D, CEA, | carcinoembryonic |
| | | | molecule 3 | CGM1, CGM1a, | antigen-related cell |
| | | | | W264, W282 | adhesion molecule |
| | | | | | 3, |
| | | | | | carcinoembryonicc |
| | | | | | antigen CGM1, |
| | | | | | CD66d |
| MCEMP1 | MCEMP1 | Q8IX19 | mast cell | C19orf59 | mast cell-expressed |
| | | | expressed | | membrane protein 1 |
| | | | membrane protein | | |
| | | | 1 | | |
| FABP2 | FABP2 | P12104 | fatty acid binding | FABPI, I-FABP | fatty acid-binding |
| | | | protein 2 | | protein intestinal, |
| | | | | | fatty-acid binding |
| | | | | | protein 2, intestinal- |
| | | | | | type fatty acid- |
| | | | | | binding protein (I- |
| | | | | | FABP |
| Plexin B3 | PLXNB3 | Q9ULL4 | plexin B3 | PLEXB3, PLEXR, | plexin-B3 |
| | | | | PLXN6, | |
| | | | | KIAA1206 | |
| MEPE | MEPE | Q9NQ76 | matrix | OF45 | osteoblast/osteocyte |
| | | | extracellular | | factor 45 (OF45), |
| | | | phosphoglycoprotein | | osteoregulin |
| Activin | ACVR2A | P27037 | activin A receptor | ACTRII, ACVR2, | activin receptor |
| RIIA | | | type 2A | ACTRIIA, ActR2a | type-2A, activin |
| | | | | | receptor type IIA |
| | | | | | (ACTR-IIA, |
| | | | | | ACTRIIA), activin |
| | | | | | A receptor type II, |
| | | | | | activin RIIA, |
| | | | | | EC: 2.7.11.30 |
| ANG-2 | ANGPT2 | O15123 | angiopoietin 2 | AGPT2, ANG2, | angiopoietin-2, |
| | | | | LMPHM10 | ANG-2 |
| Cochlin | COCH | O43405 | cochlin | COCH-5B2, | COCH-5B2 |
| | | | | DFNA9, | |
| | | | | DFNB110, | |
| | | | | COCH5B2, | |
| | | | | UNQ257/PRO294 | |
| Presenilin 1 | PSEN1 | P49768 | presenilin 1 | ACNINV3, AD3, | presenilin-1, PS-1, |
| | | | | CMD1U, FAD, | protein S182, |
| | | | | PS-1, PS1, | presenilin-1 NTF |
| | | | | PSNL1, S182 | subunit, presenilin-1 |
| | | | | | CTF subunit, |
| | | | | | presenilin-1 CTF12 |
| | | | | | (PS1-CTF12), |
| | | | | | EC: 3.4.23.- |
| NPTXR | NPTXR | O95502 | neuronal pentraxin | NPR | NPTXR isoform 1 |
| | | | receptor | | |
| SLAM | SLAMF1 | Q13291 | signaling | CD150, SLAM, | signaling |
| | | | lymphocytic | CDw150 | lymphocytic |
| | | | activation | | activation molecule, |
| | | | molecule family | | CDw150, IPO-3, |
| | | | member 1 | | SLAM family |
| | | | | | member 1, CD150 |
| | CD48 | P09326 | CD48 molecule | SLAMF2, BCM1, | CD48 antigen, B- |
| | | | | BLAST, BLAST1, | lymphocyte |
| | | | | MEM-102, | activation marker |
| | | | | hCD48, mCD48 | BLAST-1, BCM1 |
| | | | | | surface antigen, |
| | | | | | leukocyte antigen |
| | | | | | MEM-102, SLAM |
| | | | | | family member 2 |
| | | | | | (SLAMF2), |
| | | | | | signaling |
| | | | | | lymphocytic |
| | | | | | activation molecule |
| | | | | | 2, TCT.1, CD48 |
| | LY9 | Q9HBG7 | lymphocyte | SLAMF3, CD229, | T-lymphocyte |
| | | | antigen 9 | hly9, mLY9, | surface antigen Ly- |
| | | | | CDABP0070 | 9, cell surface |
| | | | | | molecule Ly-9, |
| | | | | | lymphocyte antigen |
| | | | | | 9, SLAM family |
| | | | | | member 3 (SLMF3), |
| | | | | | signaling |
| | | | | | lymphocytic |
| | | | | | activation molecule |
| | | | | | 3, CD229 |
| | CD244 | Q9BZW8 | CD244 molecule | SLAMF4, 2B4, | CD244 antigen, |
| | | | | NAIL, NKR2B4, | natural killer cell |
| | | | | Nmrk, h2B4 | receptor 2B4, NK |
| | | | | | cell activation- |
| | | | | | inducing ligand |
| | | | | | (NAIL), NK cell |
| | | | | | type I receptor |
| | | | | | protein 2B4 |
| | | | | | (NKR2B4, h2B4), |
| | | | | | SLAM family |
| | | | | | member 4 |
| | | | | | (SLAMF4), |
| | | | | | signaling |
| | | | | | lymphocytic |
| | | | | | activation molecule |
| | | | | | 4, CD244 |
| | CD84 | Q9UIB8 | CD84 molecule | SLAMF5, LY9B, | SLAM family |
| | | | | hCD84, mCD84, | member 5, |
| | | | | CDW84 | leukocyte antigen |
| | | | | | CD84, cell surface |
| | | | | | antigen MAX.3, |
| | | | | | Hly9-beta, |
| | | | | | leukocyte |
| | | | | | differentiation |
| | | | | | antigen CD84, |
| | | | | | signaling |
| | | | | | lymphocytic |
| | | | | | activation molecule |
| | | | | | 5, CD84 |
| | SLAMF6 | Q96DU3 | SLAM family | NTB-A(h), | activating NK |
| | | | member 6 | Ly108(m), CD352, | receptor, NK-T-B- |
| | | | | KALI, KALIb, | antigen (NTB-A), |
| | | | | Ly 108, NTB-A, | CD352 |
| | | | | NTBA, SF2000, | |
| | | | | UNQ6123/PRO20080, | |
| | | | | NK-T-B- | |
| | | | | antigen | |
| | SLAMF7 | Q9NQ25 | SLAM family | CD319, 19A, | CD2 subset 1, CD2- |
| | | | member 7 | CRACC, CS1, | like receptor- |
| | | | | UNQ576/PRO1138 | activating cytotoxic |
| | | | | | cells (CRACC), |
| | | | | | membrane protein |
| | | | | | FOAP-12, novel |
| | | | | | Ly9, protein 19A, |
| | | | | | CD319 |
| | SLAMF8 | Q9P0V8 | SLAM family | BLAME, CD353, | B-lymphocyte |
| | | | member 8 | SBBI42 | activator |
| | | | | | macrophage |
| | | | | | expressed, BCM- |
| | | | | | like membrane |
| | | | | | protein, CD353 |
| | SLAMF9 | Q96A28 | SLAM family | SF2001, CD2F-10, | CD2 family member |
| | | | member 9 | CD2F10, CD84- | 10 (CD2F-10), |
| | | | | H1, CD84H1, | CD84 homolog 1 |
| | | | | UNQ1938/PRO4421 | (CD84-H1) |
| COMT | COMT | P21964 | catechol-O- | HEL-S-98n | catechol O- |
| | | | methyltransferase | | methyltransferase, |
| | | | | | catechol-O-methyl |
| | | | | | transferase, |
| | | | | | EC: 2.1.1.6 |
| SPHK1 | SPHK1 | Q9NYA1 | sphingosine | SPHK, SK1, SPK | SK 1, SPK 1, |
| | | | kinase 1 | | acetyltransferase |
| | | | | | SPHK1 (EC: 2.3.1.-), |
| | | | | | EC: 2.7.1.91 |
| RBP4 | RBP4 | P02753 | retinol binding | MCOPCB10, | retinol-binding |
| | | | protein 4 | RDCCAS, APP- | protein 4, plasma |
| | | | | BP, PRBP, RBP, | retinol-binding |
| | | | | PRO2222 | protein (PRBP, |
| | | | | | RBP), plasma |
| | | | | | retinol-binding, |
| | | | | | plasma retinol- |
| | | | | | binding protein(1- |
| | | | | | 182), plasma retinol- |
| | | | | | binding protein(1- |
| | | | | | 181), plasma retinol- |
| | | | | | binding protein(1- |
| | | | | | 179), plasma retinol- |
| | | | | | binding protein(1- |
| | | | | | 176) |
| Nectin-1 | NECTIN1 | Q15223 | nectin cell | CD111, CLPED1, | nectin-1, Herpes |
| | | | adhesion molecule | ED4, HIgR, | virus entry mediator |
| | | | 1 | HV1S, HVEC, | C, Herpesvirus entry |
| | | | | OFC7, PRR, | mediator C (HveC), |
| | | | | PRR1, PVRL1, | Herpesvirus Ig-like |
| | | | | PVRR, PVRR1, | receptor (HIgR), |
| | | | | SK-12, nectin-1 | nectin cell adhesion |
| | | | | | molecule 1, |
| | | | | | Poliovirus receptor- |
| | | | | | related protein 1, |
| | | | | | CD111 |
| GUSB | GUSB | P08236 | glucuronidase beta | BG, MPS7 | beta-glucuronidase, |
| | | | | | glucuronidase β, β |
| | | | | | glucuronidase, beta- |
| | | | | | G1, EC: 3.2.1.31 |
| Nidogen-2 | NID2 | Q14112 | nidogen 2 | NID-2 | nidogen-2, |
| | | | | | osteonidogen, NID- |
| | | | | | 2 |
| IL-17F | IL17F | Q96PD4 | interleukin 17F | CANDF6, IL-17F, | interleukin-17F, IL- |
| | | | | IL17A, ML-1, | 17F, cytokine ML-1 |
| | | | | ML1 | |
| SR-AI | MSR1 | P21757 | macrophage | CD204, SCARA1, | macrophage |
| | | | scavenger receptor | SR-A, SR-AI, SR- | scavenger receptor |
| | | | 1 | AII, SR-AIII, | types I and II, |
| | | | | SRA, phSR1, | macrophage |
| | | | | phSR2, MSRA1, | acetylated LDL |
| | | | | SRAI/II | receptor I and II, |
| | | | | | scavenger receptor |
| | | | | | class A member 1, |
| | | | | | CD204, macrophage |
| | | | | | scavenger receptor |
| TAFA2 | TAFA2 | Q8N3H0 | TAFA chemokine | FAM19A2, | chemokine-like |
| | | | like family | TAFA-2, | protein TAFA-2 |
| | | | member 2 | LOC102724280, | |
| | | | | LOC338811 | |
| N-Cadherin | CDH2 | P19022 | cadherin 2 | ACOGS, ADHD8, | cadherin-2, neural |
| | | | | ARVD14, CD325, | cadherin, N- |
| | | | | CDHN, CDw325, | cadherin, CDw325, |
| | | | | NCAD | CD325 |
| IL-17B | IL17RB | Q9NRM6 | interleukin 17 | CRL4, EVI27, | interleukin-17 |
| | | | receptor B | IL17BR, | receptor B, IL-17B |
| | | | | IL17RH1, | receptor, IL-17 |
| | | | | UNQ2501/PRO19612, | receptor B (IL- |
| | | | | IL25R | 17RB), cytokine |
| | | | | | receptor-like 4, IL- |
| | | | | | 17 receptor homolog |
| | | | | | 1 (IL-17Rh1, |
| | | | | | IL17Rh1), |
| | | | | | interleukin-17B |
| | | | | | receptor |
| IL-17 RC | IL17RC | Q8NAC3 | interleukin 17 | CANDF9, IL17- | interleukin-17 |
| | | | receptor C | RL, IL17RL, | receptor C, IL-17 |
| | | | | UNQ6118/PRO20040/ | receptor C, IL- |
| | | | | PRO38901 | 17RC, interleukin- |
| | | | | | 17 receptor homolog |
| | | | | | (IL17Rhom), |
| | | | | | interleukin-17 |
| | | | | | receptor-like protein |
| | | | | | (IL-17RL), |
| | | | | | ZcytoR14 |
| MIP-3b | CCL19 | Q99731 | C-C motif | CKb11, ELC, | C-C motif |
| | | | chemokine ligand | MIP-3b, MIP3B, | chemokine 19, beta- |
| | | | 19 | SCYA19, CK β-11 | chemokine exodus- |
| | | | | | 3, CK beta-11, |
| | | | | | Epstein-Barr virus- |
| | | | | | induced molecule 1 |
| | | | | | ligand chemokine |
| | | | | | (EBIl ligand |
| | | | | | chemokine, ELC), |
| | | | | | macrophage |
| | | | | | inflammatory |
| | | | | | protein 3 beta (MIP- |
| | | | | | 3-beta), small- |
| | | | | | inducible cytokine |
| | | | | | A19 |
| Cystatin C | CST3 | P01034 | cystatin C | ARMD11, HEL-S- | cystatin-C, cystatin- |
| | | | | 2, gamma- | 3, gamma-trace, |
| | | | | TRACE, γ- | neuroendocrine |
| | | | | TRACE | basic polypeptide, |
| | | | | | post-gamma- |
| | | | | | globulin |
| Cystatin D | CST5 | P28325 | cystatin D | | cystatin-D, cystatin- |
| | | | | | 5 |
| AMSH | STAMBP | O95630 | STAM binding | AMSH, MICCAP, | STAM-binding |
| | | | protein | LOC100507148 | protein, associated |
| | | | | | molecule with the |
| | | | | | SH3 domain of |
| | | | | | STAM, endosome- |
| | | | | | associated ubiquitin |
| | | | | | isopeptidase, |
| | | | | | EC: 3.4.19.- |
| FcERI | FCER1A | P12319 | Fc epsilon | FCE1A, FCERIA, | high affinity |
| | | | receptor Ia | FcERI | immunoglobulin |
| | | | | | epsilon receptor |
| | | | | | subunit alpha, Fc- |
| | | | | | epsilon RI-alpha |
| | | | | | (FcERI), IgE Fc |
| | | | | | receptor subunit |
| | | | | | alpha, FC epsilon |
| | | | | | R1 alpha, Fc epsilon |
| | | | | | RI, FC ε R1 α, Fc E |
| | | | | | receptor Ia, Fc ε RI, |
| | | | | | Ig epsilon RI, Ig ε |
| | | | | | RI |
| CLEC10A | CLEC10A | Q8IUN9 | C-type lectin | CD301, | C-type lectin |
| | | | domain containing | CLECSF13, | domain family 10 |
| | | | 10A | CLECSF14, HML, | member A, C-type |
| | | | | HML2, MGL | lectin superfamily |
| | | | | | member 14, |
| | | | | | macrophage lectin 2, |
| | | | | | CD301 |
| HGF R | MET | P08581 | MET proto- | AUTS9, DA11, | hepatocyte growth |
| | | | oncogene, | DFNB97, HGFR, | factor receptor, HGF |
| | | | receptor tyrosine | RCCP2, c-Met, | binding, HGF |
| | | | kinase | MetR | receptor, HGF/SF |
| | | | | | receptor, proto- |
| | | | | | oncogene c-Met, |
| | | | | | scatter factor |
| | | | | | receptor (SF |
| | | | | | receptor), tyrosine- |
| | | | | | protein kinase Met, |
| | | | | | EC: 2.7.10.1 |
| ANG-1 | ANGPT1 | Q15389 | angiopoietin 1 | AGP1, AGPT, | angiopoietin-1, |
| | | | | AGPT-1, ANG1, | ANG-1 |
| | | | | HAE5, KIAA0003 | |
| Prolactin R | PRLR | P16471 | prolactin receptor | HPRL, MFAB, | PRL-R |
| | | | | RI-PRLR, hPRLrI, | |
| | | | | FLJ11027 | |
| FGF-20 | FGF20 | Q9NP95 | fibroblast growth | FGF-20, RHDA2 | FGF-20 |
| | | | factor 20 | | |
| CD28 | CD28 | P10747 | CD28 molecule | IMD123, Tp44 | T-cell-specific |
| | | | | | surface glycoprotein |
| | | | | | CD28, CD28 |
| | | | | | antigen, TP44, |
| | | | | | CD28 |
| Nogo-A | RTN4 | Q9NQC3 | reticulon 4 | ASY, NI220/250, | reticulon-4, foocen, |
| | | | | NOGO, NSP, | neurite outgrowth |
| | | | | NSP-CL, | inhibitor (Nogo |
| | | | | Nbla00271, | protein), |
| | | | | Nbla10545, RTN- | neuroendocrine- |
| | | | | X-A, RTN4-B1, | specific protein |
| | | | | RTN4-B2, RTN4- | (NSP), |
| | | | | C, RTN4, | neuroendocrine- |
| | | | | KIAA0886, | specific protein C |
| | | | | My043, SP1507, | homolog, RTN-x, |
| | | | | RTN-X | reticulon-5 |
| HSD17B1 | HSD17B1 | P14061 | hydroxysteroid | 17-beta-HSD, 20- | 17-beta- |
| | | | 17-beta | alpha-HSD, 20-α- | hydroxysteroid |
| | | | dehydrogenase 1 | HSD, E2DH, | dehydrogenase type |
| | | | | EDH17B1, | 1, 17-beta-hydroxy- |
| | | | | EDH17B2, | steroid- |
| | | | | EDHB17, HSD17, | dehydrogenase |
| | | | | SDR281, | isoform I, 17-beta- |
| | | | | E17KSR, | HSD 1, 20 alpha- |
| | | | | SDR28C1 | hydroxysteroid |
| | | | | | dehydrogenase (20- |
| | | | | | alpha-HSD), E2DH, |
| | | | | | estradiol 17-beta- |
| | | | | | dehydrogenase 1 |
| | | | | | (EC: 1.1.1.62), |
| | | | | | placental 17-beta- |
| | | | | | hydroxysteroid |
| | | | | | dehyrogenase, short |
| | | | | | chain |
| | | | | | dehydrogenase/ |
| | | | | | reductase family 28C |
| | | | | | member 1, 17-β- |
| | | | | | HSD, 17-β-hydroxy- |
| | | | | | steroid- |
| | | | | | dehydrogenase |
| | | | | | isoform I, |
| | | | | | hydroxysteroid 17-β |
| | | | | | dehydrogenase 1 |
| IL-19 | IL19 | Q9UHD0 | interleukin 19 | IL-10C, MDA1, | interleukin-19, IL- |
| | | | | NG.1, ZMDA1 | 19, melanoma |
| | | | | | differentiation- |
| | | | | | associated protein- |
| | | | | | like protein, NG.1 |
| Enteropeptidase | TMPRSS15 | P98073 | transmembrane | ENTK, PRSS7 | enteropeptidase, |
| | | | serine protease 15 | | enterokinase, serine |
| | | | | | protease 7, |
| | | | | | proenterokinase, |
| | | | | | transmembrane |
| | | | | | protease serine 15, |
| | | | | | enteropeptidase non- |
| | | | | | catalytic heavy |
| | | | | | chain, |
| | | | | | enteropeptidase |
| | | | | | catalytic light chain, |
| | | | | | EC: 3.4.21.9 |
| Cathepsin E | CTSE | P14091 | cathepsin E | CATE | cathepsin E form I, |
| | | | | | cathepsin E form II, |
| | | | | | EC: 3.4.23.34 |
| TSLP | TSLP | Q969D9 | thymic stromal | | |
| | | | lymphopoietin | | |
| TCN2 | TCN2 | P20062 | transcobalamin 2 | D22S676, | transcobalamin-2, |
| | | | | D22S750, II, TC, | TC-2, |
| | | | | TC II, TC-2, TC2, | transcobalamin II |
| | | | | TCII | (TC II, TCII) |
| GDF-15 | GDF15 | Q99988 | growth | GDF-15, HG, | growth/differentiation |
| | | | differentiation | MIC-1, MIC1, | factor 15, GDF- |
| | | | factor 15 | NAG-1, PDF, | 15, macrophage |
| | | | | PLAB, PTGFB, | inhibitory cytokine |
| | | | | LOC100292463, | 1 (MIC-1), NSAID- |
| | | | | Ptgf β | activated gene 1 |
| | | | | | protein (NAG-1), |
| | | | | | NSAID-regulated |
| | | | | | gene 1 protein |
| | | | | | (NRG-1), placental |
| | | | | | TGF-beta, placental |
| | | | | | bone morphogenic |
| | | | | | protein, prostate |
| | | | | | differentiation |
| | | | | | factor, placental |
| | | | | | TGF-β |
| Epimorphin | STX2 | P32856 | syntaxin 2 | EPM, EPIM, | syntaxin-2, |
| | | | | STX2A, STX2B, | epimorphin |
| | | | | STX2C | |
| GRKS | GRK1 | Q15835 | G protein-coupled | GPRK1, RHOK, | rhodopsin kinase |
| | | | receptor kinase 1 | RK | GRK1, RK, |
| | | | | | EC: 2.7.11.14 |
| | GRK2 | P25098 | G protein-coupled | ADRBK1, BARK, | beta-adrenergic |
| | | | receptor kinase 2 | BARK1, BETA- | receptor kinase 1, |
| | | | | ARK1, β-ARK1 | beta-ARK-1, |
| | | | | | EC: 2.7.11.15 |
| | GRK3 | P35626 | G protein-coupled | ADRBK2, | beta-adrenergic |
| | | | receptor kinase 3 | BARK2, | receptor kinase 2 |
| | | | | FLJ31125, β ark2 | (Beta-ARK-2), |
| | | | | | EC: 2.7.11.15 |
| | GRK4 | P32298 | G protein-coupled | IT11, GPRK4, | G protein-coupled |
| | | | receptor kinase 4 | GRK4a, GPRK2L | receptor kinase |
| | | | | | GRK4, ITI1, |
| | | | | | EC: 2.7.11.16 |
| | GRK5 | P34947 | G protein-coupled | FP2025, GPRK5, | G protein-coupled |
| | | | receptor kinase 5 | LOC100131848 | receptor kinase |
| | | | | | GRK5, |
| | | | | | EC: 2.7.11.16 |
| | GRK6 | P43250 | G protein-coupled | GPRK6 | G protein-coupled |
| | | | receptor kinase 6 | | receptor kinase |
| | | | | | GRK6, |
| | | | | | EC: 2.7.11.16 |
| | GRK7 | Q8WTQ7 | G protein-coupled | GPRK7 | G protein-coupled |
| | | | receptor kinase 7 | | receptor kinase 7, G |
| | | | | | protein-coupled |
| | | | | | receptor kinase |
| | | | | | GRK7, |
| | | | | | EC: 2.7.11.14 |
| PD-1 | PDCD1 | Q15116 | programmed cell | CD279, PD-1, | programmed cell |
| | | | death 1 | PD1, SLEB2, | death protein 1, |
| | | | | hPD-1, hPD-I, | protein PD-1, hPD- |
| | | | | hSLE1, B7H1 | 1, CD279 |
| Serpin A4 | SERPINA4 | P29622 | serpin family A | KAL, KLST, KST, | kallistatin, kallikrein |
| | | | member 4 | PI-4, PI4, | inhibitor, peptidase |
| | | | | kallistatin | inhibitor 4 (PI-4), |
| | | | | | serpin A4 |
| ADAM23 | ADAM23 | O75077 | ADAM | MDC-3, MDC3 | disintegrin and |
| | | | metallopeptidase | | metalloproteinase |
| | | | domain 23 | | domain-containing |
| | | | | | protein 23, ADAM |
| | | | | | 23, |
| | | | | | metalloproteinase- |
| | | | | | like disintegrin-like |
| | | | | | and cysteine-rich |
| | | | | | protein 3 (MDC-3) |
| NOV | CCN3 | P48745 | cellular | IBP-9, IGFBP-9, | CCN family |
| | | | communication | IGFBP9, NOV, | member 3, insulin- |
| | | | network factor 3 | NOVh, NOVH, | like growth factor- |
| | | | | IGFBP-RP3 | binding protein 9 |
| | | | | | (IBP-9, IGF-binding |
| | | | | | protein 9, IGFBP-9), |
| | | | | | nephro blastoma- |
| | | | | | overexpressed gene |
| | | | | | protein homolog, |
| | | | | | protein NOV |
| | | | | | homolog (NovH) |
| Galectin-2 | LGALS2 | P05162 | galectin 2 | HL14, GAL2 | galectin-2, Gal-2, |
| | | | | | beta-galactoside- |
| | | | | | binding lectin L-14- |
| | | | | | II, HL14, lactose- |
| | | | | | binding lectin 2, S- |
| | | | | | Las lectin 2 |
| Neurexin 3 | NRXN3 | Q9HDB5 | neurexin 3 | C14orf60, | neurexin-3-beta, |
| beta | | | | KIAA0743, | neurexin III-beta, |
| | | | | LOC100418878 | neurexin-3-beta |
| | | | | | solbule form, |
| | | | | | neurexin-3-beta C- |
| | | | | | terminal fragment |
| | | | | | (NRXN3-CTF) |
| TLR3 | TLR3 | O15455 | toll like receptor 3 | CD283, IIAE2, | toll-like receptor 3, |
| | | | | IMD83 | CD283 |
| Sirtuin 2 | SIRT2 | Q8IXJ6 | sirtuin 2 | SIR2, SIR2L, | NAD-dependent |
| | | | | SIR2L2 | protein deacetylase |
| | | | | | sirtuin-2, NAD- |
| | | | | | dependent protein |
| | | | | | defatty-acylase |
| | | | | | sirtuin-2 (EC: 2.3.1.-), |
| | | | | | regulatory protein |
| | | | | | SIR2 homolog 2, |
| | | | | | SIR2-like protein 2, |
| | | | | | EC: 2.3.1.286 |
| Numb | NUMB | P49757 | NUMB endocytic | C14orf41, S171, | protein numb |
| | | | adaptor protein | c14_5527 | homolog, h-Numb, |
| | | | | | protein S171 |
| IL-28 R | IFNLR1 | Q9IU57 | interferon lambda | CRF2/12, IFNLR, | IFN-lambda |
| alpha | | | receptor 1 | IL-28R1, IL28RA, | receptor 1, IFN- |
| | | | | LICR2 | lambda-R1, |
| | | | | | cytokine receptor |
| | | | | | class-II member 12, |
| | | | | | cytokine receptor |
| | | | | | family 2 member 12 |
| | | | | | (CRF2-12), |
| | | | | | interleukin-28 |
| | | | | | receptor subunit |
| | | | | | alpha (IL-28 |
| | | | | | receptor subunit |
| | | | | | alpha, IL-28R-alpha, |
| | | | | | IL-28RA), likely |
| | | | | | interleukin or |
| | | | | | cytokine receptor 2 |
| | | | | | (LICR2), IFN |
| | | | | | lambda R1, IFN λ |
| | | | | | R1, interferon λ |
| | | | | | receptor 1 |
| IL-33 | IL33 | O95760 | interleukin 33 | C9orf26, DVS27, | interleukin-33, IL- |
| | | | | IL1F11, NF-HEV, | 33, interleukin-1 |
| | | | | NFEHEV, | family member 11 |
| | | | | NFHEV | (IL-1F11), nuclear |
| | | | | | factor form high |
| | | | | | endothelial venules |
| | | | | | (NF-HEV), |
| | | | | | iinterleukin-33 (95- |
| | | | | | 270), interleukin-33 |
| | | | | | (99-270), |
| | | | | | interleukin-33 (109- |
| | | | | | 270) |
| Lin28 | LIN28A | Q9H9Z2 | lin-28 homolog A | CSDD1, LIN-28, | protein lin-28 |
| | | | | LIN28, ZCCHC1, | homolog A, lin- |
| | | | | lin-28A | 28A, zinc finger |
| | | | | | CCHC domain- |
| | | | | | containing protein 1 |
| FCRL1 | FCRL1 | Q96LA6 | Fc receptor like 1 | CD307a, FCRH1, | Fc receptor-like |
| | | | | IFGP1, IRTA5 | protein 1, FcR-like |
| | | | | | protein 1, FcRL1, Fc |
| | | | | | receptor homolog 1 |
| | | | | | (FcRH1), IFGP |
| | | | | | family protein 1 |
| | | | | | (hIFGP1), immune |
| | | | | | receptor |
| | | | | | translocation- |
| | | | | | associated protein 5, |
| | | | | | CD307a |
| KLF4 | KLF4 | O43474 | KLF transcription | EZF, GKLF | Krueppel-like factor |
| | | | factor 4 | | 4, epithelial zinc |
| | | | | | finger protein EZF, |
| | | | | | gut-enriched |
| | | | | | krueppel-like factor |
| NKp30 | NCR3 | O14931 | natural | 1C7, CD337, | activating natural |
| | | | cytotoxicity | LY117, MALS, | killer receptor p30, |
| | | | triggering receptor | NKp30 | natural killer cell |
| | | | 3 | | p30-related protein |
| | | | | | (NK-p30, NKp30), |
| | | | | | C337 |
| Lymphotactin | XCL1 | P47992 | X-C motif | ATAC, LPTN, | lymphotactin, |
| | | | chemokine ligand | LTN, SCM-1, | ATAC, C motif |
| | | | 1 | SCM-1a, SCM1, | chemokine 1, |
| | | | | SCM1A, SCYC1, | cytokine SCM-1, |
| | | | | SCM-1-alpha, | lymphotaxin, SCM- |
| | | | | SCM-1-α | 1-alpha, small- |
| | | | | | inducible cytokine |
| | | | | | C1, XC chemokine |
| | | | | | ligand 1, cytokine |
| | | | | | SCM-1 |
| Cystatin SN | CST1 | P01037 | cystatin SN | | cystatin-SN, |
| | | | | | cystain-SA-I, |
| | | | | | cystatin-SA-I, |
| | | | | | cystatin-1, salivary |
| | | | | | cystatin-SA-1 |
| JAM-A | F11R | Q9Y624 | F11 receptor | CD321, JAM, | junctional adhesion |
| | | | | JAM1, JAMA, | molecule A |
| | | | | JCAM, KAT, | |
| | | | | PAM-1 | |
| Calreticulin- | CALR | P27797 | calreticulin | CALR1, CRT, | CRP55, calregulin, |
| 2 | | | | HEL-S-99n, RO, | endoplasmic |
| | | | | SSA, cC1qR, | reticulum resident |
| | | | | CRTC | protein 60 (ERp60), |
| | | | | | HACBP, grp60, |
| | | | | | calreticulin isoform |
| | | | | | 1 |
| ErbB4 | ERBB4 | Q15303 | erb-b2 receptor | ALS19, HER4, | receptor tyrosine- |
| | | | tyrosine kinase 4 | p180erbB4 | protein kinase erbB- |
| | | | | | 4, proto-oncogene- |
| | | | | | like protein c-ErbB- |
| | | | | | 4, tyrosine kinase- |
| | | | | | type cell surface |
| | | | | | receptor HER4, |
| | | | | | p180erbB4, ERBB4 |
| | | | | | intracellular domain |
| | | | | | (4ICD, E4ICD, |
| | | | | | s80HER4), |
| | | | | | EC: 2.7.10.1 |
| BMP-8 | BMP8A | Q7Z5Y6 | bone morphogenic | OP-2, Op2, | bone morphogenic |
| | | | protein 8a | FLJ45264 | protein 8A, BMP- |
| | | | | | 8A |
| | BMP8B | P34820 | bone morphogenic | BMP8, OP2 | bone morphogenic |
| | | | protein 8b | | protein 8B, BMP-8, |
| | | | | | BMP-8B, |
| | | | | | osteogenic protein 2 |
| | | | | | (OP-2) |
| IL-27 Ra | IL27RA | Q6UWB1 | interleukin 27 | CRL1, IL-27RA, | interleukin 27 |
| | | | receptor subunit | IL27R, TCCR, | receptor subunit α, |
| | | | alpha | WSX1, zcytor1, | interleukin-27 |
| | | | | UNQ296/PRO336, | receptor subunit |
| | | | | IL-27R-alpha, IL- | alpha, IL-27 |
| | | | | 27R-α | receptor subunit |
| | | | | | alpha, IL-27R |
| | | | | | subunit alpha, IL- |
| | | | | | 27R-alpha, IL- |
| | | | | | 27RA, cytokine |
| | | | | | receptor WSX-1, |
| | | | | | cytokine receptor- |
| | | | | | like 1, type I T-cell |
| | | | | | cytokine receptor |
| | | | | | (TCCR), ZcytoR1 |
| Fas | FAS | P25445 | Fas cell surface | ALPS1A, APO-1, | tumor necrosis |
| | | | death receptor | APT1, CD95, | factor receptor |
| | | | | CD95L, CD95 | superfamily member |
| | | | | receptor, | 6, CD95 receptor, |
| | | | | FAS/APO1, | Apo-1 antigen, |
| | | | | FASTM, | apoptosis-mediating |
| | | | | TNFRSF6, FAS1 | surface antigen |
| | | | | | FAS, FASLG |
| | | | | | receptor, CD95 |
| IL-4 Ra | IL4R | P24394 | interleukin 4 | CD124, IL-4RA, | interleukin-4 |
| | | | receptor | IL4RA, IL4R | receptor subunit |
| | | | | alpha, IL4R α, Il4r | alpha, IL4R alpha, |
| | | | | β | IL4R α, IL-4 |
| | | | | | receptor subunit |
| | | | | | alpha, IL-4R subunit |
| | | | | | alpha, IL-4R-alpha, |
| | | | | | IL-4RA, soluble |
| | | | | | interleukin-4 |
| | | | | | receptor subunit |
| | | | | | alpha, soluble IL-4 |
| | | | | | receptor subunit |
| | | | | | alpha, soluble IL- |
| | | | | | 4R-alpha, |
| | | | | | sIL4Ralpha/prot, IL- |
| | | | | | 4-binding protein |
| | | | | | (IL4-BP), CD124 |
| Kallikrein | KLK14 | Q9P0G3 | kallikrein related | KLK-L6, KLKL6 | kallikrein-14, hK14, |
| 14 | | | peptidase 14 | | kallikrein-like |
| | | | | | protein 6 (KLK-L6), |
| | | | | | EC: 3.4.21.- |
| Matrilin-3 | MATN3 | O15232 | matrilin 3 | DIPOA, EDM5, | matrilin-3 |
| | | | | HOA, OADIP, | |
| | | | | OS2, SEMDBCD | |
| Olig2 | OLIG2 | Q13516 | oligodendrocyte | BHLHB1, | Oligo2, class B |
| | | | transcription | OLIGO2, | basic helix-loop- |
| | | | factor 2 | PRKCBP2, | helix protein 1 |
| | | | | RACK17, | (bHLHb1), class E |
| | | | | bHLHe19, | basic helix-loop- |
| | | | | BHLHE19 | helix protein 19 |
| | | | | | (bHLHe19), protein |
| | | | | | kinase C-binding |
| | | | | | protein 2, protein |
| | | | | | kinase C-binding |
| | | | | | protein RACK17 |
| Kallikrein | KLK12 | Q9UKR0 | kallikrein related | KLK-L5, KLKL5, | kallikrein-12, |
| 12 | | | peptidase 12 | UNQ669/PRO1303 | allikrein-like protein |
| | | | | | 5 (KLK-L5), |
| | | | | | EC: 3.4.21.- |
| CA13 | CA13 | Q8N1Q1 | carbonic | CAXIII | carbonate |
| | | | anhydrase 13 | | dehydratase XIII, |
| | | | | | carbonic anhydrase |
| | | | | | XIII (CA-XIII), |
| | | | | | EC: 4.2.1.1 |
| IL-9 | IL9 | P15248 | interleukin 9 | HP40, IL-9, P40 | interleukin-9, IL-9, |
| | | | | | cytokine P40, T-cell |
| | | | | | growth factor P40 |
| Nectin-3 | NECTIN | Q9NQS3 | nectin cell | CD113, CDW113, | nectin-3, CDw113, |
| | 3 | | adhesion molecule | NECTIN-3, PPR3, | nectin cell adhesion |
| | | | 3 | PRR3, PVRL3, | molecule 3, |
| | | | | PVRR3, | Poliovirus receptor- |
| | | | | LOC100506575 | related protein 3, |
| | | | | | CD113 |
| MPIF-1 | CCL23 | P55773 | C-C motif | CK-BETA-8, | C-C motif |
| | | | chemokine ligand | CKb8, Ckb-8, | chemokine 23, CK- |
| | | | 23 | Ckb-8-1, MIP-3, | beta-8 (CKB-8), |
| | | | | MIP3, MPIF-1, | macrophage |
| | | | | MPIF1, SCYA23, | inflammatory |
| | | | | hmrp-2a, CK- | protein 3 (MIP-3), |
| | | | | BETA-8, CK-β-8 | myeloid progenitor |
| | | | | | inhibitory factor 1 |
| | | | | | (MPIF-1), small- |
| | | | | | inducible cytokine |
| | | | | | A23, CCL23(19- |
| | | | | | 99), CCL23(22-99), |
| | | | | | CCL23(27-99), |
| | | | | | CCL23(30-99) |
| Cystatin S | CST4 | P01036 | cystatin S | cystatin SA-III | cystatin-S, cystatin- |
| | | | | | 4, cystatin-SA-III, |
| | | | | | salivary acidic |
| | | | | | protein 1 |
| ADA | ADA | P00813 | adenosine | ADA1 | adenosine |
| | | | deaminase | | aminohydrolase, |
| | | | | | EC: 3.5.4.4 |
| IL-2 Rb | IL2RB | P14784 | interleukin 2 | CD122, IL15RB, | interleukin 2 |
| | | | receptor subunit | IMD63, P70-75, | receptor subunit β, |
| | | | beta | Il2r β, Il2r β C | interleukin-2 |
| | | | | | receptor subunit |
| | | | | | beta, IL-2 receptor |
| | | | | | subunit beta, IL-2R |
| | | | | | subunit beta, IL- |
| | | | | | 2RB, high affinity |
| | | | | | IL-2 receptor |
| | | | | | subunit beta, |
| | | | | | interleukin-15 |
| | | | | | receptor subunit |
| | | | | | beta, p70-75 (p75), |
| | | | | | CD122 |
| GFR alpha- | GFRA1 | P56159 | GDNF family | GDNFR, GDNFR- | GDNF family |
| 1 | | | receptor alpha 1 | alpha-1, | receptor α 1, GDNF |
| | | | | GDNFRA, GFR- | family receptor |
| | | | | ALPHA-1, | alpha-1, GDNF |
| | | | | GDNFR-α-1, | receptor alpha-1, |
| | | | | GFRalpha-1, | GDNFR-alpha-1, |
| | | | | RET1L, RETL1, | GFR-alpha-1, RET |
| | | | | RHDA4, TRNR1, | ligand 1, TGF-beta- |
| | | | | Gfr α 1, GFR-α-1, | related neurotrophic |
| | | | | GRFA1 | factor receptor 1 |
| | | | | LOC143381 | |
| Smad4 | SMAD4 | Q13485 | SMAD family | DPC4, JIP, | Mothers against |
| | | | member 4 | MADH4, MYHRS | decapentaplegic |
| | | | | | homolog 4, MAD |
| | | | | | homolog 4, Mothers |
| | | | | | against DPP |
| | | | | | homolog 4, deletion |
| | | | | | target in pancreatic |
| | | | | | carcinoma 4, SMAD |
| | | | | | 4, Smad4, hSMAD4 |
| ICAM-1 | ICAM1 | P05362 | intercellar | BB2, CD54, P3.58 | intercellular |
| | | | adhesion molecule | | adhesion molecule |
| | | | | | 1, ICAM-1, major |
| | | | | | group rhinovirus |
| | | | | | receptor, CD54 |
| MEF2C | MEF2C | Q06413 | myocyte enhancer | C5DELq14.3, | myocyte-specific |
| | | | factor 2C | DEL5q14.3, | enhancer factor 2C |
| | | | | NEDHSIL | |
| TREM-1 | TREM1 | Q9NP99 | triggering receptor | CD354, TREM-1 | TREM-1, triggering |
| | | | expressed on | | receptor expressed |
| | | | myeloid cells 1 | | on monocytes 1, |
| | | | | | CD354 |
| L-Selectin | SELL | P14151 | selectin L | CD62L, LAM1, | L-selectin, |
| | | | | LECAM1, LEU8, | lymphocyte |
| | | | | LNHR, LSEL, | adhesion molecule |
| | | | | LYAM1, PLNHR, | 1, CD62 antigen- |
| | | | | TQ1, l-selectin | like family member |
| | | | | | L, leukocyte |
| | | | | | adhesion molecule 1 |
| | | | | | (LAM-1), leukocyte |
| | | | | | surface antigen Leu- |
| | | | | | 8, leukocyte- |
| | | | | | endothelial cell |
| | | | | | adhesion molecule 1 |
| | | | | | (LECAM1), lymph |
| | | | | | node homing |
| | | | | | receptor, TQ1, |
| | | | | | gp90-MEL |
| Hepsin | HPN | P05981 | hepsin | TMPRSS1 | serine protease |
| | | | | | hepsin, |
| | | | | | transmembrane |
| | | | | | protease serine 1, |
| | | | | | serine protease |
| | | | | | hepsin non-catalytic |
| | | | | | chain, serine |
| | | | | | protease hepsin |
| | | | | | catalytic chain, |
| | | | | | EC: 3.4.21.106 |
| CD42b | GP1BA | P07359 | glycoprotein Ib | BDPLT1, | platelet glcoprotein |
| | | | platelet subunit | BDPLT3, BSS, | Ib alpha chain, GP- |
| | | | alpha | CD42B, CD42b- | Ib alpha, GPIb- |
| | | | | alpha, CD42b-α, | alpha, GPIbA, |
| | | | | DBPLT3, GP1B, | glycoprotein |
| | | | | GPIbA, | Ibalpha, antigen |
| | | | | GPIbalpha, | CD42b-alpha, |
| | | | | VWDP, GP1B | glycocalicin, |
| | | | | alpha, GP1B α, | CD42b, |
| | | | | GPIb-α, GP-Ib α | glycoprotein 1b α, |
| | | | | | glycoprotein Ib |
| | | | | | platelet subunit α |
| MCSF | CSF1 | P09603 | colony stimulating | CSF-1, MCSF, | macrophage colony- |
| | | | factor 1 | PG-M-CSF | stimulating factor 1, |
| | | | | | CSF1 isoform 1, |
| | | | | | CSF-1, M-CSF, |
| | | | | | MCSF, lanimostim, |
| | | | | | proteoglycan |
| | | | | | macropphage |
| | | | | | colony -stimulating |
| | | | | | factor (PG-M-CSF), |
| | | | | | processed |
| | | | | | macrophage colony- |
| | | | | | stimulating factor 1, |
| | | | | | macrophage colony- |
| | | | | | stimulating factor 1 |
| | | | | | 43 kDa subunit |
| RANK | TNFRSF | Q9Y6Q6 | TNF receptor | CD265, FEO, | tumor necrosis |
| | 11A | | superfamily | LOH18CR1, | factor receptor |
| | | | member 11a | ODFR, OFE, | superfamily member |
| | | | | OPTB7, OSTS, | 11A, osteoclast |
| | | | | PDB2, RANK, | differentiation factor |
| | | | | TRANCE-R, | receptor (ODFR), |
| | | | | TRANCER, | receptor activator of |
| | | | | ODAR | NF-KB, CD265 |
| CHST4 | CHST4 | Q8NCG5 | carbohydrate | GST3, | carbohydrate |
| | | | sulfotransferase 4 | GlcNAc6ST2, | sulphotransferase 4, |
| | | | | HECGLCNAC6S | galactose/N- |
| | | | | T, LSST, | acetylglucosamine/ |
| | | | | GLCNAC6ST2, | N- |
| | | | | Gn6st-2, Hec-6st, | acetylglucosamine |
| | | | | GST-3 | 6-O-sulfotransferase |
| | | | | | 3 (GST-3), high |
| | | | | | endothelial cells N- |
| | | | | | acetylglucosamine |
| | | | | | 6-O-sulfotransferase |
| | | | | | (HEC-GlcNAc6ST), |
| | | | | | L-selectin ligand |
| | | | | | sulfotransferase |
| | | | | | (LSST), N- |
| | | | | | acetylglucosamine |
| | | | | | 6-O-sulfotransferase |
| | | | | | 2 (GlcNAc6ST-2, |
| | | | | | Gn6st-2) |
| CA8 | CA8 | P35219 | carbonic | CA-RP, CA-VIII, | carbonic anhydrase- |
| | | | anhydrase 8 | CALS, CAMRQ3, | related protein, |
| | | | | CARP, SCAR34 | CARP, carbonic |
| | | | | | anhydrase VIII (CA- |
| | | | | | VIII) |
| FCRL3 | FCRL3 | Q96P31 | Fc receptor like 3 | CD307c, FCRH3, | Fc receptor-like |
| | | | | IFGP3, IRTA3, | protein 3, FcR-like |
| | | | | MAIA, SPAP2 | protein 3, FcRL3, Fc |
| | | | | | receptor homolog 3 |
| | | | | | (FcRH3), IFGP |
| | | | | | family protein 3 |
| | | | | | (hIFGP3), immune |
| | | | | | receptor |
| | | | | | translocation- |
| | | | | | associated protein 3, |
| | | | | | MAIA, SH2 |
| | | | | | domain-containing |
| | | | | | phosphatase anchor |
| | | | | | protein 2, CD307c |
| ASAH2 | ASAH2 | Q9NR71 | N-acylsphingosine | BCDase, HNAC1, | neutral ceramidase, |
| | | | amidohydrolase 2 | LCDase, N- | N-CDase, NCDase, |
| | | | | CDase, NCDase, | acylsphingosine |
| | | | | AL/NCDASE, | deacylase 2, |
| | | | | CDASE | BCDase, LCDase |
| | | | | | (hCD), N- |
| | | | | | acylsphingosine |
| | | | | | amidohydrolase 2, |
| | | | | | non-lysosomal |
| | | | | | ceramidase, neutral |
| | | | | | ceramidase soluble |
| | | | | | form, EC: 3.5.1.- , |
| | | | | | EC: 3.5.1.23 |
| CF XIV | PROC | P04070 | protein C, | APC, PC, THPH3, | vitamin K- |
| | | | inactivator of | THPH4, PROC1, | dependent protein C, |
| | | | coagulation | protein C | protein C, |
| | | | factors Va and | | anticoagulant |
| | | | VIIIa | | protein C, |
| | | | | | autoprothrombin |
| | | | | | IIA, blood |
| | | | | | coagulation factor |
| | | | | | XIV, vitamin K- |
| | | | | | dependent protein C |
| | | | | | light chain, vitamin |
| | | | | | K-dependent protein |
| | | | | | C heavy chain, |
| | | | | | activation peptide, |
| | | | | | EC: 3.4.21.69 |
| PYY | PYY | P10082 | peptide YY | PYY-I, PYY1 | PYY, PYY-I, |
| | | | | | peptide tyrosine |
| | | | | | tyrosine, peptide |
| | | | | | YY(3-36), PYY-II |
| HGF | HGF | P14210 | hepatocyte growth | DFNB39, F-TCF, | hepatopoietin-A, |
| | | | factor | HPTA, SF, HGFB | scatter factor (SF), |
| | | | | | hepatocyte growth |
| | | | | | factor alpha chain, |
| | | | | | hepatocyte growth |
| | | | | | factor beta chain |
| I-TAC | CXCL11 | O14625 | C-X-C motif | H174, I-TAC, IP- | C-X-C motif |
| | | | chemokine ligand | 9, IP9, SCYB11, | chemokine 11, beta- |
| | | | 11 | SCYB9B, b-R1 | R1, H174, interferon |
| | | | | | gamma-inducible |
| | | | | | protein 9 (IP-9), |
| | | | | | interferon-inducible |
| | | | | | T-cell alpha |
| | | | | | chemoattractant (I- |
| | | | | | TAC), small- |
| | | | | | inducible cytokine |
| | | | | | B11, IFN stimulated |
| | | | | | T-cell alpha |
| | | | | | chemoattractant |
| | | | | | precursor, IFN |
| | | | | | stimulated T-cell α |
| | | | | | chemoattractant |
| | | | | | precursor |
| Semaphorin | SEMA4C | Q9C0C4 | semaphorin 4C | M-SEMA-F, | semaphorin-4C |
| 4C | | | | SEMACL1, | |
| | | | | SEMAF, SEMAI, | |
| | | | | KIAA1739, | |
| | | | | UNQ5855/PRO34487 | |
| SorCS3 | SORCS3 | Q9UPU3 | sortilin related | SORCS, | VPS10 domain- |
| | | | VPS10 domain | KIAA1059 | containing receptor |
| | | | containing | | SorCS3 |
| | | | receptor 3 | | |
| Tie-1 | TIE1 | P35590 | tyrosine kinase | JTK14, | tyrosine-protein |
| | | | with | LMPHM11, TIE | kinase receptor Tie- |
| | | | immunoglobulin | | 1, EC: 2.7.10.1 |
| | | | like and EGF like | | |
| | | | domains 1 | | |
| IL-31 RA | IL31RA | Q8NI17 | interleukin 31 | CRL, CRL3, | interleukin-31 |
| | | | receptor A | GLM-R, GLMR, | receptor subunit |
| | | | | GPL, IL-31RA, | alpha, IL-31 |
| | | | | PLCA2, | receptor subunit |
| | | | | PRO21384, | alpha, IL-31R |
| | | | | hGLM-R, | subunit alpha, IL- |
| | | | | zcytoR17, | 31R-alpha, IL- |
| | | | | UNQ6368/PRO21073/ | 31RA, cytokine |
| | | | | PRO21384 | receptor-like 3, |
| | | | | | GLM-R (hGLM-R), |
| | | | | | Gp130-like |
| | | | | | monocyte receptor |
| | | | | | (GP130-like |
| | | | | | receptor), zcytoR17 |
| Arginase 1 | ARG1 | P05089 | arginase 1 | | arginase-1, |
| | | | | | hyperargininaemia, |
| | | | | | hyperargininemia, |
| | | | | | liver-type arginase, |
| | | | | | type I arginase, |
| | | | | | EC: 3.5.3.1 |
| POGLUT1 | POGLUT1 | Q8NBL1 | protein O- | C3orf9, CLP46, | CAP10-like 46 kDa |
| | | | glucosyltransferase | KDELCL1, | protein (hCLP46), |
| | | | 1 | KTELC1, | KTEL motif- |
| | | | | LGMD2Z, | containing protein 1, |
| | | | | LGMDR21, | myelodysplastic |
| | | | | MDS010, | syndromes relative |
| | | | | MDSRP, Rumi, | protein, O- |
| | | | | hCLP46, | glucosyltransferase |
| | | | | UNQ490/PRO1006 | Rumi homolog |
| | | | | | (hRumi), protein O- |
| | | | | | xylosyltransferase |
| | | | | | POGLUTI |
| | | | | | (EC: 2.4.2.63), |
| | | | | | EC: 2.4.1.376 |
| IL-lra | IL1RN | P18510 | interleukin 1 | CRMO2, DIRA, | IL1 receptor |
| | | | receptor | ICIL-1RA, IL- | antagonist, |
| | | | antagonist | 1RN, IL-1ra, IL- | interleukin-1 |
| | | | | 1Ra, IL-1ra3, | receptor antagonist |
| | | | | IL1F3, IL1RA, | protein, IL-1RN, IL- |
| | | | | IRAP, MVCD4 | 1ra, IRAP, ICIL- |
| | | | | | 1RA, IL1 inhibitor, |
| | | | | | anakinra |
| Podoplanin | PDPN | Q86YL7 | podoplanin | AGGRUS, D2-40, | aggrus, glycoprotein |
| | | | | GP36, GP40, | 36 (Gp36), PA2.26 |
| | | | | Gp38, HT1A-1, | antigen, T1-alpha |
| | | | | OTS8, PA2.26, | (TIA), 29 kDa |
| | | | | T1A, T1A-2, | cytosolic podoplanin |
| | | | | T1A2, TI1A, | intracellular domain |
| | | | | PSEC0003, | (PICD) |
| | | | | PSEC0025 | |
| TIM-3 | HAVCR2 | Q8TDQ0 | hepatitis A virus | CD366, HAVcr-2, | HAVcr-2, T-cell |
| | | | cellular receptor 2 | KIM-3, SPTCL, | immunoglobulin and |
| | | | | TIM3, TIMD-3, | mucin domain- |
| | | | | TIMD3, Tim-3 | containing protein 3 |
| | | | | | (TIMD-3), T-cell |
| | | | | | immunoglobulin |
| | | | | | mucin receptor 3 |
| | | | | | (TIM-3), T-cell |
| | | | | | membrane protein 3, |
| | | | | | CD366 |
| CREG | CREG1 | O75629 | cellular repressor | CREG, | protein CREG1, |
| | | | of E1A stimulated | UNQ727/PRO1409 | cellular repressor of |
| | | | genes 1 | | E1A-stimulated |
| | | | | | genes 1 |
| CD300f | CD300LF | Q8TDQ1 | CD300 molecule | CD300f, CD300F, | CMRF35-like |
| | | | like family | CLM-1, CLM1, | molecule 1, CLM-1, |
| | | | member f | IREM-1, IREM1, | CD300 antigen-like |
| | | | | IgSF13, IGSF13, | family member F, |
| | | | | LMIR3, NKIR, | immune receptor |
| | | | | UNQ3105/PRO10111 | expressed on |
| | | | | | myeloid cells 1 |
| | | | | | (IREM-1), |
| | | | | | immunoglobulin |
| | | | | | superfamily member |
| | | | | | 13 (IgSF13), NK |
| | | | | | inhibitory receptor, |
| | | | | | CD300f |
| uPA | PLAU | P00749 | plasminogen | ATF, BDPLT5, | urokinase-type |
| | | | activator, | QPD, UPA, URK, | plasminogen |
| | | | urokinase | u-PA | activator, u- |
| | | | | | plasminogen |
| | | | | | activator, uPA, |
| | | | | | urokinase-type |
| | | | | | plasminogen |
| | | | | | activator long chain |
| | | | | | A, urokinase-type |
| | | | | | plasminogen |
| | | | | | activator short chain |
| | | | | | A, urokinase-type |
| | | | | | plasminogen |
| | | | | | activator chain B, |
| | | | | | EC: 3.4.21.73 |
| EphA2 | EPHA2 | P29317 | EPH receptor A2 | ARCC2, CTPA, | ephrin type-A |
| | | | | CTPP1, CTRCT6, | receptor 2, epithelial |
| | | | | ECK | cell kinase (ECK), |
| | | | | | tyrosine-protein |
| | | | | | kinase receptor |
| | | | | | ECK, EC: 2.7.10.1 |
| LRRTM4 | LRRTM4 | Q86VH4 | leucine rich repeat | UNQ3075/PRO9907 | leucine-rich repeat |
| | | | transmembrane | | transmembrane |
| | | | neuronal 4 | | neuronal protein 4 |
| LIMPII | SCARB2 | Q14108 | scavenger receptor | AMRF, CD36L2, | lysosome membrane |
| | | | class B member 2 | EPM4, HLGP85, | protein 2, 85 kDa |
| | | | | LGP85, LIMP-2, | lysosomal |
| | | | | LIMP2, LIMPII, | membrane |
| | | | | SR-BII | sialoglycoprotein |
| | | | | | (LGP85), CD36 |
| | | | | | antigen-like 2, |
| | | | | | lysosome membrane |
| | | | | | protein II (LIMP II), |
| | | | | | scavenger receptor |
| | | | | | class B member 2, |
| | | | | | CD36 |
| Tenascin R | TNR | Q92752 | tenascin R | NEDSTO, TN-R | tenascin-R, TN-R, |
| | | | | | janusin, restrictin |
| CPE | CPE | P16870 | carboxypeptidase | BDVS, CPH, | CPE, |
| | | | E | IDDHH | carboxypeptidase H |
| | | | | | (CPH), enkephalin |
| | | | | | convertase, |
| | | | | | prohormone- |
| | | | | | processing |
| | | | | | carboxypeptidase, |
| | | | | | EC: 3.4.17.10 |
| PECAM-1 | PECAM1 | P16284 | platelet and | CD31, | platelet endothelial |
| | | | endothelial cell | CD31/EndoCAM, | cell adhesion |
| | | | adhesion molecule | GPIIA′, PECA1, | molecule, PECAM- |
| | | | 1 | PECAM-1, | 1, EndoCAM, |
| | | | | endoCAM, | GPIIA′, PECA1, |
| | | | | PECAM2 | CD31 |
| DNAM-1 | CD226 | Q15762 | CD226 molecule | DNAM-1, | CD226 antigen, |
| | | | | DNAM1, PTA1, | DNAX accessory |
| | | | | TLiSA1 | molecule 1 |
| | | | | | (DNAM-1), CD226 |
| DKK-1 | DKK1 | O94907 | dickkopf WNT | DKK-1, SK, | Dickkopf-related |
| | | | signaling pathway | UNQ492/PRO1008 | protein 1, Dickkopf- |
| | | | inhibitor 1 | | 1, Dkk-1, hDkk-1, |
| | | | | | SK |
| OPG | TNFRSF | O00300 | TNF receptor | OCIF, OPG, | tumor necrosis |
| | 11B | | superfamily | PDB5, TR1, | factor receptor |
| | | | member 11B | TNFR11 | superfamily member |
| | | | | | 11B, |
| | | | | | osteoprotegerin, |
| | | | | | osteoclastogenesis |
| | | | | | inhibitory factor |
| CPB1 | CPB1 | P15086 | carboxypeptidase | CPB, PASP, | carboxypeptidase B, |
| | | | B1 | PCPB | pancrease-specific |
| | | | | | protein (PASP), |
| | | | | | EC: 3.4.17.2 |
| TSH | CGA | P01215 | glycoprotein | CG-ALPHA, | glycoprotein |
| | | | hormones, alpha | FSHA, GPA1, | hormones alpha |
| | | | polypeptide | GPHA1, GPHa, | chain, anterior |
| | | | | HCG, LHA, | pituitary |
| | | | | TSHA, alpha, | glycoprotein |
| | | | | alphaGSU, CG-α, | hormones common |
| | | | | Fsh α, Hcg α, Tsh | subunit alpha, |
| | | | | α, α, α gsu | choriogonadotropin |
| | | | | | alpha chain, |
| | | | | | chorionic |
| | | | | | gonadotrophin |
| | | | | | subunit alpha (CG- |
| | | | | | alpha), follicle- |
| | | | | | stimulating hormone |
| | | | | | alpha chain (FSH- |
| | | | | | alpha), follitropin |
| | | | | | alpha chain, |
| | | | | | luteinizing hormone |
| | | | | | alpha chain (LSH- |
| | | | | | alpha), lutropin |
| | | | | | alpha chain, thyroid- |
| | | | | | stimulating hormone |
| | | | | | alpha chain (TSH- |
| | | | | | alpha), thyrotropin |
| | | | | | alpha chain, |
| | | | | | glycoprotein |
| | | | | | hormone alpha, |
| | | | | | glycoprotein |
| | | | | | hormones α |
| | | | | | polypeptide, |
| | | | | | glycoprotein |
| | | | | | hormone α, |
| | | | | | glycoprotein |
| | | | | | hormone α chain, |
| | | | | | glycoprotein |
| | | | | | hormone α subunit, |
| | | | | | gonadotropin alpha, |
| | | | | | gonadotropin α, LH |
| | | | | | and FSH common |
| | | | | | alpha subunit, LH |
| | | | | | and FSH common a |
| | | | | | subunit |
| | TSHB | P01222 | thyroid | TSH-B, TSH- | thyrotropin subunit |
| | | | stimulating | BETA, TSH-β, | beta, thyroid- |
| | | | hormone subunit | Tsh β | stimulating hormone |
| | | | beta | | subunit beta (TSH- |
| | | | | | B, TSH-beta), |
| | | | | | thyrotropin beta |
| | | | | | chain, thyrotropin |
| | | | | | alfa, thyroid |
| | | | | | stimulating hormone |
| | | | | | subunit β |
| MMP-2 | MMP2 | P08253 | matrix | CLG4, CLG4A, | 72 kDa type IV |
| | | | metallopeptidase 2 | MMP-2, MMP-II, | collagenase, |
| | | | | MONA, TBE-1 | gelatinase, 72 kDa |
| | | | | | gelatinase, |
| | | | | | gelatinase A, matrix |
| | | | | | metalloproteinase-2 |
| | | | | | (MMP-2), TBE-1, |
| | | | | | PEX |
| Siglec-9 | SIGLEC9 | Q9Y336 | sialic acid binding | CD329, CDw329, | sialic acid-binding |
| | | | Ig like lectin 9 | FOAP-9, OBBP- | Ig-like lectin 9, |
| | | | | LIKE, siglec-9, | siglec-9, CDw329, |
| | | | | UNQ668/PRO1302 | protein FOAP-9, |
| | | | | | CD329 |
| ICAM-3 | ICAM3 | P32942 | intercellular | CD50, CDW50, | ICAM-3, CDw50, |
| | | | adhesion molecule | ICAM-R | ICAM-R, CD50 |
| | | | 3 | | |
| Cystatin SA | CST2 | P09228 | cystatin SA | | cystatin 2, cystatin- |
| | | | | | SA, cystatin-2, |
| | | | | | cystatin-S5 |
| Galectin-4 | LGALS4 | P56470 | galectin 4 | GAL4, L36LBP, | galectin-4, Gal-4, |
| | | | | L-36, L36LBI | antigen NY-CO-27, |
| | | | | | L-36 lactose-binding |
| | | | | | protein (L36LBP), |
| | | | | | lactose-binding |
| | | | | | lectin 4 |
| Pepsinogen | PGC | P20142 | progastricsin | PEPC, PGII | gastricsin, |
| II | | | | | pepsinogen C, |
| | | | | | EC: 3.4.23.3 |
| Desmoglein- | DSG3 | P32926 | desmoglein 3 | ABOLM, CDHF6, | desmoglein-3, 130 |
| 3 | | | | PVA | kDa pemphigus |
| | | | | | vulgaris antigen |
| | | | | | (PVA), cadherin |
| | | | | | family member 6 |
| Nectin-4 | NECTIN4 | Q96NY8 | nectin cell | EDSS1, LNIR, | nectin-4, Ig |
| | | | adhesion molecule | PRR4, PVRL4, | superfamily receptor |
| | | | 4 | nectin-4 | LNIR, Poliovirus |
| | | | | | receptor-related |
| | | | | | protein 4, processed |
| | | | | | poliovirus receptor- |
| | | | | | related protein 4 |
| SCF | KITLG | P21583 | KIT ligand | DCUA, DFNA69, | Kit ligand, mast cell |
| | | | | FPH2, FPHH, KL- | growth factor |
| | | | | 1, Kitl, MGF, | (MGF), stem cell |
| | | | | SCF, SF, SHEP7, | factor (SCF), c-Kit |
| | | | | SLF, WS2F | ligand, soluble KIT |
| | | | | | ligand (sKITLG), |
| | | | | | steel factor |
| Serpin A5 | SERPIN | P05154 | serpin family A | PAI-3, PAI3, PCI, | plasma serine |
| | A5 | | member 5 | PCI-B, PLANH3, | protease inhibitor, |
| | | | | PROCI | acrosomal serine |
| | | | | | protease inhibitor, |
| | | | | | plasminogen |
| | | | | | activator inhibitor 3 |
| | | | | | (PAI-3, PAI3), |
| | | | | | protein C inhibitor |
| | | | | | (PCI), serpin A5 |
| PTH | PTH | P01270 | parathyroid | FIH1, PTH1, | PTH, parathormone, |
| | | | hormone | PPTH | parathyrin |
| FGF-19 | FGF19 | O95750 | fibroblast growth | UNQ334/PRO533 | FGF-19 |
| | | | factor 19 | | |
| MSP | MST1 | P26927 | macrophage | D3F15S2, | hepatocyte growth |
| | | | stimulating 1 | DNF15S2, HGFL, | factor-like protein, |
| | | | | MSP, NF15S2 | macrophage |
| | | | | | stimulatory protein, |
| | | | | | macrophage- |
| | | | | | stimulating protein |
| | | | | | (MSP), hepatocyte |
| | | | | | growth factor-like |
| | | | | | protein alpha chain, |
| | | | | | hepatocyte growth |
| | | | | | factor-like protein |
| | | | | | beta chain |
| IL-28A | IFNL2 | Q8IZJ0 | interferon lambda | IFNL2a, IFNL3a, | interferon lambda-2, |
| | | | 2 | IL-28A, IL28A, | iinterferon λ 2, IFN |
| | | | | ZCYTO20, INF λ | lambda 2, IFN λ 2, |
| | | | | 2 | INF-lambda-2, |
| | | | | | cytokine Zcyto20, |
| | | | | | interleukin-28A (IL- |
| | | | | | 28A) |
| FGF-12 | FGF12 | P61328 | fibroblast growth | FHF1, DEE47, | FGF-12, fibroblast |
| | | | factor 12 | EIEE47, FGF12B, | growth factor |
| | | | | LOC100505888 | homologous factor 1 |
| | | | | | (FHF-1), myocyte- |
| | | | | | activating factor |
| METAP2 | METAP2 | P50579 | methionyl | MAP2, MNPEP, | methionine |
| | | | aminopeptidase 2 | p67eIF2, P67EIF2 | aminopeptidase 2, |
| | | | | | MAP 2, MetAP 2, |
| | | | | | initiation factor 2- |
| | | | | | associated 67 kDa |
| | | | | | glycoprotein (p67, |
| | | | | | p67eIF2), peptidase |
| | | | | | M, EC: 3.4.11.18 |
| ASAHL | NAAA | Q02083 | N- | ASAHL, PLT | N- |
| | | | acylethanolamine | | acylethanolamine- |
| | | | acid amidase | | hydrolyzing acid |
| | | | | | amidase, acid |
| | | | | | ceramidase-like |
| | | | | | protein, |
| | | | | | acylsphingosine |
| | | | | | deacylase NAAA |
| | | | | | (EC: 3.5.1.23), N- |
| | | | | | acylsphingosiine |
| | | | | | amidohydrolase-like |
| | | | | | (ASAH-like |
| | | | | | protein), N- |
| | | | | | acylethanolamine- |
| | | | | | hydrolyzing acid |
| | | | | | amidase subunit |
| | | | | | alpha, N- |
| | | | | | acylethanolamine- |
| | | | | | hydrolyzing acid |
| | | | | | amidase subunit |
| | | | | | beta, EC: 3.5.1.60 |
| EDIL3 | EDIL3 | O43854 | EGF like repeats | DEL1 | EGF-like repeat and |
| | | | and discoidin | | discoidin I-like |
| | | | domains 3 | | domain-containing |
| | | | | | protein 3, |
| | | | | | developmentally- |
| | | | | | regulated |
| | | | | | endothelial cell |
| | | | | | locus 1 protein, |
| | | | | | integrin-binding |
| | | | | | protein DEL1 |
| NTAL | LAT2 | Q9GZY6 | linker for | HSPC046, LAB, | linker for activation |
| | | | activation of T | NTAL, | of T-cells family |
| | | | cells family | WBSCR15, | member 2, linker for |
| | | | member 2 | WBSCR5, | activation of B-cells, |
| | | | | WSCR5 | linker for B-cell |
| | | | | | activation, |
| | | | | | membrane- |
| | | | | | associated adapter |
| | | | | | molecule, non-T- |
| | | | | | cell activation |
| | | | | | linker, Williams- |
| | | | | | Beuren syndrome |
| | | | | | chromosomal region |
| | | | | | 15 protein, |
| | | | | | Williams-Beuren |
| | | | | | syndrome |
| | | | | | chromosomal region |
| | | | | | 5 protein |
| EGF R | EGFR | P00533 | epidermal growth | ERBB, ERBB1, | proto-oncogene c- |
| | | | factor receptor | ERRP, HER1, | ErbB-1, receptor |
| | | | | NISBD2, NNCIS, | tyrosine-protein |
| | | | | PIG61, mENA, C- | kinase erbB-1, |
| | | | | ERBB, EGFR1, | EC: 2.7.10.1 |
| | | | | Erbb1, MENA | |
| TAFAS | TAFA1 | Q7Z5A9 | TAFA chemokine | FAM19A1, | chemokine-like |
| | | | like family | TAFA-1 | protein TAFA-1 |
| | | | member 1 | | |
| | TAFA2 | Q8N3H0 | TAFA chemokine | FAM19A2, | chemokine-like |
| | | | like family | TAFA-2, | protein TAFA-2 |
| | | | member 2 | LOC102724280, | |
| | | | | LOC338811 | |
| | TAFA3 | Q7Z5A8 | TAFA chemokine | FAM19A3, | chemokine-like |
| | | | like family | TAFA-3 | protein TAFA-3 |
| | | | member 3 | | |
| | TAFA4 | Q96LR4 | TAFA chemokine | FAM19A4, | chemokine-like |
| | | | like family | TAFA-4 | protein TAFA-4 |
| | | | member 4 | | |
| | TAFA5 | Q7Z5A7 | TAFA chemokine | FAM19A5, | chemokine-like |
| | | | like family | QLLK5208, | protein TAFA-5 |
| | | | member 5 | TAFA-5, | |
| | | | | UNQ5208, | |
| | | | | UNQ5208/PRO34524 | |
| Galectin-9 | LGALS9 | O00182 | galectin 9 | HUAT, | galectin-9, Gal-9, |
| | | | | LGALS9A, HOM- | ecalectin, tumor |
| | | | | HD-21 | antigen HOM-HD- |
| | | | | | 21 |
| vWF-A2 | VWA2 | Q5GFL6 | von Willebrand | AMACO, CCSP- | von Willebrand |
| | | | factor A domain | 2, CCSP2, NET42, | factor A domain- |
| | | | containing 2 | RGD1562000, | containing protein 2, |
| | | | | Vwa2-ps1 | a domain-containing |
| | | | | | protein similar to |
| | | | | | matrilin and |
| | | | | | collagen (AMACO), |
| | | | | | colon cancer |
| | | | | | secreted protein 2 |
| | | | | | (CCSP-2) |
| TACE | ADAM17 | P78536 | ADAM | ADAM18, | disintegrin and |
| | | | metallopeptidase | CD156B, CSVP, | metalloproteinase |
| | | | domain 17 | NISBD, NISBD1, | domain-containing |
| | | | | TACE | protein 17, ADAM |
| | | | | | 17, snake venom- |
| | | | | | like protease, TNF- |
| | | | | | alpha convertase, |
| | | | | | Tnfa convertase, |
| | | | | | TNF-α-converting |
| | | | | | enzyme, TNF-alpha- |
| | | | | | converting enzyme, |
| | | | | | CD156b, |
| | | | | | EC: 3.4.24.86 |
| Cathepsin S | CTSS | P25774 | cathepsin S | | EC: 3.4.22.27 |
| LDL R | LDLR | P01130 | low density | FH, FHC, FHCL1, | low-density |
| | | | lipoprotein | LDLCQ2 | lipoprotein receptor, |
| | | | receptor | | LDL receptor |
| BMPR-IA | BMPR1A | P36894 | bone morphogenic | 10q23del, | bone morphogenic |
| | | | protein receptor | ACVRLK3, ALK- | protein receptor |
| | | | type 1A | 3, ALK3, BMPR- | type-1A, BMP type- |
| | | | | 1A, CD292, | 1A receptor, BMPR- |
| | | | | SKR5, JIP | 1A, activin receptor- |
| | | | | | like kinase 3 (ALK- |
| | | | | | 3), serine/threonine- |
| | | | | | protein kinase |
| | | | | | receptor R5 (SKR5), |
| | | | | | CD292, |
| | | | | | EC: 2.7.11.30 |
| OX40 | TNFRSF4 | P43489 | TNF receptor | ACT35, CD134, | tumor necrosis |
| | | | superfamily | IMD16, OX40, | factor receptor |
| | | | member 4 | TXGP1L | superfamily member |
| | | | | | 4, ACT35 antigen, |
| | | | | | OX40L receptor, |
| | | | | | TAX |
| | | | | | transcriptionally- |
| | | | | | activated |
| | | | | | glycoprotein 1 |
| | | | | | receptor, CD134 |
| IL-13 R2 | IL13RA2 | Q14627 | interleukin 13 | CD213A2, CT19, | interleukin-13 |
| | | | receptor subunit | IL-13R, IL13R, | receptor subunit |
| | | | alpha 2 | IL13BP | alpha-2, interleukin |
| | | | | | 13 receptor subunit |
| | | | | | α 2, IL-13 receptor |
| | | | | | subunit alpha-2, IL- |
| | | | | | 13R subunit alpha-2, |
| | | | | | IL-13R-alpha-2, IL- |
| | | | | | 13RA2, interleukin- |
| | | | | | 13 binding protein, |
| | | | | | CD213a2 |
| B7-H4 | VTCN1 | Q7Z7D3 | V-set domain | B7-H4, B7H4, | V-set domain- |
| | | | containing T-cell | B7S1, B7X, | containing T-cell |
| | | | activation | B7h.5, PRO1291, | activation inhibitor |
| | | | inhibitor 1 | VCTN1, | 1, B7 homolog 4 |
| | | | | UNQ659/PRO1291 | (B7-H4), B7h.5, |
| | | | | | immune |
| | | | | | costimulatory |
| | | | | | protein B7-H4, |
| | | | | | protein B7S1, T-cell |
| | | | | | costimulatory |
| | | | | | molecule B7x |
| MMP-13 | MMP13 | P45452 | matrix | CLG3, MANDP1, | collagenase 3, |
| | | | metallopeptidase | MDST, MMP-13 | matrix |
| | | | 13 | | metalloproteinase- |
| | | | | | 13 (MMP-13), |
| | | | | | EC: 3.4.24.- |
| ANGPTL7 | ANGPTL7 | O43827 | angiopoietin like 7 | AngX, CDT6, | angiopoietin-related |
| | | | | dJ647M16.1, | protein 7, |
| | | | | UNQ313/PRO356 | angiopoietin-like |
| | | | | | factor, angiopoietin- |
| | | | | | like protein 7, |
| | | | | | cornea-derived |
| | | | | | transcript 6 protein |
| TRAIL R4 | TNFRSF | Q9UBN6 | TNF receptor | CD264, DCR2, | tumor necrosis |
| | 10D | | superfamily | TRAIL-R4, | factor receptor |
| | | | member 10d | TRAILR4, | superfamily member |
| | | | | TRUNDD, | 10D, decoy receptor |
| | | | | UNQ251/PRO288 | 2 (DcR2), TNF- |
| | | | | | related apoptosis- |
| | | | | | inducing ligand |
| | | | | | receptor 4 (TRAIL |
| | | | | | receptor 4, TRAIL- |
| | | | | | R4), TRAIL |
| | | | | | receptor with a |
| | | | | | truncated death |
| | | | | | domain, CD264 |
| IGSF4B | CADM3 | Q8N126 | cell adhesion | BIgR, CMT2FF, | brain |
| | | | molecule 3 | IGSF4B, NECL1, | immunoglobulin |
| | | | | Necl-1, TSLL1, | receptor, |
| | | | | synCAM3, | immunoglobulin |
| | | | | SYNCAM3, | superfamily member |
| | | | | UNQ225/PRO258 | 4B (IgSF4B), |
| | | | | | nectin-like protein 1 |
| | | | | | (NECL-1), synaptic |
| | | | | | cell adhesion |
| | | | | | molecule 3 |
| | | | | | (SynCAM3), |
| | | | | | TSLC1-like protein |
| | | | | | 1 (TSLL1) |
| Sirtuin 5 | SIRT5 | Q9NXA8 | sirtuin 5 | SIR2L5, | NAD-dependent |
| | | | | LOC285813 | protein deacylase |
| | | | | | sirtuin-5 |
| | | | | | mitochondrial, |
| | | | | | regulatory protein |
| | | | | | SIR2 homolog 5, |
| | | | | | SIR2-like protein 5, |
| | | | | | EC: 2.3.1.- |
| PEAR1 | PEAR1 | Q5VY43 | platelet | JEDI, MEGF12 | hPEAR1, multiple |
| | | | endothelial | | epidermal growth |
| | | | aggregation | | factor-like domains |
| | | | receptor 1 | | protein 12, multiple |
| | | | | | EGF-like domains |
| | | | | | protein 12 |
| SH2D1A | SH2D1A | O60880 | SH2 domain | DSHP, EBVS, | SH2 domain- |
| | | | containing 1A | IMD5, LYP, | containing protein |
| | | | | MTCP1, SAP, | 1A, Duncan disease |
| | | | | SAP/SH2D1A, | SH2-protein, |
| | | | | XLP, XLPD, | signaling |
| | | | | XLPD1, SLAM | lymphocytic |
| | | | | | activation molecule- |
| | | | | | associated protein |
| | | | | | (SLAM-associated |
| | | | | | protein), T-cell |
| | | | | | signal transduction |
| | | | | | molecule SAP |
| Cerberus 1 | CER1 | O95813 | cerberus 1, DAN | DAND4 | cerberus, cerberus 1, |
| | | | family BMP | | cerberus-related |
| | | | antagonist | | protein, DAN |
| | | | | | domain family |
| | | | | | member 4 |
| GDF-11 | GDF11 | O95390 | growth | BMP-11, BMP11, | growth/differentiation |
| | | | differentiation | VHO | factor 11, GDF- |
| | | | factor 11 | | 11, bone |
| | | | | | morphogenic protein |
| | | | | | 11 (BMP-11) |
| Nrf2 | NFE2L2 | Q16236 | NFE2 like bZIP | HEBP1, | nuclear factor |
| | | | transcription | IMDDHH, NRF2, | erythroid 2-related |
| | | | factor 2 | Nrf-2, BM974200, | factor 2, NF-E2- |
| | | | | Nrf2 | related factor 2, |
| | | | | | NFE2-related factor |
| | | | | | 2, Nrf-2, nuclear |
| | | | | | factor erythroid |
| | | | | | derived 2 like 2 |
| TROP-2 | TACSTD2 | P09758 | tumor associated | EGP-1, EGP1, | tumor-associated |
| | | | calcium signal | GA733-1, | calcium signal |
| | | | transducer 2 | GA7331, GP50, | transducer 2, tumour |
| | | | | M1S1, TROP2, | associated calcium |
| | | | | Trop-2 | signal transducer 2, |
| | | | | | cell surface |
| | | | | | glycoprotein Trop-2, |
| | | | | | membrane |
| | | | | | component |
| | | | | | chromosome 1 |
| | | | | | surface marker 1, |
| | | | | | pancreatic |
| | | | | | carcinoma marker |
| | | | | | protein GA733-1 |
| NUDTS | NUDT2 | P50583 | nudix hydrolase 2 | APAH1, IDDPN | bis(5′-nucleosyl)- |
| | | | | | tetraphosphatase |
| | | | | | [asymetrical], |
| | | | | | diadenosine 5′,5″′- |
| | | | | | P1,P4- |
| | | | | | tetraphosphate |
| | | | | | asymmetrical |
| | | | | | hydrolase (Ap4A |
| | | | | | hydrolase, |
| | | | | | AP4Aase, |
| | | | | | diadenosine |
| | | | | | tetraphosphatase), |
| | | | | | nucleoside |
| | | | | | diphosphate-linked |
| | | | | | moiety X motif 2 |
| | | | | | (Nudix motif 2) |
| ROR2 | ROR2 | Q01974 | receptor tyrosine | BDB, BDB1, | tyrosine-protein |
| | | | kinase like orphan | NTRKR2, | kinase |
| | | | receptor 2 | LOC101927935 | transmembrane |
| | | | | | receptor ROR2, |
| | | | | | neurotrophic |
| | | | | | tyrosine kinase |
| | | | | | receptor-related 2, |
| | | | | | EC: 2.7.10.1 |
| EphB4 | EPHB4 | P54760 | EPH receptor B4 | CMAVM2, | ephrin type-B |
| | | | | HFASD, HTK, | receptor 4, |
| | | | | LMPHM7, | hepatoma |
| | | | | MYK1, TYRO11 | transmembrane |
| | | | | | kinase, tyrosine- |
| | | | | | protein kinase |
| | | | | | TYRO11, |
| | | | | | EC: 2.7.10.1 |
| Glypican 1 | GPC1 | P35052 | glypican 1 | glypican | glypican-1, secreted |
| | | | | | glypican-1 |
| LAP(TGFb1) | TGFB1 | P01137 | transforming | CED, DPD1, | transforming growth |
| | | | growth factor beta | IBDIMDE, LAP, | factor beta-1 |
| | | | 1 | TGF-beta1, TGFB, | proprotein, |
| | | | | TGFbeta, TGF β 1 | transforming beta-1 |
| | | | | | growth factor, |
| | | | | | transforming β-1 |
| | | | | | growth factor, |
| | | | | | transforming growth |
| | | | | | factor β 1, latency- |
| | | | | | associated peptide |
| | | | | | (LAP), transforming |
| | | | | | growth factor beta-1 |
| | | | | | (TGF-beta-1) |
| Contactin-1 | CNTN1 | Q12860 | contactin 1 | CMYO12, | contactin-1, |
| | | | | CMYP12, F3, | glycoprotein gp135, |
| | | | | GP135, MYPCN | neural cell surface |
| | | | | | protein F3 |
| IL-27 | EBI3 | Q14213 | Epstein-Barr virus | IL-27B, IL27B, | interleukin-27 |
| | | | induced 3 | IL35B | subunit beta, IL-27 |
| | | | | | subunit beta, IL- |
| | | | | | 27B, Epstein-Barr |
| | | | | | virus-induced gene |
| | | | | | 3 protein, EBV- |
| | | | | | induced gene 3 |
| | | | | | protein |
| | IL27 | Q8NEV9 | interleukin 27 | IL-27, IL-27A, | interleukin-27 |
| | | | | IL27A, IL27p28, | subunit alpha, IL-27 |
| | | | | p28, IL30 | subunit alpha, IL- |
| | | | | | 27-A, IL27-A, |
| | | | | | interleukin-30, p28 |
| UNC5H4 | UNC5D | Q6UXZ4 | unc-5 netrin | PRO34692, | netrin receptor |
| | | | receptor D | Unc5h4, | UNC5D, protein |
| | | | | UNC5H4, | unc-5 homolog 4, |
| | | | | KIAA1777, | protein unc-5 |
| | | | | UNQ6012/PRO34692 | homolog D |
| ICAM-2 | ICAM2 | P13598 | intercellular | CD102 | ICAM-2, CD102 |
| | | | adhesion molecule | | |
| | | | 2 | | |
| MBL | MBL2 | P11226 | mannose binding | MBL, MBP, | mannose-binding |
| | | | lectin 2 | MBP1, MBPD, | protein C, MBP-C, |
| | | | | MBL2D, MBP-C, | collectin-1, MBP1, |
| | | | | COLEC1, | mannan-binding |
| | | | | HSMBPC | protein, mannose- |
| | | | | | binding lectin |
| HS3ST3B1 | HS3ST3B1 | Q9Y662 | heparan sulfate- | 3-OST-3B, | heparan sulfate |
| | | | glucosamine 3- | 3OST3B1, h3- | glucosamine 3-O- |
| | | | sulfotransferase | OST-3B, | sulfotransferase |
| | | | 3B1 | HS3ST3B, 3- | 3B1, heparan |
| | | | | Ost3b | sulphate- |
| | | | | | glucosamine 3- |
| | | | | | sulphotransferase |
| | | | | | 3B1, heparan sulfate |
| | | | | | D-glucosaminy1 3- |
| | | | | | O-sulfotransferase |
| | | | | | 3B1 (3-OST-3B), |
| | | | | | heparan sulfate 3-O- |
| | | | | | sulfotransferase 3B1 |
| | | | | | (h3-OST-3B) |
| RCOR1 | RCOR1 | Q9UKL0 | REST corepressor | COREST, RCOR, | protein CoREST |
| | | | 1 | KIAA0071 | |
| IL-10 Rb | IL10RB | Q08334 | interleukin 10 | CDW210B, | interleukin-10 |
| | | | receptor subunit | CRF2-4, CRFB4, | receptor subunit |
| | | | beta | D21S58, D21S66, | beta, IL-10 receptor |
| | | | | IBD25, IL-10R2, | subunit beta, IL-10R |
| | | | | IL-10RB, IL-10R- | subunit beta, IL- |
| | | | | β, Il 10r β | 10RB, interleukin |
| | | | | | 10 receptor subunit |
| | | | | | β, interleukin 10 |
| | | | | | receptor β chain, |
| | | | | | cytokine receptor |
| | | | | | class-II member 4, |
| | | | | | cytokine receptor |
| | | | | | family 2 member 4 |
| | | | | | (CRF2-4), |
| | | | | | interleukin-10 |
| | | | | | receptor subunit 2 |
| | | | | | (IL-10R subunit 2, |
| | | | | | IL-10R2), |
| | | | | | CDw210b |
| XEDAR | EDA2R | Q9HAV5 | ectodysplasin A2 | EDA-A2R, | tumor necrosis |
| | | | receptor | EDAA2R, | factor receptor |
| | | | | TNFRSF27, | superfamily member |
| | | | | XEDAR, | 27, X-linked |
| | | | | UNQ2448/PRO5727/ | ectodysplasin-A2 |
| | | | | PRO34080 | receptor (EDA-A2 |
| | | | | | receptor) |
| IL-22 | IL22 | Q9GZX6 | interleukin 22 | IL-21, IL-22, IL- | interleukin-22, IL- |
| | | | | D110, IL-TIF, | 22, cytokiine |
| | | | | ILTIF, TIFIL-23, | Zcyto18, IL-10- |
| | | | | TIFa, zcyto18, | related T-cell- |
| | | | | ZCYTO18, | derived-inducible |
| | | | | UNQ3099/PRO10096 | factor (IL-TIF) |
| PILR-alpha | PILRA | Q9UKJ1 | paired | FDF03 | paired |
| | | | immunoglobulin | | immunoglobulin- |
| | | | like type 2 | | like type 2 receptor |
| | | | receptor alpha | | alpha, paired |
| | | | | | immunoglobulin |
| | | | | | like type 2 receptor |
| | | | | | α, cell surface |
| | | | | | receptor FDF03, |
| | | | | | inhibitory receptor |
| | | | | | PILR-alpha |
| NRG1-131 | NRG1 | Q02297 | neuregulin 1 | GGF, HGL, HRG, | pro-neuregulin-1 |
| | | | | NDF, ARIA, | membrane-bound |
| | | | | GGF2, HRGA, | isoform, pro-NRG1, |
| | | | | SMDF, MST131, | neuregulin-1, |
| | | | | MSTP131, NRG1- | acetylcholine |
| | | | | IT2, GP30, HRG1, | receptor-inducing |
| | | | | NAF | activity (ARIA), |
| | | | | | breast cancer cell |
| | | | | | differentiation factor |
| | | | | | p45, gIial growth |
| | | | | | factor, heregulin |
| | | | | | (HRG), Neu |
| | | | | | differentiation |
| | | | | | factor, sensory and |
| | | | | | motor neuron- |
| | | | | | derived factor, |
| | | | | | NRG1 secreted |
| FABP4 | FABP4 | P15090 | fatty acid binding | A-FABP, AFABP, | fatty acid-binding |
| | | | protein 4 | ALBP, HEL-S- | protein adipocyte, |
| | | | | 104, aP2, AP2 | adipocyte lipid- |
| | | | | | binding protein |
| | | | | | (ALBP), adipocyte- |
| | | | | | type fatty acid- |
| | | | | | binding protein (A- |
| | | | | | FABP, AFABP), |
| | | | | | fatty acid-binding |
| | | | | | protein 4 |
| RGM-A | RGMA | Q96B86 | repulsive guidance | RGM, LOC56963 | repulsive guidance |
| | | | molecule BMP | | molecule A, RGM |
| | | | co-receptor a | | domain family |
| | | | | | member A |
| RELT | RELT | Q969Z4 | RELT TNF | AI3C, | tumor necrosis |
| | | | receptor | TNFRSF19L, | factor receptor |
| | | | | TRLT | superfamily member |
| | | | | | 19L, receptor |
| | | | | | expressed in |
| | | | | | lymphoid tissues |
| TrkC | NTRK3 | Q16288 | neurotrophic | GP145-TrkC, | NT-3 growth factor |
| | | | receptor tyrosine | TRKC, | receptor, GP145- |
| | | | kinase 3 | gp145(trkC) | TrkC (Trk-C), |
| | | | | | neurotrophic |
| | | | | | tyrosine kinase |
| | | | | | receptor type 3, |
| | | | | | TrkC tyrosine |
| | | | | | kinase, EC: 2.7.10.1 |
| Csa | ERCC8 | Q13216 | ERCC excision | CKN1, CSA, | DNA excision repair |
| | | | repair 8, CSA | UVSS2, Csb | protein ERCC-8, |
| | | | ubiquitin ligase | | Cockayne syndrome |
| | | | complex subunit | | WD repeat protein |
| | | | | | CSA |
| SREC-I | SCARF1 | Q14162 | scavenger receptor | SREC, SREC-I, | acetyl LDL receptor, |
| | | | class F member 1 | SREC1, | scavenger receptor |
| | | | | KIAA0149 | expressed by |
| | | | | | endothelial cells 1 |
| | | | | | (SREC-I) |
| Nestin | NES | P48681 | nestin | Nbla00170, | |
| | | | | Nestin 1 | |
| TPO | TPO | P07202 | thyroid peroxidase | MSA, TDH2A, | TPO, EC: 1.11.1.8 |
| | | | | TPX | |
| ErbB3 | ERBB3 | P21860 | erb-b2 receptor | ErbB-3, FERLK, | receptor tyrosine- |
| | | | tyrosine kinase 3 | HER3, LCCS2, | protein kinase erbB- |
| | | | | MDA-BF-1, | 3, proto-oncogene- |
| | | | | VSCN1, c-erbB-3, | like protein c-ErbB- |
| | | | | c-erbB3, erbB3-S, | 3, tyrosine kinase- |
| | | | | p180-ErbB3, p45- | type cell surface |
| | | | | sErbB3, p85- | receptor HER3 |
| | | | | sErbB3, EGFR3, | |
| | | | | erbB3-S | |
| Kirre13 | KIRREL3 | Q8IZU9 | kirre like nephrin | KIRRE, MRD4, | kin of IRRE-like |
| | | | family adhesion | NEPH2, | protein 3, kin of |
| | | | molecule 3 | PRO4502, | irregular chiasm-like |
| | | | | KIAA1867, | protein 3, nephrin- |
| | | | | UNQ5923/PRO4502/ | like protein 2, |
| | | | | PRO19814 | Nephrin-like 2, |
| | | | | | processed kin of |
| | | | | | IRRE-like protein 3 |
| FLRT1 | FLRT1 | Q9NZU1 | fibronectin leucine | SPG68, | leucine-rich repeat |
| | | | rich | RP11_21A7A2, | transmembrane |
| | | | transmembrane | UNQ752/PRO1483 | protein FLRT1, |
| | | | protein 1 | | fibronectin-like |
| | | | | | domain-containing |
| | | | | | leucine-rich |
| | | | | | transmembrane |
| | | | | | protein 1 |
| Galectin-3 | LGALS3 | P17931 | galectin 3 | CBP35, GAL3, | galectin-3, Gal-3, 35 |
| | | | | GALBP, GALIG, | kDa lectin, |
| | | | | L31, LGALS2, | carbohydrate- |
| | | | | MAC2, LEG3, | binding protein 35 |
| | | | | MTIOGALIGIN | (CBP 35), galactose- |
| | | | | | specific lectin 3, |
| | | | | | galatoside-binding |
| | | | | | protein (GALBP), |
| | | | | | IgE-binding protein, |
| | | | | | L-31, laminin- |
| | | | | | binding protein, |
| | | | | | lectin L-29, mac-2 |
| | | | | | antigen |
| CXCL16 | CXCL16 | Q9H2A7 | C-X-C motif | CXCLG16, SR- | C-X-C motif |
| | | | chemokine ligand | PSOX, SRPSOX, | chemokine 16, |
| | | | 16 | SCYB16, | scavenger receptor |
| | | | | UNQ2759/PRO67 | for |
| | | | | 14, CX3CL16 | phosphatidylserine |
| | | | | | and oxidized low |
| | | | | | density lipoprotein |
| | | | | | (SR-PSOX), small- |
| | | | | | inducible cytokine |
| | | | | | B16, transmembrane |
| | | | | | chemokine CXCL16 |
| JAM-B | JAM2 | P57087 | junctional | C21orf43, CD322, | junctional adhesion |
| | | | adhesion molecule | IBGC8, JAM-B, | molecule B, JAM-B, |
| | | | 2 | JAMB, PRO245, | JAM-2, vascular |
| | | | | VE-JAM, | endothelial junction- |
| | | | | VEJAM, | associated molecule |
| | | | | UNQ219/PRO245 | (VE-JAM), CD322 |
| DR6 | TNFRSF | O75509 | TNF receptor | BM-018, CD358, | tumor necrosis |
| | 21 | | superfamily | DR6, | factor receptor |
| | | | member 21 | UNQ437/PRO868 | superfamily member |
| | | | | | 21, death receptor 6 |
| | | | | | (DR6), CD358 |
| Nogo | RTN4R | Q9BZR6 | reticulon 4 | NGR, NOGOR, | reticulon-4 receptor, |
| Receptor | | | receptor | UNQ330/PRO526 | Nogo receptor |
| | | | | | (NgR), Nogo-66 |
| | | | | | receptor |
| TLR4 | TLR4 | O00206 | toll like receptor 4 | ARMD10, CD284, | toll-like receptor 4, |
| | | | | TLR-4, TOLL, | TOLL receptor, |
| | | | | HTOLL | hToll, CD284 |
| VEGF R2 | KDR | P35968 | kinase insert | CD309, FLK1, | vacular endothelial |
| | | | domain receptor | VEGFR, | growth factor |
| | | | | | receptor 2, VEGFR- |
| | | | | VEGFR2, | 2, fetal liver kinase |
| | | | | VEGFR-2 | 1 (FLK-1), kinase |
| | | | | | insert domain |
| | | | | | receptor (KDR), |
| | | | | | protein-tyrosine |
| | | | | | kinase receptor flk- |
| | | | | | 1, CD309 |
| Tie-2 | TEK | Q02763 | TEK receptor | CD202B, GLC3E, | angiopoietin-1 |
| | | | tyrosine kinase | TIE-2, TIE2, | receptor, endothelial |
| | | | | VMCM, VMCM1 | tyrosine kinase, |
| | | | | | tunica interna |
| | | | | | endothelial cell |
| | | | | | kinase, tyrosine |
| | | | | | kinase with Ig and |
| | | | | | EGF homology |
| | | | | | domains-2, tyrosine- |
| | | | | | protein kinase |
| | | | | | receptor TEK, |
| | | | | | tyrosine-protein |
| | | | | | kinase receptor TIE- |
| | | | | | 2 (hTIE2), p140 |
| | | | | | TEK, CD202b |
| IL-15 R | IL15RA | Q13261 | interleukin 15 | CD215, IL-15R, | interleukin-15 |
| | | | receptor subunit | IL-15Ralpha | receptor subunit |
| | | | alpha | | alpha, interleukin 15 |
| | | | | | receptor subunit α, |
| | | | | | IL-15 reeptor |
| | | | | | subunit alpha, IL-15 |
| | | | | | receptor α, IL-15R- |
| | | | | | alpha, IL-15RA, |
| | | | | | soluble interleukin- |
| | | | | | 15 receptor subunit |
| | | | | | alpha (sIL-15 |
| | | | | | receptor subunit |
| | | | | | alpha, sIL-15R- |
| | | | | | alpha, sIL-15RA), |
| | | | | | CD215 |
| Caspr2 | CNTNAP2 | Q9UHC6 | contactin | AUTS15, | contactin-associated |
| | | | associated protein | CASPR2, CDFE, | protein-like 2, cell |
| | | | 2 | NRXN4, PTHSL1, | recognition |
| | | | | KIAA0868 | molecule Caspr2 |
| LTbR | LTBR | P36941 | lymphotoxin beta | D12S370, LT- | lymphotoxin β |
| | | | receptor | BETA-R, TNF-R- | receptor, tumor |
| | | | | III, TNFCR, | necrosis factor |
| | | | | TNFR-RP, | receptor superfamily |
| | | | | TNFR2-RP, | member 3, |
| | | | | TNFR3, | lymphotoxin-beta |
| | | | | TNFRSF3, Lt β R, | receptor, tumor |
| | | | | LT-β-R | necrosis factor C |
| | | | | | receptor, tumor |
| | | | | | necrosis factor |
| | | | | | receptor 2-related |
| | | | | | protein, tumor |
| | | | | | necrosis factor |
| | | | | | receptor type III |
| | | | | | (TNF-RIII, TNFR- |
| | | | | | III) |
| LAMP | LAMP1 | P11279 | lysosomal | CD107a, LAMPA, | lysosome-associated |
| | | | associated | LGP120 | membrane |
| | | | membrane protein | | glycoprotein 1, |
| | | | 1 | | LAMP-1, lysosome- |
| | | | | | associated |
| | | | | | membrane protein 1, |
| | | | | | CD107 antigen-like |
| | | | | | family member A, |
| | | | | | CD107a |
| ALCAM | ALCAM | Q13740 | activated | CD166, MEMD, | CD166 antigen, |
| | | | leukocyte cell | 201951_at, | ALCAM isoform 1, |
| | | | adhesion molecule | ALCAM isoform | CD166 |
| | | | | 1 | |
| GLP-1 | GCG | P01275 | glucagon | GLP-1, GLP1, | pro-glucagon, |
| | | | | GLP2, GRPP | proglucagon, |
| | | | | | glicentin, glicentin- |
| | | | | | related polypeptide |
| | | | | | (GRPP), |
| | | | | | oxyntomodulin |
| | | | | | (OXM, OXY), |
| | | | | | glucagon-like |
| | | | | | peptide 1 (GLP-1, |
| | | | | | incretin hormone), |
| | | | | | glucagon-like |
| | | | | | peptide 1(7-37) |
| | | | | | (GLP-1(7-37)), |
| | | | | | glucagon-like |
| | | | | | peptide 1(7-36) |
| | | | | | (GLP-1(7-36)), |
| | | | | | glucagon-like |
| | | | | | peptide 2 (GLP-2) |
| NG2 | CSPG4 | Q6UVK1 | chondroitin sulfate | CSPG4A, HMW- | chondroitin sulphate |
| | | | proteoglycan 4 | MAA, MCSP, | proteoglycan 4, |
| | | | | MCSPG, MEL- | chondroitin sulfate |
| | | | | CSPG, MSK16, | proteoglycan NG2, |
| | | | | NG2 | melanoma |
| | | | | | chondroitin sulfate |
| | | | | | proteoglycan, |
| | | | | | melanoma-associate |
| | | | | | chondroitin sulfate |
| | | | | | proteoglycan |
| IL-22 R | IL22RA1 | Q8N6P7 | interleukin 22 | CRF2-9, IL22R, | interleukin-22 |
| alpha 1 | | | receptor subunit | IL22R1 | receptor subunit |
| | | | alpha 1 | | alpha-1, interleukin |
| | | | | | 22 receptor subunit |
| | | | | | α 1, IL-22 receptor |
| | | | | | subunit alpha-1, IL- |
| | | | | | 22R-alpha-1, IL- |
| | | | | | 22RA1, cytokine |
| | | | | | receptor class-II |
| | | | | | member 9, cytokine |
| | | | | | receptor family 2 |
| | | | | | member 9 (CRF2- |
| | | | | | 9), zcytoR11 |
| AMIGO2 | AMIGO2 | Q86SJ2 | adhesion molecule | ALI1, AMIGO-2, | amphoterin-induced |
| | | | with Ig like | DEGA, | protein 2, AMIGO- |
| | | | domain 2 | AC004010, | 2, alivin-2, |
| | | | | LOC102724147 | differentially |
| | | | | | expressed in gastric |
| | | | | | adenocarcinomas |
| | | | | | (DEGA) |
| HCC-1 | CCL14 | Q16627 | C-C motif | CC-1, CC-3, | C-C motif |
| | | | chemokine ligand | CKB1, HCC-1, | chemokine 14, |
| | | | 14 | HCC-1(1-74), | chemokine CC- |
| | | | | HCC-1/HCC-3, | 1/CC-3 (HCC- |
| | | | | HCC-3, MCIF, | 1/HCC-3), HCC- |
| | | | | NCC-2, NCC2, | 1(1-74), NCC-2, |
| | | | | SCYA14, SCYL2, | small-inducible |
| | | | | SY14 | cytokine A14, HCC- |
| | | | | | 1(3-74), HCC-1(4- |
| | | | | | 74), HCC-1(9-74) |
| TFPI-2 | TFPI2 | P48307 | tissue factor | PP5, REF1, TFPI- | TFPI-2, placental |
| | | | pathway inhibitor | 2, TFPI2 | protein 5 (PP5) |
| | | | 2 | | |
| ULBP-2 | ULBP2 | Q8BZM5 | UL16 binding | ALCAN-alpha, | UL16-binding |
| | | | protein 2 | ALCAN-α, | protein 2, ALCAN- |
| | | | | N2DL2, | alpha, NKG2D |
| | | | | NKG2DL2, | ligand 2 (N2DL-2, |
| | | | | RAET1H, | NKG2DL2), |
| | | | | RAET1L, | retinoic acid early |
| | | | | UNQ463/PRO791 | transcript 1H |
| | | | | | (RAET1H) |
| Desmoglein2 | DSG2 | Q14126 | desmoglein 2 | CDHF5, HDGC | desmoglein-2, |
| | | | | | cadherin family |
| | | | | | member 5, HDGC |
| Aggrecan | ACAN | P16112 | aggrecan | AGC1, AGCAN, | aggrecan core |
| | | | | CSPG1, CSPGCP, | protein, cartilage- |
| | | | | MSK16, SEDK, | specific |
| | | | | SSOAOD | proteoglycan core |
| | | | | | protein (CSPCP), |
| | | | | | chondroitin sulfate |
| | | | | | proteoglycan core |
| | | | | | protein 1 |
| | | | | | (chondroitin sulfate |
| | | | | | proteoglycan 1), |
| | | | | | aggrecan core |
| | | | | | protein 2 |
| Syntaxin 4 | STX4 | Q12846 | syntaxin 4 | DFNB123, p35-2, | syntaxin-4, renal |
| | | | | STX4A, | carcinoma antigen |
| | | | | LOC101928762 | NY-REN-31 |
| VAMP-1 | VAMP1 | P23763 | vesicle associated | CMS25, SAX1, | vesicle-associated |
| | | | membrane protein | SPAX1, SYB1, | membrane protein 1, |
| | | | 1 | VAMP-1 | VAMP-1, |
| | | | | | synaptobrevin 1, |
| | | | | | synaptobrevin-1 |
| Nectin-2 | NECTIN2 | Q92692 | nectin cell | CD112, HVEB, | nectin-2, Herpes |
| | | | adhesion molecule | PRR2, PVRL2, | virus entry mediator |
| | | | 2 | PVRR2 | B (Herpesvirus |
| | | | | | entry mediator B, |
| | | | | | HveB), Poliovirus |
| | | | | | receptor-related |
| | | | | | protein 2, CD112 |
| FGF-21 | FGF21 | Q9NSA1 | fibroblast growth | UNQ3115/PRO10 | FGF-21 |
| | | | factor 21 | 196 | |
| Flt-3 | FLT3 | P36888 | fms related | CD135, FLK-2, | receptor-type |
| | | | receptor tyrosine | FLK2, STK1, | tyrosine-protein |
| | | | kinase 3 | FIT3 | kinase FLT3, FL |
| | | | | | cytokine receptor, |
| | | | | | fetal liver kinase-2 |
| | | | | | (FLK-2), fms-like |
| | | | | | tyrosine kinase 3 |
| | | | | | (FLT-3), stem cell |
| | | | | | tyrosine kinase 1 |
| | | | | | (STK-1), CD135, |
| | | | | | EC: 2.7.10.1 |
| GFAP | GFAP | P14136 | glial fibrillary | ALXDRD | GFAP, glial |
| | | | acidic protein | | filament protein (50 |
| | | | | | kDa) |
| TIM-1 | HAVCR1 | Q96D42 | hepatitis A virus | CD365, HAVCR, | HAVcr-1, kidney |
| | | | cellular receptor 1 | HAVCR-1, KIM- | injury molecule 1 |
| | | | | 1, KIM1, HKIM-1, | (KIM-1), kidney |
| | | | | TIM, TIM-1, | injury molecule-1, |
| | | | | TIM1, TIMD-1, | T-cell |
| | | | | TIMD 1 | immunoglobulin and |
| | | | | | mucin domain- |
| | | | | | containing protein 1 |
| | | | | | (TIMD-1), T-cell |
| | | | | | immunoglobulin |
| | | | | | mucin receptor 1 |
| | | | | | (TIM, TIM-1), T- |
| | | | | | cell membrane |
| | | | | | protein 1, CD365 |
| Inhibin A | INHBA | P08476 | inhibin subunit | EDF, FRP | inhibin beta A |
| | | | beta A | | chain, activin beta-A |
| | | | | | chain, activin β A, |
| | | | | | activin A, beta A |
| | | | | | inhibin, inhibin A, |
| | | | | | inhibin β A, inhibin |
| | | | | | subunit β A, inhibin |
| | | | | | beta A, inhibin β a |
| | | | | | subunit, β A inhibin, |
| | | | | | erythroid |
| | | | | | differentiation |
| | | | | | protein (EDF) |
| Cadherin-4 | CDH4 | P55283 | cadherin 4 | CAD4, R-CAD, | cadherin-4, retinal |
| | | | | RCAD, FLJ22202, | cadherin (R-CAD, |
| | | | | LOC101928097 | R-cadherin) |
| P1GF-2 | PGF | P49763 | placental growth | PGFL, PIGF, | PIGF |
| | | | factor | PLGF, PIGF-2, | |
| | | | | D12S1900, | |
| | | | | SHGC-10760 | |
| Neurogranin | NRGN | Q92686 | neurogranin | RC3, hng | Ng, RC3, |
| | | | | | NEUG(55-78) |
| HE4 | WFDC2 | Q14508 | WAP four- | BENP, EDDM4, | WAP four-disulfide |
| | | | disulfide core | HE4, WAP5, | core domain protein |
| | | | domain 2 | dJ461P17.6 | 2, epididymal |
| | | | | | secretory protein E4, |
| | | | | | major epididymis- |
| | | | | | specific protein E4, |
| | | | | | putative protease |
| | | | | | inhibitor WAP5, |
| | | | | | WFDC2 isoform 3 |
| IL-23 R | IL23R | Q5VWK5 | interleukin 23 | PSORS7 | interleukin-23 |
| | | | receptor | | receptor, IL-23 |
| | | | | | receptor, IL-23R |
| Galectin-7 | LGALS7 | P47929 | galectin 7 | GAL7, | galectin-7, Gal-7, |
| | | | | LGALS7A, | HKL-14, PI7, p53- |
| | | | | LGALS7B, PIG1, | induced gene 1 |
| | | | | HKL-14 | protein |
| GALNT3 | GALNT3 | Q14435 | polypeptide N- | GalNAc-T3, | polypeptide GalNAc |
| | | | acetylgalactosami | HFTC, HFTC1, | transferase 3 |
| | | | nyltransferase 3 | HHS, | (GalNac-T3, pp- |
| | | | | LOC102724230 | GaNTase 3), |
| | | | | | protein-UDP |
| | | | | | acetylgalactosaminy |
| | | | | | Itransferase 3, UDP- |
| | | | | | GalNAc: polypeptide |
| | | | | | N- |
| | | | | | acetylgalactosaminy |
| | | | | | Itransferase 3, |
| | | | | | EC: 2.4.1.41 |
| GITR L | TNFSF18 | Q9UNG2 | TNF superfamily | AITRL, GITRL, | tumor necrosis |
| | | | member 18 | TL6, TNLG2A, | factor ligand |
| | | | | hGITRL, | superfamily member |
| | | | | TEASRL, | 18, activation- |
| | | | | UNQ149/PRO175 | inducible TNF- |
| | | | | | related ligand |
| | | | | | (AITRL), |
| | | | | | glucocorticoid- |
| | | | | | induced TNF-related |
| | | | | | ligand (hGITRL) |
| CD14 | CD14 | P08571 | CD14 molecule | | monocyte |
| | | | | | differentiation |
| | | | | | antigen CD14, |
| | | | | | CD14 antigen, |
| | | | | | My23 antigen, |
| | | | | | myeloid cell- |
| | | | | | specific leucine-rich |
| | | | | | glycoprotein, |
| | | | | | monocyte |
| | | | | | differentiation |
| | | | | | antigen CD14 |
| | | | | | urinary form, |
| | | | | | monocyte |
| | | | | | differentiation |
| | | | | | antigen CD14 |
| | | | | | membrane-bound |
| | | | | | form, CD14 |
| R-Spondin 2 | RSPO2 | Q6UXX9 | R-spondin 2 | CRISTIN2, | R-spondin-2, roof |
| | | | | HHRRD, | plate-specific |
| | | | | TETAMS2, | spondin-2 (hRspo2) |
| | | | | UNQ9384/PRO34209 | |
| CK19 | KRT19 | P08727 | keratin 19 | CK19, K19, K1CS | keratin type I |
| | | | | | cytoskeletal 19, |
| | | | | | cytokeratin-19 (CK- |
| | | | | | 19), keratin-19 |
| | | | | | (K19) |
| Cardiotrophin- | CTF1 | Q16619 | cardiotrophin 1 | CT-1, CT1 | cardiotrophin-1, CT- |
| 1 | | | | | 1 |
| TREML1 | TREML1 | Q86YW5 | triggering receptor | GLTL1825, | trem-like transcript |
| | | | expressed on | PRO3438, TLT-1, | 1 protein, TLT-1, |
| | | | myeloid cells like | TLT1, | triggering receptor |
| | | | 1 | dJ238O23.3, | expressed on |
| | | | | UNQ1825/PRO34 | myeloid cells-like |
| | | | | 38 | protein 1 |
| HAPLN1 | HAPLN1 | P10915 | hyaluronan and | CRT1, CRTL1, | cartilage-linking |
| | | | proteoglycan link | Link | protein 1 (cartilage- |
| | | | protein 1 | | link protein), |
| | | | | | proteoglycan link |
| | | | | | protein |
| CD27 | CD27 | P26842 | CD27 molecule | S152, S152. | CD27 antigen, |
| | | | | LPFS2, T14, | CD27L receptor, T- |
| | | | | TNFRSF7, Tp55 | cell activation |
| | | | | | antigen CD27, T14, |
| | | | | | tumor necrosis |
| | | | | | factor receptor |
| | | | | | superfamily member |
| | | | | | 7 (TNFRSF7), |
| | | | | | CD27 |
| ANG-4 | ANGPT4 | Q9Y264 | angiopoietin 4 | ANG3, ANG4 | angiopoietin-4, |
| | | | | | ANG-4, |
| | | | | | angiopoietin-3 |
| | | | | | (ANG-3) |
| Siglec-7 | SIGLEC7 | Q9Y286 | sialic acid binding | AIRM-1, AIRM1, | sialic acid-binding |
| | | | Ig like lectin 7 | CD328, CDw328, | Ig-like lectin 7, |
| | | | | D-siglec, QA79, | siglec-7, adhesion |
| | | | | SIGLEC-7, | inhibitory receptor |
| | | | | SIGLEC19P, | molecule 1 (AIRM- |
| | | | | SIGLECP2, p75, | 1), CDw328, D- |
| | | | | p75/AIRM1 | siglec, QA79 |
| | | | | | membrane protein, |
| | | | | | p75, CD328 |
| CD155 | PVR | P15151 | PVR cell adhesion | CD155, HVED, | Poliovirus receptor, |
| | | | molecule | NECL5, Necl-5, | nectin-like protein 5 |
| | | | | PVS, TAGE4 | (NECL-5), CD155 |
| VEGF-C | VEGFC | P49767 | vascular | Flt4-L, LMPH1D, | VEGF-C, Flt4 |
| | | | endothelial growth | LMPHM4, VRP, | ligand (Flt4-L), |
| | | | factor C | Flt4 ligand | vascular endothelial |
| | | | | | growth factor- |
| | | | | | related protein |
| | | | | | (VRP) |
| TNF RII | TNFRSF | P20333 | TNF receptor | CD120b, TBPII, | tumor necrosis |
| | 1B | | superfamily | TNF-R-II, TNF- | factor receptor |
| | | | member 1B | R75, TNFBR, | superfamily member |
| | | | | TNFR1B, TNFR2, | 1B, tumor necrosis |
| | | | | TNFR80, p75, | factor receptor 2 |
| | | | | p75TNFR | (TNF-R2), tumor |
| | | | | | necrosis factor |
| | | | | | receptor type II |
| | | | | | (TNF-RII, TNFR- |
| | | | | | II), p75, p80 TNF- |
| | | | | | alpha receptor, |
| | | | | | tumor necrosis |
| | | | | | factor receptor |
| | | | | | superfamily member |
| | | | | | 1b membrane form, |
| | | | | | tumor necrosis |
| | | | | | factor-binding |
| | | | | | protein 2 (TBP-2, |
| | | | | | TBPII), CD120b, |
| | | | | | etanercept |
| PGRP-S | PGLYRP | O75594 | peptidoglycan | PGLYRP, PGRP, | peptidoglycan |
| | 1 | | recognition | PGRP-S, PGRPS, | recognition protein |
| | | | protein 1 | TAG7, TNFSF3L, | short (PGRP-S) |
| | | | | SBBI68, | |
| | | | | UNQ639/PRO126 | |
| | | | | 9 | |
| SDF-la | CXCL12 | P48061 | C-X-C motif | IRH, hIRH, PBSF, | stromal cell-derived |
| | | | chemokine ligand | SCYB12, SDF1, | factor 1, SDF-1, |
| | | | 12 | SDF1A, SDF1B, | hSDF-1, C-X-C |
| | | | | TLSF, TPAR1, | motif chemokine 12, |
| | | | | LOC105378278 | intercrine reduced in |
| | | | | | hepatomas (IRH, |
| | | | | | hIRH), pre-B cell |
| | | | | | growth-stimulating |
| | | | | | factor (PBSF), SDF- |
| | | | | | 1-beta(3-72), SDF- |
| | | | | | 1-alpha(3-67), |
| | | | | | CXCL12 isoform 1 |
| | PDGFA | P04085 | platelet derived | PDGF-A, PDGF1 | platelet-derived |
| | | | growth factor | | growth factor |
| | | | subunit A | | subunit A, PDGF |
| | | | | | subunit A, PDGF |
| | | | | | alpha, PDGF α, |
| | | | | | PDGF-1, platelet- |
| | | | | | derived growth |
| | | | | | factor A chain, |
| | | | | | platelet-derived |
| | | | | | growth factor alpha |
| | | | | | polypeptide |
| PDGF-AB | PDGFB | P01127 | platelet derived | IBGC5, PDGF-2, | platelet-derived |
| | | | growth factor | PDGF2, SIS, SSV, | growth factor |
| | | | subunit B | c-sis, PDGF-BB | subunit B, PDGF |
| | | | | | subunit B, PDGF |
| | | | | | beta, PDGFbetaR, |
| | | | | | PDGFRbeta, PDGF |
| | | | | | β, PDGF-2, platelet- |
| | | | | | derived growth |
| | | | | | factor B chain, |
| | | | | | platelet-derived |
| | | | | | growth factor beta |
| | | | | | polypeptide, proto- |
| | | | | | oncogene c-Sis, |
| | | | | | becaplermin |
| GPVI | GP6 | Q9HCN6 | glycoprotein VI | BDPLT11, GPIV, | platelet glycoprotein |
| | | | platelet | GPVI | VI, GPVI, |
| | | | | | glycoprotein 6 |
| CD40 | CD40 | P25942 | CD40 molecule | Bp50, CDW40, | tumor necrosis |
| | | | | TNFRSF5, p50 | factor receptor |
| | | | | | superfamily member |
| | | | | | 5 (TNFRSF5), |
| | | | | | CD40 antigen, B- |
| | | | | | cell surface antigen |
| | | | | | CD40, Bp50, |
| | | | | | CD40L receptor, |
| | | | | | CDw40, CD40 |
| SCF R | KIT | P10721 | KIT proto- | C-Kit, CD117, | mast/stem cell |
| | | | oncogene, | MASTC, PBT, | growth factor |
| | | | receptor tyrosine | SCFR, c-Kit, SCR | receptor Kit, SCFR, |
| | | | kinase | | piebald trait protein |
| | | | | | (PBT), proto- |
| | | | | | oncogene c-Kit, |
| | | | | | tyrosine-protein |
| | | | | | kinase Kit, p145 c- |
| | | | | | kit, v-kit Hardy- |
| | | | | | Zuckerman 4 feline |
| | | | | | sarcoma viral |
| | | | | | oncogene homolog, |
| | | | | | CD117, EC: 2.7.10.1 |
| Thrombospondin- | COMP | P49747 | cartilage | CTS2, EDM1, | thrombospondin-5 |
| 5 | | | oligomeric matrix | EPD1, MED, | (TSP5) |
| | | | protein | PSACH, THBS5, | |
| | | | | TSP-5, TSP5 | |
| IL-1 RII | IL1R2 | P27930 | interleukin 1 | IL1RB, CD121b, | interleukin-1 |
| | | | receptor type 2 | IL1R2c, | receptor type 2, IL- |
| | | | | CDw121b, | 1R-2, IL-1RT-2, IL- |
| | | | | CDW121B, IL- | 1RT2, CD121 |
| | | | | 1R-2, IL-1RT2, | antigen-like family |
| | | | | IL-1RT-2, | member B, |
| | | | | IL1bRb, IL-1R | CDw121b, IL-1 type |
| | | | | type II | II receptor, IL-1R |
| | | | | | type II, interleukin-1 |
| | | | | | receptor beta (IL- |
| | | | | | 1R-beta), |
| | | | | | interleukin-1 |
| | | | | | receptor type II, |
| | | | | | interleukin-1 |
| | | | | | receptor type 2 |
| | | | | | membrane form |
| | | | | | (mIL-1R2, mIL- |
| | | | | | 1RII), interleukin-1 |
| | | | | | receptor type 2 |
| | | | | | soluble form (sIL- |
| | | | | | 1R2, sIL-1RII), |
| | | | | | CD121b |
| Neuropilin-2 | NRP2 | O60462 | neuropilin 2 | NP2, NPN2, | neuropilin-2, |
| | | | | PRO2714, | vascular endothelial |
| | | | | VEGF165R2 | cell growth factor |
| | | | | | 165 receptor 2 |
| | | | | | (VEGF165R2) |
| Cadherin-13 | CDH13 | P55290 | cadherin 13 | CDHH, P105, T- | cadherin-13, heart |
| | | | | CAD | cadherin (H- |
| | | | | | cadherin), P105, |
| | | | | | truncated cadherin |
| | | | | | (T-cad, T-cadherin), |
| E-Selectin | SELE | P16581 | selectin E | CD62E, ELAM, | E-selectin, selectin- |
| | | | | ELAM1, ESEL, | e, selectin E |
| | | | | LECAM2, | precursor, CD62 |
| | | | | selectin-e | antigen-like family |
| | | | | | member E, |
| | | | | | endothelial |
| | | | | | leukocyte adhesion |
| | | | | | molecule 1 (ELAM- |
| | | | | | 1), leukocyte- |
| | | | | | endothelial cell |
| | | | | | adhesion molecule 2 |
| | | | | | (LEAM2), CD62E |
| GITR | TNFRSF | Q9Y5U5 | TNF receptor | AITR, CD357, | tumor necrosis |
| | 18 | | superfamily | ENERGEN, | factor receptor |
| | | | member 18 | GITR, GITR-D, | superfamily member |
| | | | | TR11, | 18, activation- |
| | | | | UNQ319/PRO364 | inducible TNFR |
| | | | | | family receptor, |
| | | | | | glucocorticoid- |
| | | | | | induced TNFR- |
| | | | | | related protein, |
| | | | | | CD357 |
| WISP-1 | CCN4 | O95388 | cellular | WISP1, WISP1- | CCN family |
| | | | communication | OT1, WISP1-UT1, | member 4, WNT1- |
| | | | network factor 4 | WISP1c, WISP1i, | inducible-signaling |
| | | | | WISP1tc | pathway protein 1 |
| | | | | | (WISP-1), Wnt-1- |
| | | | | | induced secreted |
| | | | | | protein |
| Renin | REN | P00797 | renin | ADTKD4, HNFJ2, | pro-renin, renin |
| | | | | RTD | precursor, |
| | | | | | angiotensinogenase, |
| | | | | | EC: 3.4.23.15 |
| AgRP | AGRP | O00253 | agouti related | AGRT, ART, | agouti-related |
| | | | neuropeptide | ASIP2 | protein |
| MDL-1 | CLEC5A | Q9NY25 | C-type lectin | CLECSF5, MDL- | C-type lectin |
| | | | domain containing | 1, MDL1, MDL | domain family 5 |
| | | | 5A | | member A, C-type |
| | | | | | lectin superfamily |
| | | | | | member 5, myeloid |
| | | | | | DAP12-associating |
| | | | | | lectin 1 (MDL-1) |
| ROBO3 | ROBO3 | Q96MS0 | roundabout | HGPPS, HGPPS1, | roundabout homolog |
| | | | guidance receptor | HGPS, RBIG1, | 3, roundabout-like |
| | | | 3 | RIG1 | protein 3 |
| RANTES | CCL5 | P13501 | C-C motif | D17S136E, | C-C motif |
| | | | chemokine ligand | RANTES, | chemokine 5, eoCP, |
| | | | 5 | SCYA5, SIS-delta, | eosinophil |
| | | | | SIS-δ, SISd, | chemotactic |
| | | | | TCP228, eoCP, | cytokine, SIS-delta, |
| | | | | RNTES | small-inducible |
| | | | | | cytokine A5, T-cell- |
| | | | | | specific protein |
| | | | | | P228 (TCP228), T- |
| | | | | | cell-specific protein |
| | | | | | RANTES, |
| | | | | | RANTES(3-68), |
| | | | | | RANTES(4-68) |
| Endocan | ESM1 | Q9NQ30 | endothelial cell | endocan | endothelial cell- |
| | | | specific molecule | | specific molecule 1, |
| | | | 1 | | endothelial cell- |
| | | | | | specific molecule, |
| | | | | | ESM-1 |
| Granulysin | GNLY | P22749 | granulysin | D2S69E, LAG-2, | lymphokine LAG-2, |
| | | | | LAG2, NKG5, | protein NKG5, T- |
| | | | | TLA519 | cell activation |
| | | | | | protein 519 |
| hCGb | CGB3; | P0DN86 | chorionic | CGB, CGB5, | choriogonadotropin |
| | CGB5; | | gonadotropin | CGB7, CGB8, | subunit beta 3, |
| | CGB8 | | subunit beta 3, | LHB, hCGB, HCG | choriogonadotropin |
| | | | chorionic | | subunit beta 5, |
| | | | gonadotropin | | choriogonadotropin |
| | | | subunit beta 5, | | subunit beta 8, |
| | | | chorionic | | choriogonadotropin |
| | | | gonadotropin | | subunit beta (CG- |
| | | | subunit beta 8 | | beta, CG β), |
| | | | | | chorionic |
| | | | | | gonadotropin chain |
| | | | | | beta, chorionic |
| | | | | | gonadotropin |
| | | | | | subunit β 3, |
| | | | | | chorionic |
| | | | | | gonadotropin |
| | | | | | subunit β 5, |
| | | | | | chorionic |
| | | | | | gonadotropin |
| | | | | | subunit β 8, Hcg-β, |
| | | | | | beta HCG5, β |
| | | | | | HCG5 |
| | CGB7 | P0DN87 | chorionic | CG-beta-a, CGB6, | choriogonadotropin |
| | | | gonadotropin | CG-β-a, | subunit beta 7, |
| | | | subunit beta 7 | LOC105376902 | chorionic |
| | | | | | gonadotropin |
| | | | | | subunit β 7 |
| Mesothelin | MSLN | Q13421 | mesothelin | MPF, SMRP | CAK1 antigen, pre- |
| | | | | | pro-megakaryocyte- |
| | | | | | potentiating factor, |
| | | | | | megakaryocyte- |
| | | | | | potentiating factor |
| | | | | | (MPF), mesothelin |
| | | | | | cleaved form |
| TLR1 | TLR1 | Q15399 | toll like receptor 1 | CD281, TIL, TIL. | toll-like receptor 1, |
| | | | | LPRS5, rsc786, | toll/interleukin-1 |
| | | | | KIAA0012 | receptor-like protein |
| | | | | | (TIL), CD281 |
| TRAIL | TNFSF10 | P50591 | TNF superfamily | APO2L, Apo-2L, | tumor necrosis |
| | | | member 10 | CD253, TANCR, | factor ligand |
| | | | | TL2, TNLG6A, | superfamily member |
| | | | | TRAIL | 10, Apo-2 ligand |
| | | | | | (Apo-2L), TNF- |
| | | | | | related apoptosis- |
| | | | | | inducing ligand |
| | | | | | (protein TRAIL), |
| | | | | | CD253 |
| MOG | MOG | Q16653 | myelin | BTN6, BTNL11, | myelin- |
| | | | oligodendrocyte | NRCLP7, | oligodendrocyte |
| | | | glycoprotein | MOGIG2 | glycoprotein, |
| | | | | | oligodendrocyte |
| | | | | | myelin glycoprotein |
| DDR1 | DDR1 | Q08345 | discoidin domain | CAK, CD167, | epithelial discoidin |
| | | | receptor tyrosine | DDR, EDDR1, | domain-containing |
| | | | kinase 1 | HGK2, MCK10, | receptor 1, epithelial |
| | | | | NEP, NTRK4, | discoidin domain |
| | | | | PTK3, PTK3A, | receptor 1, CD167 |
| | | | | RTK6, TRKE | antigen-like family |
| | | | | | member A, CD167a |
| | | | | | antigen, cell |
| | | | | | adhesion kinase, |
| | | | | | discoidin receptor |
| | | | | | tyrosine kinase, |
| | | | | | tyrosine kinase |
| | | | | | DDR, HGK2, |
| | | | | | mammary |
| | | | | | carcinoma kinase 10 |
| | | | | | (MCK-10), protein- |
| | | | | | tyrosine kinase 3A, |
| | | | | | protein-tyrosine |
| | | | | | kinase RTK-6, TRK |
| | | | | | E, tyrosine-protein |
| | | | | | kinase CAK, |
| | | | | | CD167a |
| NGF R | NGFR | P08138 | nerve growth | CD271, Gp80- | tumor necrosis |
| | | | factor receptor | LNGFR, | factor receptor |
| | | | | TNFRSF16, | superfamily member |
| | | | | p75(NTR), | 16, Gp80-LNGFR, |
| | | | | p75NTR, | low affinity |
| | | | | p75LNGFR | neurotrophin |
| | | | | | receptor p75NTR, |
| | | | | | low-affinity nerve |
| | | | | | growth factor |
| | | | | | receptor (NGF |
| | | | | | receptor), low- |
| | | | | | affinity nerve |
| | | | | | growth factor |
| | | | | | receptor p75NGFR, |
| | | | | | low-affinity nerve |
| | | | | | growth factor |
| | | | | | receptor p75NGR, |
| | | | | | p75 ICD, CD271, |
| | | | | | p75 neurotrophin |
| | | | | | receptor |
| TRAIL R3 | TNFRSF | O14798 | TNF receptor | CD263, DCR1, | tumor necrosis |
| | 10C | | superfamily | DCR1-TNFR, | factor receptor |
| | | | member 10c | LIT, TRAIL-R3, | superfamily member |
| | | | | TRAILR3, TRID, | 10C, antagonist |
| | | | | UNQ321/PRO366 | decoy receptor for |
| | | | | | TRAIL/Apo-2L, |
| | | | | | decoy TRAIL |
| | | | | | receptor without |
| | | | | | death domain, decoy |
| | | | | | receptor 1 (DcR1), |
| | | | | | lymphocyte |
| | | | | | inhibitor of TRAIL, |
| | | | | | TNF-related |
| | | | | | apoptosis-inducing |
| | | | | | ligand receptor 3 |
| | | | | | (TRAIL receptor 3, |
| | | | | | TRAIL-R3), TRAIL |
| | | | | | receptor without an |
| | | | | | intracellular domain, |
| | | | | | CD263 |
| Trypsin 3 | PRSS3 | P35030 | serine protease 3 | MTG, PRSS4, T9, | trypsin-3, brain |
| | | | | TRY3, TRY4 | trypsinogen, |
| | | | | | mesotrypsin, |
| | | | | | mesotrypsinogen, |
| | | | | | serine protease 3, |
| | | | | | serine protease 4, |
| | | | | | trypsin III, trypsin |
| | | | | | IV |
| ARSB | ARSB | P15848 | arylsulfatase B | ASB, G4S, MPS6 | 4-sulfatase, 4- |
| | | | | | sulphatase, ASB, N- |
| | | | | | acetylgalactosamine- |
| | | | | | 4-sulfatase (G4S), |
| | | | | | EC: 3.1.6.12 |
| LIF R alpha | LIFR | P42702 | LIF receptor | CD118, LIF-R, | leukemia inhibitory |
| | | | subunit alpha | SJS2, STWS, | factor receptor, LIF |
| | | | | SWS, | receptor, LIF-R, |
| | | | | LOC105374734 | CD118, LIFR beta, |
| | | | | | LIF receptor subunit |
| | | | | | alpha, LIF receptor |
| | | | | | subunit α, Lifr α, |
| | | | | | LIFR β |
| BAFF R | TNFRSF | Q96RJ3 | TNF receptor | BAFF-R, BAFFR, | tumor necrosis |
| | 13C | | superfamily | BR3, BROMIX, | factor receptor |
| | | | member 13C | CD268, CVID4, | superfamily member |
| | | | | prolixin | 13C, B-cell- |
| | | | | | activating factor |
| | | | | | receptor, BAFF |
| | | | | | receptor (BAFF-R), |
| | | | | | BLyS receptor 3, |
| | | | | | CD268 |
| CD157 | BST1 | Q10588 | bone marrow | CD157, cADPR2 | ADP-ribosyl |
| | | | stromal cell | | cyclase/cyclic ADP- |
| | | | antigen 1 | | ribose hydrolase 2, |
| | | | | | ADP-ribosyl cyclase |
| | | | | | 2, bone marrow |
| | | | | | stromal cell antigen |
| | | | | | 1 (BST-1), cyclic |
| | | | | | ADP-ribose |
| | | | | | hydrolase 2 (cADPR |
| | | | | | hydrolase 2), |
| | | | | | CD157, EC: 3.2.2.6 |
| Granzyme A | GZMA | P12544 | granzyme A | CTLA3, HFSP | CTL tryptase, |
| | | | | | cytotoxic T- |
| | | | | | lymphocyte |
| | | | | | proteinase 1, |
| | | | | | fragmentin-1, |
| | | | | | granzyme-1, |
| | | | | | Hanukkah factor (H |
| | | | | | factor, HF), |
| | | | | | EC: 3.4.21.78 |
| 2B4 | CD244 | Q9BZW8 | CD244 molecule | 2B4, NAIL, | natural killer cell |
| | | | | NKR2B4, Nmrk, | receptor 2B4, |
| | | | | SLAMF4, h2B4 | CD244 antigen, NK |
| | | | | | cell activation- |
| | | | | | inducing ligand |
| | | | | | (NAIL), NK cell |
| | | | | | type I receptor |
| | | | | | protein 2B4 |
| | | | | | (NKR2B4, h2B4), |
| | | | | | SLAM family |
| | | | | | member 4 |
| | | | | | (SLAMF4), |
| | | | | | signaling |
| | | | | | lymphocytic |
| | | | | | activation molecule |
| | | | | | 4, CD244 |
| ESAM | ESAM | Q96AP7 | endothelial cell | NEDIHSS, | endothelial cell- |
| | | | adhesion molecule | W117m, | selective adhesion |
| | | | | UNQ220/PRO246 | molecule |
| IL-1 R4 | IL1RL1 | Q01638 | interleukin 1 | DER4, FIT-1, | interleukin-1 |
| | | | receptor like 1 | IL33R, ST2, | receptor-like 1, |
| | | | | ST2L, ST2V, T1 | protein ST2, |
| | | | | | EC: 3.2.2.6 |
| CXCL14 | CXCL14 | O95715 | C-X-C motif | BMAC, BRAK, | C-X-C motif |
| | | | chemokine ligand | KEC, KS1, MIP- | chemokine 14, |
| | | | 14 | 2g, MIP2G, Mip2 | chemokine BRAK, |
| | | | | γ, NJAC, | MIP-2G, small- |
| | | | | SCYB14, | inducible cytokine |
| | | | | PSEC0212, | B14 |
| | | | | UNQ240/PRO273 | |
| IL-31 | IL31 | Q6EBC2 | interleukin 31 | IL-31 | interleukin-31, IL- |
| | | | | | 31 |
| SIRP alpha | SIRPA | P78324 | signal regulatory | BIT, CD172A, | tyrosine-protein |
| | | | protein alpha | MFR, MYD-1, | phosphatase non- |
| | | | | P84, PTPNS1, | receptor type |
| | | | | SHPS-1, SHPS1, | substrate 1, SIRPA |
| | | | | SIRP, MYD1, | isoform 2, signal |
| | | | | SIRPα, Sirp-α-1 | regulatory protein α, |
| | | | | | SHP substrate 1, |
| | | | | | SHPS-1, brain Ig- |
| | | | | | like molecule with |
| | | | | | tyrosine-based |
| | | | | | activation motifs |
| | | | | | (Bit), CD172 |
| | | | | | antigen-like family |
| | | | | | member A, |
| | | | | | inhibitory receptor |
| | | | | | SHPS-1, |
| | | | | | macrophage fusion |
| | | | | | receptor, MyD-1 |
| | | | | | antigen, signal- |
| | | | | | regulatory protein |
| | | | | | alpha-1 (Sirp-alpha- |
| | | | | | 1), signal-regulatory |
| | | | | | protein alpha-2 |
| | | | | | (Sirp-alpha-2), |
| | | | | | signal-regulatory |
| | | | | | protein alpha-3 |
| | | | | | (Sirp-alpha-3), p84, |
| | | | | | CD172a |
| Uromodulin | UMOD | P07911 | uromodulin | ADMCKD2, | Tamm-Horsfall |
| | | | | ADTKD1, FJHN, | urinary glycoprotein |
| | | | | HNFJ, HNFJ1, | (THP), uromodulin |
| | | | | MCKD2, THGP, | secreted form |
| | | | | THP | |
| CTRC | CTRC | Q99895 | chymotrypsin C | CLCR, ELA4 | chymotrypsin-C, |
| | | | | | caldecrin, |
| | | | | | EC: 3.4.21.2 |
| CEACAM-1 | CEACA | P13688 | CEA cell adhesion | BGP, BGP1, BGPI | carcinoembryonic |
| | M1 | | molecule 1 | | antien-related cell |
| | | | | | adhesion molecule |
| | | | | | 1, biliary |
| | | | | | glycoprotein 1 |
| | | | | | (BGP-1), CD66a |
| TARC | CCL17 | Q92583 | C-C motif | A-152E5.3, | C-C motif |
| | | | chemokine ligand | ABCD-2, | chemokine 17, CC |
| | | | 17 | SCYA17, TARC | chemokine TARC, |
| | | | | | small-inducible |
| | | | | | cytokine A17, |
| | | | | | thymus and |
| | | | | | activation-regulated |
| | | | | | chemokine |
| MIP-3a | CCL20 | P78556 | C-C motif | CKb4, Exodus, | C-C motif |
| | | | chemokine ligand | LARC, MIP-3- | chemokine 20, beta- |
| | | | 20 | alpha, MIP-3-α, | chemokine exodus- |
| | | | | MIP-3a, Mip3 α, | 1, CC chemokine |
| | | | | MIP3A, SCYA20, | LARC, liver and |
| | | | | ST38 | activation-regulated |
| | | | | | chemokine, |
| | | | | | macrophage |
| | | | | | inflammatory |
| | | | | | protein 3 alpha |
| | | | | | (MIP-3-alpha), |
| | | | | | small-inducible |
| | | | | | cytokine A20, |
| | | | | | CCL20(1-67), |
| | | | | | CCL20(1-64), |
| | | | | | CCL20(2-70), |
| | | | | | chemokine exudus 1 |
| SDF-lb | CXCL12 | P48061 | C-X-C motif | IRH, hIRH, PBSF, | stromal cell-derived |
| | | | chemokine ligand | SCYB12, SDF1, | factor 1, SDF-1, |
| | | | 12 | SDF1A, SDF1B, | hSDF-1, C-X-C |
| | | | | TLSF, TPAR1, | motif chemokine 12, |
| | | | | LOC105378278 | CXCL12 isoform 1, |
| | | | | | intercrine reduced in |
| | | | | | hepatomas (IRH, |
| | | | | | hIRH), pre-B-cell |
| | | | | | growth-stimulating |
| | | | | | factor (PBSF), SDF- |
| | | | | | 1-beta(3-72), SDF- |
| | | | | | 1 -alpha(3-67) |
| NKp46 | NCR1 | O76036 | natural | CD335, LY94, | lymphocyte antigen |
| | | | cytotoxicity | NK-p46, NKP46 | 94 homolog, NK |
| | | | triggering receptor | | cell-activating |
| | | | 1 | | receptor, natural |
| | | | | | killer cell p46- |
| | | | | | related protein (NK- |
| | | | | | p46, NKp46, |
| | | | | | hNKp46), CD335 |
| MCP-3 | CCL7 | P80098 | C-C motif | FIC, MARC, | C-C motif |
| | | | chemokine ligand | MCP-3, MCP3, | chemokine 7, |
| | | | 7 | NC28, SCYA6, | monocyte |
| | | | | SCYA7 | chemoattractant |
| | | | | | protein 3, monocyte |
| | | | | | chemotactic protein |
| | | | | | 3 (MCP-3), NC28, |
| | | | | | small-inducible |
| | | | | | cytokine A7 |
| IL-32 alpha | IL32 | P24001 | interleukin 32 | IL-32alpha, IL- | interleukin-32, IL- |
| | | | | 32beta, IL-32delta, | 32, natural killer |
| | | | | IL-32gamma, | cells protein 4, NK |
| | | | | NK4, TAIF, | cell transcript 4, |
| | | | | TAIFa, TAIFb, | tumor necrosis |
| | | | | TAIFc, TAIFd | factor alpha- |
| | | | | | inducing factor |
| TGFb3 | TGFB3 | P10600 | transforming | ARVD, ARVD1, | transforming growth |
| | | | growth factor beta | LDS5, RNHF, | factor beta-3 |
| | | | 3 | TGF-beta3, PTGF | proprotein, |
| | | | | β | transforming growth |
| | | | | | factor β 3, latency- |
| | | | | | associated peptide |
| | | | | | (LAP), transforming |
| | | | | | growth factor beta-3 |
| | | | | | (TGF-beta-3) |
| FOLR2 | FOLR2 | P14207 | folate receptor | BETA-HFR, FBP, | folate receptor β, |
| | | | beta | FBP/PL-1, | FR-beta, folate |
| | | | | FOLR1, FR- | receptor 2, folate |
| | | | | BETA, FR-P3, | receptor |
| | | | | FRbeta, Fr-β, β- | fetal/placental, |
| | | | | HFR | placental folate- |
| | | | | | binding protein |
| | | | | | (FBP), folate |
| | | | | | receptor foetal |
| CD58 | CD58 | P19256 | CD58 molecule | LFA-3, LFA3, ag3 | lymphocyte |
| | | | | | function-associated |
| | | | | | antigen 3, Ag3, |
| | | | | | surface glycoprotein |
| | | | | | LFA-3, CD58 |
| IL-23 | IL23A | Q9NPF7 | interleukin 23 | IL-23, IL-23A, | interleukin-23 |
| | | | subunit alpha | IL23P19, P19, | subunit alpha, IL-23 |
| | | | | SGRF, | subunit alpha, Il-23- |
| | | | | UNQ2498/PRO57 | A, interleukin-23 |
| | | | | 98 | subunit p19 (IL- |
| | | | | | 23p19), interleukin |
| | | | | | 23 subunit α, |
| | | | | | interleukin 23 p19 |
| | | | | | subunit |
| | IL12B | P29460 | interleukin 12B | CLMF, CLMF2, | interleukin-12 |
| | | | | IL-12, IL-12 p40, | subunit beta, IL- |
| | | | | p40, IL-12B, | 12B, cytotoxic |
| | | | | IMD28, IMD29, | lymphocyte |
| | | | | NKSF, NKSF2 | maturation factor 40 |
| | | | | | kDa subunit (CLMF |
| | | | | | p40), IL-12 subunit |
| | | | | | p40, NK cell |
| | | | | | stimulatory factor |
| | | | | | chain 2 (NKSF2) |
| CD36 | CD36 | P16671 | CD36 molecule | BDPLT10, | platelet glycoprotein |
| | | | (CD36 blood | CHDS7, FAT, | 4, fatty acid |
| | | | group) | GP3B, GP4, | translocase (FAT), |
| | | | | GPIV, PASIV, | glycoprotein IIIb |
| | | | | SCARB3 | (GPIIIB), leukocyte |
| | | | | | differentiation |
| | | | | | antigen CD36, PAS |
| | | | | | IV, PAS-4, platelet |
| | | | | | collagen receptor, |
| | | | | | platelet glycoprotein |
| | | | | | IV (GPIV), |
| | | | | | thrombospondin |
| | | | | | receptor |
| TNFb | LTA | P01374 | lymphotoxin alpha | LT, TNFB, | lymphotoxin-alpha, |
| | | | | TNFSF1, | lymphotoxin α, LT- |
| | | | | TNLG1E, Lt-α, | alpha, TNF-beta, |
| | | | | TNF-beta, Tnf β, | tumor necrosis |
| | | | | TNF-β | factor ligand |
| | | | | | superfamily member |
| | | | | | 1 (TNFSF1) |
| Shh-N | SHH | Q15465 | sonic hedgehog | HHG1, HLP3, | sonic hedgehog |
| | | | signaling | HPE3, | protein, HHG-1, |
| | | | molecule | MCOPCB5, | Shh unprocessed N- |
| | | | | SMMCI, ShhNC, | terminal signaling |
| | | | | TPT, TPTPS, | and C-terminal |
| | | | | LOC105375595 | autoprocessing |
| | | | | | domains (ShhNC), |
| | | | | | sonic hedgehog |
| | | | | | protein N-product |
| | | | | | (ShhN), Shh N- |
| | | | | | terminal processed |
| | | | | | signaling domains |
| | | | | | (ShhNp), EC: 3.1.-.- |
| Ficolin-1 | FCN1 | O00602 | ficolin 1 | FCNM, M- | ficolin-1, |
| | | | | FICOLIN | collagen/fibrinogen |
| | | | | | domain-containing |
| | | | | | protein 1, ficolin-A, |
| | | | | | ficolin-alpha, M- |
| | | | | | ficolin |
| Reg4 | REG4 | Q9BYZ8 | regenerating | GISP, REG-IV, | regenerating islet- |
| | | | family member 4 | RELP, FLJ32545 | derived protein 4, |
| | | | | | REG-4, |
| | | | | | gastrointestinal |
| | | | | | secretory protein, |
| | | | | | REG-like protein, |
| | | | | | regenerating islet- |
| | | | | | derived protein IV |
| | | | | | (Reg IV) |
| ILT2 | LILRB1 | Q8NHL6 | leukocyte | CD85J, ILT-2, | leukocyte |
| | | | immunoglobulin | ILT2, LIR-1, | immunoglobulin- |
| | | | like receptor B1 | LIR1, MIR-7, | like receptor |
| | | | | MIR7, PIR-B, | subfamily B |
| | | | | PIRB, CL7 | member 1, LIR-1, |
| | | | | | leukocyte |
| | | | | | immunoglobulin- |
| | | | | | like receptor 1, |
| | | | | | CD85 antigen-like |
| | | | | | family member J, |
| | | | | | immunoglobulin- |
| | | | | | like transcript 2 |
| | | | | | (ILT-2), |
| | | | | | monocyte/macropha |
| | | | | | ge immunoglobulin- |
| | | | | | like receptor 7 |
| | | | | | (MIR-7), CD85j |
| Mer | MERTK | Q12866 | MER proto- | MER, RP38, | tyrosine-protein |
| | | | oncogene, | Tyro12, c-Eyk, c- | kinase Mer, proto- |
| | | | tyrosine kinase | mer, Nyk | oncogene c-Mer, |
| | | | | | receptor tyrosine |
| | | | | | kinase MerTK, |
| | | | | | EC: 2.7.10.1 |
| TREM-2 | TREM2 | Q9NZC2 | triggering receptor | AD17, PLOSL2, | TREM-2, triggering |
| | | | expressed on | TREM-2, Trem2a, | receptor expressed |
| | | | myeloid cells 2 | Trem2b, Trem2c, | on monocytes 2 |
| | | | | DSP-7 | |
| Flt-3L | FLT3LG | P49771 | fms related | FL, FLG3L, | fms-related tyrosine |
| | | | receptor tyrosine | FLT3L, IMD125 | kinase 3 ligand, Flt3 |
| | | | kinase 3 ligand | | ligand, FLT3 ligand, |
| | | | | | Flt3L, SL cytokine |
| IL-6 | IL6 | P05231 | interleukin 6 | BSF-2, BSF2, | interleukin-6, IL-6, |
| | | | | CDF, HGF, HSF, | B-cell stimulatory |
| | | | | IFN-beta-2, | factor 2 (BSF-2), |
| | | | | IFNB2, IL-6, | CTL differentiation |
| | | | | FDGI, IFN-β-2, | factor (CDF), |
| | | | | IFN β 2A | hybridoma growth |
| | | | | | factor, interferon |
| | | | | | beta-2 (IFN-beta-2) |
| CD229 | LY9 | Q9HBG7 | lymphocyte | CD229, SLAMF3, | T-lymphocyte |
| | | | antigen 9 | hly9, mLY9, | surface antigen Ly- |
| | | | | CDABP0070 | 9, cell surface |
| | | | | | molecule Ly-9, |
| | | | | | lymphocyte antigen |
| | | | | | 9, SLAM family |
| | | | | | member 3 |
| | | | | | (SLAMF3), |
| | | | | | signaling |
| | | | | | lymphocytic |
| | | | | | activation molecule |
| | | | | | 3, CD229 |
| Insulin | INS | P01308 | insulin | IDDM, IDDM1, | insulin B chain, |
| | | | | IDDM2, ILPR, | insulin A chain, |
| | | | | IRDN, MODY10, | humuline, iletin, |
| | | | | PNDM4, EINECS | novolin, oral insulin, |
| | | | | 234-279-7, | preproinsulin, |
| | | | | ORMD-0801 | regular insulin, |
| | | | | | soluble insulin, |
| | | | | | technosphere |
| | | | | | insulin, ultraphane |
| Syntaxin 6 | STX6 | O43752 | syntaxin 6 | LOC102724791 | syntaxin-6 |
| GRO | CXCL1 | P09341 | C-X-C motif | FSP, GRO, GRO1, | growth-regulated |
| | | | chemokine ligand | GROa, GROA, | alpha protein, C-X- |
| | | | 1 | MGSA, MGSA-a, | C motif chemokine |
| | | | | NAP-3, SCYB1, | 1, GRO-alpha(1-73), |
| | | | | KC | melanoma growth |
| | | | | | stimulatory activity |
| | | | | | (MGSA), |
| | | | | | neutrophil- |
| | | | | | activating protein 3 |
| | | | | | (NAP-3), GRO- |
| | | | | | alpha(4-73), GRO- |
| | | | | | alpha(5-73), GRO- |
| | | | | | alpha(6-73) |
| Bcl-w | BCL2L2 | Q92843 | BCL2 like 2 | BCL-2, BCL2-L- | Bcl-2-like protein 2, |
| | | | | 2, BCLW, BCL- | Bcl2-L-2, apoptosis |
| | | | | W, PPP1R51, | regulator Bcl-W |
| | | | | KIAA0271 | |
| Lipocalin-2 | LCN2 | P80188 | lipocalin 2 | 24p3, MSFI, | neutrophil |
| | | | | NGAL, p25, HNL | gelatinase- |
| | | | | | associated lipocalin, |
| | | | | | NGAL, 25 kDa |
| | | | | | alpha-2- |
| | | | | | microglobulin- |
| | | | | | related subunit of |
| | | | | | MMP-9, lipocalin-2, |
| | | | | | oncogene 24p3, |
| | | | | | siderocalin, p25, |
| | | | | | neutrophil lipocalin |
| PDGF-AA | PDGF-AA is a dimeric isoform | | | | |
| | consisting of two PDGFA | | | | |
| | subunits. PDGFA is on line | | | | |
| | 665 of this document. | | | | |
| IL-2 Ra | IL2RA | P01589 | interleukin 2 | CD25, IDDM10, | interleukin-2 |
| | | | receptor subunit | IL2R, IMD41, | receptor subunit |
| | | | alpha | TCGFR, p55, Tac, | alpha, IL-2 receptor |
| | | | | I12r α | subunit alpha, IL-2- |
| | | | | | RA, IL-2R subunit |
| | | | | | alpha, IL2-RA, TAC |
| | | | | | antigen, p55, CD25, |
| | | | | | IL 2 receptor α |
| | | | | | subunit, interleukin |
| | | | | | 2 receptor subunit α, |
| | | | | | interleukin 2 |
| | | | | | receptor α chain |
| Angiogenin | ANG | P03950 | angiogenin | ALS9, HEL168, | angiogenin 2, |
| | | | | RAA1, RNASE4, | ribonuclease 5 |
| | | | | RNASE5 | (RNase 5), |
| | | | | | EC: 3.1.27.- |
| LYVE-1 | LYVE1 | Q9Y5Y7 | lymphatic vessel | CRSBP-1, HAR, | lymphatic vessel |
| | | | endothelial | LYVE-1, XLKD1, | endothelial |
| | | | hyaluranon | CRSBP1, | hyaluronic acid |
| | | | receptor 1 | UNQ230/PRO263 | receptor 1, LYVE-1, |
| | | | | | cell surface |
| | | | | | retention sequence- |
| | | | | | binding protein 1 |
| | | | | | (CRSBP-1), |
| | | | | | extracellular link |
| | | | | | domain-containing |
| | | | | | protein 1, hyaluronic |
| | | | | | acid receptor |
| CD4 | CD4 | P01730 | CD4 molecule | CD4mut, IMD79, | CD4 antigen, CD4 |
| | | | | Leu-3, OKT4D, | receptor, T-cell |
| | | | | T4, CD4v4 | surface glycoprotein |
| | | | | | CD4, T-cell surface |
| | | | | | antigen T4/Leu-3, |
| | | | | | CD4 |
| RAGE | AGER | Q15109 | advanced | RAGE, SCARJ1, | advanced |
| | | | glycosylation end- | sRAGE | glycosylation end |
| | | | product specific | | product-specific |
| | | | receptor | | receptor, receptor |
| | | | | | for advanced |
| | | | | | glycosylation end |
| | | | | | products |
| CDNF | CDNF | Q49AH0 | cerebral dopamine | ARMETL1 | ARMET-like |
| | | | neurotrophic | | protein 1, conserved |
| | | | factor | | dopamine |
| | | | | | neurotrophic factor |
| Brevican | BCAN | Q96GW7 | brevican | BEHAB, CSPG7, | brevican core |
| | | | | UNQ2525/PRO60 | protein, BCAN |
| | | | | 18 | isoform 1, brain- |
| | | | | | enriched |
| | | | | | hyaluronan-binding |
| | | | | | protein (BEHAB), |
| | | | | | chondroitin sulfate |
| | | | | | proteoglycan 7 |
| NAP-2 | PPBP | P02775 | pro-platelet basic | B-TG1, Beta-TG, | platelet basic |
| | | | protein | CTAP-III, CTAP3, | protein, PBP, C-X-C |
| | | | | CTAPIII, CXCL7, | motif chemokine 7, |
| | | | | LA-PF4, LDGF, | leukocyte-derived |
| | | | | MDGF, NAP-2, | growth factor |
| | | | | PBP, SCYB7, | (LDGF), |
| | | | | TC1, TC2, TGB, | macrophage-derived |
| | | | | TGB1, THBGB, | growth factor |
| | | | | THBGB1, β-TG | (MDGF), small- |
| | | | | | inducible cytokine |
| | | | | | B7, neutrophil- |
| | | | | | activating peptide 2, |
| | | | | | pro-platelet basic, |
| | | | | | connective tissue- |
| | | | | | activating peptide |
| | | | | | III (CTAP-III), low- |
| | | | | | affinity platelet |
| | | | | | factor IV (LA-PF4), |
| | | | | | TC-2, connective |
| | | | | | tissue activating |
| | | | | | peptide III(1-81) |
| | | | | | (CTAP-III(1-81)), |
| | | | | | beta- |
| | | | | | thromboglobulin |
| | | | | | (Beta-TG), |
| | | | | | neutrophil- |
| | | | | | activating peptide |
| | | | | | 2(74) (NAP-2(74)), |
| | | | | | neutrophil- |
| | | | | | activating peptide |
| | | | | | 2(73) (NAP-2(73)), |
| | | | | | neutrophil- |
| | | | | | activating peptide 2 |
| | | | | | (NAP-2), TC-1, |
| | | | | | neutrophil- |
| | | | | | activating peptide |
| | | | | | 2(1-66) (NAP-2(1- |
| | | | | | 66)), neutrophil- |
| | | | | | activating peptide |
| | | | | | 2(1-63) (NAP-2(1- |
| | | | | | 63)) |
| PU.1 | SPI1 | P17947 | Spi-1 proto- | AGM10, OF, | transcription factor |
| | | | oncogene | PU.1, SFPI1, SPI- | PU.1, 31 kDa- |
| | | | | 1, SPI-A | transforming protein |
| EDAR | EDAR | Q9UNE0 | ectodysplasin A | DL, ECTD10A, | tumor necrosis |
| | | | receptor | ECTD10B, ED1R, | factor receptor |
| | | | | ED3, ED5, EDA- | superfamily member |
| | | | | A1R, EDA1R, | EDAR, anhidrotic |
| | | | | EDA3, HRM1 | ectodysplasin |
| | | | | | receptor 1, downless |
| | | | | | homolog, EDA-A1 |
| | | | | | receptor, ectodermal |
| | | | | | dysplasia receptor, |
| | | | | | ectodysplasin-A |
| | | | | | receptor |
| ADAMTS13 | ADAMTS13 | Q76LX8 | ADAM | ADAM-TS13, | a disintegrin and |
| | | | metallopeptidase | ADAMTS-13, | metalloproteinase |
| | | | with | C9orf8, VWFCP, | with |
| | | | thrombospondin | vWF-CP, | thrombospondin |
| | | | type 1 motif 13 | UNQ6102/PRO20 | motifs 13, ADAM- |
| | | | | 085 | TS 13, ADAM- |
| | | | | | TS13, ADAMTS- |
| | | | | | 13, von Willebrand |
| | | | | | factor-cleaving |
| | | | | | protease (vWF-CP, |
| | | | | | vWF-cleaving |
| | | | | | protease), |
| | | | | | EC: 3.4.24.87 |
| Kynureninase | KYNU | Q16719 | kynureninase | KYNUU, VCRL2 | L-kynurenine |
| | | | | | hydrolase, |
| | | | | | EC: 3.7.1.3 |
| PTH1R | PTH1R | Q03431 | parathyroid | EKNS, PFE, | parathyroid |
| | | | hormone 1 | PTHR, PTHR1, | hormone/parathyroi |
| | | | receptor | HKRK | d hormone-related |
| | | | | | peptide receptor, |
| | | | | | PTH/PTHrP type I |
| | | | | | receptor (PTH/PTHr |
| | | | | | receptor), |
| | | | | | parathyroid |
| | | | | | hormone 1 receptor |
| | | | | | (PTH1 receptor), |
| | | | | | PLP/PTH receptor |
| IFN-gamma | IFNGR1 | P15260 | interferon gamma | CD119, IFNGR, | IFN-gamma |
| R1 | | | receptor 1 | IMD27A, | receptor 1, IFN- |
| | | | | IMD27B, IFN-γR1 | gamma-R1, |
| | | | | | interferon gamma |
| | | | | | receptor alpha chain, |
| | | | | | interferon γ receptor |
| | | | | | 1, interferon γ |
| | | | | | receptor α chain, |
| | | | | | CDw119, interferon |
| | | | | | gamma receptor |
| | | | | | alpha-chain (IFN- |
| | | | | | gamma-R-alpha), |
| | | | | | CD119 |
| CrkL | CRKL | P46109 | CRK like proto- | | Crk-like protein |
| | | | oncogene, adaptor | | |
| | | | protein | | |
| B7-1 | CD80 | P33681 | CD80 molecule | B7, B7-1, B7.1, | T-lymphocyte |
| | | | | BB1, CD28LG, | activation antigen |
| | | | | CD28LG1, LAB7 | CD80, activation |
| | | | | | B7-1 antigen, BB1, |
| | | | | | CTLA-4 counter- |
| | | | | | receptor B7.1 (B7), |
| | | | | | CD80 |
| PARC | CCL18 | P55774 | C-C motif | AMAC-1, | C-C motif |
| | | | chemokine ligand | AMAC1, CKb7, | chemokine 18, CC |
| | | | 18 | DC-CK1, DCCK1, | chemokine PARC, |
| | | | | MIP-4, MIP4, | alternative |
| | | | | PARC, SCYA18 | macrophage |
| | | | | | activation- |
| | | | | | associated CC |
| | | | | | chemokine 1 |
| | | | | | (AMAC-1), |
| | | | | | dendritic cell |
| | | | | | chemokine 1 (DC- |
| | | | | | CK1), macrophage |
| | | | | | inflammatory |
| | | | | | protein 4 (MIP-4), |
| | | | | | pulmonary and |
| | | | | | activation-regulated |
| | | | | | chemokine, small- |
| | | | | | inducible cytokine |
| | | | | | A18, CCL18(1-68), |
| | | | | | CCL18(3-69), |
| | | | | | CCL18(4-69) |
| Draxin | DRAXIN | Q8NBI3 | dorsal inhibitory | UNQ3119, | draxin, dorsal |
| | | | axon guidance | neucrin, | repulsive axon |
| | | | protein | AGPA3119, | guidance protein, |
| | | | | C1orf187, | neucrin |
| | | | | PSEC0258, | |
| | | | | UNQ3119/PRO10 | |
| | | | | 268 | |
| VE- | CDH5 | P33151 | cadherin 5 | 7B4, CD144 | cadherin-5, 7B4 |
| Cadherin | | | | | antigen, vascular |
| | | | | | endothelial cadherin |
| | | | | | (VE-cadherin), |
| | | | | | CD144 |
| Procalcitonin | CALCA | P06881 | calcitonin related | CALC1, CGRP, | calcitonin gene- |
| | | | polypeptide alpha | CGRP-I, CGRP- | related peptide 1, |
| | | | | alpha, CGRP1, | calcitonin related |
| | | | | CT, KC, PCT | polypeptide α, |
| | | | | | procalcitonin, alpha- |
| | | | | | type CGRP, |
| | | | | | calcitonin gene- |
| | | | | | related peptide I |
| | | | | | (CGRP-I) |
| SOX15 | SOX15 | O60248 | SRY-box | SOX20, SOX26, | transcription factor |
| | | | transcription | SOX27, SOX12 | SOX-15, protein |
| | | | factor 15 | | SOX-12, protein |
| | | | | | SOX-20 |
| Kallikrein | KLK11 | Q9UBX7 | kallikrein related | IEKD, PRSS20, | kallikrein-11, |
| 11 | | | peptidase 11 | TLSP, | kallikrein 11, hK11, |
| | | | | UNQ649/PRO127 | hippostasin, serine |
| | | | | 9 | protease 20, trypsin- |
| | | | | | like protease, |
| | | | | | kallikrein-11 |
| | | | | | inactive chain 1, |
| | | | | | kallikrein-11 |
| | | | | | inactive chain 2, |
| | | | | | EC: 3.4.21.- |
| BCMA | TNFRSF | Q02223 | TNF receptor | BCM, BCMA, | tumor necrosis |
| | 17 | | superfamily | CD269, | factor receptor |
| | | | member 17 | TNFRSF13A, | superfamily member |
| | | | | TNFR4 | 17, B-cell |
| | | | | | maturation protein, |
| | | | | | BCMA antigen, |
| | | | | | CD269 |
| Dectin-2 | CLEC6A | Q6EIG7 | C-type lectin | CLEC4N, | C-type lectin |
| | | | domain containing | CLECSF10, | domain family 6 |
| | | | 6A | dectin-2, | member A, C-type |
| | | | | hDECTIN-2, | lectin superfamily |
| | | | | DECTIN2 | member 10, |
| | | | | | dendritic cell- |
| | | | | | associated C-type |
| | | | | | lectin 2 (DC- |
| | | | | | associated C-type |
| | | | | | lectin 2, Dectin-2) |
| EpCAM | EPCAM | P16422 | epithelial cell | Ber-Ep4, BerEp4, | Ep-CAM, |
| | | | adhesion | DIAR5, EGP-2, | adenocarcinoma- |
| | | | | EGP314, EGP40, | associated antigen, |
| | | | | ESA, HNPCC8, | cell surface |
| | | | | KS1/4, KSA, | glycoprotein Trop-1, |
| | | | | LYNCH8, M4S1, | epithelial cell |
| | | | | MIC18, MK-1, | surface antigen, |
| | | | | MOC-31, | epithelial |
| | | | | TACSTD1, | glycoprotein (EGP), |
| | | | | TROP1, GA733-2, | epithelial |
| | | | | M1S2, CD326 | glycoprotein 314 |
| | | | | | (EGP314, |
| | | | | | hEGP314), KS 1/4 |
| | | | | | antigen, KSA, major |
| | | | | | gastrointestinal |
| | | | | | tumor-associated |
| | | | | | protein GA733-2, |
| | | | | | tumor-associated |
| | | | | | calcium signal |
| | | | | | transducer 1, CD326 |
| HCC-4 | CCL16 | O15467 | C-C motif | CKb12, HCC-4, | C-C motif |
| | | | chemokine ligand | ILINCK, LCC-1, | chemokine 16, |
| | | | 16 | LEC, LMC, Mtn- | chemokine CC-4 |
| | | | | 1, NCC-4, NCC4, | (HCC-4), |
| | | | | SCYA16, SCYL4 | chemokine LEC, IL- |
| | | | | | 10-inducible |
| | | | | | chemokine, |
| | | | | | lymphocyte and |
| | | | | | monocyte |
| | | | | | chemoattractant |
| | | | | | (LMC), monotactin- |
| | | | | | 1 (MTN-1), NCC-4, |
| | | | | | small-inducible |
| | | | | | cytokine A16 |
| TGFa | TGFA | P01135 | transforming | TFGA, ETGF, Tgf | transforming growth |
| | | | growth factor | α | factor α, |
| | | | alpha | | transforming growth |
| | | | | | factor-α, |
| | | | | | protransforming |
| | | | | | growth factor alpha, |
| | | | | | TGF-alpha, EGF- |
| | | | | | like TGF (ETGF), |
| | | | | | TGF type 1 |
| IP-10 | CXCL10 | P02778 | C-X-C motif | C7, IFI10, INP10, | C-X-C motif |
| | | | chemokine ligand | IP-10, SCYB10, | chemokine 10, 10 |
| | | | 10 | crg-2, gIP-10, | kDa interferon |
| | | | | mob-1 | gamma-induced |
| | | | | | protein (Gamma- |
| | | | | | IP10, IP-10), small- |
| | | | | | inducible cytokine |
| | | | | | B10, CXCL10(1- |
| | | | | | 73), gamma-IFN |
| | | | | | inducible early |
| | | | | | reasponse, IFNG |
| | | | | | inducible protein- |
| | | | | | 10, interferon- |
| | | | | | inducible protein- |
| | | | | | 10, small inducible |
| | | | | | cytokine subfamily |
| | | | | | B (Cyx-X-Cys) |
| | | | | | member 10, γ-IFN |
| | | | | | inducible early |
| | | | | | response |
| BLAME | SLC6A4 | P31645 | solute carrier | 5-HTT, 5- | sodium-dependent |
| | | | family 6 member | HTTLPR, 5HTT, | serotonin |
| | | | 4 | HTT, OCD1, | transporter, SERT, |
| | | | | SERT, SERT1, | 5HT transporter |
| | | | | hSERT | (5HTT), 5-HT |
| | | | | | transporter, |
| | | | | | serotonin transporter |
| CILP-1 | CILP | O75339 | cartilage | CILP-1, | cartilage |
| | | | intermediate layer | HsT18872, CILP1, | intermediate layer |
| | | | protein | UNQ602/PRO118 | protein 1, CILP-1, |
| | | | | 8 | cartilage |
| | | | | | intermediate-layer |
| | | | | | protein, cartilage |
| | | | | | intermediate layer |
| | | | | | protein 1 C1, |
| | | | | | cartilage |
| | | | | | intermediate layer |
| | | | | | protein 1 C2 |
| PIGF | PIGF | Q07326 | phosphatidylinosit | OORS | phosphatidylinositol- |
| | | | ol glycan anchor | | glycan biosynthesis |
| | | | biosynthesis class | | class F protein, PIG- |
| | | | F | | F, GPI11 homolog |
| LOX-1 | OLR1 | P78380 | oxidized low | CLEC8A, LOX1, | oxidized low- |
| | | | density lipoprotein | LOXIN, SCARE1, | density lipoprotein |
| | | | receptor 1 | SLOX1 | receptor 1, Ox-LDL |
| | | | | | receptor 1, C-type |
| | | | | | lectin domain family |
| | | | | | 8 member A, lectin- |
| | | | | | like oxidized LDL |
| | | | | | receptor 1 (LOX-1, |
| | | | | | lectin-like oxLDL |
| | | | | | receptor 1, hLOX- |
| | | | | | 1), lectin-type |
| | | | | | oxidized LDL |
| | | | | | receptor 1, oxidized |
| | | | | | low-density |
| | | | | | lipoprotein receptor |
| | | | | | 1 soluble form |
| MCP-2 | CCL8 | P80075 | C-C motif | HC14, MCP-2, | C-C motif |
| | | | chemokine ligand | MCP2, SCYA10, | chemokine 8, HC14, |
| | | | 8 | SCYA8 | monocyte |
| | | | | | chemoattractant |
| | | | | | protein 2, monocyte |
| | | | | | chemotactic protein |
| | | | | | 2 (MCP-2), small- |
| | | | | | inducible cytokine |
| | | | | | A8, MCP-2(6-76) |
| Resistin | RETN | Q9HD89 | resistin | ADSF, FIZZ3, | adipose tissue- |
| | | | | RENT, RSTN, | specific secretory |
| | | | | XCP1, RETN1, | factor (ADSF), |
| | | | | HXCP1, | C/EBP-epsilon- |
| | | | | UNQ407/PRO119 | regulated myeloid- |
| | | | | 9 | specific secreted |
| | | | | | cysteine-rich |
| | | | | | protein, cysteine- |
| | | | | | rich secreted protein |
| | | | | | A12-alpha-like 2, |
| | | | | | cysteine-rich |
| | | | | | secreted protein |
| | | | | | FIZZ3 |
| HVEM | TNFRSF | Q92956 | TNF receptor | ATAR, CD270, | tumor necrosis |
| | 14 | | superfamily | HVEA, HVEM, | factor receptor |
| | | | member 14 | LIGHTR, TR2, | superfamily member |
| | | | | UNQ329/PRO509, | 14, Herpes virus |
| | | | | LOC100131742 | entry mediator A |
| | | | | | (Herpesvirus entry |
| | | | | | mediator A, HveA), |
| | | | | | tumor necrosis |
| | | | | | factor receptor-like |
| | | | | | 2 (TR2), CD270 |
| ENPP-7 | ENPP7 | Q6UWV6 | ectonucleotide | ALK-SMase, E- | ectonucleotide |
| | | | pyrophosphatase/ | NPP 7, NPP-7, | pyrophosphatase/ph |
| | | | phosphodiesterase | NPP7, | osphodiesterase |
| | | | 7 | UNQ3077/PRO99 | family member 7, E- |
| | | | | 12, HSPDE7A1 | NPP 7, NPP-7, |
| | | | | | alkaline |
| | | | | | sphingomyelin |
| | | | | | phosphodiesterase, |
| | | | | | intestinal alkaline |
| | | | | | shingomyelinase |
| | | | | | (Alk-SMase), |
| | | | | | EC: 3.1.4.12 |
| Syndecan-4 | SDC4 | P31431 | syndecan 4 | SYND4, | syndecan-4, |
| | | | | RYUDOCAN | SYND4, |
| | | | | | amphiglycan, |
| | | | | | ryudocan core |
| | | | | | protein |
| IL-2 Rg | IL2RG | P31785 | interleukin 2 | CD132, CIDX, IL- | cytokine receptor |
| | | | receptor subunit | 2RG, IMD4, P64, | common subunit |
| | | | gamma | SCIDX, SCIDX1, | gamma, interleukin- |
| | | | | IL15RG, IL2R | 2 receptor subunit |
| | | | | gamma, IL2R γ | gamma (IL-2 |
| | | | | | receptor subunit |
| | | | | | gamma, IL-2R |
| | | | | | subunit gamma, IL- |
| | | | | | 2RG), gammaC, |
| | | | | | p64, CD132, |
| | | | | | common gamma |
| | | | | | chain receptor, |
| | | | | | common γ chain |
| | | | | | receptor, IL2R |
| | | | | | gamma, IL2R γ, |
| | | | | | interleukin 2 |
| | | | | | receptor subunit γ, |
| | | | | | γ(c), γ C, γ chain |
| MICA | MICA | Q29983 | MHC class I | MIC-A, | MIC-A |
| | | | polypeptide- | PERB11.1, | |
| | | | related sequence | XXbac- | |
| | | | A | BPG181B23.1 | |
| Dopa | DDC | P20711 | dopa | AADC | aromatic-L-amino- |
| Decarboxylase | | | decarboxylase | | acid decarboxylase, |
| | | | | | aromatic L-amino |
| | | | | | acid decarboxylase, |
| | | | | | AADC, DOPA |
| | | | | | decarboxylase |
| | | | | | (DDC), EC: 4.1.1.28 |
| NPDC-1 | NPDC1 | Q9NQX5 | neural | CAB, CAB-, | neural proliferation |
| | | | proliferation, | CAB-1, CAB1, | differentiation and |
| | | | | NPDC-1 | |
| | | | differentiation and | | control protein 1, |
| | | | control 1 | | NPDC-1 |
| MCP-4 | CCL13 | Q99616 | C-C motif | CKb10, MCP-4, | C-C motif |
| | | | chemokine ligand | MCP4, NCC-1, | chemokine 13, CK- |
| | | | 13 | NCC1, SCYA13, | beta-10, monocyte |
| | | | | SCYL1 | chemoattractant |
| | | | | | protein 4, monocyte |
| | | | | | chemotactic protein |
| | | | | | 4 (MCP-4), NCC-1, |
| | | | | | small-inducible |
| | | | | | cytokine A13, C-C |
| | | | | | motif chemokine 13 |
| | | | | | long chain, C-C |
| | | | | | motif chemokine 13 |
| | | | | | medium chain, C-C |
| | | | | | motif chemokine 13 |
| | | | | | short chain |
| EG-VEGF | PROK1 | P58294 | prokineticin 1 | EGVEGF, PK1, | prokineticin-1, |
| | | | | PRK1, | endocrine-gland- |
| | | | | UNQ600/PRO118 | derived vascular |
| | | | | 6 | endothelial growth |
| | | | | | factor (EG-VEGF), |
| | | | | | mambakine |
| Glycoprotein | GP5 | P40197 | glycoprotein V | CD42d, GPV | platelet glycoprotein |
| V | | | platelet | | V, GPV, |
| | | | | | glycoprotein 5, |
| | | | | | CD42d, |
| | | | | | glycoprotein V |
| | | | | | precursor |
| Semaphorin | SEMA4G | Q9NTN9 | semaphorin 4G | KIAA1619 | semaphorin-4G |
| 4G | | | | | |
| IL-12p40 | IL12B | P29460 | interleukin 12B | CLMF, CLMF2, | interleukin-12 |
| | | | | IL-12B, IMD28, | subunit beta, IL- |
| | | | | IMD29, NKSF, | 12B, cytotoxic |
| | | | | NKSF2, IL-12, IL- | lymphocyte |
| | | | | 12 p40, p40 | maturation factor 40 |
| | | | | | kDa subunit (CLMF |
| | | | | | p40), IL-12 subunit |
| | | | | | p40, NK cell |
| | | | | | stimulatory factor |
| | | | | | chain 2 (NKSF2) |
| PSA-total | KLK3 | P07288 | kallikrein related | APS, KLKA2, | prostate-specific |
| | | | peptidase 3 | PSA, hK3, | antigen, PSA, |
| | | | | KLK2A1 | gamma- |
| | | | | | seminoprotein |
| | | | | | (seminin), |
| | | | | | kallikrein-3, P-30 |
| | | | | | antigen, |
| | | | | | semenogelase, γ- |
| | | | | | seminoprotein, |
| | | | | | EC: 3.4.21.77 |
| IL-15 | IL15 | P40933 | interleukin 15 | IL-15, IL-T | interleukin-15, IL- |
| | | | | | 15 |
| MAP1D | METAP1D | Q6UB28 | methionyl | MAP 1D, | methionine |
| | | | aminopeptiase | MAP1D, MetAP | aminopeptiase 1D |
| | | | type 1D, | 1D, Metap1l | mitochondrial, MAP |
| | | | mitochondrial | | 1D, MetAP 1D, |
| | | | | | peptidase M 1D, |
| | | | | | EC: 3.4.11.18 |
| Clq | C1QA | P02745 | complement | C1QD1, Hs.9641 | complement C1q |
| | | | C1qA chain | | subcomponent |
| | | | | | subunit A |
| TNF4 | TNFSF4 | P23510 | TNF superfamily | CD134L, CD252, | tumor necrosis |
| | | | member 4 | GP34, OX-40L, | factor ligand |
| | | | | OX4OL, | superfamily member |
| | | | | TNLG2B, TXGP1, | 4, glycoprotein |
| | | | | OX40 ligand | Gp34, OX40 ligand |
| | | | | | (OX40L), TAX |
| | | | | | transcriptionally - |
| | | | | | activated |
| | | | | | glycoprotein 1, |
| | | | | | CD252 |
| Dtk | TYRO3 | Q06418 | TYRO3 protein | BYK, Dtk, DTK, | tyrosine-protein |
| | | | tyrosine kinase | Etk-2, RSE, Rek, | kinase receptor |
| | | | | Sky, SKY, Tif, | TYRO3, tyrosine- |
| | | | | TIF | protein kinase BYK, |
| | | | | | tyrosine-protein |
| | | | | | kinase DTK, |
| | | | | | tyrosine-protein |
| | | | | | kinase RSE, |
| | | | | | tyrosine-protein |
| | | | | | kinase SKY, |
| | | | | | tyrosine-protein |
| | | | | | kinase TIF, |
| | | | | | EC: 2.7.10.1 |
| Endoglin | ENG | P17813 | endoglin | END, HHT1, | CD105, cell |
| | | | | ORW1, CD105 | adhesion regulator |
| ENA-78 | CXCL5 | P42830 | C-X-C motif | ENA-78, SCYB5 | C-X-C motif |
| | | | chemokine ligand | | chemokine 5, ENA- |
| | | | 5 | | 78(1-78), epithelial- |
| | | | | | derived neutrophil- |
| | | | | | activating protein |
| | | | | | 78, neutrophil- |
| | | | | | activating peptide |
| | | | | | ENA-78, small- |
| | | | | | inducible cytokine |
| | | | | | B5, ENA-78(8-78), |
| | | | | | ENA-78(9-78) |
| Reg3A | REG3A | Q06141 | regenerating | HIP, HIP/PAP, | regenerating family |
| | | | family member 3 | INGAP, PAP, | member 3 α, |
| | | | alpha | PAP-H, PAP1, | regenerating islet- |
| | | | | PBCGF, REG-III, | derived protein 3- |
| | | | | REG3 | alpha, REG-3-alpha, |
| | | | | | hepatointestinal |
| | | | | | pancreatic protein |
| | | | | | (HIP/PAP), human |
| | | | | | proislet peptide |
| | | | | | (HIP), pancreatitis- |
| | | | | | associated protein 1, |
| | | | | | regenerating islet- |
| | | | | | derived protein III- |
| | | | | | alpha (Reg III- |
| | | | | | alpha), regenerating |
| | | | | | islet-derived protein |
| | | | | | 3-alpha 16.5 kDa |
| | | | | | form, regenerating |
| | | | | | islet-derived protein |
| | | | | | 3-alpha 15 kDa form |
| MIP-lb | CCL4 | P13236 | C-C motif | ACT2, AT744.1, | C-C motif |
| | | | chemokine ligand | G-26, HC21, | chemokine 4, |
| | | | 4 | LAG-1, LAG1, | macrophage |
| | | | | MIP-1-beta, | inflammatory |
| | | | | MIP1B, MIP1B1, | protein 1-β, G-26 T- |
| | | | | SCYA2, SCYA4, | lymphocyte-secreted |
| | | | | Mip1 β, Mip-1-β | protein, HC21, |
| | | | | | lymphocyte |
| | | | | | activation gene 1 |
| | | | | | protein (LAG-1), |
| | | | | | MIP-1-beta(1-69), |
| | | | | | macrophage |
| | | | | | inflammatory |
| | | | | | protein 1-beta (MIP- |
| | | | | | 1-beta), PAT 744, |
| | | | | | protein H400, SIS- |
| | | | | | gamma, small- |
| | | | | | inducible cytokine |
| | | | | | A4, T-cell activation |
| | | | | | protein 2 (ACT-2), |
| | | | | | MIP-1-beta(3-69) |
| FGF-17 | FGF17 | O60258 | fibroblast growth | FGF-13, FGF-17, | FGF-17 |
| | | | factor 17 | HH20, | |
| | | | | UNQ161/PRO187 | |
| IL-6R | IL6R | P08887 | interleukin 6 | IL6Q, gp80, | interleukin-6 |
| | | | receptor | CD126, HIES5, | receptor subunit |
| | | | | IL-6R, IL6RA, | alpha, IL-6 receptor |
| | | | | IL6RQ, IL-1Ra, | subunit alpha, IL-6R |
| | | | | | subunit alpha, IL- |
| | | | | IL6QTL, IL-6R-1, | 6R-alpha, IL-6RA, |
| | | | | IL-6RA, Il6r α | IL-6R 1, membrane |
| | | | | | glycoprotein 80 |
| | | | | | (gp80), soluble |
| | | | | | interleukin-6 |
| | | | | | receptor subunit |
| | | | | | alpha (sIL6R), |
| | | | | | CD126, interleukin- |
| | | | | | 6 receptors, |
| | | | | | interleukin 6 |
| | | | | | receptor α chain |
| IL-8 | CXCL8 | P10145 | C-X-C motif | GCP-1, GCP1, | interleukin-8, IL-8, |
| | | | chemokine ligand | IL8, LECT, | C-X-C motif |
| | | | 8 | LUCT, LYNAP, | chemokine 8, |
| | | | | MDNCF, | chemokine (C-X-C |
| | | | | MONAP, NAF, | motif) ligand 8, |
| | | | | NAP-1, NAP1, | emoctakin, |
| | | | | SCYB8, hnIL-8 | granulocyte |
| | | | | | chemotactic protein |
| | | | | | 1 (GCP-1), |
| | | | | | monocyte-derived |
| | | | | | neutrophil |
| | | | | | chemotactic factor |
| | | | | | (MDNCF), |
| | | | | | monocyte-derived |
| | | | | | neutrophil- |
| | | | | | activating peptide |
| | | | | | (MONAP), |
| | | | | | neutrophil- |
| | | | | | activating protein 1 |
| | | | | | (NAP-1), protein 3- |
| | | | | | 10C, T-cell |
| | | | | | chemotactic factor, |
| | | | | | MDNCF-a |
| | | | | | (GCP/IL-8 protein |
| | | | | | IV, IL8/NAP1 form |
| | | | | | I), interleukin-8 |
| | | | | | ((Ala-IL-8)77, |
| | | | | | GCP/IL-8 protein II, |
| | | | | | IL-8(1-77), |
| | | | | | IL8/NAP1 form II, |
| | | | | | MDNCF-b), IL-8(5- |
| | | | | | 77), IL-8(6-77) |
| | | | | | ((Ser-IL-8)72, |
| | | | | | GCP/IL-8 protein I, |
| | | | | | IL8/NAP1 form III, |
| | | | | | lymphocyte-derived |
| | | | | | neutrophil- |
| | | | | | activating factor |
| | | | | | (LYNAP), |
| | | | | | MDNCF-c, |
| | | | | | neutrophil- |
| | | | | | activating factor |
| | | | | | (NAF)), IL-8(7-77) |
| | | | | | (GCP/IL-8 protein |
| | | | | | V, IL8/NAP1 form |
| | | | | | IV), IL-8(7-77) |
| | | | | | (GCP/IL-8 protein |
| | | | | | V, IL8/NAP1 form |
| | | | | | IV), IL-8(8-77) |
| | | | | | (GCP/IL-8 protein |
| | | | | | VI, IL8/NAP1 form |
| | | | | | V), IL-8(9-77) |
| | | | | | (GCP/IL-8 protein |
| | | | | | III, IL8/NAP1 form |
| | | | | | VI) |
| Galectin-8 | LGALS8 | O00214 | galectin 8 | Gal-8, PCTA-1, | galectin-8, Gal-8, |
| | | | | PCTA1, Po66- | Po66 carbohydrate- |
| | | | | CBP, PCTA | binding protein |
| | | | | | (Po66-CBP), |
| | | | | | prostate carcinoma |
| | | | | | tumor antigen 1 |
| | | | | | (PCTA-1) |
| CA4 | CA4 | P22748 | carbonic | CAIV, Car4, RP17 | carbonate |
| | | | anhydrase 4 | | dehydratase IV, |
| | | | | | carbonic anhydrase |
| | | | | | IV (CA-IV), |
| | | | | | carbonic anhydrase |
| | | | | | isozyme IV, |
| | | | | | EC: 4.2.1.1 |
| Cystatin EM | CST6 | Q15828 | cystatin E/M | ECTD15 | cystatin-M, cystatin- |
| | | | | | 6, cystatin-E |
| FUT8 | FUT8 | Q9BYC5 | fucosyltransferase | CDGF, CDGF1, | alpha-(1,6)- |
| | | | 8 | LOC105370537 | fucosyltransferase, |
| | | | | | alpha1-6FucT, |
| | | | | | fucosyltransferase 8, |
| | | | | | GDP-L-Fuc: N- |
| | | | | | acetyl-beta-D- |
| | | | | | glucosaminide |
| | | | | | alpha1,6- |
| | | | | | fucosultransferase, |
| | | | | | GDP-fucose- |
| | | | | | glycoprotein |
| | | | | | fucosyltransferase, |
| | | | | | glycoprotein 6- |
| | | | | | alpha-L- |
| | | | | | fucosyltransferase, |
| | | | | | N-acetyl-beta-D- |
| | | | | | glucosaminide |
| | | | | | alpha1,6- |
| | | | | | fucosultransferase, |
| | | | | | N-acetyl-β-D- |
| | | | | | glucosaminide |
| | | | | | alpha1,6- |
| | | | | | fucosultransferase |
| B7-H3 | CD276 | Q5ZPR3 | CD276 molecule | 4Ig-B7-H3, B7- | CD276 antigen, 4Ig- |
| | | | | H3, B7H3, B7RP- | B7-H3, B7 homolog |
| | | | | 2, PSEC0249, | 3 (B7-H3), |
| | | | | UNQ309/PRO352 | costimulatory |
| | | | | | molecule, CD276 |
| GCP-2 | CXCL6 | P80162 | C-X-C motif | CKA-3, GCP-2, | C-X-C motif |
| | | | chemokine ligand | GCP2, SCYB6 | chemokine 6, |
| | | | 6 | | chemokine alpha 3 |
| | | | | | (CKA-3), |
| | | | | | chemokine α 3, |
| | | | | | granulocyte |
| | | | | | chemotactic protein |
| | | | | | 2 (GCP-2), small- |
| | | | | | inducible cytokine |
| | | | | | B6, small-inducible |
| | | | | | cytokine B6 N- |
| | | | | | processed variant 1, |
| | | | | | small-inducible |
| | | | | | cytokine B6 N- |
| | | | | | processed variant 2, |
| | | | | | small-inducible |
| | | | | | cytokine B6 N- |
| | | | | | processed variant 3 |
| CD40L | CD40LG | P29965 | CD40 ligand | CD154, CD40L, | CD40 antigen, |
| | | | | HIGM1, IGM, | CD40-L, T-cell |
| | | | | IMD3, T-BAM, | antigen Gp39, TNF- |
| | | | | TNFSF5, TRAP, | related activation |
| | | | | gp39, hCD40L | protein (TRAP), |
| | | | | | tumor necrosis |
| | | | | | factor ligand |
| | | | | | superfamily member |
| | | | | | 5, CD40 ligand |
| | | | | | membrane form, |
| | | | | | CD40 ligand soluble |
| | | | | | form (sCD40L), |
| | | | | | CD154 |
| MDC | CCL22 | O00626 | C-C motif | A-152E5.1, | C-C motif |
| | | | chemokine ligand | ABCD-1, DC/B- | chemokine 22, CC |
| | | | 22 | CK, MDC, | chemokine STCP-1, |
| | | | | SCYA22, STCP-1 | MDC(1-69), |
| | | | | | macrophage-derived |
| | | | | | chemokine, small- |
| | | | | | inducible cytokine |
| | | | | | A22, stimulated T- |
| | | | | | cell chemotactic |
| | | | | | protein 1, MDC(3- |
| | | | | | 69), MDC(5-69), |
| | | | | | MDC(7-69) |
| 4-1BB | TNFRSF9 | Q07011 | TNF receptor | 4-1BB, CD137, | tumor necrosis |
| | | | superfamily | CDw137, ILA, | factor receptor |
| | | | member 9 | IMD109 | superfamily member |
| | | | | | 9, 4-1BB ligand |
| | | | | | receptor, CDw137, |
| | | | | | T-cell antigen 4- |
| | | | | | 1BB homolog, T- |
| | | | | | cell antien ILA, |
| | | | | | CD137 |
| HO-1 | HMOX1 | P09601 | heme oxygenase 1 | HMOX1D, HO-1, | heme oxygenase, |
| | | | | HSP32, | heme oxygenase |
| | | | | bK286B10, | (decyclizing), HO-1, |
| | | | | HMOX, HO, HO1 | heme oxygenase 1 |
| | | | | | soluble form, |
| | | | | | EC: 1.14.14.18 |
| SOST | SOST | Q9BQB4 | sclerostin | CDD, VBCH, | |
| | | | | DAND6, SOST1, | |
| | | | | UNQ2976/PRO74 | |
| | | | | 88/PRO7476 | |
| S100A13 | S100A13 | Q99584 | S100 calcium | RP1_178F155 | protein S100-A13, |
| | | | binding protein | | S100 calcium- |
| | | | A13 | | binding protein A13 |
| Kallikrein 7 | KLK7 | P49862 | kallikrein related | PRSS6, SCCE, | kallikrein-7, hK7, |
| | | | peptidase 7 | hK7 | serine protease 6, |
| | | | | | stratum corneum |
| | | | | | chymotryptic |
| | | | | | enzyme (hSCCE), |
| | | | | | EC: 3.4.21.117 |
| IL-13 | IL13 | P35225 | interleukin 13 | IL-13, P600, | interleukin-13, IL- |
| | | | | NC30 | 13 |

In some embodiments, the FV comprises one or more nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 65, 70, 75, 80, 85, 90, 95, or 100). Non-limiting examples of the one or more nucleic acids include: hsa-miR-125b-5p, hsa-miR-145-5p, hsa-miR-191-5p, hsa-miR-199a-3p, hsa-miR-21-5p, hsa-miR-22l-3p, hsa-miR-222-3p, hsa-miR-22-3p, hsa-miR-23a-3p, hsa-miR-23b-3p, hsa-miR-27a-3p, hsa-miR-27b-3p, hsa-miR-29a-3p, hsa-miR-29c-3p, hsa-miR-31-5p, hsa-miR-320a, hsa-miR-34a-5p, hsa-miR-423-3p, hsa-miR-424-5p, and hsa-miR-940. miRNA sequences may be obtained from www.mirbase.org. An EV that comprises the one or more of the nucleic acids may include the one or more nucleic acids within the EV.

### Proteins

Disclosed herein are compositions comprising a protein. The protein may be independent of an EV. For instance, the protein may not be present within the EV or within the membrane of the EV. The composition may comprises one or more proteins (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 65, 70, 75, 80, 85, 90, 95, or 100). The protein may be present as a monomer or multimer. The protein may have a molecular weight (as a monomer or multimer as applicable) of at least about 10 kDa (kilodalton). Non-limiting examples of the one or more proteins include: Ferritin, IGFBP-4 (Insulin-like growth factor binding protein-4), IL-1 R6 (Interleukin 1 Receptor 6), LAMP2 (Lysosome-associated membrane glycoprotein 2), bIG-H3 (Transforming growth factor-beta-induced protein ig-h3), GPR115 (Adhesion G protein-coupled receptor F4), CD63 antigen, CD109 antigen, Serpin F1 (Pigment epithelium-derived factor), IGFBP-6 (Insulin-like growth factor binding protein-6), HS3ST4 (Heparan sulfate glucosamine 3-O-sulfotransferase 4), OPN (Osteopontin), PAI-1 (Plasminogen activator inhibitor-1 or SERPINE 1), Cathepsin B, IGFBP-2 (Insulin-like growth factor binding protein-2), Semaphorin 6C, IGF-2 (Insulin-like growth factor-2), Sortilin, Serpin B6, Dkk-3 (Dickkopf-related protein 3), CNTF (Ciliary neurotrophic factor), TSP-1 (Thrombospondin 1), GM-CSF Ra (Granulocyte-macrophage colony-stimulating factor receptor subunit alpha), Thrombomodulin, Endoglycan, (podocalyxin-like protein 2) IGFBP-3 (Insulin-like binding protein-3), RGM-C(Hemojuvelin), PF4 (Platelet Factor 4), MIF (Macrophage migration inhibitory factor), TGM4 (Protein-glutamine gamma-glutamyltransferase 4), Periostin, Furin, TIMP-1 (Tissue inhibitor of MMPs 1), Decorin, PCK1 (Phosphoenolpyruvate carboxykinase, cytosolic) CD9 antigen, CD99 antigen, CA2 (Carbonic anhydrase 2), PRDX4 (Peroxidredoxin-4), Transferrin, DcR3 (Tumor necrosis factor receptor superfamily member 6B), GP73 (Golgi membrane protein 1), CD81 antigen, Lumican, or TIMP-2 (Tissue Inhibitor of MMPs 2), and the proteins of Table 1.

In some embodiments, the one or more proteins comprises TIMP1. In some embodiments, the TIMP1 is present at about 200 pg/mL to about 80 ng/mL of the composition or about 30 pg/mL to about 12 ng/mL. In some embodiments, the one or more proteins comprises OPN. In some embodiments, the OPN is present at about 200 pg/mL to about 80 ng/mL of the composition or about 30 pg/mL to about 12 ng/mL. In some embodiments, the one or more proteins comprises IGFBP4. In some embodiments, the IGFBP4 is present at about 200 pg/mL to about 80 ng/mL of the composition or about 30 pg/mL to about 12 ng/mL. In some embodiments, the one or more proteins comprises osteonectin. In some embodiments, the osteonectin is present at about 200 pg/mL to about 80 ng/mL of the composition or about 30 pg/mL to about 12 ng/mL. In some embodiments, the concentration of the one or more proteins is measured by ELISA.

In some embodiments, the total protein concentration of the one or more proteins is about 10 to about 40 μg per ml of the composition. For example, the concentration in frozen product. In some embodiments, the total protein concentration of the one or more proteins is about 1.5 to about 6 μg per ml of the composition. For example, the concentration in an intravenous solution for administration.

In some embodiments, compositions described herein can safely target one or more (e.g., more than hundreds) different biomolecular interactions or signaling pathways. In some embodiments, compositions described herein can treat one or more injuries or diseases caused by multiple etiologies. In some embodiments, compositions described herein can be used for treating a disease or condition without identifying the pathogen underlying the disease or condition, thus offering an advantage for treating a disease or condition caused by an emerging or previously unknown pathogen.

### A. Pharmaceutical Compositions

Compositions described herein may be administered in vivo in a pharmaceutically acceptable carrier. A pharmaceutically acceptable carrier may be not biologically or otherwise undesirable, i.e., the material may be administered to a subject, along with the EV and/or protein, without causing any undesirable biological effects or interacting in a deleterious manner with any of the other components of the pharmaceutical composition in which it is contained. As a non-limiting example, the composition comprises sodium chloride.

Parenteral administration of the composition, if used, is generally characterized by injection. The injection may be an intravenous injection. The injection may involve administration of the composition over a period of about 60 minutes. The composition for injection may comprise saline, e.g., 0.9% saline (NaCl). The 0.9% saline may be about 85% to about 99% or more of the composition by volume. For instance, the composition comprises about 85% of 0.9% saline and about 15% of an EV and/or protein component. The EV and/or protein component may be formulated in an isotonic infusion solution. The EV and/or protein component may comprise sodium chloride. The EV and/or protein component may comprise sodium chloride, sodium lactate, potassium chloride, and calcium chloride. The EV and/or protein component may comprise sodium chloride (NaCl) at 100-150 mEq/L, sodium lactate (C₃H₅NaO₃) at 25-35 mEq/L, potassium chloride (KCl) at 2-6 mEq/L, calcium chloride (CaCl₂)) at 1-5 mEq/L, and chloride ions (Cl⁻) at 80-130 mEq/L. The EV and/or protein component may comprise 0.1 M to 0.8 M saccharide, optionally a polysaccharide. Non-limiting example saccharides and polysaccharides include, but are not limited to, glucose, aldose (D-allose, D-altrose, D-mannose, etc.), glucopyranose, pentahydroxyhexanal, alpha-D-glucopyranoside dihydrate, a-D-glucopyranosyl-D-glucose, a-D-glucopyranosyl-dihydrate, polymer of P-D-glycopyranosyl units, P-D-fructofuranosyl a-D-glucopyranoside (anhydrous/dihydrate), β-D-galactopyranosyl-D-glucose, a-D-glucopyranosyl-a-D-glucopyranoside (anhydrous/dihydrate), galactose, pentoses (ribose, xylose, lyxose), dextrose, dodecacarbon monodecahydrate, fructose, sucrose, lactose, maltose, trehalose, agarose, D-galactosyl-0-(1-4)-anhydro-L-galactosyl, cellulose, starch, polyhydric alcohol, polyalcohol, alditol, erythritol, glycitol, glycerol, xylitol, and sorbitol. In some embodiments, the composition has a pH of about 6 to about 7.5.

In some embodiments, the composition is sterile by USP <71>. In some embodiments, the composition is endotoxin USP <85> free. In some embodiments, the composition is negative for mycoplasma DNA. In some embodiments, the composition is cell-free. In some embodiments, the composition is stored between −80° C. and −60° C. In some embodiments, the composition is administered within 6 hours of thaw when maintained at ambient temperature. In some embodiments, the composition is present in a glass vial.

### B. Methods of Composition Production

Compositions herein comprising an EV and a protein may include components produced from a MSC. The EV of the composition may be produced from a MSC. The protein of the composition may be produced from a MSC. The EV and/or protein may be produced from a MSC cultured at one or more of the following conditions: about 0.10% to about 5% oxygen, reduced or no serum, pH of about 5-7.5, reduced glucose, increased temperature, or these elements in various combinations. The MSC may be cultured under the aforementioned one or more conditions after the cell achieves confluency. The MSC may be cultured under the aforementioned one or more conditions after the cell is cultured at 37° C. and 5% CO2. The EV and/or protein of the composition may be obtained from a method comprising: (1) growing the MSCs to 50-80% confluency, then (2) culturing the MSCs at one or more of the following conditions: about 1% to about 5% oxygen, reduced or no serum, reduced glucose, or increased temperature. The growing at step (1) may be performed at about 37° C. and about 5% CO2. Culturing the MSCs may comprise introducing the cells to a culture media. The culture media may comprise basal media. For instance, basal media comprising amino acids, vitamins, and inorganic salts, and optionally a carbon source such as glucose. The culture media may comprise an isotonic infusion solution. In a non-limiting example embodiment, the culture media comprises a basal media and an isotonic infusion solution. The culture media may comprise basal media and sodium chloride. The culture media may comprise basal media, sodium chloride, sodium lactate, potassium chloride, and calcium chloride. The culture media may comprise a basal media and an isotonic infusion solution. The culture media may comprise basal media and sodium chloride, without serum. The culture media may comprise basal media, sodium chloride, sodium lactate, potassium chloride, and calcium chloride, without serum. The culturing at step (2) may occur over a period of 1, 2, 3, 4, 5, 6, or 7 days, wherein the oxygen and pH may change over time. At any time during the culturing step, the pH may be about 6.5 to about 7. At any time during the culturing step, the oxygen may be at about 0.5%, 1%, 1.5%, or 2%.

The EV and/or protein produced in the second culturing step (2) may be purified (e.g., partially or entirely), from the culture media of the second culturing step (2). The purification may comprise formulating the EV and/or the protein into an EV and/or protein component. The EV and/or protein component may comprise an isotonic infusion solution. The EV and/or protein component may comprise sodium chloride. The EV and/or protein component may comprise sodium chloride, sodium lactate, potassium chloride, and calcium chloride. The EV and/or protein component may comprise sodium chloride (NaCl) at 100-150 mEq/L, sodium lactate (C₃H₅NaO₃) at 25-35 mEq/L, potassium chloride (KCl) at 2-6 mEq/L, calcium chloride (CaCl₂)) at 1-5 mEq/L, and chloride ions (Cl⁻) at 80-130 mEq/L. The EV and/or protein component may comprise a saccharide, optionally a polysaccharide, e.g., 0.1 M to 0.8 M saccharide. Non-limiting example saccharides and polysaccharides include, but are not limited to, glucose, aldose (D-allose, D-altrose, D-mannose, etc.), glucopyranose, pentahydroxyhexanal, alpha-D-glucopyranoside dihydrate, a-D-glucopyranosyl-D-glucose, a-D-glucopyranosyl-dihydrate, polymer of P-D-glycopyranosyl units, P-D-fructofuranosyl a-D-glucopyranoside (anhydrous/dihydrate), β-D-galactopyranosyl-D-glucose, a-D-glucopyranosyl-a-D-glucopyranoside (anhydrous/dihydrate), galactose, pentoses (ribose, xylose, lyxose), dextrose, dodecacarbon monodecahydrate, fructose, sucrose, lactose, maltose, trehalose, agarose, D-galactosyl-0-(1-4)-anhydro-L-galactosyl, cellulose, starch, polyhydric alcohol, polyalcohol, alditol, erythritol, glycitol, glycerol, xylitol, and sorbitol. The amount of saccharide in the EV and/or protein component may be about 0.2 M to about 0.6 M, or about 0.4 M. In some embodiments, the EV and/or protein component is frozen. The frozen material may be thawed and combined with saline (e.g., 0.9% saline or sodium chloride) to generate an IV formulation for IV administration. The amount of saccharide in the IV formulation may be about 40 mM to about 80 mM, or about 60 mM.

The EV and/or protein component may be filter-sterilized. The EV and/or protein component may be filter sterilized after and/or during formulation from the culture media into the EV and/or protein component. The EV and/or protein may be concentrated. The concentration may occur during and/or after purification and/or exchange from the culture media into the EV and/or protein component. The EV and/or protein may be frozen, e.g., after and/or during purification. If frozen, the EV and/or protein may be formulated with a cryoprotectant prior to freezing. The cryoprotectant may comprises a saccharide, optionally a polysaccharide, e.g., as described above.

In some embodiments, reduced glucose is less than a normal control (e.g., 4.5 g/L). For example, the glucose reduction may be about 5% to about 15%, from about 10% to about 20%, from about 15% to about 25%, from about 20% to about 30%, or from about 25% to about 35% of the glucose in a normal control. In some embodiments, the reduced glucose is present in the MSC culture media (e.g., at step (2) as noted above) at a concentration of about 0.1, 0.5, 1.0, 1.5, 2.0, 2.5, 3.0, 3.5, or 4.0 g/L, or a range between any two of these values. In some embodiments, glucose is present at a concentration of less than or no more than 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, or 4.5 g/L.

In some embodiments, the MSC is cultured, e.g., at step (2) at about 0.1% to about 5% oxygen, for example, about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5% oxygen. In an example embodiment, the oxygen is about 0.5% to about 1.5%, or about 1%.

The pH at which the MSC is cultured, e.g., at step (2), can be from about 6.0 to about 7.4, for example, from 6.5 to about 7, or about 6.0, 6.1, 6.2, 6.3, 6.4, 6.5, 6.6, 6.7, 6.8, 6.9, 7.0, 7.1, 7.2, 7.3, or 7.4.

The temperature of the culture environment, e.g., at step (2), may be raised relative to physiologic homeostasis temperature (e.g., 37° C.). In one aspect, the temperature of the culture can be 35.0, 35.1, 35.2, 35.3, 36.4, 35.5, 35.6, 35.7, 35.8, 35.9, 36.0, 36.1, 36.2, 36.3, 36.4, 36.5, 36.6, 36.7, 36.8, 36.9, 37.0, 37.1, 37.2, 37.3, 37.4, 37.5, 37.6, 37.7, 37.8, 37.9, 38.0, 38.1, 38.2, 38.3, 38.4, 38.5, 38.6, 38.7, 38.8, 38.9, 39.0, 39.1, 39.2, 39.3, 39.4, 39.5, 39.6, 39.7, 39.8, 39.9, or 40.0° C.

In some embodiments, compositions described herein can be lyophilized for packaging and storing. In some embodiments, compositions described herein can be stored at ambient or room temperature (e.g., between 60° F. and 75° F. or between 15° C. and 24° C.). In some embodiments, the stability of compositions described herein at ambient or room temperature can provide advantages for logistics and/or delivery.

### II. Methods of Treating ARDS

In some embodiments, compositions disclosed herein are used in methods of treating ARDS in a subject.

In some embodiments, the subject has met the Berlin criteria for moderate to severe ARDS.

In some embodiments, the ARDS is acute ARDS. In some embodiments, the acute ARDS comprises dyspnea or worsening of hypoxemic respiratory failure following a predisposing risk factor. In some embodiments, the predisposing risk factor is pneumonia, nonpulmonary infection, trauma, transfusion, aspiration or shock.

In some embodiments, the subject is not infected with COVID-19. In some embodiments, the subject does not have Long COVID-19 (Post Acute Sequalae of COVID-19, PASC), e.g., does not have signs, symptoms, and conditions that continue or develop after acute COVID-19 infection. In some embodiments, the subject is not diagnosed with an infection within 1 week of the administering. In some embodiments, the subject is not suffering from an infection.

In some embodiments, the subject has influenza.

In some embodiments, the ARDS is not caused by bacterial pneumonia, viral pneumonia, fungal pneumonia, or parasitic pneumonia.

In some embodiments, the ARDS subtype is hyperinflammatory ARDS.

In some embodiments, the ARDS subtype is hypoinflammatory ARDS.

In some embodiments, the ARDS is characterized by serum bicarbonate, sTNFR1, and IL-6.

In some embodiments, the ARDS is caused by a pulmonary insult. In some embodiments, the pulmonary insult is aspiration. In some embodiments, the pulmonary insult is smoking. In some embodiments, the pulmonary insult is non-protective ventilation. In some embodiments, the pulmonary insult is lung contusion from trauma. In some embodiments, the pulmonary insult is thoracic surgery. In some embodiments, the pulmonary insult is drowning. In some embodiments, the pulmonary insult is pulmonary vasculitis. In some embodiments, the pulmonary insult is fat embolism. In some embodiments, the administering occurs within 7-14 days of the pulmonary insult.

In some embodiments, the ARDS is caused by a nonpulmonary insult. In some embodiments, the nonpulmonary insult is a blood transfusion. In some embodiments, the nonpulmonary insult is trauma. In some embodiments, the nonpulmonary insult is pancreatitis. In some embodiments, the nonpulmonary insult is drug reaction. In some embodiments, the nonpulmonary insult is a burn. In some embodiments, the nonpulmonary insult is a cardiopulmonary bypass. In some embodiments, the nonpulmonary insult is noncardiogenic shock. In some embodiments, the administering occurs within 7-14 days of the nonpulmonary insult.

Methods of treating ARDS in a subject comprising administering to the subject a composition comprising an EV and a protein, wherein the EV and/or the protein comprises one or more proteins selected from: Ferritin, IGFBP-4 (Insulin-like growth factor binding protein-4), IL-1 R6 (Interleukin 1 Receptor 6), LAMP2 (Lysosome-associated membrane glycoprotein 2), bIG-H3 (Transforming growth factor-beta-induced protein ig-h3), GPR115 (Adhesion G protein-coupled receptor F4), CD63 antigen, CD109 antigen, Serpin F1 (Pigment epithelium-derived factor), IGFBP-6 (Insulin-like growth factor binding protein-6), HS3ST4 (Heparan sulfate glucosamine 3-O-sulfotransferase 4), OPN (Osteopontin), PAI-1 (Plasminogen activator inhibitor-1 or SERPINE 1), Cathepsin B, IGFBP-2 (Insulin-like growth factor binding protein-2), Semaphorin 6C, IGF-2 (Insulin-like growth factor-2), Sortilin, Serpin B6, Dkk-3 (Dickkopf-related protein 3), CNTF (Ciliary neurotrophic factor), TSP-1 (Thrombospondin 1), GM-CSF Ra (Granulocyte-macrophage colony-stimulating factor receptor subunit alpha), Thrombomodulin, Endoglycan, (podocalyxin-like protein 2) IGFBP-3 (Insulin-like binding protein-3), RGM-C (Hemojuvelin), PF4 (Platelet Factor 4), MIF (Macrophage migration inhibitory factor), TGM4 (Protein-glutamine gamma-glutamyltransferase 4), Periostin, Furin, TIMP-1 (Tissue inhibitor of MMPs 1), Decorin, PCK1 (Phosphoenolpyruvate carboxykinase, cytosolic) CD9 antigen, CD99 antigen, CA2 (Carbonic anhydrase 2), PRDX4 (Peroxidredoxin-4), Transferrin, DcR3 (Tumor necrosis factor receptor superfamily member 6B), GP73 (Golgi membrane protein 1), CD81 antigen, Lumican, or TIMP-2 (Tissue Inhibitor of MMPs 2), and the proteins of Table 1. In some embodiments, the EV comprises one or more nucleic acids selected from hsa-miR-125b-5p, hsa-miR-145-5p, hsa-miR-191-5p, hsa-miR-199a-3p, hsa-miR-21-5p, hsa-miR-221-3p, hsa-miR-222-3p, hsa-miR-22-3p, hsa-miR-23a-3p, hsa-miR-23b-3p, hsa-miR-27a-3p, hsa-miR-27b-3p, hsa-miR-29a-3p, hsa-miR-29c-3p, hsa-miR-31-5p, hsa-miR-320a, hsa-miR-34a-5p, hsa-miR-423-3p, hsa-miR-424-5p, or hsa-miR-940.

In some embodiments, the composition is administered intravenously. As a non-limiting example, it is administered intravenously over about 30 min, 60 min, or 90 min. The intravenous dose may have a total volume of about 100 mL. The administration may occur 1, 2, 3, 4, or 5 times. Each subsequent dose may be administered about 1 to 2 days after the previous dose. In some embodiments, the administering occurs within 7 days of a clinical insult. In some embodiments, the administering occurs after chest imaging, wherein the chest imaging is indicative of a bilateral opacity. In some embodiments, the bilateral opacity is not due to effusion, atelectasis, or nodule.

In some embodiments, the administering comprises infusion over 60 minutes on a first day. In some embodiments, the administering further comprises infusion of the composition on a second day. In some embodiments, the second day is one day after the first day (e.g., Day 1 and Day 2), two days after the first day (e.g., Day 1 and Day 3), or three days after the first day (e.g., Day 1 and Day 4). In some embodiments, the infusion on the second day occurs if the subject does not have a SpO2 of at least about 93% between the administering and the infusion on the second day. In some embodiments, the administering comprises infusion of the composition on a third day. In some embodiments, the third day is two days after the second day (e.g., Day 3 and Day 5).

In some embodiments, the composition is administered at a cell-equivalent dose. For instance, extracellular vesicle (EV) concentration may be divided from a known number of source MSCs to determine the average quantity of EVs generated per cell. This value may be multiplied by the low and high range values of MSC concentrations (e.g., 1 million to 10 million cells/kg), and a mid-range EV concentration equivalent to *40,000,000 cells/mL can be determined. A composition comprising EVs may be administered at a cell equivalent dose range of 1 to 10 million cells/kg.

In some embodiments, the composition is administered at a dose that provides about 10 billion to about 250 billion EVs per mL. The total amount of EVs administered in a dose may be about 10 billion to about 250 billion. The total amount of EVs administered in a dose may be about 150 billion to about 3750 billion.

In some embodiments, prior to the administering the subject has a PaO2/FiO2 (P/F ratio) of less than or equal to 200 mm Hg.

In some embodiments, prior to the administering, the subject is treated with invasive or noninvasive mechanical ventilation. In some embodiments, the mechanical ventilation has a minimum Positive End Expiratory Pressure (PEEP) of about 5 cm H2O.

In some embodiments, prior to the administering, the subject is treated with continuous positive airway pressure (CPAP). In some embodiments, the continuous positive airway pressure is performed at 5 cm H2O.

In some embodiments, prior to the administering, the subject is treated with high flow nasal oxygen (HFNO) at a level at least about 30 L/min.

In some embodiments, prior to the administering, the subject is in respiratory failure. In some embodiments, the respiratory failure is not due to cardiac failure or fluid overload.

In some embodiments, after the administering, a biomarker in the subject is lower than prior to the administering. In some embodiments, the biomarker is measured from the blood of the subject. In some embodiments, after the administering the level of C-reactive protein (CRP) in the subject is less than prior to the administering. In some embodiments, after the administering the level of plasminogen activator inhibitor-1 (PAI-1) in the subject is less than prior to the administering. In some embodiments, after the administering the level of interleukin-8 (IL-8) in the subject is less than prior to the administering. In some embodiments, after the administering the level of interleukin-6 (IL-6) in the subject is less than prior to the administering. In some embodiments, after the administering the level of interleukin-1beta (IL-1b) in the subject is less than prior to the administering. In some embodiments, after the administering the level of sTNFrc in the subject is less than prior to the administering. In some embodiments, after the administering the level of D-dimer in the subject is less than prior to the administering. In some embodiments, after the administering the level of ferritin in the subject is less than prior to the administering. In some embodiments, after the administering the level of neutrophils in the subject is less than prior to the administering. In some embodiments, after the administering the Sequential Organ Failure Assessment (SOFA) Score change from prior to the administering decreases. In some embodiments, after the administering is about 15 to about 29 days after the administering. In some embodiments, the administering occurs within 72 hours of the subject being diagnosed with ARDS. In some embodiments, the administering occurs within 48 hours of the subject being diagnosed with ARDS.

In some embodiments, after the administering, the subject experiences improved tissue oxygenation. In some embodiments, after the administering, the subject experiences improved end-organ functioning. In some embodiments, after the administering, the subject has an improvement in partial pressure of arterial oxygen to fraction of inspired oxygen (PaO2/FiO2) ratio as compared to prior to the administering (e.g., as measured from arterial blood gas or imputed from SpO2 daily). In some embodiments, after the administering, the subject has an oxygenation saturation of at least about 93% on room air.

In some embodiments, within 28 days after the administering, the subject does not undergo ventilator treatment for at least about 1, 2, 3, 4, 5, 6, or 7 days. In some embodiments, within 28 days after the administering, the subject is not in the intensive care unit for at least about 1, 2, 3, 4, 5, 6, or 7 days. In some embodiments, within 28 days after the administering, the subject is not treated with oxygen for at least about 1, 2, 3, 4, 5, 6, or 7 days.

### III. Definitions

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

The term “about” or “approximately” can mean within an acceptable error range for the particular value, which may depend in part on how the value is measured or determined, e.g., the limitations of the measurement system. For example, “about” can mean within 1 or more than 1 standard deviation. Alternatively, “about” can mean a range of up to 20%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures.

### EXAMPLES

The following examples are put forth so as to provide those of ordinary skill in the art with a complete disclosure and description of how the compounds, compositions, articles, devices and/or methods claimed herein are made and evaluated and are intended to be purely exemplary and are not intended to limit the disclosure. Efforts have been made to ensure accuracy with respect to numbers (e.g., amounts, temperature, etc.), but some errors and deviations should be accounted for. These examples are provided for illustrative purposes only and not to limit the scope of the claims provided herein.

### Example 1—Production of Therapeutic Composition

An MSC secretome therapeutic composition (investigational medicinal product or “IMP”) was made by the following method: human bone marrow-derived MSCs were cultured in culture vessels with growth media to expand the MSC population. Growth media was then removed, and the cells were washed with PBS. The MSCs were then cultured at a pH below 7.0, less than 5% oxygen (e.g., to a final concentration of about 1% oxygen), and in a culture media comprising basal media and sodium chloride. The culture media comprising the MSC secretome was formulated into an isotonic infusion solution, such as sodium chloride, and filter sterilized. The production process for the IMP was done under current Good Manufacturing Practices and Current Good Tissue Practices.

The IMP was manufactured from the banked hBM-MSCs of a single donor under CGMP conditions and according to FDA Master File protocols. Each lot of the IMP was characterized by proteomic and miRNA characterization. Additionally, the size and quantity of EVs and the presence of a specific surface marker expression profile were confirmed.

The IMP comprises extracellular vesicles (EVs) that are acellular and nonimmunogenic, containing no nucleus or deoxyribonucleic acid (DNA).

The tetraspanin profile of extracellular vesicles present in the IMP was determined, and it was found that greater than 95% of the extracellular vesicles present in the composition were CD63⁺ CD9⁻ CD81⁻. The EVs were measured via Nanoparticle tracking analysis (NTA), having a median diameter of about 100 nm. At least 10 billion EVs per mL were calculated using NTA with fluorescent staining of EV membranes.

Protein content of the IMP was determined, and the following proteins were found to be present. Proteins were detected using an antibody-based sandwich ELISAs or by Luminex or Nanoview/Unchained Labs-based methods. The total concentration of certain proteins in the IMP was measured via ELISA, with each protein having a concentration of about 200 pg/mL to about 80 ng/mL. For instance, TIMP1, OPN, IGFBP4, and osteonectin were characterized. Non-limiting examples of proteins present in the IMP are shown in Table 2 below.

TABLE 2

| Protein ID | Full Name |
|---|---|
| Ferritin | Ferritin |
| IGFBP-4 | Insulin-like growth factor binding protein-4 |
| IL-1 R6 | Interleukin 1 Receptor 6 |
| LAMP2 | Lysosome-associated membrane glycoprotein 2 |
| bIG-H3 | Transforming growth factor-beta-induced protein ig-h3 |
| GPR115 | Adhesion G protein-coupled receptor F4 |
| CD63 | CD63 antigen |
| CD109 | CD109 Antigen |
| Serpin F1 | Pigment epithelium-derived factor |
| IGFBP-6 | Insulin-like growth factor binding protein-6 |
| HS3ST4 | Heparan sulfate glucosamine 3-O-sulfotransferase 4 |
| OPN | Osteopontin |
| PAI-1 | Plasminogen activator inhibitor-1 or SERPINE 1 |
| Cathepsin B | Cathepsin B |
| IGFBP-2 | Insulin-like growth factor binding protein-2 |
| Semaphorin 6C | Semaphorin 6C |
| IGF-2 | Insulin-like growth factor-2 |
| Sortilin | Sortilin |
| Serpin B6 | Serpin B6 |
| Dkk-3 | Dickkopf-related protein 3 |
| CNTF | Ciliary neurotrophic factor |
| TSP-1 | Thrombospondin 1 |
| GM-CSF Ra | Granulocyte-macrophage colony-stimulating |
| | factor receptor subunit alpha |
| Thrombomodulin | Thrombomodulin |
| Endoglycan | AKA podocalyxin-like protein 2 |
| IGFBP-3 | Insulin-like binding protein-3 |
| RGM-C | Hemojuvelin |
| PF4 | Platelet Factor 4 |
| MIF | Macrophage migration inhibitory factor |
| TGM4 | Protein-glutamine gamma-glutamyltransferase 4 |
| Periostin | Periostin |
| Furin | Furin |
| TIMP-1 | Tissue inhibitor of MMPs 1 |
| Decorin | Decorin |
| PCK1 | Phosphoenolpyruvate carboxykinase, cytosolic |
| CD9 | CD9 antigen |
| CD99 | CD99 antigen |
| CA2 | Carbonic anhydrase 2 |
| PRDX4 | Peroxidredoxin-4 |
| Transferrin | Transferrin |
| DcR3 | Tumor necrosis factor receptor superfamily |
| | member 6B |
| GP73 | Golgi membrane protein 1 |
| CD81 | CD81 antigen |
| Lumican | Lumican |
| TIMP-2 | Tissue Inhibitor of MMPs 2 |

The nucleic acid content of the IMP was determined, and the following nucleic acids were found to be present: hsa-miR-125b-5p, hsa-miR-145-5p, hsa-miR-191-5p, hsa-miR-199a-3p, hsa-miR-21-5p, hsa-miR-221-3p, hsa-miR-222-3p, hsa-miR-22-3p, hsa-miR-23a-3p, hsa-miR-23b-3p, hsa-miR-27a-3p, hsa-miR-27b-3p, hsa-miR-29a-3p, hsa-miR-29c-3p, hsa-miR-31-5p, hsa-miR-320a, hsa-miR-34a-5p, hsa-miR-423-3p, hsa-miR-424-5p, and hsa-miR-940. RNA were measured using semi-quantitative PCR. Total RNA was analyzed by UV detection using a ClarioStar Plus microplate reader and BMG Labtech LVis plate adapter for nucleic acid quantification. Using this method, the amount of each RNA may be less than the limit of detection, or about 50 ng/mL.

### Example 2—Treatment of Acute Respiratory Distress Syndrome (ARDS)

This is a randomized, double blind, placebo-controlled trial to determine the safety and efficacy of IV administration of the composition of Example 1 (investigational medicinal product or “IMP”), versus placebo for the treatment of hospitalized patients with moderate-to-severe ARDS. The parallel group design allows a comparison of the IMP versus standard of care. Approximately 970 participants will be randomized in the trial at global sites. Potential participants will be screened within seven days prior to Day 1. Eligible participants will be randomized on Day 1 and receive IV IMP or Placebo on Days 1, 3 and 5 (Day 1 is the first calendar day, from midnight to the following midnight, of treatment with IMP or Placebo). Participants will be followed for 59 days after randomization for a total duration of 60 days. Participants will be randomly assigned to either treatment arm: Treatment Arm 1: Placebo (100 mL 0.9% NaCl, IV), and Treatment Arm 2: IMP (15 mL IMP in 85 mL of 0.9% NaCl, IV). The treatment is administered as an IV over 60 minutes on Day 1 via a central or peripheral line, and repeated on Days 3 and 5.

### Subject Inclusion and Exclusion Criteria

### Inclusion Criteria

1) Written informed consent from participant or participant's legally authorized representative (LAR)
2) Adults aged 18 years or older
3) Presence of the following criteria for moderate to severe ARDS within 48 hours prior to the first infusion:
   Acute (within 7 days) onset of dyspnea or worsening of hypoxemic respiratory failure following a predisposing risk factor (pneumonia, nonpulmonary infection, trauma, transfusion, aspiration, or shock)
   Chest imaging (Computed Tomography (CT), Chest X-Ray (CXR), not including ultrasound) with bilateral opacities—not fully explained by effusions, atelectasis, or nodules
   PaO2/FiO2 (P/F ratio)≤200 mm Hg
   Invasive or noninvasive mechanical ventilation (MV) with a minimum Positive End Expiratory Pressure (PEEP) 5 cm H2O or minimum of continuous positive airway pressure (CPAP) 5 cm H2O, or HFNO at ≥30 L/min, and
   Respiratory failure not fully explained by cardiac failure or fluid overload.

The 72 hour enrollment time window begins when all of the above inclusion criteria are met. Criteria may be met at either the ICU or referring hospital. The first qualifying PaO2/FiO2 is used to determine this 72 hour window.

### Exclusion Criteria

Greater than 72 hours since ARDS onset; or
Lack of informed consent; or
Pregnant woman, woman of childbearing potential without a documented negative urine or serum pregnancy test during the current hospitalization, or woman who is breast feeding; or
Receiving extracorporeal membrane oxygenation (ECMO) therapy during the 72 hour eligibility window; or
Home oxygen therapy at >3 liters per minute or MV (non-invasive ventilation or via tracheotomy) except for CPAP/BiPAP used solely for sleep-disordered breathing; or
Actual body weight exceeding 1 kg per centimeter of height; or
Severe chronic liver disease defined as a Child-Pugh score of 12-15; or
Active malignancy requiring treatment in the past 2 years; or
Bone marrow transplantation within the last 1 year; or
Expected duration of MV <48 hours; or
Decision to withhold life-sustaining treatment; except in those participants committed to full support except cardiopulmonary resuscitation; or
Moribund participant not expected to survive 24 hours or treatment withdrawal imminent; or
Diffuse alveolar hemorrhage from vasculitis; or
Burns involving >70% total body surface; or
ARDS due primarily to trauma; or
Neuromuscular conditions that may impair neuromuscular function and/or impair spontaneous ventilation; or
Neurologic conditions undergoing treatment for intracranial hypertension; or
PaO2/FiO2 (if available) >200 after meeting inclusion criteria and before randomization.
Use of investigational therapy or treatment within 30 days prior to the initial infusion.

### Endpoints

Primary endpoint: 60-day all-cause mortality.

Secondary endpoints:
Time to death within 60 days
Ventilator free days at Day 28: Ventilator-free Days within 28 days is the sum of days for which participants are not on mechanical ventilation to 28 days. VFDs will begin to accrue on the first calendar day (midnight to midnight) the participant remains free of mechanical ventilation to Day 28. VFDs will be summarized by randomized treatment arm and compared (Control vs IMP 15 ml) using a general linear model. Participants who die prior to Day 28 will be considered as on mechanical ventilation on and after the date of death up to Day 28 (e.g. assigned zero VFDs). If a participant receives non-invasive MV solely for sleep disordered breathing, those days will be counted as ventilator free.
Oxygen free days at Day 28: Oxygen free Days within 28 days is the sum of days for which participants are not receiving any types of oxygen support within 28 days of the follow-up. The result will be summarized by randomized treatment arm and compared (control vs IMP 15 ml) using a general linear model. Participants who die prior to Day 28 will be considered as on Oxygen Support on and after the date of death up to Day 28.
Intensive care unit (ICU) free days at Day 28: ICU free days within 28 days is the sum of days for which participants are no longer in the ICU within 28 days of the follow-up. The result will be summarized by randomized treatment arm and compared (control vs IMP 15 ml) using a general linear model. Participants who die prior to Day 28 will be considered as in the ICU on and after the date of death up to Day 28.
Initiation of new invasive mechanical ventilation to 28 days: Participants enrolled on HFNO will be assessed for this endpoint. The proportion of participants who are intubated and treated with new MV (defined new invasive MV) will be to Day 28. If a participant receives non-invasive MV for any indication, this will not count as invasive MV. The result will be summarized by randomized treatment arm and compared (control vs IMP 15 ml) using a general linear model.
Incidence of TESAEs (Treatment-Emergent Serious Adverse Event): Incidence rates of TESAEs will be measured as the binomial proportion of participants who experienced a TESAE within 60 days from the study infusion and will be estimated for each treatment arm as participants received.
Change in biomarkers from baseline: C-reactive protein (CRP), Plasminogen Activator Inhibitor-1 (PAI-1), Interleukin-8 (IL-8), Interleukin-6 (IL-6) and Interleukin-I1β (IL-1β) from screening to Days 1, 3, and 5
Change in Sequential Organ Failure Assessment (SOFA) from baseline (defined as the highest score in the 24 hours prior to IMP administration) to Day 14 to Day 28 in participants who are hospitalized
Evaluation of efficacy in ARDS subtypes (hyperinflammatory and hypoinflammatory, identified with prerandomization serum bicarbonate, sTNFR1, and IL-6)
Improvement in partial pressure of arterial oxygen to fraction of inspired oxygen (PaO2/FiO2) ratio from pre-infusion baseline to Day 7; partial pressure of arterial oxygen (PaO2) must be obtained from arterial blood gas (ABG); FiO2 will be the set FiO2 on the invasive or noninvasive mechanical ventilator or the set FiO2 for high flow nasal oxygen (HFNO) at flow rates ≥30 liters per minute (L/min)

### Example 3—Exosome Derived from Bone Marrow Mesenchymal Stem Cell as Treatment for Severe COVID-19

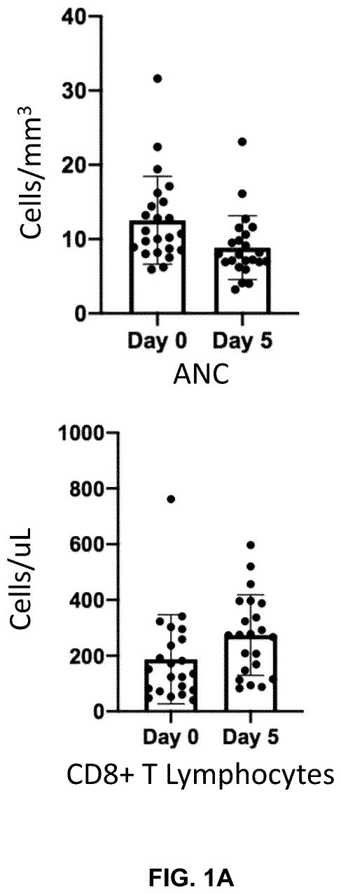

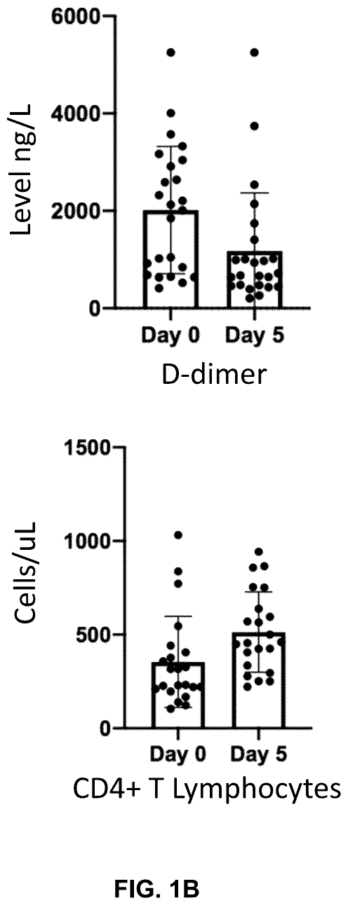

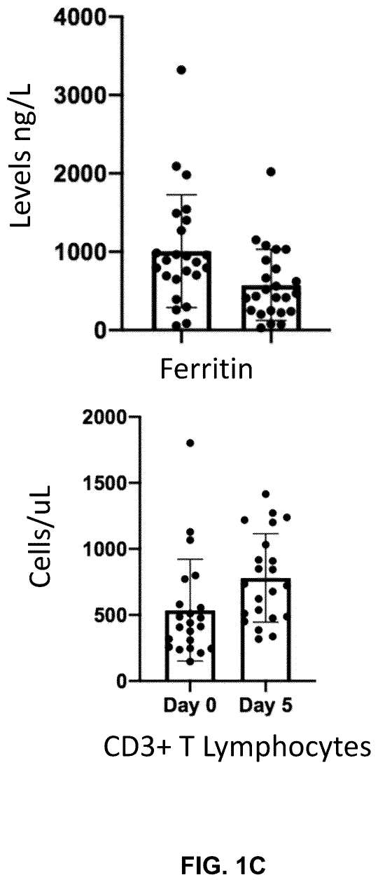

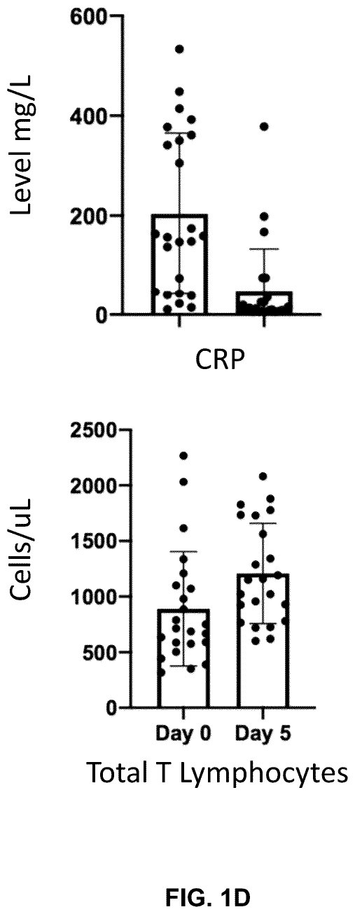

An investigator-initiated, prospective non-randomized open-label cohort study to address the safety and efficacy of allogeneic bone marrow mesenchymal stem cell secretome (investigational medicinal product or “IMP” as described in Example 1) as treatment for severe COVID-19 ARDS was performed under hospital IRB approval with a patient group of 24 (n=24). Acute phase reactants and lymphocyte numbers were evaluated at Day 0 and at Day 5 and the results are shown below and in FIGS. 1A-D.

Acute phase reactants (CRP, ferritin, and D-dimer)

$$C - Reactive Protein ( CRP ) = 77 \% reduction ( p < 0.001 )$$

$$Ferritin = 43 \% reduction ( p < 0.001 )$$

$$D - Dimer = 42 \% reduction ( p < 0.05 )$$

$$Absolute Nuetrophil ( ANC ) = 32 \% reduction ( p < 0.001 )$$

### Immune Cell Populations

$$Total Lymphocytes = 36 \% increase ( P < 0.05 )$$

$$CD 3 + T Lymphocytes = 46 \% increase ( p < 0.05 )$$

$$CD 4 + T Lymphocytes = 45 \% increase ( p < 0.05 )$$

$$CD 8 + T Lymphocytes = 46 \% increase ( p < 0.001 )$$

### Example 4—Bone Marrow Mesenchymal Stem Cell Derived Extracellular Vesicle Infusion as Treatment of Respiratory Failure from COVID-19

A double-blind, placebo controlled, dosing clinical trial determined that two BM-MSC derived EV (investigational medicinal product or “IMP” as described in Example 1) treatments of 2.4 g (15 mL), delivered by intravenous infusion on day 1 and day 4, were safe and able to reduce mortality in moderate to severe COVID-19 induced ARDS patients compared to placebo. No treatment-related adverse events were reported. Mortality (60-day) in the intention-to-treat population trended to be less using 15 mL IMP mixed with 85 mL normal saline compared with placebo (p=0.134).

Post-hoc subgroup analyses for all participants aged 18 to 65 years' 60-day mortality was decreased with treatment using 15 mL IMP mixed with 85 mL normal saline compared with placebo (relative risk, 0.39; p=0.034; n=50). With 15 mL IMP mixed with 85 mL normal saline treatment, a relative risk of 0.423 (p=0.059; n=24) was determined for participants with moderate to severe ARDS. Ventilation-free days improved in that cohort by 7-days with 15 mL IMP mixed with 85 mL normal saline treatment (p=0.046; n=50).

### Example 5—Safety and Efficacy Study

The clinical safety and efficacy of an advanced bone marrow mesenchymal stem cell (BM-MSC)-derived extracellular vesicle (EV) investigational medicinal product (IMP) as described in Example 1 was evaluated in a multi-site, prospective, expanded access trial as potential treatment of hospitalized patients with respiratory failure from severe or critical COVID-19, regardless of severity of ARDS (mild, moderate, severe, need for ECMO). BM-MSC derived EV preparation exhibits immunomodulatory properties to reset and redirect inflammation as well as regenerative properties to restore lung tissue structure and function.

### Summary

Subjects (n=103) received up to three doses of 15 mL IMP every 72 hours. The primary outcome was all-cause 60-day mortality. Secondary outcomes included serious adverse events. One Treatment-Emergent Adverse Events (TEAE; grade 1 hyperpigmentation at the infusion site) related to IMP occurred. 60-day mortality was 22.4% in patients <65 years and 41.7% in patients ≥65 years. Mean ventilation free days was 44.7 (SD: 24.1) days in patients <65 years and 33.4 (26.3) days in patients ≥65 years. Median time to hospital discharge by Kaplan-Meier was 9 (5.0—NR) days in patients <65 years and 19 (5.5—NR) days in patients ≥65 years. The IMP (15 mL dose up to 3 times) is safe in patients with severe or critical COVID-19 respiratory failure.

### Experimental Procedures

### Design

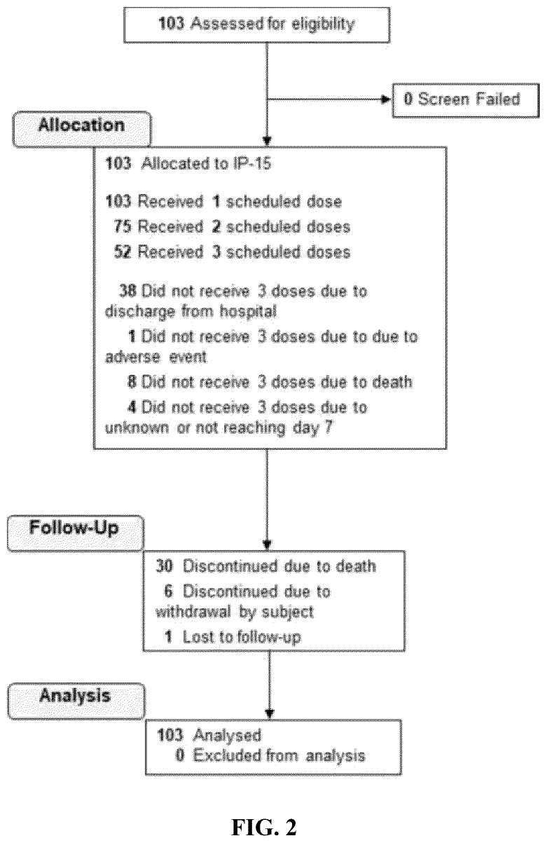

A prospective, multi-center, expanded access trial was conducted. Five clinical trial sites in the United States actively participated in patient recruitment and enrollment. Patients with severe or critical COVID-19 who met modified Berlin criteria for acute respiratory distress syndrome (ARDS) as defined by onset of symptoms within one week of insult, respiratory failure not fully explained by cardiac failure or fluid overload, PaO₂/FiO₂≤200, PEEP >5 cm H₂O, and bilateral opacities not fully explained by effusions or lung collapse were offered enrollment (see Inclusion and Exclusion Criteria below). See FIG. 2 for Consolidated Standards of Reporting Trials (CONSORT) diagram for patient screening and enrollment.

### Inclusion Criteria

1. Provision of informed consent by self or proxy.
2. Stated willingness to comply with study protocol.
3. Male or female of any age ≥18 years of age
4. May be pregnant unless the patient has one or more conditions listed under Exclusion Criteria #4.
5. Positive Reverse Transcriptase Polymerase Chain Reaction (RT-PCR) SARS-CoV-2.
6. Moderate to severe ARDS as defined by timing within 1 week of known clinical insult or new or worsening respiratory symptoms, bilateral opacities not fully explained by effusions or lung collapse, and respiratory failure not fully explained by cardiac failure or fluid overload, and PaO₂/FiO₂≤200 mmHg.
7. Acute presentation of hypoxic respiratory failure requiring noninvasive oxygen support OR mechanical ventilation (MV).
8. Agreement to use highly effective birth control contraception if of reproductive age and potential.

### Exclusion Criteria

1. Active malignancy requiring treatment within the last five years.
2. Patients who are not full code.
3. Pregnant patients with current or past history of eclampsia, preeclampsia, hemolysis, elevated liver enzymes, low platelet count (HELLP) syndrome during pregnancy.
4. New York Heart Association (NYHA) Functional Class III (symptoms present during ordinary activities) or IV Heart Failure (symptoms present at rest) or listed for heart transplant.
5. Chronic Kidney Disease (CKD) Stage IV (GFR 15-29 mL/min/1.73 m²) and Stage V (GFR <15 mL/min/1.73 m²) or listed for kidney transplant.
6. Hepatic Impairment with Model for End-Stage Liver Disease (MELD) score ≥30 or listed for liver transplant.
7. Patients on Extracorporeal Membrane Oxygenation.

Each lot of the IMP met stringent release specifications, including proteomic, mRNA and miRNA characterization as previously described in greater detail. Briefly, the IMP was manufactured per FDA guidance for Current Good Manufacturing Practices (cGMP) with all manufacturing processes controlled under a Quality Management System, and implementation of lot-specific master batch records and specified release criteria for each lot of IP. The size and quantity of EVs and the presence of a specific surface marker expression profile were confirmed. Identity assays were combined with validated potency assays to demonstrate the mechanism of action is functional. A 15 mL dose provides 1.2×10¹² EV particles. The IMP contains the proteins and miRNA as described in Example 1.

Patients (n=103) were enrolled in the open label prospective study described herein. After informed consent, patients received a 100 mL intravenous infusion over 60 minutes: 15 mL IMP with 85 mL normal saline (NS). A repeat of the same study treatment occurred on Day 4 and Day 7 if the patient had not recovered (SpO₂ ≥93% on room air or PaO₂/FiO₂ ≥300 mmHg). All patients were followed for 60 days, or until hospital discharge or death. The trial protocol was approved by the institutional review board (IRB) at each site (or a centralized IRB as applicable) and overseen by an independent data and safety monitoring board (DSMB). Written informed consent (or consent by other IRB-approved process) was obtained from each patient or patient's legally authorized representative if the patient was unable to provide consent.

### Objectives and Outcomes

The primary endpoint was all-cause 60-day binomial mortality. Secondary endpoints included 1) treatment-emergent (TE) serious adverse events (SAEs) defined as any SAEs starting on or after the first dose date up to 30 days after the last dose, 2) ventilation-free days (VFDs) within the first 60-day follow-up, and 3) time to hospital discharge. Exploratory outcome measurements for acute phase reactants including C-Reactive Protein (CRP), D-dimer and ferritin and immune cell subset counts measured up to Day 61, Sequential Organ Failure Assessment (SOFA) scores measured as change from baseline to Day 15, and PaO₂/FiO₂ change from baseline to Day 7.

### Measures

Patients were assessed daily from Day 1 to Day 60 during hospitalization. All treatment emergent adverse events (TEAEs), serious adverse events (SAEs) and grade 3 or 4 adverse events (AEs) representing increased severity from Day 1, and any grade suspected drug-related hypersensitivity reactions were recorded.

### Statistical Analysis

This study used all treated participants who received at least a partial dose of the IMP treatment regimen for efficacy endpoints. Safety analysis was also conducted on participants who had any exposure to IP. Sub-group analysis of 60-day all-cause mortality was performed on age groups, to investigate whether treatment with IMP had more of an effect on outcome in specific patient groups. Missing data was not imputed, and the results were summarized based on available data.

### Results

### Trial Participants

A total of 103 patients were enrolled and treated (Table 3). Patients were evenly balanced by gender and 35% were above age 65. A majority of patients was white (75%), and the mean body mass index (BMI) was 34.1 (SD: 9.0). Screening exams were performed at a mean (SD) of 1.5 (0.56) days prior to the first infusion and ranged from 1 to 4 days. The mean time between COVID diagnosis and first infusion was 8.5 (7.8) days with a range of 1 to 50 days. Over 50% had received prior remdesivir treatment while 95% had received prior dexamethasone treatment. Of the 103 patients, 110% were on mechanical ventilation at the time of first IMP infusion. Many patients were maximized on high flow nasal cannula in an attempt to achieve non mechanical ventilatory oxygen support. Thus, there were several patients in the cohort that did not yet need mechanical ventilation. However, many patients had comorbidities that increased the SOFA score regardless. Proportions of all treated subjects who received any amount of 1, 2, and 3 doses are 100%, 73%, and 51%, respectively (Table 4). Median time from dose 1 to doses 2 and 3 are 2.9 days (Interquartile range (IQR): 2.80-3.04) and 6.0 days (5.84-6.03), respectively, indicating high compliance with the per-protocol dose schedule.

TABLE 3
Demographics and Baseline Characteristics (Safety Analysis Set)

| | | (IMP 15 mL up |
|---|---|---|
| | | to 3 Doses) |
| | Statistics | (N = 103) |
| Age | n | 103 |
| | Mean (SD) | 56.7 (16.42) |
| | Min, Max | 22, 90 |
| Age ≥ 65 | n (%) | 36 (35.0) |
| Age < 65 | n (%) | 67 (65.0) |
| Gender | | |
| Male | n (%) | 58 (56.3) |
| Female | n (%) | 45 (43.7) |
| Race | | |
| American Indian or Alaska Native | n (%) | 0 |
| Asian | n (%) | 8 (7.8) |
| Black or African American | n (%) | 13 (12.6) |
| Native Hawaiian or Other Pacific | n (%) | 0 |
| Islander | | |
| White | n (%) | 77 (74.8) |
| Unknown or Other | n (%) | 5 (4.9) |
| BMI (kg/m²)^{a} | n | 102 |
| | Mean (SD) | 34.147 (8.9523) |
| | Min, Max | 18.16, 66.76 |
| Respiratory Rate (breaths/min)^{a} | n | 103 |
| | Mean (SD) | 25.0 (7.23) |
| | Min, Max | 11, 46 |
| Time from the First Covid-19 | n | 103 |
| Diagnosis to First | Mean (SD) | 8.5 (7.80) |
| IMP Dose Date (days) | Min, Max | 1, 50 |
| Time from Screening Visit to | n | 103 |
| First IMP Dose Date (days) | Mean (SD) | 1.5 (0.56) |
| | Min, Max | 1, 4 |
| Total SOFA Score^{a} | n | 103 |
| | Mean (SD) | 3.2 (1.96) |
| | Min, Max | 2, 12 |
| P/F Ratio (mmHg)^{a} | n | 99 |
| | Mean (SD) | 118.454 (50.9406) |
| | Min, Max | 45, 245 |
| Prior Therapy^{b} | n | 98 |
| Remdesivir | n (%) | 57 (55.3) |
| Plasma | n (%) | 0 |
| Dexamethasone | n (%) | 98 (95.1) |
| Mechanical Ventilation | | |
| Intubated | n (%) | 11 (10.7) |
| Non-Intubated^{c} | n (%) | 92 (89.3) |
| Intubated (MV) on Study | n | 19 |
| Time on Intubation (MV) (days) | Median | 9.0 |
| | (1st, 3^{rd} | (3.0, 17.0) |
| | Quartiles) | |
| | Min, Max | 1, 37 |

^{a}Baseline is the last measure prior to the first dose of IMP (Day 0 or Day 15 min before dosing).
^{b}If the agent started prior to the first dose of IMP regardless of its end date.
^{c}All patients that were not intubated were on non-invasive ventilation of Bilevel positive airway pressure (BiPAP) or continuous positive airway pressure (CPAP) with a minimum of 5 L 02/min.

TABLE 4
IMP Administration and Disposition (Safety Analysis Set)

| | | (IMP 15 mL up |
|---|---|---|
| | | to 3 Doses) |
| | Statistics | (N = 103) |
| IMP 15 mL | | |
| Received 1 Dose | n (%) | 103 (100) |
| Received 2 Doses | n (%) | 75 (72.8) |
| Received 3 Doses | n (%) | 52 (50.5) |
| | Median | 2.13 |
| Days from Date of Hospitalization | (1st, 3^{rd} | (1.31, 5.19) |
| to Dose 1 (n = 103) | Quartiles) | |
| | Min, Max | 0.4, 42.5 |
| Days from Dose 1 to Dose 2 (n = 75) | Median | 2.94 |
| | (1st, 3^{rd} | (2.80, 3.04) |
| | Quartiles) | |
| | Min, Max | 2.6, 3.7 |
| Days from Dose 1 to Dose 3 (n = 52) | Median | 5.95 |
| | (1st, 3^{rd} | (5.84, 6.03) |
| | Quartiles) | |
| | Min, Max | 5.7, 7.0 |
| #Subjects Who Did Not Receive A Full | n (%) | 1 (1.0) |
| Dose at Any Dose (Dose 1, 2, or 3) | | |
| Reason for Not Receiving All 3 Doses* | n (%) | 51 (49.5) |
| Discharge from the Hospital | n (%) | 38 (36.9) |
| Adverse Event | n (%) | 1 (1.0) |
| Death | n (%) | 8 (7.8) |
| Unknown or Not Reaching Day 7 | n (%) | 4 (3.9) |
| Subjects Who Completed Study | n (%) | 63 (61.2) |
| Subjects Who Are Being Followed on | n (%) | 5 (4.9) |
| Study | | |
| Reason for Discontinuation from Study | n (%) | 35 (34.0) |
| Early | | |
| AE | n (%) | 0 |
| SAE | n (%) | 0 |
| Death | n (%) | 28 (27.2) |
| Lost to Follow-up | n (%) | 1 (1.0) |
| Physician Decision | n (%) | 0 |
| Protocol Deviation | n (%) | 0 |
| Withdrawal by Subject | n (%) | 6 (5.8) |
| Other | n (%) | 0 |

*Categorize the reason by the order: 1. Discharge occurred prior to the third dose, 2. AE.AEACN = ‘Study Drug Withdrawn’ occurring prior to the third dose, 3. Death prior to the third dose, 4. None of the above, but did not receive 3 doses.

### Safety

The Safety Analysis Data Set (Table 5) consisted of all 103 enrolled subjects who received any dose of IP. No AEs or SAEs caused a pause in patient recruitment or clinical trial discontinuation. No infusion reaction or AEs were observed within the first 72 hours. Treatment-Emergent Adverse Events (TEAEs) and serious TEAEs ofany grade occurred, but only one TEAE was determined to be related to IP. This was hyperpigmentation at the infusion site and graded as a Grade.

TABLE 5
Overall Summary of Safety (Safety Analysis Set)

| | (IMP 15 mL up to 3 Doses) |
|---|---|
| | (N = 103) |
| Safety Parameter | n (%) |
| Any TEAEs^{a} | |
| Any Grade | 76 (73.8) |
| Grade 3 or 4 | 14 (13.6) |
| Serious TEAEs^{a} | |
| Any Grade | 41 (39.8) |
| Grade 3 or 4 | 13 (12.6) |
| IP-Related TEAEs | 1 (1.0) |
| IP-Related Serious TEAEs | 0 |
| TEAEs That Led to Dose Interruption | 2 (1.9) |
| TEAEs That Led to Missing Dose or | 1 (1.0) |
| Discontinued the Treatment Early | |
| TEAEs That Led to Death | 28 (27.2) |
| IMP Infusion Reaction | 0 |

TEAE = Treatment-Emergent Adverse Events, are defined as any adverse event that started between the first dose date and 30 days post the last dose date, inclusively.
^{a}Toxicity grades of adverse events are evaluated based on criteria of NCI-CTCAE v5.0. Each subject is counted once to the worst grade at subject-level.
Note:
Related = Definitely, Probably, or Potentially Related.

The relationship between safety events and the number of doses of IMP received was analyzed in the Safety Analysis. As shown in Table 6, no dose-response relationship amongst the percentages of patients experiencing Grade-3 or Grade-4 safety events was observed. When the duration of hospitalization was controlled to those subjects who either died or were hospitalized until day 10, the percentage of subjects experiencing safety events are also evenly distributed among the three dosage groups (Table 7).

TABLE 6
Overall Summary of Safety by Number of
Doses Received (Safety Analysis Set)

| | (IMP 15 mL up to 3 Doses) | | |
|---|---|---|---|
| | Received | Received | Received |
| | 1 Dose | 2 Doses | 3 Doses |
| | (N = 28) | (N = 23) | (N = 52) |
| Safety Parameter | n (%) | n (%) | n (%) |
| Any TEAEs^{a} | | | |
| Any Grade | 18 (64.3) | 15 (65.2) | 43 (82.7) |
| Grade 3 or 4 | 5 (17.9) | 2 (8.7) | 7 (13.5) |
| Serious TEAEs^{a} | | | |
| Any Grade | 7 (25.0) | 9 (39.1) | 25 (48.1) |
| Grade 3 or 4 | 5 (17.9) | 2 (8.7) | 6 (11.5) |
| IMP-Related TEAEs | 0 | 1 (4.3) | 0 |
| IMP-Related Serious TEAEs | 0 | 0 | 0 |
| TEAEs That Led to Dose | 0 | 1 (4.3) | 1 (1.9) |
| Interruption | | | |
| TEAEs That Led to Missing | 0 | 1 (4.3) | 0 |
| Dose or Discontinued the | | | |
| Treatment Early | | | |
| TEAEs That Led to Death | 2 (7.1) | 7 (30.4) | 19 (36.5) |
| IMP Infusion Reaction | 0 | 0 | 0 |

TEAE = Treatment-Emergent Adverse Events, are defined as any adverse event that started between the first dose date and 30 days post the last dose date, inclusively.
^{a}Toxicity grades of adverse events are evaluated based on criteria of NCI-CTCAE v5.0. Each subject is counted once to the worst grade at subject-level.
Note:
Related = Definitely, Probably, or Potentially Related.

TABLE 7
Overall Summary of Safety by Number of Doses
Received (Safety Analysis Set - Subjects
Who Died or Were Hospitalized up to Day 10)

| | (IMP 15 mL up to 3 Doses) | | |
|---|---|---|---|
| | Received | Received | Received |
| | 1 Dose | 2 Doses | 3 Doses |
| | (N = 5) | (N = 7) | (N = 40) |
| Safety Parameter | n (%) | n (%) | n (%) |
| Any TEAEs^{a} | | | |
| Any Grade | 5 (100) | 7 (100) | 35 (87.5) |
| Grade 3 or 4 | 2 (40.0) | 0 | 6 (15.0) |
| Serious TEAEs^{a} | | | |
| Any Grade | 4 (80.0) | 7 (100) | 24 (60.0) |
| Grade 3 or 4 | 2 (40.0) | 0 | 5 (12.5) |
| IMP-Related TEAEs | 0 | 0 | 0 |
| IMP-Related Serious TEAEs | 0 | 0 | 0 |
| TEAEs That Led to Dose | 0 | 1 (14.3) | 1 (2.5) |
| Interruption | | | |
| TEAEs That Led to Missing | 0 | 1 (14.3) | 0 |
| Dose or Discontinued the | | | |
| Treatment Early | | | |
| TEAEs That Led to Death | 2 (40.0) | 7 (100) | 19 (47.5) |
| IMP Infusion Reaction | 0 | 0 | 0 |

TEAE = Treatment-Emergent Adverse Events, are defined as any adverse event that started between the first dose date and 30 days post the last dose date, inclusively.
^{a}Toxicity grades of adverse events are evaluated based on criteria of NCI-CTCAE v5.0. Each subject is counted once to the worst grade at subject-level.
Note:
Related = Definitely, Probably, or Potentially Related.

### Efficacy Parameters

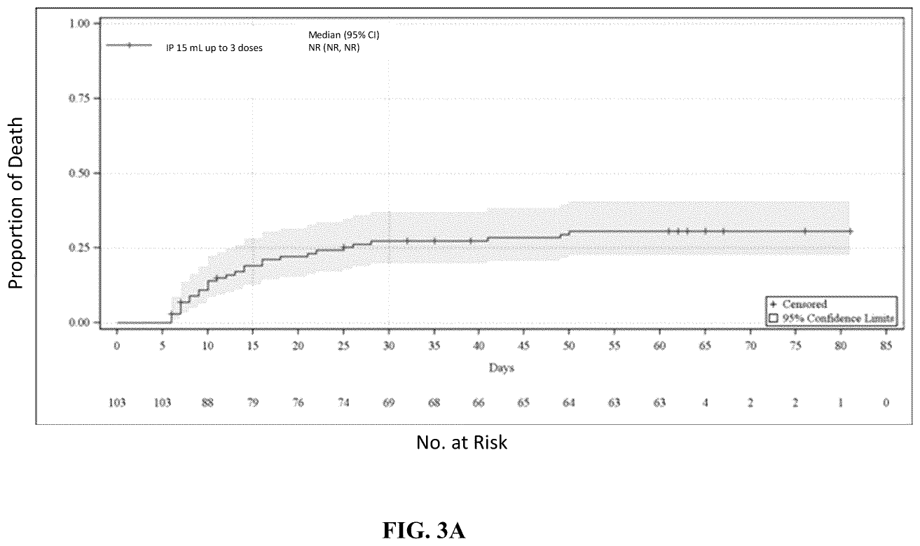

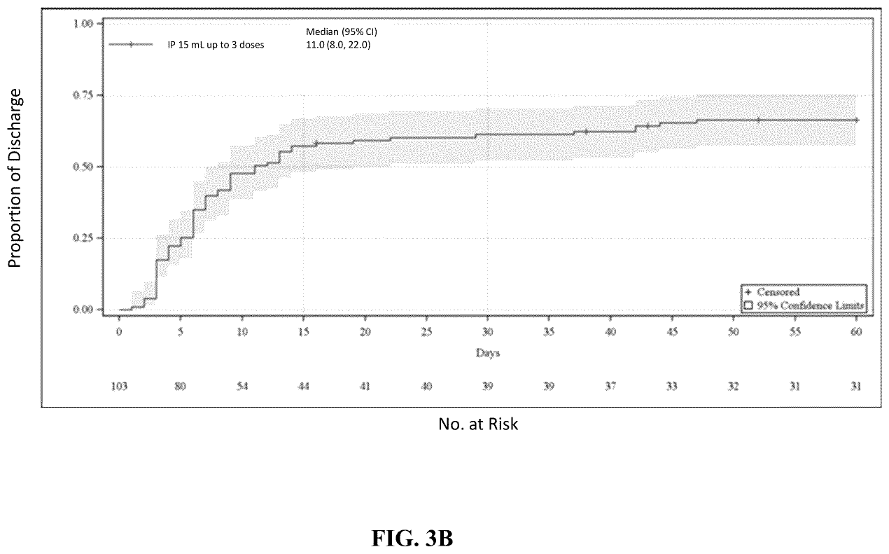

The overall mortality (OS) among all patients was 29% (Table 8). The 60-day mortality was 22% in patients <65 years and 42% in those ≥65 years (Table 9). Median time to death was not reached (NR) (Kaplan-Meier (KM), FIG. 3A) and the mortality rate was 30.6% at 60 days for all treated patients; patients <65 years had an estimated mortality rate of 23.8% versus 43.1% in aged ≥65 years. Median (IQR) time to discharge calculated by KMN method (FIG. 3B) was 11 (5.0—NR) days overall; for those age <65 years it was 9 days (5.0—NR) and for the ≥65 year-old group it was 19 days (5.5—NR).

Mean (SD) of VFDs was 40.8 (25.4) days out of the 60-day follow-up overall (Table 8), and for patients <65 and ≥65 years it was 44.7 (24.1) and 33.4 (26.3) days, respectively (Table 9). The mean (SD) increase in PaO₂/FiO₂ ratio from baseline to day 7 for the Intention-to-Treat (ITT) population was 115 (125.2) mmHg (Table 8). Unlike the other metrics, the mean PaO₂/FiO₂ increase over seven days was nearly identical between the two age groups, with those <65 achieving 116 mmHg improvement and the ≥65-year group achieving 114 mmHg (Table 9).

Among 92 treated subjects who were not intubated at baseline, 19 (20.7%) subjects received mechanical ventilation (MV) with median (IQR) of 7 (3-11) days to require MV. And these patients were on MV for a median duration of 9(3-17) days.

TABLE 8
Summary of Efficacy (Full Analysis Set)

| | | (IMP 15 mL | |
|---|---|---|---|
| | | up to 3 Doses) | |
| Study Endpoints | Statistics | (N = 103) | |
| Subjects Who Discharged from | n (%) | 68 | (66.0) |
| Hospital | | | |
| Time to Discharge (KM) | Median | 11.0 days | |
| | (1st, 3rd | (5.0, NR) | |
| | Quartiles) | | |
| Mean Time to Discharge | n | 68 | |
| (Restricted to Discharged | Mean (SD) | 9.9 days | (10.53) |
| Subjects) | Min, Max | 1, 47 | |
| Subjects Who Died Within 30 | n (%) | 27 | (26.2) |
| Subjects Who Died Within 60 | n (%) | 30 | (29.1) |
| Days | 95% CI | 20.6, 38.9 | |
| | (exact) | | |
| Median Time to Death (KM) | Median | NR | |
| Mortality Rate at 15 Days (KM) | % (95% CI) | 19.1 | (12.6, 28.3) |
| Mortality Rate at 30 Days (KM) | % (95% CI) | 27.3 | (19.6, 37.3) |
| Mortality Rate at 60 Days (KM) | % (95% CI) | 30.6 | (22.5, 40.8) |
| Mean Time to Death (Restricted | n | 30 | |
| to Subjects Who Died) | Mean (SD) | 16.2 days | (12.14) |
| | Min, Max | 6, 50 | |
| ^{a}PaO2/FiO2 Ratio Increase from | n | 97 | |
| Baseline to Day 7 (mmHg) | Mean (SD) | 115.149 | (125.2062) |
| | Min, Max | 0, 617.11 | |
| ^{b}Ventilation-Free Days (within | n | 103 | |
| 60 Days) | Mean (SD) | 40.8 | (25.36) |
| | Min, Max | 0, 61 | |
| Day 15 SOFA Score Change | n | 29 | |
| from Baseline | Mean (SD) | 1.2 | (4.24) |
| | Min, Max | −6, 9 | |
| Day 29 SOFA Score Change | n | 16 | |
| from Baseline | Mean (SD) | −0.6 | (3.42) |

| | |
|---|---|
| Min, Max | −6, 7 |

KM = Kaplan Meier method, NR = Not Reached.
^{a}PaO₂/FiO₂ ratio: All treated subjects with baseline and at least one PaO₂/FiO₂ ratio measured at Day 4 or 7. For missing Day 7 data, 380 mmHg was assigned for discharged patients, and no change (0) was assigned to patients with negative change from the baseline or died before Day 7.
^{b}Ventilation-free days: days when patients are not on mechanical ventilation within 60 days of follow-up.

TABLE 9
Summary of Efficacy by Age Group (Full Analysis Set)

| | | Age ≥ 65 | | Age < 65 | |
|---|---|---|---|---|---|
| Study Endpoints | Statistics | (N = 36) | | (N = 67) | |
| Subjects Who Discharged from | n (%) | 20 | (55.6) | 48 | (71.6) |
| Hospital | | | | | |
| Time to Discharge (KM) | Median | 19.0 days | | 9.0 days | |
| | (1st, 3rd Quartiles) | (5.5, NR) | | (5.0, NR) | |
| Mean Time to Discharge | n | 20 | | 48 | |
| (Restricted to Discharged | Mean (SD) | 10.0 days | (10.80) | 9.8 days | (10.53) |
| Subjects) | Min, Max | 3, 47 | | 1, 44 | |
| Subjects Who Died Within 30 | n (%) | 15 | (41.7) | 12 | (17.9) |
| Days | | | | | |
| Subjects Who Died Within 60 | n (%) | 15 | (41.7) | 15 | (22.4) |
| Days | 95% CI (exact) | 25.5, 59.2 | | 13.1, 34.2 | |
| Median Time to Death (KM) | Median | NR | | NR | |
| Mortality Rate at 15 Days (KM) | % (95% CI) | 37.1 | (23.5, 55.3) | 9.4 | (4.3, 19.6) |
| Mortality Rate at 30 Days (KM) | % (95% CI) | 43.1 | (28.6, 61.1) | 18.8 | (11.1, 30.7) |
| Mortality Rate at 60 Days (KM) | % (95% CI) | 43.1 | (28.6, 61.1) | 23.8 | (15.1, 36.3) |
| Mean Time to Death | n | 15 | | 15 | |
| (Restricted to Subjects | Mean (SD) | 10.5 days | (5.11) | 21.9 days | (14.51) |
| Who Died) | Min, Max | 6, 25 | | 7, 50 | |
| ^{a}PaO2/FiO2 Ratio Increase from | n | 34 | | 63 | |
| Baseline to Day 7 (mmHg) | Mean (SD) | 114.028 | (124.0509) | 115.754 | (126.8134) |
| | Min, Max | 0, 396.43 | | 0, 617.11 | |
| ^{b}Ventilation-Free Days (within | n | 36 | | 67 | |
| 60 Days) | Mean (SD) | 33.4 | (26.26) | 44.7 | (24.14) |
| | Min, Max | 0, 61 | | 0, 61 | |
| Day 15 SOFA Score Change | n | 7 | | 22 | |
| from Baseline | Mean (SD) | −2.0 | (2.94) | 2.2 | (4.12) |
| | Min, Max | −6, 3 | | −6, 9 | |
| Day 29 SOFA Score Change | n | 4 | | 12 | |
| from Baseline | Mean (SD) | −3.5 | (2.65) | 0.4 | (3.15) |

| | | |
|---|---|---|
| Min, Max | −6, 0 | −3, 7 |

KM = Kaplan Meier method, NR = Not Reached.
^{a}PaO₂/FiO₂ ratio: All treated subjects with baseline and at least one PaO₂/FiO₂ ratio measured at Day 4 or 7. For missing Day 7 data, 380 mmHg was assigned for discharged patients, and no change (0) was assigned to patients with negative change from the baseline or died before Day 7.
^{b}Ventilation-free days: days when patients are not on mechanical ventilation within 60 days of follow-up.

### Exploratory Endpoints

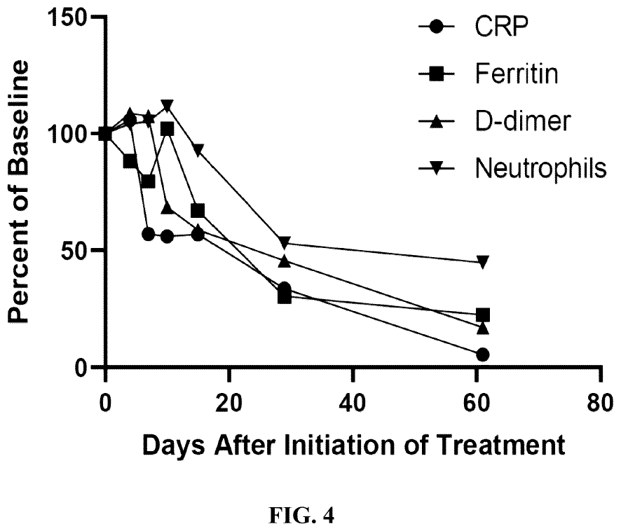

All four acute phase biomarkers declined steadily over the 61-day study with declines most pronounced from day 15 onwards (FIG. 4). The percentages of any grade TEAEs, Treatment-Emergent Serious Adverse Events (TE SAEs) or TEAEs that led to death were higher with 3 doses (Table 6).

### Discussion

At midpoint of this prospective, expanded access trial it is demonstrated that a novel biological drug candidate based on BM-MSC EV technology and in advanced stages of development is safe in the treatment of hospitalized COVID-19 patients. There was only one Grade 1 TEAE related to IMP throughout the duration of follow up for all 103 patients underscoring the safety profile in a critically ill patient population. The increased percentage of any grade TEAEs, TE SAEs and TEAEs that led to death in those receiving three doses as opposed to one or two doses was likely related to the fact that these subjects were still sick on Day 7 such that the increased any grade AEs reflected their longer hospitalization. This conclusion was supported by controlling the analysis to those subjects who died or remained hospitalized until day 10 and the observed even distribution of the subjects throughout the three dosage groups who experienced safety events. The lack of a dose-response relationship regarding safety events in either analysis argues that the IMP is safe across this dose range. These results further support that a novel technology with the potential for efficacy of intact stem cells and without the limitations of stem cells, such as the IMP studied here, can be safely administered to seriously ill COVID-19 patients.

All-cause 60-day mortality was 29.1%. In the subgroup of patients aged 18 to 65, who showed better survival in moderate to severe ARDS in the phase 2 study, mortality was 22% as compared to 42% in patients aged ≥65 in this study. This may be due to a higher occurrence of co-morbidities seen in the aging population between age 65 to 85. Alternatively, younger patients may be better able to recover from the physiologic burden of ARDS/COVID-19. Although an age difference was not apparent for the PaO₂/FiO₂ change from baseline, more VFDs and a more rapid time to discharge was evident for the age 18 to 65 population. These age-dependent differences in response to IMP suggest there may be opportunity to improve outcomes for older patients by increasing the dose amount and/or frequency, a possibility to be evaluated in the next study.

EVs from MSC offer a safe and effective means of scaling MSC therapy to deliver higher, consistent doses without cell death from the cryorecovery process observed with MSC therapy. EVs are a natural product that play important cell-cell communication roles in daily physiology and play key roles in times of stress. The IMP is a bone marrow derived EV product that contains many different classes of biomolecules capable of modifying cell function such as chemokines, cytokines, growth factors and RNA species capable of immunomodulatory and regenerative activity. The EVs may act through multiple mechanisms of action to direct the regenerative and anti-inflammatory processes of the BM-MSC from which they are derived. The reduction of all four measured markers of systemic inflammation is consistent with a potential immunomodulatory effect of the IP (FIG. 4) and further study of this potential mechanism will be pursued. This secretome based therapeutic approach bypasses the challenges of direct allogeneic or autologous viable BM-MSC transplantation. EVs easily pass through capillaries, making them a safer option for intravenous administration in contrast to cellular therapies. The IMP is not subject to cell therapy associated issues of viability and unregulatable shifts in potency and efficacy profiles once delivered into the patient. As shown here, the IMP is extremely safe when administered intravenously. In addition, the trial herein is the first to show an EV product with potential survival benefit that, in phase 3, may prove superior to the clinical trial results from the aforementioned therapeutic candidates.

### Non-Limiting Numbered Embodiments

Embodiment 1. A composition comprising one or more proteins and/or one or more extracellular vesicles (EVs).

Embodiment 2. The composition of embodiment 1, wherein the total protein concentration of the one or more proteins is about 10 to about 40 μg per ml of the composition.

Embodiment 3. The composition of embodiment 1, wherein the total protein concentration of the one or more proteins is about 1.5 to about 6 μg per ml of the composition.

Embodiment 4. The composition of any one of embodiments 1-3, wherein the concentration of the one or more extracellular vesicles is about 10 billion to about 250 billion EVs per ml of the composition.

Embodiment 5. The composition of any one of embodiments 1-3, wherein the concentration of the one or more extracellular vesicles is at about 1 billion to about 40 billion EVs per ml of the composition.

Embodiment 6. The composition of any one of embodiments 1-5, wherein the one or more proteins comprise Ferritin, IGFBP-4 (Insulin-like growth factor binding protein-4), IL-1 R6 (Interleukin 1 Receptor 6), LAMP2 (Lysosome-associated membrane glycoprotein 2), bIG-H3 (Transforming growth factor-beta-induced protein igh3), GPR115 (Adhesion G protein-coupled receptor F4), CD63 antigen, CD109 antigen, Serpin F1 (Pigment epithelium-derived factor), IGFBP-6 (Insulin-like growth factor binding protein-6), HS3ST4 (Heparan sulfate glucosamine 3-O-sulfotransferase 4), OPN (Osteopontin), PAI-1 (Plasminogen activator inhibitor-1 or SERPINE 1), Cathepsin B, IGFBP-2 (Insulin-like growth factor binding protein-2), Semaphorin 6C, IGF-2 (Insulin-like growth factor-2), Sortilin, Serpin B6, Dkk-3 (Dickkopf-related protein 3), CNTF (Ciliary neurotrophic factor), TSP-1 (Thrombospondin 1), GM-CSF Ra (Granulocyte-macrophage colony-stimulating factor receptor subunit alpha), Thrombomodulin, Endoglycan, (podocalyxin-like protein 2) IGFBP-3 (Insulin-like binding protein-3), RGM-C (Hemojuvelin), PF4 (Platelet Factor 4), MIF (Macrophage migration inhibitory factor), TGM4 (Protein-glutamine gamma-glutamyltransferase 4), Periostin, Furin, TIMP-1 (Tissue inhibitor of MMPs 1), Decorin, PCK1 (Phosphoenolpyruvate carboxykinase, cytosolic) CD9 antigen, CD99 antigen, CA2 (Carbonic anhydrase 2), PRDX4 (Peroxidredoxin-4), Transferrin, DcR3 (Tumor necrosis factor receptor superfamily member 6B), GP73 (Golgi membrane protein 1), CD81 antigen, Lumican, or TIMP-2 (Tissue Inhibitor of MMPs 2), or a combination of two or more thereof.

Embodiment 7. The composition of any one of embodiments 1-6, wherein the one or more proteins comprise uPAR (CD87), VEGF (vascular endothelial growth factor), thrombomodulin (CD141, thrombin cofactor), CD97 (G protein-coupled receptor), IGFBP2 (insulin growth factor binding protein 2), TSLP (thymic stromal lymphoprotein), NCAM (neuronal cell adhesion molecule), NUP85 (nucleoporin 85), MIF (macrophage inhibitory factor), TNF-alpha RI (tumor necrosis factor-alpha receptor inhibitor), IL1-R6 (interleukin 1 receptor 6), PF4 (platelet factor 4), IGFBP-4 (insulin growth factor binding), bIG-H3 (TGFB induced protein), serpin F1 (secreted multifunctional protein), DKK3 (dickkopf-related protein 3), cathepsin B (catabolic protease), TIMP-1 (collagenase inhibitor), TIMP-2 (collagenase inhibitor), FAPA (fibroblast activation protein), semaphoring 6c (signal regulator of tissue formation), IGF2 (insulin-like growth factor 2), or FGF-16 (fibroblast growth factor 16), or a combination of two or more thereof.

Embodiment 8. The composition of any one of embodiments 1-7, wherein the one or more proteins comprise at lease one of the proteins of Table 1.

Embodiment 9. The composition of any one of embodiments 1-8, wherein the one or more proteins comprises TIMP1.

Embodiment 10. The composition of embodiment 9, wherein the TIMP1 is present at about 200 pg/mL to about 80 ng/mL of the composition or about 30 pg/mL to about 12 ng/mL.

Embodiment 11. The composition of any one of embodiments 1-10, wherein the one or more pro-teins comprises OPN.

Embodiment 12. The composition of embodiment 11, wherein the OPN is present at about 200 pg/mL to about 80 ng/mL of the composition or about 30 pg/mL to about 12 ng/mL.

Embodiment 13. The composition of any one of embodiments 1-8, wherein the one or more proteins comprises IGFBP4.

Embodiment 14. The composition of embodiment 9, wherein the IGFBP4 is present at about 200 pg/mL to about 80 ng/mL of the composition or about 30 pg/mL to about 12 ng/mL.

Embodiment 15. The composition of any one of embodiments 1-8, wherein the one or more proteins comprises osteonectin.

Embodiment 16. The composition of embodiment 15, wherein the osteonectin is present at about 200 pg/mL to about 80 ng/mL of the composition or about 30 pg/mL to about 12 ng/mL.

Embodiment 17. The composition of any one of embodiments 2, 3, 10, 12, 14, or 16, wherein the concentration of the one or more proteins is measured by ELISA.

Embodiment 18. The composition of embodiment 4 or embodiment 5, wherein the concentration of the one or more extracellular vesicles is measured by nanoparticle tracking analysis (NTA).

Embodiment 19. The composition of any one of embodiments 1-8, wherein the one or more extracellular vesicles are CD63+, CD9−, and CD81−.

Embodiment 20. The composition of embodiment 19, wherein at least about 85%, 86%, 87%, 88%, 89%, 90%, 91%, 92%, 93%, 94%, or 95% of the extracellular vesicles are CD63+, CD9−, and CD81−.

Embodiment 21. The composition of any one of embodiments 1-20, wherein the one or more extracellular vesicles have an average diameter of about 30 nm to about 170 nm.

Embodiment 22. The composition of embodiment 21, wherein the diameter is measured by nanoparticle tracking analysis (NTA).

Embodiment 23. The composition of embodiment 22, wherein the analysis comprises light scatter and fluorescence evaluation (e.g., NanoSight, Malvern Panalytical Ltd., United Kingdom).

Embodiment 24. The composition of any one of embodiments 1-23, wherein the EVs are characterized by single particle interferometric reflectance imaging sensor technology to visualize and quantify fluorescent antibody-labeled particles (e.g., NanoView Biosciences, Boston, MA)

Embodiment 25. The composition of any one of embodiments 1-24, wherein the one or more extracellular vesicles are obtained from a bone marrow MSC (BM-MSC) cell.

Embodiment 26. The composition of embodiment 25, wherein the BM-MSC is obtained from an iliac crest aspiration of a single donor.

Embodiment 27. The composition of embodiment 25 or embodiment 26, wherein the BM-MSC is capable of undergoing trilineage differentiation in vitro toward adipocyte, osteoblast, and chondrocyte phenotypes.

Embodiment 28. The composition of any one of embodiments 25-27, wherein the BM-MSCs are positive for CD73, CD105, CD166, and CD90.

Embodiment 29. The composition of any one of embodiments 25-28, wherein the BM-MSCs are negative for CD14, CD31, CD34, and CD45.

Embodiment 30. The composition of any one of embodiments 1-29, comprising one or more RNA molecules.

Embodiment 31. The composition of embodiment 30, wherein the one or more RNA molecules comprises hsa-miR-125b-5p, hsa-miR-145-5p, hsa-miR-191-5p, hsa-miR-199a-3p, hsa-miR-21-5p, hsa-miR-221-3p, hsa-miR-222-3p, hsa-miR-22-3p, hsa-miR-23a-3p, hsa-miR-23b-3p, hsa-miR-27a-3p, hsa-miR-27b-3p, hsa-miR-29a-3p, hsa-miR-29c-3p, hsa-miR-31-5p, hsa-miR-320a, hsa-miR-34a-5p, hsa-miR-423-3p, hsa-miR-424-5p, or hsa-miR-940, or a combination of two or more thereof.

Embodiment 32. The composition of embodiment 30 or embodiment 31, wherein the one or more RNA molecules are present within the one or more exosomes and/or attached to the one or more extracellular vesicles.

Embodiment 33. The composition of any one of embodiments 1-32, wherein the composition comprises saline (0.9% sodium chloride).

Embodiment 34. The composition of embodiment 33, wherein the saline is present in the composition at about 80% to about 95%, e.g., about 85% saline.

Embodiment 35. The composition of any one of embodiments 1-34, wherein the composition comprises sodium chloride, sodium lactate, potassium chloride, and calcium chloride.

Embodiment 36. The composition of any one of embodiments 1-35, wherein the molecular weight of the one or more proteins and the one or more extracellular vesicles is greater than about 10 kDa (kilodaltons).

Embodiment 37. The composition of any one of embodiments 1-36, wherein the composition comprises a saccharide, optionally a polysaccharide.

Embodiment 38. The composition of embodiment 37, wherein the saccharide is present in the composition at about 0.4 M or about 60 mM.

Embodiment 39. The composition of any one of embodiments 1-38, wherein the one or more proteins and/or the one or more extracellular vesicles have a size of less than about 0.2 microns.

Embodiment 40. The composition of any one of embodiments 1-39, wherein the composition is sterile by USP <71>.

Embodiment 41. The composition of any one of embodiments 1-40, wherein the composition is endotoxin USP <85> free.

Embodiment 42. The composition of any one of embodiments 1-41, wherein the composition is negative for mycoplasma DNA.

Embodiment 43. The composition of any one of embodiments 1-42, wherein the composition is cell-free.

Embodiment 44. The composition of any one of embodiments 1-43, wherein the composition is stored between −80° C. and −60° C.

Embodiment 45. The composition of embodiment 44, wherein the composition is administered within 6 hours of thaw when maintained at ambient temperature.

Embodiment 46. The composition of any one of embodiments 1-45, present in a glass vial.

Embodiment 47. The composition of any one of embodiments 1-46, formulated for intravenous administration.

Embodiment 48. The composition of any one of embodiments 1-47, wherein the composition has a pH of about 6 to about 7.5.

Embodiment 49. A method of treating ARDS in a subject in need thereof, the method comprising administering to the subject the composition of any one of embodiments 1-48.

Embodiment 50. The method of embodiment 49, wherein the subject has met the Berlin criteria for moderate to severe ARDS.

Embodiment 51. The method of embodiment 49 or embodiment 50, wherein the composition is administered intravenously.

Embodiment 52. The method of any one of embodiments 49-51, wherein the administering comprises infusion over 60 minutes on a first day.

Embodiment 53. The method of embodiment 52, wherein the administering further comprises infusion of the composition on a second day.

Embodiment 54. The method of embodiment 53, wherein the second day is one day after the first day (e.g., Day 1 and Day 2), two days after the first day (e.g., Day 1 and Day 3), or three days after the first day (e.g., Day 1 and Day 4).

Embodiment 55. The method of embodiment 53 or embodiment 54, wherein the infusion on the second day occurs if the subject does not have a SpO2 of at least about 93% between the administering and the infusion on the second day.

Embodiment 56. The method of any one of embodiments 53-55, wherein the administering comprises infusion of the composition on a third day.

Embodiment 57. The method of embodiment 56, wherein the third day is two days after the second day (e.g., Day 3 and Day 5).

Embodiment 58. The method of any one of embodiments 49-57, wherein within 28 days after the administering, the subject does not undergo ventilator treatment for at least about 1, 2, 3, 4, 5, 6, or 7 days.

Embodiment 59. The method of any one of embodiments 49-58, wherein within 28 days after the administering, the subject is not in the intensive care unit for at least about 1, 2, 3, 4, 5, 6, or 7 days.

Embodiment 60. The method of any one of embodiments 49-59, wherein within 28 days after the administering, the subject is not treated with oxygen for at least about 1, 2, 3, 4, 5, 6, or 7 days.

Embodiment 61. The method of any one of embodiments 49-60, wherein after the administering, the subject experiences improved tissue oxygenation.

Embodiment 62. The method of any one of embodiments 49-61, wherein after the administering, the subject experiences improved end-organ functioning.

Embodiment 63. The method of any one of embodiments 49-62, wherein after the administering, the subject has an improvement in partial pressure of arterial oxygen to fraction of inspired oxygen (PaO2/FiO2) ratio as compared to prior to the administering (e.g., as measured from arterial blood gas or imputed from SpO2 daily).

Embodiment 64. The method of any one of embodiments 49-63, wherein after the administering, the subject has an oxygenation saturation of at least about 93% on room air.

Embodiment 65. The method of any one of embodiments 49-64, wherein after the administering, a biomarker in the subject is lower than prior to the administering.

Embodiment 66. The method of embodiment 65, wherein the biomarker is measured from the blood of the subject.

Embodiment 67. The method of any one of embodiments 49-66, wherein after the administering the level of C-reactive protein (CRP) in the subject is less than prior to the administering.

Embodiment 68. The method of any one of embodiments 49-67, wherein after the administering the level of plasminogen activator inhibitor-1 (PAI-1) in the subject is less than prior to the administering.

Embodiment 69. The method of any one of embodiments 49-68, wherein after the administering the level of interleukin-8 (IL-8) in the subject is less than prior to the administering.

Embodiment 70. The method of any one of embodiments 49-69, wherein after the administering the level of interleukin-6 (IL-6) in the subject is less than prior to the administering.

Embodiment 71. The method of any one of embodiments 49-70, wherein after the administering the level of interleukin-1beta (IL-1b) in the subject is less than prior to the administering.

Embodiment 72. The method of any one of embodiments 49-71, wherein after the administering the level of sTNFrc in the subject is less than prior to the administering.

Embodiment 73. The method of any one of embodiments 49-72, wherein after the administering the level of D-dimer in the subject is less than prior to the administering.

Embodiment 74. The method of any one of embodiments 49-73, wherein after the administering the level of ferritin in the subject is less than prior to the administering.

Embodiment 75. The method of any one of embodiments 49-74, wherein after the administering the level of neutrophils in the subject is less than prior to the administering.

Embodiment 76. The method of any one of embodiments 49-75, wherein after the administering the Sequential Organ Failure Assessment (SOFA) Score change from prior to the administering decreases.

Embodiment 77. The method of any one of embodiments 61-76, wherein the after the administering is about 15 to about 29 days after the administering.

Embodiment 78. The method of any one of embodiments 49-77, wherein the administering occurs within 72 hours of the subject being diagnosed with ARDS.

Embodiment 79. The method of any one of embodiments 49-78, wherein the administering occurs within 48 hours of the subject being diagnosed with ARDS.

Embodiment 80. The method of any one of embodiments 49-79, wherein the ARDS is acute ARDS.

Embodiment 81. The method of embodiment 80, wherein the acute ARDS comprises dyspnea or worsening of hypoxemic respiratory failure following a predisposing risk factor.

Embodiment 82. The method of embodiment 81, wherein the predisposing risk factor is pneumonia, nonpulmonary infection, trauma, transfusion, aspiration or shock.

Embodiment 83. The method of any one of embodiments 49-82, wherein the administering occurs within 7 days of a clinical insult.

Embodiment 84. The method of any one of embodiments 49-83, wherein the administering occurs after chest imaging, wherein the chest imaging is indicative of a bilateral opacity.

Embodiment 85. The method of embodiment 84, wherein the bilateral opacity is not due to effusion, atelectasis, or nodule.

Embodiment 86. The method of any one of embodiments 49-85, wherein prior to the administering the subject has a PaO2/FiO2 (P/F ratio) of less than or equal to 200 mm Hg.

Embodiment 87. The method of any one of embodiments 49-86, wherein prior to the administering, the subject is treated with invasive or noninvasive mechanical ventilation.

Embodiment 88. The method of embodiment 87, wherein the mechanical ventilation has a minimum Positive End Expiratory Pressure (PEEP) of about 5 cm H2O.

Embodiment 89. The method of any one of embodiments 49-88, wherein prior to the administering, the subject is treated with continuous positive airway pressure (CPAP).

Embodiment 90. The method of embodiment 89, wherein the continuous positive airway pressure is performed at 5 cm H2O.

Embodiment 91. The method of any one of embodiments 49-90, wherein prior to the administering, the subject is treated with high flow nasal oxygen (HFNO) at a level at least about 30 L/min.

Embodiment 92. The method of any one of embodiments 49-91, wherein prior to the administering, the subject is in respiratory failure.

Embodiment 93. The method of embodiment 92, wherein the respiratory failure is not due to cardiac failure or fluid overload.

Embodiment 94. The method of any one of embodiments 49-93, wherein the subject is not infected with COVID-19.

Embodiment 95. The method of any one of embodiments 49-94, wherein the subject does not have Long COVID-19 (Post Acute Sequalae of COVID-19, PASC), e.g., does not have signs, symptoms, and conditions that continue or develop after acute COVID-19 infection.

Embodiment 96. The method of any one of embodiments 49-95, wherein the subject is not diagnosed with an infection within 1 week of the administering.

Embodiment 97. The method of any one of embodiments 49-96, wherein the subject is not suffering from an infection.

Embodiment 98. The method of any one of embodiments 49-97, wherein the subject has influenza.

Embodiment 99. The method of any one of embodiments 49-98, wherein the ARDS is not caused by bacterial pneumonia, viral pneumonia, fungal pneumonia, or parasitic pneumonia.

Embodiment 100. The method of any one of embodiments 49-99, wherein the ARDS subtype is hyperinflammatory ARDS.

Embodiment 101. The method of any one of embodiments 49-99, wherein the ARDS subtype is hypoinflammatory ARDS.

Embodiment 102. The method of any one of embodiments 49-101, wherein the ARDS is characterized by serum bicarbonate, sTNFR1, and IL-6.

Embodiment 103. The method of any one of embodiments 49-102, wherein the ARDS is caused by a pulmonary insult.

Embodiment 104. The method of embodiment 103, wherein the pulmonary insult is aspiration.

Embodiment 105. The method of embodiment 103, wherein the pulmonary insult is smoking.

Embodiment 106. The method of embodiment 103, wherein the pulmonary insult is non-protective ventilation.

Embodiment 107. The method of embodiment 103, wherein the pulmonary insult is lung contusion from trauma.

Embodiment 108. The method of embodiment 103, wherein the pulmonary insult is thoracic surgery.

Embodiment 109. The method of embodiment 103, wherein the pulmonary insult is drowning.

Embodiment 110. The method of embodiment 103, wherein the pulmonary insult is pulmonary vasculitis.

Embodiment 111. The method of embodiment 103, wherein the pulmonary insult is fat embolism.

Embodiment 112. The method of any one of embodiments 102-111, wherein the administering occurs within 7-14 days of the pulmonary insult.

Embodiment 113. The method of any one of embodiments 49-102, wherein the ARDS is caused by a nonpulmonary insult.

Embodiment 114. The method of embodiment 113, wherein the nonpulmonary insult is a blood transfusion.

Embodiment 115. The method of embodiment 113, wherein the nonpulmonary insult is trauma.

Embodiment 116. The method of embodiment 113, wherein the nonpulmonary insult is pancreatitis.

Embodiment 117. The method of embodiment 113, wherein the nonpulmonary insult is drug reaction.

Embodiment 118. The method of embodiment 113, wherein the nonpulmonary insult is a burn.

Embodiment 119. The method of embodiment 113, wherein the nonpulmonary insult is a cardio-pulmonary bypass.

Embodiment 120. The method of embodiment 113, wherein the nonpulmonary insult is noncardiogenic shock.

Embodiment 121. The method of any one of embodiments 112-120, wherein the administering occurs within 7-14 days of the nonpulmonary insult.

### Additional Non-Limiting Numbered Embodiments

Embodiment 1. A method of treating ARDS in a subject in need thereof, the method comprising administering to the subject a composition, wherein the ARDS is caused by a pulmonary insult or non-pulmonary insult, wherein the nonpulmonary insult is a blood transfusion, trauma, pancreatitis, drug reaction, burn, cardiopulmonary bypass, or noncardiogenic shock.

Embodiment 2. The method of embodiment 1, wherein the ARDS is caused by the pulmonary insult.

Embodiment 3. The method of embodiment 2, wherein the pulmonary insult is aspiration, smoking, ventilation, lung contusion from trauma, thoracic surgery, drowning, pulmonary vasculitis, or fat embolism.

Embodiment 4. The method of any one of embodiments 1-3, wherein the administering occurs within 7 days, or within 7-14 days of the pulmonary or nonpulmonary insult.

Embodiment 5. The method of any one of embodiments 1-4, wherein the subject is not infected with COVID-19.

Embodiment 6. The method of any one of embodiments 1-5, wherein the subject does not have Long COVID-19 (Post Acute Sequalae of COVID-19, PASC), optionally wherein the subject does not have signs, symptoms, and conditions that continue or develop after acute COVID-19 infection.

Embodiment 7. The method of any one of embodiments 1-6, wherein the subject is not diagnosed with an infection within 1 week of the administering.

Embodiment 8. The method of any one of embodiments 1-7, wherein the subject is not suffering from an infection.

Embodiment 9. The method of any one of embodiments 1-6, wherein the subject has influenza.

Embodiment 10. The method of any one of embodiments 1-9, wherein the ARDS is not caused by bacterial pneumonia, viral pneumonia, fungal pneumonia, or parasitic pneumonia.

Embodiment 11. The method of any one of embodiments 1-10, wherein the subject has met the Berlin criteria for moderate to severe ARDS.

Embodiment 12. The method of any one of embodiments 1-11, wherein the composition is administered intravenously.

Embodiment 13. The method of any one of embodiments 1-12, wherein the administering comprises infusion over 60 minutes on a first day.

Embodiment 14. The method of embodiment 13, wherein the administering further comprises infusion of the composition on a second day.

Embodiment 15. The method of embodiment 14, wherein the second day is one day after the first day, two days after the first day, or three days after the first day.

Embodiment 16. The method of embodiment 14 or embodiment 15, wherein the infusion on the second day occurs if the subject does not have a SpO2 of at least about 93% between the administering and the infusion on the second day.

Embodiment 17. The method of any one of embodiments 14-16, wherein the administering comprises infusion of the composition on a third day.

Embodiment 18. The method of embodiment 17, wherein the third day is two days after the second day.

Embodiment 19. The method of any one of embodiments 1-18, wherein within 28 days after the administering, the subject does not undergo ventilator treatment for at least about 1, 2, 3, 4, 5, 6, or 7 days.

Embodiment 20. The method of any one of embodiments 1-19, wherein within 28 days after the administering, the subject is not in the intensive care unit for at least about 1, 2, 3, 4, 5, 6, or 7 days.

Embodiment 21. The method of any one of embodiments 1-20, wherein within 28 days after the administering, the subject is not treated with oxygen for at least about 1, 2, 3, 4, 5, 6, or 7 days.

Embodiment 22. The method of any one of embodiments 1-21, wherein after the administering, the subject experiences improved tissue oxygenation.

Embodiment 23. The method of any one of embodiments 1-22, wherein after the administering, the subject experiences improved end-organ functioning.

Embodiment 24. The method of any one of embodiments 1-23, wherein after the administering, the subject has an improvement in partial pressure of arterial oxygen to fraction of inspired oxygen (PaO2/FiO2) ratio as compared to prior to the administering, optionally as measured from arterial blood gas or imputed from SpO2 daily.

Embodiment 25. The method of any one of embodiments 1-24, wherein after the administering, the subject has an oxygenation saturation of at least about 93% on room air.

Embodiment 26. The method of any one of embodiments 1-25, wherein after the administering, a biomarker in the subject is lower than prior to the administering.

Embodiment 27. The method of embodiment 26, wherein the biomarker is measured from the blood of the subject.

Embodiment 28. The method of any one of embodiments 1-27, wherein after the administering the level of C-reactive protein (CRP) in the subject is less than prior to the administering.

Embodiment 29. The method of any one of embodiments 1-28, wherein after the administering the level of plasminogen activator inhibitor-1 (PAI-1) in the subject is less than prior to the administering.

Embodiment 30. The method of any one of embodiments 1-29, wherein after the administering the level of interleukin-8 (IL-8) in the subject is less than prior to the administering.

Embodiment 31. The method of any one of embodiments 1-30, wherein after the administering the level of interleukin-6 (IL-6) in the subject is less than prior to the administering.

Embodiment 32. The method of any one of embodiments 1-31, wherein after the administering the level of interleukin-1beta (IL-1b) in the subject is less than prior to the administering.

Embodiment 33. The method of any one of embodiments 1-32, wherein after the administering the level of sTNFrc in the subject is less than prior to the administering.

Embodiment 34. The method of any one of embodiments 1-33, wherein after the administering the level of D-dimer in the subject is less than prior to the administering.

Embodiment 35. The method of any one of embodiments 1-34, wherein after the administering the level of ferritin in the subject is less than prior to the administering.

Embodiment 36. The method of any one of embodiments 1-35, wherein after the administering the level of neutrophils in the subject is less than prior to the administering.

Embodiment 37. The method of any one of embodiments 1-36, wherein after the administering the Sequential Organ Failure Assessment (SOFA) Score change from prior to the administering decreases.

Embodiment 38. The method of any one of embodiments 22-37, wherein the after the administering is about 15 to about 29 days after the administering.

Embodiment 39. The method of any one of embodiments 1-38, wherein the administering occurs within 72 hours of the subject being diagnosed with ARDS.

Embodiment 40. The method of any one of embodiments 1-39, wherein the administering occurs within 48 hours of the subject being diagnosed with ARDS.

Embodiment 41. The method of any one of embodiments 1-40, wherein the ARDS is acute ARDS.

Embodiment 42. The method of embodiment 41, wherein the acute ARDS comprises dyspnea or worsening of hypoxemic respiratory failure following a predisposing risk factor.

Embodiment 43. The method of embodiment 42, wherein the predisposing risk factor is pneumonia, nonpulmonary infection, trauma, transfusion, aspiration or shock.

Embodiment 44. The method of any one of embodiments 1-43, wherein the administering occurs after chest imaging, wherein the chest imaging is indicative of a bilateral opacity.

Embodiment 45. The method of embodiment 44, wherein the bilateral opacity is not due to effusion, atelectasis, or nodule.

Embodiment 46. The method of any one of embodiments 1-45, wherein prior to the administering the subject has a PaO2/FiO2 (P/F ratio) of less than or equal to 200 mm Hg.

Embodiment 47. The method of any one of embodiments 1-46, wherein prior to the administering, the subject is treated with invasive or noninvasive mechanical ventilation.

Embodiment 48. The method of embodiment 47, wherein the mechanical ventilation has a minimum Positive End Expiratory Pressure (PEEP) of about 5 cm H2O.

Embodiment 49. The method of any one of embodiments 1-48, wherein prior to the administering, the subject is treated with continuous positive airway pressure (CPAP).

Embodiment 50. The method of embodiment 49, wherein the continuous positive airway pressure is performed at 5 cm H2O.

Embodiment 51. The method of any one of embodiments 1-50, wherein prior to the administering, the subject is treated with high flow nasal oxygen (HFNO) at a level at least about 30 L/min.

Embodiment 52. The method of any one of embodiments 1-51, wherein prior to the administering, the subject is in respiratory failure.

Embodiment 53. The method of embodiment 52, wherein the respiratory failure is not due to cardiac failure or fluid overload.

Embodiment 54. The method of any one of embodiments 1-53, wherein the ARDS subtype is hyperinflammatory ARDS.

Embodiment 55. The method of any one of embodiments 1-53, wherein the ARDS subtype is hypoinflammatory ARDS.

Embodiment 56. The method of any one of embodiments 1-55, wherein the ARDS is characterized by serum bicarbonate, sTNFR1, and IL-6.

Embodiment 57. The method of any one of embodiments 1-56, wherein the composition comprises one or more proteins and/or one or more extracellular vesicles (EVs).

Embodiment 58. The method of embodiment 57, wherein the total protein concentration of the one or more proteins is about 10 to about 40 μg per ml of the composition.

Embodiment 59. The method of embodiment 57, wherein the total protein concentration of the one or more proteins is about 1.5 to about 6 μg per ml of the composition.

Embodiment 60. The method of any one of embodiments 57-59, wherein the concentration of the one or more extracellular vesicles is about 10 billion to about 250 billion EVs per ml of the composition.

Embodiment 61. The method of any one of embodiments 57-59, wherein the concentration of the one or more extracellular vesicles is at about 1 billion to about 40 billion EVs per ml of the composition.

Embodiment 62. The method of any one of embodiments 57-61, wherein the one or more proteins comprise Ferritin, IGFBP-4 (Insulin-like growth factor binding protein-4), IL-1 R6 (Interleukin 1 Receptor 6), LAMP2 (Lysosome-associated membrane glycoprotein 2), bIG-H3 (Transforming growth factor-beta-induced protein ig-h3), GPR115 (Adhesion G protein-coupled receptor F4), CD63 antigen, CD109 antigen, Serpin F1 (Pigment epithelium-derived factor), IGFBP-6 (Insulin-like growth factor binding protein-6), HS3ST4 (Heparan sulfate glucosamine 3-O-sulfotransferase 4), OPN (Osteopontin), PAI-1 (Plasminogen activator inhibitor-1 or SERPINE 1), Cathepsin B, IGFBP-2 (Insulin-like growth factor binding protein-2), Semaphorin 6C, IGF-2 (Insulin-like growth factor-2), Sortilin, Serpin B6, Dkk-3 (Dickkopf-related protein 3), CNTF (Ciliary neurotrophic factor), TSP-1 (Thrombospondin 1), GM-CSF Ra (Granulocyte-macrophage colony-stimulating factor receptor subunit alpha), Thrombomodulin, Endoglycan, (podocalyxin-like protein 2), IGFBP-3 (Insulin-like binding protein-3), RGM-C(Hemojuvelin), PF4 (Platelet Factor 4), MIF (Macrophage migration inhibitory factor), TGM4 (Protein-glutamine gamma-glutamyltransferase 4), Periostin, Furin, TIMP-1 (Tissue inhibitor of MMPs 1), Decorin, PCK1 (Phosphoenolpyruvate carboxykinase, cytosolic), CD9 antigen, CD99 antigen, CA2 (Carbonic anhydrase 2), PRDX4 (Peroxidredoxin-4), Transferrin, DcR3 (Tumor necrosis factor receptor superfamily member 6B), GP73 (Golgi membrane protein 1), CD81 antigen, Lumican, or TIMP-2 (Tissue Inhibitor of MMPs 2), or a combination of two or more thereof.

Embodiment 63. The method of any one of embodiments 57-61, wherein the one or more proteins comprise uPAR (CD87), VEGF (vascular endothelial growth factor), thrombomodulin (CD141, thrombin cofactor), CD97 (G protein-coupled receptor), IGFBP2 (insulin growth factor binding protein 2), TSLP (thymic stromal lymphoprotein), NCAM (neuronal cell adhesion molecule), NUP85 (nucleoporin 85), MIF (macrophage inhibitory factor), TNF-alpha RI (tumor necrosis factor-alpha receptor inhibitor), IL1-R6 (interleukin 1 receptor 6), PF4 (platelet factor 4), IGFBP-4 (insulin growth factor binding), bIG-H3 (TGFB induced protein), serpin F1 (secreted multifunctional protein), DKK3 (dickkopf-related protein 3), cathepsin B (catabolic protease), TIMP-1 (collagenase inhibitor), TIMP-2 (collagenase inhibitor), FAP-A (fibroblast activation protein), semaphoring 6c (signal regulator of tissue formation), IGF2 (insulin-like growth factor 2), or FGF-16 (fibroblast growth factor 16), or a combination of two or more thereof.

Embodiment 64. The method of any one of embodiments 57-61, wherein the one or more proteins comprise at least one of the proteins of Table 1.

Embodiment 65. The method of any one of embodiments 57-64, wherein the one or more proteins comprises TIMP1.

Embodiment 66. The method of embodiment 65, wherein the TIMP1 is present at about 200 pg/mL to about 80 ng/mL of the composition or about 30 pg/mL to about 12 ng/mL.

Embodiment 67. The method of any one of embodiments 57-66, wherein the one or more proteins comprises OPN.

Embodiment 68. The method of embodiment 67, wherein the OPN is present at about 200 pg/mL to about 80 ng/mL of the composition or about 30 pg/mL to about 12 ng/mL.

Embodiment 69. The method of any one of embodiments 57-68, wherein the one or more proteins comprises IGFBP4.

Embodiment 70. The method of embodiment 69, wherein the IGFBP4 is present at about 200 pg/mL to about 80 ng/mL of the composition or about 30 pg/mL to about 12 ng/mL.

Embodiment 71. The method of any one of embodiments 57-70, wherein the one or more proteins comprises osteonectin.

Embodiment 72. The method of embodiment 71, wherein the osteonectin is present at about 200 pg/mL to about 80 ng/mL of the composition or about 30 pg/mL to about 12 ng/mL.

Embodiment 73. The method of any one of embodiments 57-72, wherein the concentration of the one or more proteins is measured by ELISA.

Embodiment 74. The method of embodiment 57-73, wherein the concentration of the one or more extracellular vesicles is measured by nanoparticle tracking analysis (NTA).

Embodiment 75. The method of any one of embodiments 57-74, wherein the one or more extracellular vesicles comprise extracellular vesicles that are CD63+, CD9−, and CD81−.

Embodiment 76. The method of embodiment 75, wherein at least 50% of the extracellular vesicles are CD63+, and fewer than 50% of the extracellular vesicles are CD9+ or CD81+.

Embodiment 77. The method of any one of embodiments 57-76, wherein the one or more extracellular vesicles have an average diameter of about 30 nm to about 170 nm.

Embodiment 78. The method of embodiment 77, wherein the diameter is measured by nanoparticle tracking analysis (NTA).

Embodiment 79. The method of embodiment 78, wherein the analysis comprises light scatter and fluorescence evaluation, optionally via NanoSight.

Embodiment 80. The method of any one of embodiments 57-79, wherein the EVs are characterized by single particle interferometric reflectance imaging sensor technology to visualize and quantify fluorescent antibody-labeled particles.

Embodiment 81. The method of any one of embodiments 57-80, wherein the molecular weight of the one or more proteins and the one or more extracellular vesicles is greater than about 10 kDa (kilodaltons).

Embodiment 82. The method of any one of embodiments 57-81, wherein the one or more proteins and/or the one or more extracellular vesicles have a size of less than about 0.2 microns.

Embodiment 83. The method of any one of embodiments 1-82, wherein the one or more extracellular vesicles are obtained from a bone-marrow MSC (BM-MSC) cell.

Embodiment 84. The method of embodiment 83, wherein the BM-MSC is obtained from an iliac crest aspiration of a single donor.

Embodiment 85. The method of embodiment 83 or embodiment 84, wherein the BM-MSC is capable of undergoing trilineage differentiation in vitro toward adipocyte, osteoblast, and chondrocyte phenotypes.

Embodiment 86. The method of any one of embodiments 83-85, wherein the BM-MSCs are positive for CD73, CD105, CD166, and CD90.

Embodiment 87. The method of any one of embodiments 83-86, wherein the BM-MSCs are negative for CD14, CD31, CD34, and CD45.

Embodiment 88. The method of any one of embodiments 1-87, wherein the composition comprises one or more RNA molecules.

Embodiment 89. The method of embodiment 88, wherein the one or more RNA molecules comprises hsa-miR-125b-5p, hsa-miR-145-5p, hsa-miR-191-5p, hsa-miR-199a-3p, hsa-miR-21-5p, hsa-miR-221-3p, hsa-miR-222-3p, hsa-miR-22-3p, hsa-miR-23a-3p, hsa-miR-23b-3p, hsa-miR-27a-3p, hsa-miR-27b-3p, hsa-miR-29a-3p, hsa-miR-29c-3p, hsa-miR-31-5p, hsa-miR-320a, hsa-miR-34a-5p, hsa-miR-423-3p, hsa-miR-424-5p, or hsa-miR-940, or a combination of two or more thereof.

Embodiment 90. The method of embodiment 88 or embodiment 89, wherein the one or more RNA molecules are present within the one or more exosomes and/or attached to the one or more extracellular vesicles.

Embodiment 91. The method of any one of embodiments 1-90, wherein the composition comprises saline, optionally 0.9% sodium chloride.

Embodiment 92. The method of embodiment 91, wherein the saline is present in the composition at about 80% to about 95%, optionally about 85% saline.

Embodiment 93. The method of any one of embodiments 1-92, wherein the composition comprises sodium chloride, sodium lactate, potassium chloride, and calcium chloride.

Embodiment 94. The method of any one of embodiments 1-93, wherein the composition comprises a saccharide optionally a polysaccharide.

Embodiment 95. The method of embodiment 94, wherein the saccharide is present in the composition at about 0.4 M.

Embodiment 96. The method of embodiment 94, wherein the saccharide is present in the composition at about 60 mM.

Embodiment 97. The method of any one of embodiments 1-96, wherein the composition is sterile by USP <71>.

Embodiment 98. The method of any one of embodiments 1-97, wherein the composition is endotoxin USP <85> free.

Embodiment 99. The method of any one of embodiments 1-98, wherein the composition is negative for mycoplasma DNA.

Embodiment 100. The method of any one of embodiments 1-99, wherein the composition is cell-free.

Embodiment 101. The method of any one of embodiments 1-100, wherein the composition is stored between −80° C. and −60° C. prior to administration.

Embodiment 102. The method of embodiment 1-101, wherein the composition is administered within 6 hours of thaw when maintained at ambient temperature.

Embodiment 103. The method of any one of embodiments 1-101, wherein the composition has a pH of about 6 to about 7.5.

The examples and embodiments described herein are for illustrative purposes only and various modifications or changes suggested to persons skilled in the art are to be included within the spirit and purview of this application.

1-19. (canceled)

20. A composition comprising:
a secretome of a cultured bone marrow-derived mesenchymal stem cell (MSC),
wherein said secretome comprises:
(a) one or more MSC-derived growth factors; and
(b) MSC-derived extracellular vesicles;
wherein said one or more MSC-derived growth factors comprise neuregulin1-B1 (NRG1-B1) or urokinase type plasminogen activator receptor (uPAR), and
wherein said composition is a cell-free composition.

21. The composition of claim 20, wherein said one or more MSC-derived growth factors comprise NRG1-B1 and uPAR.

22. The composition of claim 20, wherein said one or more MSC-derived growth factors further comprise at least one of platelet derived growth factor receptor B (PDGFR-B), tissue inhibitor of metalloprotease 1 (TIMP-1), tissue inhibitor of metalloprotease 2 (TIMP-2), plasminogen activator inhibitor 1 (PAI-1), and tumor necrosis factor receptor (TNF RI).

23. The composition of claim 22, wherein said one or more MSC-derived growth factors comprise PDGFR-B, and wherein the PDGFR-B is present at a higher concentration than one or more of TIMP-1, TIMP-2, PAI-1, and TNF RI.

24. The composition of claim 23, wherein the PDGFR-B is present at a higher concentration than TIMP-1, TIMP-2, PAI-1, and TNF RI.

25. The composition of claim 20, wherein said composition comprises about 0.01 to about 20 wt. % of said secretome.

26. The composition of claim 25, wherein said composition comprises about 0.01 to about 10 wt. % of said secretome.

27. The composition of claim 20, wherein said composition is a conditioned media of said cultured bone marrow derived mesenchymal stem cell (MSC).

28. The composition of claim 20, further comprising a protective coating encapsulating said extracellular vesicles.

29. The composition of claim 28, wherein said protective coating comprises an oligosaccharide, a protein, a carbohydrate, a polyester, or a polymer.

30. The composition of claim 28, wherein said protective coating comprises a disaccharide.

31. The composition of claim 20, wherein said composition is formulated for subcutaneous, intramuscular, intravenous, or topical administration.

32. The composition of claim 20, in a frozen liquid form.

33. The composition of claim 20, in a powdered form.
